SEQUENCE LISTING (197 sequences)

SEQ ID NO: 1 - protein, 23 aa, synthetic construct
  SITE 1..23: note = Signal peptide
MRPTWAWWLFLVLLLALWAPARG

SEQ ID NO: 2 - protein, 115 aa, synthetic construct [sequence omitted]

SEQ ID NO: 3 - protein, 111 aa, synthetic construct [sequence omitted]

SEQ ID NO: 4 - protein, 5 aa, synthetic construct
  SITE 1..5: note = Bria VHCDR1
SYGMH

SEQ ID NO: 5 - protein, 17 aa, synthetic construct
  SITE 1..17: note = Bria VHCDR2
FIRYDGSNKYYADSVKG

SEQ ID NO: 6 - protein, 6 aa, synthetic construct
  SITE 1..6: note = Bria VHCDR3
HGSHDN

SEQ ID NO: 7 - protein, 13 aa, synthetic construct
  SITE 1..13: note = Bria VLCDR1
SGSRSNIGSNTVK

SEQ ID NO: 8 - protein, 7 aa, synthetic construct
  SITE 1..7: note = Bria VLCDR2
YNDQRPS

SEQ ID NO: 9 - protein, 12 aa, synthetic construct
  SITE 1..12: note = Bria VLCDR3
QSYDRYTHPALL

SEQ ID NO: 10 - protein, 306 aa, synthetic construct
  SITE 1..306: note = p40 (mature)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 11 - protein, 197 aa, synthetic construct
  SITE 1..197: note = p35 (mature)
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 12 - protein, 306 aa, synthetic construct
  SITE 1..306: note = Mutated p40
IWELKKDVYVVELDHYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 13 - protein, 306 aa, synthetic construct
  SITE 1..306: note = Mutated p40
IWELKKDVYVVELDKYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 14 - protein, 306 aa, synthetic construct
  SITE 1..306: note = Mutated p40
IWELKKDVYVVELDRYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 15 - protein, 306 aa, synthetic construct
  SITE 1..306: note = Mutated p40
IWELKKDVYVVELDWYPDAPGEMVVLTCDTPEEDGITWTLDQSSRVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 16 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVHEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 17 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVSEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 18 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKDFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 19 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKRFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 20 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKSFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 21 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEDGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 22 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEKGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 23 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKERGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 24 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHEKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 25 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHIKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 26 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHLKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 27 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHWKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 28 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHYKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 29 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHKKRDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 30 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHKKWDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 31 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTEILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 32 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTHILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 33 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTRILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 34 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGRNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 35 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHDLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 36 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLDYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 37 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLQYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 38 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLTYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 39 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLWYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 40 - protein, 306 aa, synthetic construct
  SITE 1..306: note = Mutated p40
IWELKKDVYVVELDRYPDAPGEMVVLTCDTPEEDGITWTLDQSSEVLGSGKTLTIQVKDDGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLWYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 41 - protein, 306 aa, synthetic construct
  SITE 1..306: note = Mutated p40
IWELKKDVYVVELDHYPDAPGEMVVLTCDTPEEDGITWTLDQSSEVLGSGKTLTIQVKEFGDAGQYTCHKGGEVLSHSLLLLHLKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 42 - protein, 306 aa, synthetic construct
  SITE 1..306: note = Mutated p40
IWELKKDVYVVELDHYPDAPGEMVVLTCDTPEEDGITWTLDQSSEVLGSGKTLTIQVKEFGDAGQYTCHKGGEVLSHSLLLLHLKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLQYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 43 - protein, 306 aa, synthetic construct
  SITE 1..306: note = Mutated p40
IWELKKDVYVVELDWYPDAPGEMVVLTCDTPEEDGITWTLDQSSRVLGSGKTLTIQVSSFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHDLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 44 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVHEFGDAGQYTCHKGGEVLSHSLLLLHKKRDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLDYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 45 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVSSFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHDLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 46 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKDFGDAGQYTCHKGGEVLSHSLLLLHWKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLWYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 47 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKDFGDAGQYTCHKGGEVLSHSLLLLHKKEDGIWSTHILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 48 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKRFGDAGQYTCHKGGEVLSHSLLLLHKKEDGIWSTRILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 49 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEKGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLWYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 50 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKERGDAGQYTCHKGGEVLSHSLLLLHEKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLWYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 51 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKERGDAGQYTCHKGGEVLSHSLLLLHYKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 52 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHIKRDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 53 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHIKRDGIWSTHILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 54 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHWKEDGIWSTDILKDQKEPKNKTFLRCEAKNYSGRFTCWWLTTISTDLTFSVKSSRGSSDPQGVTCGAATLSAERVRGRNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 55 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHWKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLWYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 56 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHKKRDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLDYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 57 - protein, 306 aa, synthetic construct
  SITE 1..306: note = Mutated p40
IWELKKDVYVVELDWYPDAPGEMVVLTCDTPEEDGITWTLDQSSEVLGSGKTLTIQVKEFGDAGQYTCHKGGEVLSHSLLLLHKKWDGIWSTEILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 58 - protein, 231 aa, synthetic construct
  SITE 1..231: note = Clone 12153; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 59 - protein, 231 aa, synthetic construct
  SITE 1..231: note = Clone 12155; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 60 - protein, 505 aa, synthetic construct
  SITE 1..505: note = Clone 26503; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGSGGGGSGGGSGGGSGGGGSGGGGSQVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSGGSGGGSGGGSGGGSGGGSGQSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVL

SEQ ID NO: 61 - protein, 754 aa, synthetic construct
  SITE 1..754: note = Clone 22279: Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 62 - protein, 754 aa, synthetic construct
  SITE 1..754: note = Clone 28046; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKRFGDAGQYTCHKGGEVLSHSLLLLHKKEDGIWSTRILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 63 - protein, 754 aa, synthetic construct
  SITE 1..754: note = Clone 28047; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKERGDAGQYTCHKGGEVLSHSLLLLHYKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 64 - protein, 754 aa, synthetic construct
  SITE 1..754: note = Clone 28048; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKEFGDAGQYTCHKGGEVLSHSLLLLHKKRDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLDYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 65 - protein, 754 aa, synthetic construct
  SITE 1..754: note = Clone 28049; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKEFGDAGQYTCHKGGEVLSHSLLLLHWKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLWYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 66 - protein, 754 aa, synthetic construct
  SITE 1..754: note = Clone 28050; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKEKGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLWYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 67 - protein, 754 aa, synthetic construct
  SITE 1..754: note = Clone 28051; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKEFGDAGQYTCHKGGEVLSHSLLLLHIKRDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 68 - protein, 754 aa, synthetic construct
  SITE 1..754: note = Clone 28053; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKEFGDAGQYTCHKGGEVLSHSLLLLHKKWDGIWSTEILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 69 - protein, 754 aa, synthetic construct
  SITE 1..754: note = Clone 28054; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKDFGDAGQYTCHKGGEVLSHSLLLLHKKEDGIWSTHILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 70 - protein, 754 aa, synthetic construct
  SITE 1..754: note = Clone 28055; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKEFGDAGQYTCHKGGEVLSHSLLLLHWKEDGIWSTDILKDQKEPKNKTFLRCEAKNYSGRFTCWWLTTISTDLTFSVKSSRGSSDPQGVTCGAATLSAERVRGRNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 71 - protein, 754 aa, synthetic construct
  SITE 1..754: note = Clone 28056; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKEFGDAGQYTCHKGGEVLSHSLLLLHEKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 72 - protein, 754 aa, synthetic construct
  SITE 1..754: note = Clone 28057; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKRFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 73 - protein, 754 aa, synthetic construct
  SITE 1..754: note = Clone 28058; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLTYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 74 - protein, 754 aa, synthetic construct
  SITE 1..754: note = Clone 28059; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGRNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 75 - protein, 754 aa, synthetic construct
  SITE 1..754: note = Clone 28060; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKSFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 76 - protein, 754 aa, synthetic construct
  SITE 1..754: note = Clone 28061; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKEFGDAGQYTCHKGGEVLSHSLLLLHWKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 77 - protein, 754 aa, synthetic construct
  SITE 1..754: note = Clone 28062; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKEFGDAGQYTCHKGGEVLSHSLLLLHKKWDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 78 - protein, 754 aa, synthetic construct
  SITE 1..754: note = Clone 28063; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLWYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 79 - protein, 754 aa, synthetic construct
  SITE 1..754: note = Clone 28064; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDRYPDAPGEMVVLTCDTPEEDGITWTLDQSSEVLGSGKTLTIQVKDDGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLWYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 80 - protein, 754 aa, synthetic construct
  SITE 1..754: note = Clone 28065; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKERGDAGQYTCHKGGEVLSHSLLLLHEKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLWYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 81 - protein, 754 aa, synthetic construct
  SITE 1..754: note = Clone 28066; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDHYPDAPGEMVVLTCDTPEEDGITWTLDQSSEVLGSGKTLTIQVKEFGDAGQYTCHKGGEVLSHSLLLLHLKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLQYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 82 - protein, 754 aa, synthetic construct
  SITE 1..754: note = Clone 28067; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKDFGDAGQYTCHKGGEVLSHSLLLLHWKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLWYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 83 - protein, 754 aa, synthetic construct
  SITE 1..754: note = Clone 28068; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVHEFGDAGQYTCHKGGEVLSHSLLLLHKKRDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLDYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 84 - protein, 754 aa, synthetic construct
  SITE 1..754: note = Clone 28069; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVKEFGDAGQYTCHKGGEVLSHSLLLLHIKRDGIWSTHILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 85 - protein, 754 aa, synthetic construct
  SITE 1..754: note = Clone 28070; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDHYPDAPGEMVVLTCDTPEEDGITWTLDQSSEVLGSGKTLTIQVKEFGDAGQYTCHKGGEVLSHSLLLLHLKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 86 - protein, 754 aa, synthetic construct
  SITE 1..754: note = Clone 28071; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDKYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 87 - protein, 754 aa, synthetic construct
  SITE 1..754: note = Clone 28072; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDHYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 88 - protein, 754 aa, synthetic construct
  SITE 1..754: note = Clone 28074; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSRVLGSGKTLTIQVSSFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHDLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 89 - protein, 754 aa, synthetic construct
  SITE 1..754: note = Clone 28075; Full Amino Acid Sequence
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGSADGGIWELKKDVYVVELDWYPDAPGEMVVLTCDTPEEDGITWTLDQSSEVLGSGKTLTIQVSSFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHDLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 90 - DNA, 693 bp, synthetic construct
  source 1..693: mol_type = other DNA
  misc_feature 1..693: note = Clone 12153; Full nucleotide sequence
gagccaaagagctccgacaagacccacacatgccccccttgtccggcgccagaggcagcaggaggaccaagcgtgttcctgtttccacccaagcccaaagacaccctgatgattagccgaacccctgaagtcacatgcgtggtcgtgtccgtgtctcacgaggacccagaagtcaagttcaactggtacgtggatggcgtcgaggtgcataatgccaagacaaaaccccgggaggaacagtacaacagcacctatagagtcgtgtccgtcctgacagtgctgcaccaggattggctgaacggcaaggaatataagtgcaaagtgtccaataaggccctgcccgctcctatcgagaaaaccatttctaaggcaaaaggccagcctcgcgaaccacaggtctacgtgctgcctccatcccgggacgagctgacaaagaaccaggtctctctgctgtgcctggtgaaaggcttctatccatcagatattgctgtggagtgggaaagcaatgggcagcccgagaacaattacctgacttggccccctgtgctggactctgatgggagtttctttctgtattctaagctgaccgtggataaaagtaggtggcagcagggaaatgtctttagttgttcagtgatgcatgaagccctgcataaccactacacccagaaaagcctgtccctgtcccccgga

SEQ ID NO: 91 - DNA, 693 bp, synthetic construct
  source 1..693: mol_type = other DNA
  misc_feature 1..115: note = Clone 12155; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 92 - DNA, 1515 bp, synthetic construct
  source 1..1515: mol_type = other DNA
  misc_feature 1..1515: note = Clone 26503; Full nucleotide sequence
gagccaaagagctccgacaagacccacacatgccccccttgtccggcgccagaggcagcaggaggaccaagcgtgttcctgtttccacccaagcccaaagacaccctgatgattagccgaacccctgaagtcacatgcgtggtcgtgtccgtgtctcacgaggacccagaagtcaagttcaactggtacgtggatggcgtcgaggtgcataatgccaagacaaaaccccgggaggaacagtacaacagcacctatagagtcgtgtccgtcctgacagtgctgcaccaggattggctgaacggcaaggaatataagtgcaaagtgtccaataaggccctgcccgctcctatcgagaaaaccatttctaaggcaaaaggccagcctcgcgagcctcaggtgtacgtgctgccaccttcccgcgacgagctgaccaagaatcaggtgtctctgctgtgcctggtgaagggcttctatccaagcgatatcgcagtggagtgggagtccaacggacagcccgagaacaattacctgacctggccacccgtgctggacagcgatggctccttctttctgtattccaagctgacagtggacaagtctagatggcagcagggcaacgtgttcagctgttccgtgatgcacgaggccctgcacaaccactacacccagaagtctctgagcttaagccctggaggaggaggaggctccggcggaggaggctccggcggcgggagcggcggcggcagcgggggcggcggctccggcggcggcggctctcaggtgcagctggtggagagcggaggaggagtggtgcagcccggcagaagcctgcggctgagctgcgcagcctctggcttcacctttagctcctacggcatgcactgggtgcggcaggcccctggcaagggactggagtgggtggccttcatcagatatgacggcagcaataagtactatgccgattccgtgaagggcaggtttaccatcagccgcgacaactccaagaatacactgtacctgcagatgaacagcctgagggccgaggacaccgccgtgtactattgtaagacacacggctcccacgataattggggccagggcaccatggtgacagtgtctagcggcggctctggcggcggcagcgggggtggctctggaggaggcagcggaggaggctccggccagtctgtgctgacccagccaccttctgtgagcggagcccccggacagagggtgacaatctcctgctctggcagccgctccaacatcggcagcaataccgtgaagtggtatcagcagctgccaggcacagcccccaagctgctgatctactataatgaccagcggccttccggcgtgccagatagattctctggcagcaagtccggcacctctgccagcctggccatcacaggcctgcaggcagaggacgaggcagattactattgtcagtcctacgatagatatacccaccccgccctgctgtttggcaccggcacaaaggtgacagtgctg

SEQ ID NO: 93 - DNA, 2262 bp, synthetic construct
  source 1..2262: mol_type = other DNA
  misc_feature 1..2262: note = Clone 22279; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 94 - DNA, 2262 bp, synthetic construct
  source 1..2262: mol_type = other DNA
  misc_feature 1..2262: note = Clone 28046; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaagagattcggcgacgccggacagtacacctgtcacaagggcggcgaggtgctgtcccactctctgctgctgctgcacaagaaggaggacggcatctggagcacaagaatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 95 - DNA, 2262 bp, synthetic construct
  source 1..2262: mol_type = other DNA
  misc_feature 1..2262: note = Clone 28047; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagagaggcgacgccggacagtacacctgtcacaagggcggcgaggtgctgtcccactctctgctgctgctgcactacaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 96 - DNA, 2262 bp, synthetic construct
  source 1..2262: mol_type = other DNA
  misc_feature 1..2262: note = Clone 28048; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaagagagacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctggactacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 97 - DNA, 2262 bp, synthetic construct
  source 1..2262: mol_type = other DNA
  misc_feature 1..2262: note = Clone 28049; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcactggaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgtggtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 98 - DNA, 2262 bp, synthetic construct
  source 1..2262: mol_type = other DNA
  misc_feature 1..2262: note = Clone 28050; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagaagggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgtggtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 99 - DNA, 2262 bp, synthetic construct
  source 1..2262: mol_type = other DNA
  misc_feature 1..2262: note = Clone 28051; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacatcaagagagacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 100 - DNA, 2262 bp, synthetic construct
  source 1..2262: mol_type = other DNA
  misc_feature 1..2262: note = Clone 28053; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaagtgggacggcatctggagcacagagatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 101 - DNA, 2262 bp, synthetic construct
  source 1..2262: mol_type = other DNA
  misc_feature 1..2262: note = Clone 28054; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggacttcggcgacgccggacagtacacctgtcacaagggcggcgaggtgctgtcccactctctgctgctgctgcacaagaaggaggacggcatctggagcacacacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 102 - DNA, 2262 bp, synthetic construct
  source 1..2262: mol_type = other DNA
  misc_feature 1..2262: note = Clone 28055; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcactggaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcagaaacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 103 - DNA, 2262 bp, synthetic construct
  source 1..2262: mol_type = other DNA
  misc_feature 1..2262: note = Clone 28056; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacgagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 104 - DNA, 2262 bp, synthetic construct
  source 1..2262: mol_type = other DNA
  misc_feature 1..2262: note = Clone 28057; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaagagattcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 105 - DNA, 2262 bp, synthetic construct
  source 1..2262: mol_type = other DNA
  misc_feature 1..2262: note = Clone 28058; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgacctacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 106 - DNA, 2262 bp, synthetic construct
  source 1..2262: mol_type = other DNA
  misc_feature 1..2262: note = Clone 28059; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcagaaacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 107 - DNA, 2262 bp, synthetic construct
  source 1..2262: mol_type = other DNA
  misc_feature 1..2262: note = Clone 28060; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaagagcttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 108 - DNA, 2262 bp, synthetic construct
  source 1..2262: mol_type = other DNA
  misc_feature 1..2262: note = Clone 28061; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcactggaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 109 - DNA, 2262 bp, synthetic construct
  source 1..2262: mol_type = other DNA
  misc_feature 1..2262: note = Clone 28062; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaagtgggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 110 - DNA, 2262 bp, synthetic construct
  source 1..2262: mol_type = other DNA
  misc_feature 1..2262: note = Clone 28063; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgtggtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 111 - DNA, 2262 bp, synthetic construct
  source 1..2262: mol_type = other DNA
  misc_feature 1..2262: note = Clone 28064; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggacagatatccagatgcaccaggagagatggtggtgctgacctgcgacacaccagaggaggatggcatcacctggacactggatcagagctccgaggtgctgggctccggcaagaccctgacaatccaggtgaaggacgacggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgtggtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 112 - DNA, 2262 bp, synthetic construct
  source 1..2262: mol_type = other DNA
  misc_feature 1..2262: note = Clone 28065; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagagaggcgacgccggacagtacacctgtcacaagggcggcgaggtgctgtcccactctctgctgctgctgcacgagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgtggtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 113 - DNA, 2262 bp, synthetic construct
  source 1..2262: mol_type = other DNA
  misc_feature 1..2262: note = Clone 28066; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggaccactatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacctgaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgcagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 114 - DNA, 2262 bp, synthetic construct
  source 1..2262: mol_type = other DNA
  misc_feature 1..2262: note = Clone 28067; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggacttcggcgacgccggacagtacacctgtcacaagggcggcgaggtgctgtcccactctctgctgctgctgcactggaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgtggtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 115 - DNA, 2262 bp, synthetic construct
  source 1..2262: mol_type = other DNA
  misc_feature 1..2262: note = Clone 28068; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgcacgagttcggcgacgccggacagtacacctgtcacaagggcggcgaggtgctgtcccactctctgctgctgctgcacaagaagagagacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctggactacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 116 - DNA, 2262 bp, synthetic construct
  source 1..2262: mol_type = other DNA
  misc_feature 1..2262: note = Clone 28069; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacatcaagagagacggcatctggagcacacacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 117 - DNA, 2262 bp, synthetic construct
  source 1..2262: mol_type = other DNA
  misc_feature 1..2262: note = Clone 28070; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggaccactatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacctgaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 118 - DNA, 2262 bp, synthetic construct
  source 1..2262: mol_type = other DNA
  misc_feature 1..2262: note = Clone 28071; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggacaagtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 119 - DNA, 2262 bp, synthetic construct
  source 1..2262: mol_type = other DNA
  misc_feature 1..2262: note = Clone 28072; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggaccactatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 120 - DNA, 2262 bp, synthetic construct
  source 1..2262: mol_type = other DNA
  misc_feature 1..2262: note = Clone 28074; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggaccagagctccagagtgctgggctccggcaagaccctgacaatccaggtgagcagcttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacgacctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 121 - DNA, 2262 bp, synthetic construct
  source 1..2262: mol_type = other DNA
  misc_feature 1..2262: note = Clone 28075; Full nucleotide sequence
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaacctcaggtgtacgtgtatccaccctcccgcgatgagctgacaaagaaccaggtgtctctgacctgtctggtgaagggcttttaccctagcgacatcgccgtggagtgggagtccaatggccagccagagaacaattataagaccacacctccagtgctggactctgatggcagcttcgccctggtgtctaagctgacagtggacaagagcagatggcagcagggcaacgtgttttcttgtagcgtgatgcacgaggccctgcacaatcactacacccagaagtccctgagcttaagccctggaggcagcgccgatggaggaatctgggagctgaagaaggacgtgtacgtggtggagctggactggtatccagatgcaccaggagagatggtggtgctgacctgcgacacaccagaggaggatggcatcacctggacactggaccagagctccgaggtgctgggctccggcaagaccctgacaatccaggtgagcagcttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacgacctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttctggaggaggaggcagcggcggaggaggctccggcggcggcggctctaatctgccagtggccacccctgacccaggcatgttcccctgcctgcaccactctcagaacctgctgcgggccgtgagcaatatgctgcagaaggccagacagacactggagttttacccatgtacctccgaggagatcgaccacgaggatatcacaaaggataagacctctacagtggaggcatgcctgccactggagctgaccaagaacgagagctgtctgaacagccgggagacatctttcatcaccaacggcagctgcctggcctccagaaagacatcttttatgatggccctgtgcctgtctagcatctacgaggacctgaagatgtatcaggtggagttcaagaccatgaacgccaagctgctgatggaccctaagaggcagatctttctggatcagaatatgctggccgtgatcgacgagctgatgcaggccctgaacttcaatagcgagacagtgccacagaagtcctctctggaggagcccgatttctacaagaccaagatcaagctgtgcatcctgctgcacgccttccggatcagagccgtgaccatcgaccgcgtgatgagctacctgaacgccagc

SEQ ID NO: 122 - protein, 22 aa, synthetic construct
  SITE 1..22: note = p40 precursor SP
MCHQQLVISWFSLVFLASPLVA

SEQ ID NO: 123 - protein, 328 aa, synthetic construct
  SITE 1..328: note = p40 precursor
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 124 - protein, 22 aa, synthetic construct
  SITE 1..22: note = p35 precursor SP
MCPARSLLLVATLVLLDHLSLA

SEQ ID NO: 125 - protein, 219 aa, synthetic construct
  SITE 1..219: note = p35 precursor
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 126 - protein, 19 aa, synthetic construct
  SITE 1..19: note = p19 precursor SP
MLGSRAVMLLLLLPWTAQG

SEQ ID NO: 127 - protein, 189 aa, synthetic construct
  SITE 1..189: note = p19 precursor
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 128 - protein, 170 aa, synthetic construct
  SITE 1..170: note = p19 mature
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 129 - DNA, 591 bp, synthetic construct
  source 1..591: mol_type = other DNA
  misc_feature 1..591: note = p35 nucleotide
agaaacctgcccgtggccacacccgatcctggcatgtttccctgcctgcaccacagccagaacctgctgcgggccgtgtccaatatgctgcagaaggccagacagaccctggagttctacccctgtacatctgaggagatcgaccacgaggatatcaccaaggacaagacctccacagtggaggcctgcctgcctctggagctgacaaagaacgagtcctgtctgaacagccgggagaccagcttcatcacaaatggctcctgcctggcctctcgcaagaccagctttatgatggccctgtgcctgagctccatctacgaggatctgaagatgtatcaggtggagttcaagacaatgaacgccaagctgctgatggaccctaagcggcagatctttctggatcagaatatgctggccgtgatcgacgagctgatgcaggccctgaactttaatagcgagaccgtgcctcagaagtctagcctggaggagccagatttctacaagacaaagatcaagctgtgcatcctgctgcacgccttccggatcagagccgtgaccatcgacagagtgatgtcctatctgaacgcctct

SEQ ID NO: 130 - DNA, 918 bp, synthetic construct
  source 1..918: mol_type = other DNA
  misc_feature 1..918: note = p40 nucleotide
atctgggagctgaagaaggacgtgtacgtggtggagctggactggtatccagatgcaccaggagagatggtggtgctgacctgcgacacaccagaggaggatggcatcacctggacactggaccagagctccgaggtgctgggcagcggcaagaccctgacaatccaggtgaaggagttcggcgatgccggacagtacacatgtcacaagggcggcgaggtgctgtctcacagcctgctgctgctgcacaagaaggaggatggcatctggtccacagacatcctgaaggatcagaaggagcccaagaacaagaccttcctgaggtgcgaggccaagaattattctggccgctttacctgttggtggctgaccacaatctctaccgacctgaccttcagcgtgaagtctagccggggctcctctgatcctcagggagtgacatgcggagccgccaccctgagcgccgagcgggtgagaggcgacaacaaggagtacgagtatagcgtggagtgccaggaggattccgcctgtcccgccgccgaggagtccctgcctatcgaagtgatggtggacgccgtgcacaagctgaagtacgagaattatacaagctccttctttatcagggacatcatcaagccagatccccctaagaacctgcagctgaagcccctgaagaacagcagacaggtggaggtgtcttgggagtaccctgatacctggtccacaccacacagctatttctccctgaccttttgcgtgcaggtgcagggcaagtctaagcgggagaagaaggacagagtgtttaccgataagacaagcgccaccgtgatctgtagaaagaacgcctccatctctgtgagggcccaggaccgctactattctagctcctggtctgagtgggccagcgtgccttgttcc

SEQ ID NO: 131 - DNA, 510 bp, synthetic construct
  source 1..510: mol_type = other DNA
  misc_feature 1..510: note = p19 nucleotide
agagccgtgcctggcggcagctccccagcctggacccagtgccagcagctgagccagaagctgtgcacactggcctggtccgcccacccactggtgggacacatggacctgcgggaggagggcgatgaggagaccacaaacgacgtgccacacatccagtgcggcgacggatgtgatcctcagggcctgcgggataattctcagttctgcctgcagagaatccaccagggcctgatcttctacgagaagctgctgggcagcgatatctttaccggagagccttctctgctgccagacagccctgtgggacagctgcacgcctccctgctgggcctgtctcagctgctgcagccagagggccaccactgggagacacagcagatcccctctctgagcccatcccagccatggcagaggctgctgctgcgctttaagatcctgaggtccctgcaggccttcgtggcagtggcagcacgcgtgtttgcccacggagccgccacactgtctccc

SEQ ID NO: 132 - protein, 6 aa, synthetic construct
  SITE 1..6: note = Linker
GSADGG

SEQ ID NO: 133 - protein, 15 aa, synthetic construct
  SITE 1..15: note = Linker
GGGGSGGGGSGGGGS

SEQ ID NO: 134 - protein, 28 aa, synthetic construct
  SITE 1..28: note = Linker
GGGGSGGGGSGGGSGGGSGGGGSGGGGS

SEQ ID NO: 135 - protein, 20 aa, synthetic construct
  SITE 1..20: note = Linker
GGSGGGSGGGSGGGSGGGSG

SEQ ID NO: 136 - protein, 217 aa, synthetic construct
  SITE 1..217: note = Human IgG1 Fc 231-447 (EU-numbering)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - DNA, 918 bp, synthetic construct
  source 1..918: mol_type = other DNA
  misc_feature 1..918: note = Mutated p40 nucleotide
atctgggagctgaagaaggacgtgtacgtggtggagctggaccactatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 138 - DNA, 918 bp, synthetic construct
  source 1..918: mol_type = other DNA
  misc_feature 1..918: note = Mutated p40 nucleotide
atctgggagctgaagaaggacgtgtacgtggtggagctggacaagtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 139 - DNA, 918 bp, synthetic construct
  source 1..918: mol_type = other DNA
  misc_feature 1..918: note = Mutated p40 nucleotide
atctgggagctgaagaaggacgtgtacgtggtggagctggacagatatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 140 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccagagtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 141 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgcacgagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 142 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgagcgagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 143 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggacttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 144 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaagagattcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 145 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaagagcttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 146 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggaggacggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 147 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagaagggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 148 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagagaggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 149 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacgagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 150 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacatcaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 151 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacctgaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 152 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcactggaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 153 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcactacaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 154 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaagagagacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 155 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaagtgggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 156 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagagatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 157 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacacacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 158 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacaagaatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 159 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcagaaacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 160 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacgacctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 161 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctggactacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 162 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgcagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 163 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgacctacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 164 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgtggtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 165 - DNA, 918 bp, synthetic construct
  source 1..918: mol_type = other DNA
  misc_feature 1..918: note = Mutated p40 nucleotide
atctgggagctgaagaaggacgtgtacgtggtggagctggacagatatccagatgcaccaggagagatggtggtgctgacctgcgacacaccagaggaggatggcatcacctggacactggatcagagctccgaggtgctgggctccggcaagaccctgacaatccaggtgaaggacgacggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgtggtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 166 - DNA, 918 bp, synthetic construct
  source 1..918: mol_type = other DNA
  misc_feature 1..918: note = Mutated p40 nucleotide
atctgggagctgaagaaggacgtgtacgtggtggagctggaccactatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacctgaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 167 - DNA, 918 bp, synthetic construct
  source 1..918: mol_type = other DNA
  misc_feature 1..918: note = Mutated p40 nucleotide
atctgggagctgaagaaggacgtgtacgtggtggagctggaccactatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacctgaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgcagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 168 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggaccagagctccagagtgctgggctccggcaagaccctgacaatccaggtgagcagcttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacgacctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 169 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgcacgagttcggcgacgccggacagtacacctgtcacaagggcggcgaggtgctgtcccactctctgctgctgctgcacaagaagagagacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctggactacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 170 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggaccagagctccgaggtgctgggctccggcaagaccctgacaatccaggtgagcagcttcggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacgacctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 171 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggacttcggcgacgccggacagtacacctgtcacaagggcggcgaggtgctgtcccactctctgctgctgctgcactggaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgtggtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 172 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggacttcggcgacgccggacagtacacctgtcacaagggcggcgaggtgctgtcccactctctgctgctgctgcacaagaaggaggacggcatctggagcacacacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 173 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaagagattcggcgacgccggacagtacacctgtcacaagggcggcgaggtgctgtcccactctctgctgctgctgcacaagaaggaggacggcatctggagcacaagaatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 174 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagaagggcgacgccggacagtacacctgtcacaagggcggcgaggtgctgtcccactctctgctgctgctgcacaagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgtggtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 175 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagagaggcgacgccggacagtacacctgtcacaagggcggcgaggtgctgtcccactctctgctgctgctgcacgagaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgtggtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 176 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagagaggcgacgccggacagtacacctgtcacaagggcggcgaggtgctgtcccactctctgctgctgctgcactacaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 177 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacatcaagagagacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 178 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacatcaagagagacggcatctggagcacacacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 179 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcactggaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcagaaacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 180 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcactggaaggaggacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgtggtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 181 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaagagagacggcatctggagcacagacatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctggactacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 182 - DNA, 918 bp, synthetic construct
  source 1..918: mol_type = other DNA
  misc_feature 1..918: note = Mutated p40 nucleotide
atctgggagctgaagaaggacgtgtacgtggtggagctggactggtatccagatgcaccaggagagatggtggtgctgacctgcgacacaccagaggaggatggcatcacctggacactggatcagagctccgaggtgctgggctccggcaagaccctgacaatccaggtgaaggagttcggcgacgccggacagtacacctgtcacaagggcggcgaggtgctgtcccactctctgctgctgctgcacaagaagtgggacggcatctggagcacagagatcctgaaggatcagaaggagcccaagaacaagaccttcctgaggtgcgaggccaagaattatagcggcagattcacctgttggtggctgaccacaatctccaccgatctgacattttctgtgaagtctagcaggggctcctctgaccctcagggagtgacatgcggagccgccaccctgagcgccgagcgggtgagaggcgataacaaggagtacgagtattccgtggagtgccaggaggactctgcctgtcccgccgccgaggagtccctgcctatcgaagtgatggtggatgccgtgcacaagctgaagtacgagaattatacaagctccttctttatcagggacatcatcaagcccgatccccctaagaacctgcagctgaagcctctgaagaatagccgccaggtggaggtgtcctgggagtaccctgacacctggagcacaccacactcctatttctctctgaccttttgcgtgcaggtgcagggcaagtctaagagggagaagaaggaccgcgtgttcaccgataagacaagcgccaccgtgatctgtcggaagaacgccagcatctccgtgcgggcccaggatagatactattctagctcctggagcgagtgggcctccgtgccatgttct

SEQ ID NO: 183 - protein, 110 aa, synthetic construct
  SITE 1..110: note = Human IgG1 CH2 domain
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 184 - protein, 107 aa, synthetic construct
  SITE 1..107: note = Human IgG1 CH3 domain
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 110 aa, synthetic construct
  SITE 1..110: note = Modified IgG1 CH2 domain
APEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAK

SEQ ID NO: 186 - protein, 106 aa, synthetic construct
  SITE 1..106: note = Modified IgG1 CH3 domain
GQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 187 - protein, 106 aa, synthetic construct
  SITE 1..106: note = Modified IgG1 CH3 domain
GQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 188 - protein, 7 aa, synthetic construct
GGGGGGS

SEQ ID NO: 189 - protein, 20 aa, synthetic construct
GGGGSGGGGSGGGGSGGGGS

SEQ ID NO: 190 - protein, 25 aa, synthetic construct
  SITE 1..20: note = This region may encompass 1-5 GGGS repeats
GGGSGGGSGGGSGGGSGGGSGGGGS

SEQ ID NO: 191 - protein, 29 aa, synthetic construct
  SITE 5..29: note = This region may encompass 1-5 GGGGS repeats
GGGSGGGGSGGGGSGGGGSGGGGSGGGGS

SEQ ID NO: 192 - protein, 45 aa, synthetic construct
  SITE 1..20: note = This region may encompass 1-5 GGGS repeats
  SITE 21..45: note = This region may encompass 1-5 GGGGS repeats
GGGSGGGSGGGSGGGSGGGSGGGGSGGGGSGGGGSGGGGSGGGGS

SEQ ID NO: 193 - protein, 25 aa, synthetic construct
  SITE 1..25: note = This sequence may encompass 1-5 GGGGS repeats
GGGGSGGGGSGGGGSGGGGSGGGGS

SEQ ID NO: 194 - protein, 422 aa, synthetic construct
  SITE 1..420: note = This region may encompass 1-20 GxS repeats, wherein X is 0-20
GGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGG

SEQ ID NO: 195 - protein, 420 aa, synthetic construct
  SITE 1..420: note = This sequence may encompass 1-20 S(G)x repeats, wherein X is 0-20
SGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGGSGGGGGGGGGGGGGGGGGGGG

SEQ ID NO: 196 - protein, 440 aa, synthetic construct
  SITE 1..440: note = This sequence may encompass 1-20 SE(G)x repeats, wherein X is 0-20
SEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGGSEGGGGGGGGGGGGGGGGGGGG

SEQ ID NO: 197 - protein, 20 aa, synthetic construct
  SITE 1..20: note = This sequence may encompass 1-4 GGGGS repeats
GGGGSGGGGSGGGGSGGGGS

(19) United States
(12) Patent Application Publication
(10) Pub. No.: US 2024/0228567 A1
(43) Pub. Date: Jul. 11, 2024

(54) MODIFIED INTERLEUKIN P40 SUBUNIT PROTEINS AND METHODS OF USE THEREOF

(71) Applicant: Zymeworks BC Inc., Vancouver (CA)

(72) Inventors: Ryan Blackler, Vancouver (CA); Thomas Spreter Von Kreudenstein, Vancouver (CA)

(21) Appl. No.: 18/620,572

(22) Filed: Mar. 28, 2024

Related U.S. Application Data
(63) Continuation of application No. PCT/CA2022/051445, filed on Sep. 29, 2022.
(60) Provisional application No. 63/249,685, filed on Sep. 29, 2021.

(51) Int. Cl. C07K 14/54; A61K 38/00

(52) U.S. Cl. CPC C07K 14/5434; A61K 38/00; C07K 2317/622; C07K 2319/30

(57) ABSTRACT

This disclosure generally relates to modified interleukin-12 (IL12) or IL23 p40 polypeptides with alterations to reduce binding affinity to its receptor subunit IL12Rβ1 in order to create an IL12 or IL23 protein that is less toxic as compared to the wild-type human IL12 or IL23 protein for the treatment of cancer.

### CROSS REFERENCE TO RELATED APPLICATIONS

This application is a continuation of International Application No. PCT/CA2022/051445, filed Sep. 29, 2022, which claims the benefit of U.S. Provisional Application No. 63/249,685, filed Sep. 29, 2021, which is hereby incorporated in its entirety by reference.

### SEQUENCE LISTING

The instant application contains a Sequence Listing which is hereby incorporated by reference in its entirety. Said XML copy, created on Oct. 31, 2022, is named ZWI-080WO_SL.xml, and is 400,050 bytes in size.

### TECHNICAL FIELD

This disclosure concerns modified Interleukin p40 subunit proteins, including fusion proteins, particularly IL12p40 and IL23p40, or any other cytokine with a p40 subunit protein, modified to reduce the binding activity of an interleukin cytokine; the compositions comprising the same and methods of using the compositions for the treatment of a variety of diseases including cancer.

### BACKGROUND

Interleukin 12 (IL12) was the first recognized member of a family of heterodimeric cytokines that includes IL12, IL23, IL27, IL35, and IL39. IL12 and IL23 are pro-inflammatory cytokines important for development of T helper 1 (Th-1) and T helper 17 (Th-17) T cell subsets respectively, while IL27 and IL35 are potent inhibitory cytokines. IL39 is an important cytokine in regulating innate and/or adaptive immune response. IL12 can directly enhance the activity of effector CD4 and CD8 T cells as well as natural killer (NK) and NK T cells.

IL12 is a heterodimeric molecule composed of an alpha chain (the p35 subunit) and a beta chain (the p40 subunit) covalently linked by a disulfide bridge to form the biologically active 70 kDa dimer. IL23 is a member of the IL12 cytokine family and is also composed of two subunits: the p40 subunit that it shares with IL12 and p19. IL-23 has the same chain IL-12p40 as IL-12. The p40 subunit is also secreted as a free monomer and disulfide-linked homodimer (p80), which have functions generally antagonistic to those of IL12 and IL-23. The IL12 receptor, or receptor complex, is composed of IL12Rβ1 and IL12Rβ2. The IL23 receptor complex (IL23R) consists of an IL23R subunit and an IL12Rβ1 subunit, which is a common subunit for the IL12 receptor and interacts with Tyrosine kinase 2 (Tyk2). The IL12Rβ1 subunit interacts with only the p40 subunit of IL12 or IL23, whereas the IL12Rβ2 subunit interacts with both the p35 and p40 subunits of IL12 and the IL23R subunit interacts with both the p19 and p40 subunits of IL23. The IL23R is mainly expressed on immune cells, in particular T cells (e.g., Thl7 and gamma delta T cells), macrophages, dendritic cells and NK cells (Duvallet et al., 2011). It has been recently shown that non-activated neutrophils express a basal amount of IL23R and that IL23R expression is increased upon cell activation (Chen et al., 2016).

Biologically, IL12 is an inflammatory cytokine that is produced in response to infection by a variety of cells of the immune system, including phagocytic cells, B cells and activated dendritic cells (Colombo and Trinchieri (2002), Cytokine and Growth Factor Reviews, 13: 155-168 and Hamza et al., “Interleukin-12 a Key Immunoregulatory Cytokine in Infection Applications” Int. J. Mol. Sci. 11; 789-806 (2010). IL12 plays an essential role in mediating the interaction of the innate and adaptive arms of the immune system, acting on T-cells and natural killer (NK) cells, enhancing the proliferation and activity of cytotoxic lymphocytes and the production of other inflammatory cytokines, especially interferon-gamma (IFN-gamma or IFNγ).

IL12 has been tested in human clinical trials as an immunotherapeutic agent for the treatment of a wide variety of cancers (Atkins et al. (1997), Clin. Cancer Res., 3: 409-17; Gollob et al. (2000), Clin. Cancer Res., 6: 1678-92; Hurteau et al. (2001), Gynecol. Oncol., 82: 7-10; and Youssoufian, et al. (2013) Surgical Oncology Clinics of North America, 22(4): 885-901), including renal, colon, and ovarian cancer, melanoma and T-cell lymphoma, and as an adjuvant for cancer vaccines (Lee et al. (2001), J. Clin. Oncol. 19: 3836-47). However, IL12 is toxic when administered systemically as a recombinant protein. Trinchieri, Adv. Immunol. 1998; 70:83-243. In order to maximize the anti-tumoral effect of IL12 while minimizing its systemic toxicity, IL12 gene therapy approaches have been proposed to allow production of the cytokine at the tumor site, thereby achieving high local levels of IL12 with low serum concentration. Qian et al., Cell Research (2006) 16: 182-188; US Patent Publication 20130195800.

Since IL12 is a heterodimeric molecule composed of an alpha chain (the p35 subunit) and a beta chain (the p40 subunit), the simultaneous expression of the two subunits is necessary for the production of the biologically active heterodimer. Recombinant IL12 expression has been achieved using bicistronic vectors containing the p40 and p35 subunits separated by an IRES (internal ribosome entry site) sequence to allow independent expression of both subunits from a single vector. However, use of IRES sequences can impair protein expression. Mizuguchi et al., Mol Ther (2000); 1: 376-382. Moreover, unequal expression of the p40 and p35 subunits can lead to the formation of homodimeric proteins (e.g., a p40-p40 dimer) which can have inhibitory effects on IL12 signaling. Gillessen et al. Eur. J. Immunol. 25(1):200-6 (1995).

As an alternative to bicistronic expression of the IL12 subunits, functional single chain IL12 fusion proteins have been produced by joining the p40 and p35 subunits with (Gly4Ser)3 or Gly6Ser linkers. Lieschke et al., (1997), Nature Biotechnology 15, 35-40; Lode et al., (1998), PNAS 95, 2475-2480. (These forms of p40-linker-p35 or p35-linker-p40 IL12 configurations may be referred to herein as “single chain IL12 (scIL12)”).

Human IL12 p70 (i.e., dimer of p35 and p40) has a reported in vivo half-life of 5-19 hours which, when administered as a therapeutic compound, can result in significant systemic toxicity. See e.g., Car et al. “The Toxicology of Interleukin-12: A Review” Toxicologic Path. 27:1, 58-63 (1999); Robertson et al. “Immunological Effects of Interleukin 12 Administered by Bolus Intravenous Injection to Patients with Cancer” Clin. Cancer Res. 5:9-16 (1999); Atkins et al. “Phase I Evaluation of Intravenous Recombinant Human Interleukin 12 in Patients with Advance Malignancies” Clin. Cancer Res. 3:409-417 (1997). Preclinical studies in murine tumor treatment models demonstrate powerful antitumor effects following the systemic administration of IL12. In humans, however, attempts to systemically administer recombinant IL12 resulted in significant toxicities including patient deaths and limited efficacy. Thus, there remains a need in the art for improved therapeutic control of in vivo delivered forms of IL12.

### SUMMARY

One aspect of the present disclosure provides cytokine fusion proteins containing modified p40 domains and, in particular, provides IL12 and IL23 fusion proteins, also referred to herein as IL12 HetFc fusion proteins. The IL12 fusion proteins described herein can comprise an IL12 polypeptide, an Fc domain comprising a first Fc polypeptide and a second Fc polypeptide; and optionally a masking moiety (MM) that reduces, inhibits or blocks IL12 activity.

One aspect of the present disclosure describes a modified p40 domain, the modified p40 domain comprising one or more amino acid substitutions relative to the wild-type human mature IL12 p40 domain sequence set forth in SEQ ID NO: 10, wherein the one or more amino acid substitutions are located at one or more positions of E45, D62 and D161, and wherein the numbering of the amino acid residues is based on the amino acid sequence set forth in SEQ ID NO: 10.

One aspect of the present disclosure describes a modified p40 domain, the modified p40 domain comprising one or more amino acid substitutions relative to the wild-type human mature IL12 p40 domain sequence set forth in SEQ ID NO: 10, wherein the one or more amino acid substitutions are W15H, W15K, W15R, D18G, E45K, K58H, K58W, E59D, E59G, E59R, F60D, F60E, F60K, F60R, F60V, D62H, D62I, D62N, K84E, K84I, K84L, K84V, K84W, K84Y, E86L, E86R, E865, E86W, D93E, D93H, D93R, D93W, D161R, D161S, K197D, K197E, K197Q, K197T, or K197W, or a combination thereof, and wherein the numbering of the amino acid residues is based on the amino acid sequence set forth in SEQ ID NO: 10.

Another aspect of the present disclosure describes an IL12 fusion protein comprising a modified p40 domain of the present disclosure.

Another aspect of the present disclosure describes an IL12 fusion protein comprising an IL12 polypeptide, wherein the IL12 polypeptide comprises a modified p40 domain of the present disclosure; coupled to a p35 domain.

Another aspect of the present disclosure describes a masked IL12 HetFc fusion protein comprising: (i) an IL-12 polypeptide comprising a modified p40 domain of the present disclosure coupled via the linker (G4S)₄ to a p35 domain comprising or consisting of the amino acid sequence set forth in SEQ ID NO: 11; (ii) a heterodimeric Fc domain comprising a first Fc polypeptide and a second Fc polypeptide; and (iii) a masking moiety (MM) comprising an anti-IL12 scFv domain, wherein: the IL12 polypeptide is coupled either directly or via a second linker to the C-terminus of the first Fc polypeptide, and the masking moiety is coupled either directly or via a third linker to the C-terminus of the second Fc polypeptide and is capable of non-covalently interacting with the IL12 polypeptide, thereby reducing the binding affinity of the IL12 polypeptide to at least one of its cognate receptors.

Another aspect of the present disclosure describes an unmasked IL12 HetFc fusion protein comprising: (i) an IL-12 polypeptide comprising the modified p40 domain according to the present disclosure coupled via the linker (G4S)₄ to the p35 domain comprising or consisting of the amino acid sequence set forth in SEQ ID NO: 11; and (ii) a heterodimeric Fc domain comprising a first Fc polypeptide and a second Fc polypeptide, wherein the IL12 polypeptide is coupled either directly or via a second linker to the C-terminus of the first Fc polypeptide.

Another aspect of the present disclosure describes a pharmaceutical composition comprising (i) a modified p40 domain of the present disclosure, (ii) a masked IL12 HetFc fusion protein of the present disclosure, and/or (iii) an unmasked IL12 HetFc fusion protein of the present disclosure, and a pharmaceutically acceptable carrier.

Another aspect of the present disclosure describes a nucleic acid molecule or a set of nucleic acid molecules encoding (i) a modified p40 domain of the present disclosure, (ii) a masked IL12 HetFc fusion protein of the present disclosure, and/or (iii) an unmasked IL12 HetFc fusion protein of the present disclosure.

Another aspect of the present disclosure describes a vector or a set of vectors comprising a nucleic acid molecule, or a set of nucleic acid molecules described herein that encode (i) a modified p40 domain of the present disclosure, (ii) a masked IL12 HetFc fusion protein of the present disclosure, and/or (iii) an unmasked IL12 HetFc fusion protein of the present disclosure.

Another aspect of the present disclosure describes a method of identifying one or more amino acid substitutions in a p40 domain amino acid sequence to produce a modified p40 domain, the method comprising performing molecular dynamics and mutagenesis simulations, thereby identifying the one or more amino acid substitutions listed in Table C, wherein the one or more amino acid substitutions in the modified p40 domain amino acid sequence are relative to the sequence set forth in SEQ ID NO: 1, and wherein the one or more amino acid substitutions reduce the binding affinity (K_{D}) of the modified p40 domain to at least one of its cognate receptors, and relative to an unmodified p40 domain that does not comprise the one or more amino acid substitutions.

The disclosure provides modifications of human IL12 and IL23 p40 subunit domains D1 and D2, specifically the modifications include engineered amino acid alterations in the D1 and D2 domain aimed to reduce binding affinity to the receptor. More specifically, the alterations/modifications occur at one or more positions corresponding amino acid residue positions 15, 18, 45, 58, 59, 60, 62, 84, 86, 93, 161, 195 and 197 of SEQ ID NO: 1. One aspect of the present disclosure provides p40 domains that are modified to have reduced binding to the IL12Rβ1 subunit, wherein the modified p40 domains contain at least one amino acid substitution.

In some embodiments the modified p40 domains are expressed as components of IL12 fusion proteins, comprising a p35 subunit and a modified p40 subunit, wherein the IL12 activity of the IL12 fusion protein containing a modified p40 domain is attenuated as compared to the IL12 activity of a corresponding IL12 fusion protein containing a non-modified p40 domain. Again, accordingly, the embodiments provide modifications of a human IL12 or IL23 p40 D1 and D2 domain. In some embodiments of the IL12 fusion proteins herein, the IL12 polypeptide is a single chain IL12 polypeptide having the orientation p40-linker-p35. In some embodiments of the IL12 fusion proteins herein, the single chain IL12 polypeptide is fused to an Fc domain comprising a first Fc polypeptide and a second Fc polypeptide, wherein the IL12 polypeptide is fused to the first Fc polypeptide by a first linker.

In some embodiments the modified p40 domains are expressed as components of masked IL12 fusion proteins, comprising: a) an Fc domain comprising a first Fc polypeptide and a second Fc polypeptide; b) a masking moiety (MM); and c) an IL12 polypeptide; wherein the masking moiety is fused to the first Fc polypeptide by a first linker; wherein the IL12 polypeptide is fused to the second Fc polypeptide by a second linker; and wherein the IL12 activity of the masked IL12 fusion protein containing a modified p40 domain is attenuated as compared to the IL12 activity of a corresponding IL12 fusion protein containing a non-modified p40 domain.

In some embodiments of the masked IL12 fusion proteins herein, the masking moiety is a single-chain Fv (scFv) antibody fragment. In certain embodiments, the scFv comprises the VHCDRI-3 having the amino acid sequences set forth in SEQ ID NOS:4-6, respectively and the VLCDRI-3 having the amino acid sequence set forth in SEQ ID NOS: 7-9, respectively. In some embodiments, the scFv comprises a VH and VL comprising the amino acid sequence set forth in SEQ ID NOs:2 and 3, respectively.

In some embodiments of the IL12 fusion proteins herein, the fusion protein further comprises a targeting domain. In some embodiments, the targeting domain specifically binds a tumor-associated antigen.

In some embodiments of the IL12 fusion proteins herein, the first Fc polypeptide comprises a first CH3 domain and the second Fc polypeptide comprises a second CH3 domain.

In some embodiments of the IL12 fusion proteins herein, the IL12 activity is determined by measuring relative cell abundance or cytokine production of a cell or a cell line that is sensitive to IL12. In some embodiments, the cell or cell line is selected from PBMC, CD8+ T cells, a CTLL-2 cell line and an NK cell line. In some embodiments, the IL12 activity is determined by measuring IFNγ release by CD8+ T cells. In some embodiments, the IL12 activity is determined by measuring the relative cell abundance of NK cells.

In some embodiments of the IL12 fusion proteins described herein, the first CH3 domain or the second CH3 domain or both comprise an asymmetric amino acid modification wherein the first and second CH3 domain preferentially pair to form a heterodimer rather than a homodimer.

One aspect of the present disclosure provides a composition comprising any of the IL12 or IL23 fusion proteins described herein and a pharmaceutically acceptable excipient.

One aspect of the present disclosure provides a method of treating cancer in a subject comprising administering to the subject a therapeutically effective amount of a composition comprising any of the IL12 fusion proteins described herein (e.g., IL12 HetFc fusion proteins) and a pharmaceutically acceptable excipient.

One aspect of the present disclosure provides an isolated nucleic acid encoding an IL12 fusion protein as described herein.

One aspect of the present disclosure provides an expression vector comprising an isolated nucleic acid encoding an IL12 fusion protein as described herein.

One aspect of the present disclosure provides an isolated host cell comprising an isolated nucleic acid encoding an IL12 fusion protein as described herein or an expression vector comprising such an isolated nucleic acid.

One aspect of the present disclosure provides a method of making an IL12 fusion protein comprising culturing a host cell comprising an isolated nucleic acid encoding an IL12 fusion protein as described herein or an expression vector comprising such an isolated nucleic acid, under conditions suitable for expression of the IL12 fusion protein and optionally, recovering the IL12 fusion protein from the host cell culture medium.

One aspect of the present disclosure provides an interleukin 23 (IL23) fusion protein, comprising a p19 domain and a modified p40 domain as described herein, wherein the IL23 activity of the IL23 fusion protein containing a modified p40 domain is attenuated as compared to the IL23 activity of a corresponding IL23 fusion protein containing a non-modified p40 domain.

### BRIEF DESCRIPTION OF THE DRAWINGS

The implementations disclosed herein are illustrated by way of example, and not by way of limitation, in the accompanying drawings. The description and drawings are only for the purpose of illustration and as an aid to understanding and are not intended as a definition of the limits of the modified p40 domains, fusion proteins, compositions and methods of the present disclosure.

FIGS. 1A-1D show schematic diagrams of p40 containing cytokines, e.g., schematic diagrams of a monomeric p40 domain (FIG. 1A), a dimeric p40 domain (p80, FIG. 1B), as well as cytokines containing such p40 domains such as IL12 (FIG. 1C) and IL23 (FIG. 1D), according to embodiments of the present disclosure.

FIGS. 2A-2B show schematic diagrams of IL12 HetFc and masked IL12 HetFc fusion protein variants according to embodiments of the present disclosure.

FIGS. 3A-3B show representative CE-SDS and UPLC-SEC profiles after Protein A and Prep-SEC purification of an IL12 HetFc fusion protein (v30806, FIG. 3A) and a masked IL12 HetFc fusion protein (v35436, FIG. 3B).

FIGS. 4A-4J show data obtained from IL12 Reporter Gene Assay (RGA) experiments using IL12 HetFc and masked IL12 HetFc fusion protein variants. The RGA experiments for each set of variants were run twice (indicated by “repeat 1” and “repeat 2” labels). Each graph shows a set of unmasked IL12 HetFc fusion proteins including the control variant 30806, and a set of masked IL12 HetFc fusion proteins including the control variant 35436. In addition, FIG. 4A shows RGA responses for variants 37172, 37174, 37175, 37485, 37487 and 37488, FIG. 4B shows RGA responses for variants 37176, 37178, 37173, 37489, 37491 and 37486, FIG. 4C shows RGA responses for variants 37157, 37158, 37156, 37470, 37471 and 37469, FIG. 4D shows RGA responses for variants 37177, 37154, 37159, 37490, 37467 and 37472, FIG. 4E shows RGA responses for variants 37179, 37163, 37492 and 37476, FIG. 4F shows RGA responses for variants 37181, 37162, 37155, 37494, 37475 and 37468, FIG. 4G shows RGA responses for variants 37161, 37182, 37164, 37474, 37495 and 37477, FIG. 4H shows RGA responses for variants 37166, 37165, 37180, 37479, 37478 and 37493, FIG. 4I shows RGA responses for variants 37169, 37171, 37168, 37482, 37484 and 37481, and FIG. 4J shows RGA responses for variants 37167, 37170, 37480 and 37483.

### DETAILED DESCRIPTION

The present disclosure relates to p40 domains that are modified to have reduced binding to the cytokine receptor IL12Rβ1 subunit. In particular, the present disclosure relates to IL12 family member cytokines containing modified p40 domains and more specifically, to IL12 and IL23 fusion proteins containing modified p40 domains. The present disclosure further provides compositions and kits comprising the cytokines containing modified p40 domains described herein and methods of using the compositions for the treatment of a variety of diseases.

IL12 is an immunostimulatory cytokine capable of driving anti-tumor responses by the innate and adaptive immune cells. The use of IL12 as a therapeutic has been extensively studied in pre-clinical models of cancer including mouse models of melanoma, renal cell carcinoma, mammary carcinoma, and colon carcinoma. The anti-tumor activity of IL12 administrations has been shown even when IL12 was administered at later stages with large, established tumors in mice. The potent anti-tumor effects of IL12 in preclinical models led to clinical trials of recombinant IL12. Unfortunately, toxicities including treatment related deaths of two patients resulted in halting of clinical trials for recombinant IL12. It is also noteworthy that recombinant cytokines have poor PK due to their small size. The present disclosure provides IL12 fusion proteins that circumvent the toxicities by reducing the cytokine activity with the use of modified p40 domains that have reduced affinity the cytokine receptor IL12Rβ1 subunit and result in reduced IL12 binding and/or activity. The reduced cytokine activity of IL12 fusion proteins containing modified p40 domains may allow for increased dosing, exposure, and clinical efficacy, as compared to IL12 proteins with non-modified p40 domains where dosing, exposure, and efficacy are limited by toxicity. The present disclosure also provides for improved pharmacokinetics of IL12 by fusion to an Fc domain.

### Definitions

Unless defined otherwise, all technical and scientific terms used herein have the same meaning as commonly understood by one of ordinary skill in the art.

As used herein, the term “about” refers to an approximately ±10% variation from a given value, unless otherwise indicated. In some embodiments, the term “about” refers to a ±10% variation from a given value. In some embodiments, the term “about” refers to a ±8% variation from a given value. In some embodiments, the term “about” refers to a ±6% variation from a given value. In some embodiments, the term “about” refers to a ±4% variation from a given value. In some embodiments, the term “about” refers to a ±2% variation from a given value. It is to be understood that such a variation is always included in any given value provided herein, whether or not it is specifically referred to.

The use of the word “a” or “an” when used herein in conjunction with the term “comprising” may mean “one,” but it is also consistent in certain embodiments with the meaning of “one or more,” “at least one” or “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

By “fused” is meant that the components (e.g., a cytokine molecule and an Fc domain polypeptide or a masking moiety and an Fc domain polypeptide) are linked by peptide bonds, either directly or via one or more peptide linkers.

As used herein, the term “single-chain” refers to a molecule comprising amino acid monomers linearly linked by peptide bonds. In certain embodiments, one of the cytokine protein or domains is a single-chain cytokine molecule, i.e., an IL12 molecule wherein the p35 and the p40 domains are connected by a peptide linker to form a single peptide chain; or an IL23 molecule wherein the p19 and the p40 domains are connected by a peptide linker to form a single peptide chain.

As used herein, the terms “IL12 polypeptide,” “single-chain (sc)IL12” and “IL12 p70” can be used interchangeably and generally refer to human IL12 that comprises a consecutive, single-chain amino acid sequence encompassing an IL12p40 domain, such as a modified p40 domain as described herein, and an IL12p35 domain.

As used herein, the term “affinity” or “binding affinity” or “binding activity” refers to the strength of the binding interaction between a single biomolecule to its ligand/binding partner (e.g., p40 domain to IL12Rβ1). Binding can be characterized by a binding constant, or equilibrium association constant (KA) or its inverse the equilibrium dissociation constant (KD). For any given equilibrium binding interaction between the p40 domain and IL12Rβ1, the higher the KA (or the lower the KD) value the more complex and less free p40 and IL12Rβ1 there will be at equilibrium. In general, when referring to KA or KD, “tighter” binding means a bigger KA or a smaller KD value. Proteins such as cytokines can bind to their receptors much more tightly, with what are known as “tight” or “very tight” kₐ values of 10¹⁰ —10¹².

The strength, or affinity, of specific binding can be expressed in terms of the equilibrium dissociation constant (KD) of the interaction, wherein a smaller KD represents greater affinity and a larger KD represents lower affinity. Binding properties can be determined by methods well known in the art such as bio-layer interferometry and surface plasmon resonance-based methods, including Biacore and Octet methodologies. Thus, both the association rate constant (ka) and the dissociation rate constant (kd) can be determined, and the ratio of kd/ka is equal to the equilibrium dissociation constant KD (See Nature 361:186-187 (1993) and Davies et al. (1990) Annual Rev Biochem 59:439-473), both of which are incorporated by reference in their entirety for the methods therein.

As used herein, the term “modification” or “mutation” herein is meant an amino acid substitution, insertion, and/or deletion in a polypeptide sequence or an alteration to a moiety chemically linked to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA. A “modified p40 domain” or a “mutated p40 domain” therefore is meant a p40 polypeptide with an amino acid substitution, insertion, and/or deletion.

As used herein, the term “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For clarity, a protein which has been engineered to change the nucleic acid coding sequence but not to change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

It is contemplated that any embodiment discussed herein can be implemented with respect to any method, use or composition disclosed herein.

Particular features, structures and/or characteristics described in connection with an embodiment disclosed herein may be combined with features, structures and/or characteristics described in connection with another embodiment disclosed herein in any suitable manner to provide one or more further embodiments.

It is also to be understood that the positive recitation of a feature in one embodiment, serves as a basis for excluding the feature in an alternative embodiment. For example, where a list of options is presented for a given embodiment or claim, it is to be understood that one or more option may be deleted from the list and the shortened list may form an alternative embodiment, whether or not such an alternative embodiment is specifically referred to.

### Modified p40 Domains

The present disclosure describes modified p40 domains. Such modified p40 domains can be modified in their amino acid sequence, e.g., relative to corresponding wildtype (WT) p40 sequences. As described herein, a modified p40 domain of the present disclosure can have a reduced binding affinity for another protein when compared to an unmodified p40 polypeptide. In some embodiments, such other protein to which a modified p40 domain binds with reduced affinity is one of the cognate IL12 receptors, e.g., the receptor IL12Rβ1. A modified p40 protein of this disclosure may also have a stability (e.g., thermostability measured as a melting temperature (Tm) and/or chemical stability in the presence of certain reagents) that is about the same (e.g., ±5% variation in Tm compared to WT p40 domain) or higher (e.g., >5% increase in Tm) than that of an unmodified (e.g., WT) p40 domain.

The present disclosure provides p40 domains that are modified to have reduced binding to the IL12Rβ1 subunit, wherein the modified p40 domains contain at least one amino acid substitution or group of substitutions from the following list (numbered according to mature p40 sequence; see SEQ ID NO:10 and Table A): W15H, W15K, W15R, D18G, E45K, E45R, K58H, K58S, K58W, E59D, E59G, E59R, E59S, F60D, F60E, F60K, F60R, F60V, D62H, D62I, D62N, K84E, K84I, K84L, K84V, K84W, K84Y, E86L, E86R, E865, E86W, D93E, D93H, D93R, D93W, D161R, D161S, K195D, K197D, K197E, K197Q, K197T, K197W, W15H_K84L, K58H_K84I, E59D_K84W, E59G_K84W, E59R_K84E, E59R_K84W, E59R_E86W, E59D_D93H, E59R_D93R, E59R_K197E, E59R_K197W, F60E_K84W, F60R_K84Y, F60K_K197W, F60R_K197W, K84I_E86R, K84E_D93H, K84I_D93H, K84V_D93H, K84W_D93W, K84I_D161R, K84W_D161R, K84W_K197E, K84W_K197Q, K84W_K197W, E86W_D93E, E86R_K197D, E86W_K197W, W15H_K84L_K197Q, K58S_E59S_K195D, K58H_E86R_K197D, E59D_K84W_K197W, F60R_K84E_K197W, K84I_E86R_D93H, W15R_E59D_F60D_K197W, E45R_K58S_E59S_K195D.

As described herein, combinations of amino acid modifications at multiple positions within a single chain are identified using “_” between each position modified. For example, “15_84” indicates that both positions 124 and 186 are modified in the p40 polypeptide chain referred to. Likewise, “15_84_197” indicates that all of positions 124, 133, and 180 are modified in the p40 polypeptide chain referred to.

The present disclosure provides p40 domains that are modified to have reduced binding to the IL12Rβ1 subunit, wherein the modified p40 domains contain at least one amino acid substitution or group of substitutions from the following list (numbered according to mature p40 sequence; see SEQ ID NO:10 and Table A); W15H, W15K, W15R, E45R, K58H, K58S, E59D, E59R, E59S, F60D, F60K, F60R, K84E, K84I, K84L, K84W, K84Y, E86R, E86W, D93E, D93H, D93R, D161R, K195D, K197D, K197Q, K197T, K197W, W15H_K84L, E59D_D93H, E59R_D93R, F60K_K197W, F60R_K84Y, K84I_E86R, K84W_D161R, K84W_K197W, E86R_K197D, E86W_D93E, W15H_K84L_K197Q, K58H_E86R_K197D, K58S_E59S_K195D, E59D_K84W_K197W, F60R_K84E_K197W, K84I_E86R_D93H, W15R_E59D_F60D_K197W, E45R_K58S_E59S_K195D; as captured in the sequences of modified p40 domains provided in Table M (SEQ ID NO: 12-57) and in nucleic acid sequences encoding modified p40 domains provided in Table M (SEQ ID NO: 137-182).

The present disclosure provides p40 domains that are modified to have reduced binding to the IL12Rβ1 subunit, wherein the modified p40 domains contain at least one amino acid substitution or group of substitutions from the following list (numbered according to mature p40 sequence; see SEQ ID NO:10 and Table A); W15H, W15K, E59R, E595, K84E, K84W, E86W, D161R, K197T, K197W, W15H_K84L, E59D_D93H, E59R_D93R, F60K_K197W, F60R_K84Y, K84I_E86R, K84W_D161R, K84W_K197W, E86R_K197D, E86W_D93E, W15H_K84L_K197Q, K58H_E86R_K197D, K58S_E59S_K195D, E59D_K84W_K197W, F60R_K84E_K197W, K84I_E86R_D93H, W15R_E59D_F60D_K197W, E45R_K58S_E59 S_K195D; as captured in the sequences of modified p40 domains provided in Table M (SEQ ID NO: 12, 13, 19, 20, 24, 27, 30, 34, 38, 39, 41, 47, 48, 49, 51, 52, 54, 55, 56, 57, 42, 44, 45, 46, 50, 53, 40, 43), respectively, and in nucleic acid sequences encoding modified p40 domains provided in Table M (SEQ ID NO: 137, 138, 144, 145, 149, 152, 155, 159, 163, 164, 166, 172, 173, 174, 176, 177, 179, 180, 181, 182, 167, 169, 170, 171, 175, 178, 165, 168), respectively.

Generally, the function of p40 domains that are modified to have reduced binding to the IL12Rβ1 subunit is to provide a biologically active IL12 family protein that has reduced toxicity and a broader therapeutic window. As used herein, “therapeutic window” refers to the range of dosages which can treat disease effectively without having toxic effects; e.g., as is in the area between adverse response and desired response is the therapeutic window. Examples of toxic effects of IL12 administration include, without limitation: skin toxicity, local inflammation, stomatitis, systemic inflammation, fatigue, weight loss, emesis, anorexia, hematologic toxicities, such as anemia, lymphopenia, neutropenia, thrombocytopenia, hypoproteinemia, hypophosphatemia, and hypocalcemia, enlargement of lymph nodes, splenomegaly, and bone marrow hyperplasia, bone marrow toxicities, muscle toxicities, neurologic toxicities, hepatic toxicities such as hepatic dysfunction, elevated transaminases, elevated aspartate aminotransferase (AST), elevated alanine aminotransferase (ALT), elevated alkaline phosphatase, hyperbilirubinemia, and hypoalbuminemia, elevated creatinine, diarrhea, dyspnea, and gastrointestinal hemorrhage. In some embodiments, toxic effects refer to dose-limiting toxicities. Other toxic effects of IL12 administration are known to those of ordinary skill in the art.

The p40 domain is a secreted protein; either as a monomer, homodimer, or heterodimer with p19 or p35; and thus possesses an N-terminal secretory signal peptide, or SP, (SEQ ID NO: 122, also residues 1-22 of SEQ ID NO: 123) that targets the newly translated protein to the endoplasmic reticulum (ER) for translocation and subsequent secretion from the cell. Upon translocation into the ER, the SP is cleaved from the remainder of the p40 protein. In this fashion, a protein that contains a SP is often referred to as a ‘preprotein’, or ‘precursor’, while the protein that is secreted after SP cleavage is referred to as a ‘mature’ protein. When applying numbering to amino acids within a protein sequence, it is common to follow one of two conventions: 1) the protein is numbered starting at 1 from the first residue at the N-terminus the SP, or 2) the protein is numbered starting at 1 from the first residue at the N-terminus of the mature protein after SP cleavage. Table A provides the numbering of amino acids within the p40 preprotein and mature protein by either convention. In the present disclosure, amino acid substitutions made to the p40 domain are numbered according to their position in the mature p40 protein (SEQ ID NO: 10).

TABLE A
Numbering of p40 amino acid sequence according
to preprotein and mature protein position

| Amino acid | Preprotein | Mature protein |
|---|---|---|
| M | 1 | NA |
| C | 2 | NA |
| H | 3 | NA |
| Q | 4 | NA |
| Q | 5 | NA |
| L | 6 | NA |
| V | 7 | NA |
| I | 8 | NA |
| S | 9 | NA |
| W | 10 | NA |
| F | 11 | NA |
| S | 12 | NA |
| L | 13 | NA |
| V | 14 | NA |
| F | 15 | NA |
| L | 16 | NA |
| A | 17 | NA |
| S | 18 | NA |
| P | 19 | NA |
| L | 20 | NA |
| V | 21 | NA |
| A | 22 | NA |
| I | 23 | 1 |
| W | 24 | 2 |
| E | 25 | 3 |
| L | 26 | 4 |
| K | 27 | 5 |
| K | 28 | 6 |
| D | 29 | 7 |
| V | 30 | 8 |
| Y | 31 | 9 |
| V | 32 | 10 |
| V | 33 | 11 |
| E | 34 | 12 |
| L | 35 | 13 |
| D | 36 | 14 |
| W | 37 | 15 |
| Y | 38 | 16 |
| P | 39 | 17 |
| D | 40 | 18 |
| A | 41 | 19 |
| P | 42 | 20 |
| G | 43 | 21 |
| E | 44 | 22 |
| M | 45 | 23 |
| V | 46 | 24 |
| V | 47 | 25 |
| L | 48 | 26 |
| T | 49 | 27 |
| C | 50 | 28 |
| D | 51 | 29 |
| T | 52 | 30 |
| P | 53 | 31 |
| E | 54 | 32 |
| E | 55 | 33 |
| D | 56 | 34 |
| G | 57 | 35 |
| I | 58 | 36 |
| T | 59 | 37 |
| W | 60 | 38 |
| T | 61 | 39 |
| L | 62 | 40 |
| D | 63 | 41 |
| Q | 64 | 42 |
| S | 65 | 43 |
| S | 66 | 44 |
| E | 67 | 45 |
| V | 68 | 46 |
| L | 69 | 47 |
| G | 70 | 48 |
| S | 71 | 49 |
| G | 72 | 50 |
| K | 73 | 51 |
| T | 74 | 52 |
| L | 75 | 53 |
| T | 76 | 54 |
| I | 77 | 55 |
| Q | 78 | 56 |
| V | 79 | 57 |
| K | 80 | 58 |
| E | 81 | 59 |
| F | 82 | 60 |
| G | 83 | 61 |
| D | 84 | 62 |
| A | 85 | 63 |
| G | 86 | 64 |
| Q | 87 | 65 |
| Y | 88 | 66 |
| T | 89 | 67 |
| C | 90 | 68 |
| H | 91 | 69 |
| K | 92 | 70 |
| G | 93 | 71 |
| G | 94 | 72 |
| E | 95 | 73 |
| V | 96 | 74 |
| L | 97 | 75 |
| S | 98 | 76 |
| H | 99 | 77 |
| S | 100 | 78 |
| L | 101 | 79 |
| L | 102 | 80 |
| L | 103 | 81 |
| L | 104 | 82 |
| H | 105 | 83 |
| K | 106 | 84 |
| K | 107 | 85 |
| E | 108 | 86 |
| D | 109 | 87 |
| G | 110 | 88 |
| I | 111 | 89 |
| W | 112 | 90 |
| S | 113 | 91 |
| T | 114 | 92 |
| D | 115 | 93 |
| I | 116 | 94 |
| L | 117 | 95 |
| K | 118 | 96 |
| D | 119 | 97 |
| Q | 120 | 98 |
| K | 121 | 99 |
| E | 122 | 100 |
| P | 123 | 101 |
| K | 124 | 102 |
| N | 125 | 103 |
| K | 126 | 104 |
| T | 127 | 105 |
| F | 128 | 106 |
| L | 129 | 107 |
| R | 130 | 108 |
| C | 131 | 109 |
| E | 132 | 110 |
| A | 133 | 111 |
| K | 134 | 112 |
| N | 135 | 113 |
| Y | 136 | 114 |
| S | 137 | 115 |
| G | 138 | 116 |
| R | 139 | 117 |
| F | 140 | 118 |
| T | 141 | 119 |
| C | 142 | 120 |
| W | 143 | 121 |
| W | 144 | 122 |
| L | 145 | 123 |
| T | 146 | 124 |
| T | 147 | 125 |
| I | 148 | 126 |
| S | 149 | 127 |
| T | 150 | 128 |
| D | 151 | 129 |
| L | 152 | 130 |
| T | 153 | 131 |
| F | 154 | 132 |
| S | 155 | 133 |
| V | 156 | 134 |
| K | 157 | 135 |
| S | 158 | 136 |
| S | 159 | 137 |
| R | 160 | 138 |
| G | 161 | 139 |
| S | 162 | 140 |
| S | 163 | 141 |
| D | 164 | 142 |
| P | 165 | 143 |
| Q | 166 | 144 |
| G | 167 | 145 |
| V | 168 | 146 |
| T | 169 | 147 |
| C | 170 | 148 |
| G | 171 | 149 |
| A | 172 | 150 |
| A | 173 | 151 |
| T | 174 | 152 |
| L | 175 | 153 |
| S | 176 | 154 |
| A | 177 | 155 |
| E | 178 | 156 |
| R | 179 | 157 |
| V | 180 | 158 |
| R | 181 | 159 |
| G | 182 | 160 |
| D | 183 | 161 |
| N | 184 | 162 |
| K | 185 | 163 |
| E | 186 | 164 |
| Y | 187 | 165 |
| E | 188 | 166 |
| Y | 189 | 167 |
| S | 190 | 168 |
| V | 191 | 169 |
| E | 192 | 170 |
| C | 193 | 171 |
| Q | 194 | 172 |
| E | 195 | 173 |
| D | 196 | 174 |
| S | 197 | 175 |
| A | 198 | 176 |
| C | 199 | 177 |
| P | 200 | 178 |
| A | 201 | 179 |
| A | 202 | 180 |
| E | 203 | 181 |
| E | 204 | 182 |
| S | 205 | 183 |
| L | 206 | 184 |
| P | 207 | 185 |
| I | 208 | 186 |
| E | 209 | 187 |
| V | 210 | 188 |
| M | 211 | 189 |
| V | 212 | 190 |
| D | 213 | 191 |
| A | 214 | 192 |
| V | 215 | 193 |
| H | 216 | 194 |
| K | 217 | 195 |
| L | 218 | 196 |
| K | 219 | 197 |
| Y | 220 | 198 |
| E | 221 | 199 |
| N | 222 | 200 |
| Y | 223 | 201 |
| T | 224 | 202 |
| S | 225 | 203 |
| S | 226 | 204 |
| F | 227 | 205 |
| F | 228 | 206 |
| I | 229 | 207 |
| R | 230 | 208 |
| D | 231 | 209 |
| I | 232 | 210 |
| I | 233 | 211 |
| K | 234 | 212 |
| P | 235 | 213 |
| D | 236 | 214 |
| P | 237 | 215 |
| P | 238 | 216 |
| K | 239 | 217 |
| N | 240 | 218 |
| L | 241 | 219 |
| Q | 242 | 220 |
| L | 243 | 221 |
| K | 244 | 222 |
| P | 245 | 223 |
| L | 246 | 224 |
| K | 247 | 225 |
| N | 248 | 226 |
| S | 249 | 227 |
| R | 250 | 228 |
| Q | 251 | 229 |
| V | 252 | 230 |
| E | 253 | 231 |
| V | 254 | 232 |
| S | 255 | 233 |
| W | 256 | 234 |
| E | 257 | 235 |
| Y | 258 | 236 |
| P | 259 | 237 |
| D | 260 | 238 |
| T | 261 | 239 |
| W | 262 | 240 |
| S | 263 | 241 |
| T | 264 | 242 |
| P | 265 | 243 |
| H | 266 | 244 |
| S | 267 | 245 |
| Y | 268 | 246 |
| F | 269 | 247 |
| S | 270 | 248 |
| L | 271 | 249 |
| T | 272 | 250 |
| F | 273 | 251 |
| C | 274 | 252 |
| V | 275 | 253 |
| Q | 276 | 254 |
| V | 277 | 255 |
| Q | 278 | 256 |
| G | 279 | 257 |
| K | 280 | 258 |
| S | 281 | 259 |
| K | 282 | 260 |
| R | 283 | 261 |
| E | 284 | 262 |
| K | 285 | 263 |
| K | 286 | 264 |
| D | 287 | 265 |
| R | 288 | 266 |
| V | 289 | 267 |
| F | 290 | 268 |
| T | 291 | 269 |
| D | 292 | 270 |
| K | 293 | 271 |
| T | 294 | 272 |
| S | 295 | 273 |
| A | 296 | 274 |
| T | 297 | 275 |
| V | 298 | 276 |
| I | 299 | 277 |
| C | 300 | 278 |
| R | 301 | 279 |
| K | 302 | 280 |
| N | 303 | 281 |
| A | 304 | 282 |
| S | 305 | 283 |
| I | 306 | 284 |
| S | 307 | 285 |
| V | 308 | 286 |
| R | 309 | 287 |
| A | 310 | 288 |
| Q | 311 | 289 |
| D | 312 | 290 |
| R | 313 | 291 |
| Y | 314 | 292 |
| Y | 315 | 293 |
| S | 316 | 294 |
| S | 317 | 295 |
| S | 318 | 296 |
| W | 319 | 297 |
| S | 320 | 298 |
| E | 321 | 299 |
| W | 322 | 300 |
| A | 323 | 301 |
| S | 324 | 302 |
| V | 325 | 303 |
| P | 326 | 304 |
| C | 327 | 305 |
| S | 328 | 306 |

The approach of iterative structure-guided in silico mutagenesis and structure evaluation described herein, specifically in Example 1, enabled the generation of a concise library of designs that span a broad range of predicted affinities between p40 and IL12Rβ1. This in silico approach offers advantages in both efficiency of design and the breadth of solutions obtained compared to other common in vitro approaches to protein mutation and selection known in the art. For example, random or semi-random mutagenesis of individual amino acids followed by protein library expression and screening for mutations with the desired properties may require screening a significantly larger number of samples, as well as multiple rounds of cloning, protein expression, and sorting to iteratively test combinations of mutations to obtain designs with the desired activity. In contrast, the iterative in silico design, mutagenesis and selection approach described herein enabled the preparation up-front of a design library containing single and multiple amino acid substitutions to achieve a broad range of predicted affinities between p40 and IL12Rβ1.

Further, the in silico structure-guided approach described herein enabled the discovery of mutations that achieve both the desired reduction in predicted affinity between p40 and IL12Rβ1 and a positive or minimally negative impact on the predicted stability of the uncomplexed p40 domain, as well as pairs or groups of mutations that are complimentary or synergistic in their reduction of the predicted affinity between p40 and IL12Rβ1 and/or maintenance or improvement of the predicted stability of the uncomplexed p40 domain.

For example, structural analyses of the p40 domain in complex with IL12Rβ1 identified the amino acids K84, E86, and K197, among others, as hotspots, i.e., key residues contributing to stability and affinity, at the p40-IL12R31 interface, as described in Example 1. However, in the structural analyses of uncomplexed p40, it was discovered that K84, E86, and K197 also make significant contributions to the predicted stability of uncomplexed p40. It was therefore important to consider the impact of mutations at these positions on both the predicted affinity between p40 and IL12Rβ1 and on the predicted stability of uncomplexed p40. A typical approach to protein mutagenesis known in the art is ‘alanine scanning’, wherein all or a selected group of amino acids within a protein of interest are individually substituted with alanine and the effect of substitution measured by a binding or functional assay using the mutated proteins. Given the contributions of K84, E86, and K197 to the predicted stability of p40, through steric and electrostatic interactions with neighboring residues, it is predicted that mutations to alanine at these positions would be detrimental to the stability and/or other biophysical properties of the mutant p40 domains (for example, increased aggregation propensity caused by increased exposure of hydrophobic residues in the vicinity of the substitutions). The in silico approach to design described herein, specifically in Example 1, thus identified several amino acid substitutions at these positions that provide similar or greater reductions to the predicted affinity between p40 and IL12Rβ1 as do substitutions with alanine, but with smaller negative, and in some cases positive, impacts on the predicted stability of uncomplexed p40. For example, modelling the amino acid substitution K84W revealed increased steric complementarity with neighboring amino acids in uncomplexed p40 relative to the non-substituted lysine at the same position, leading to an improvement in predicted stability.

It was also discovered that a substitution identified as preferred at a particular amino acid location when substituted in isolation may not be the preferred substitution at the same location if substituted in combination with another or multiple other amino acids in the same structural vicinity. For example, the substitution K84W described above may or may not be the preferred substitution at amino acid K84 when combined with substitutions of other amino acids. Indeed, when combined with the substitution E86R, the substitution K84I is preferred over K84W because its smaller size allows E86R to adopt a conformation where it forms favourable hydrogen bonds within p40 leading to improved predicted stability. These favourable hydrogen bonds would be precluded by steric clashes in the combination of E86R with K84W. Likewise, the substitution E86R in isolation is precluded from adopting this favourable conformation by steric clashes with the non-modified K84. While E86R in isolation is predicted to reduce affinity between p40 and IL12Rβ1 as desired, it is predicted to have a large detrimental impact to the stability of uncomplexed p40. The structural synergy generated by combination of the K84I and E86R substitutions instead produces a benefit to p40 stability and thus demonstrates an advantage of the in silico modelling approach described herein to discover complimentary substitutions.

In another example, the structural analyses of the p40 domain in complex with IL12Rβ1 described in Example 1 identified the amino acid W15 as a hotspot at the p40-IL12R31 interface. The molecular dynamics simulations and analyses of uncomplexed p40 revealed significant conformational changes in the region around W15, so that in the uncomplexed state W15 is observed to contribute significantly more intramolecular p40 contact area as well as a hydrogen bond between its indole nitrogen and the backbone oxygen of H83, as compared to the W15 conformation observed for the p40 domain in complex with IL12Rβ1, which allows for increased intermolecular contact area between p40 and IL12Rβ1 at the expense of intramolecular contact area within p40. In silico mutagenesis modelling as described in Example 1 enabled the discovery of mutations and mutation groups including W15 that reduce predicted affinity between p40 and IL12Rβ1 as desired but also minimize the detrimental impact to predicted stability of uncomplexed p40 by recovering or replacing the intramolecular contacts made by W15 that may be lost upon substitution. For example, the substitution W15H was designed to preserve the hydrogen bond made to the backbone oxygen of H83 and minimize the detrimental impact to predicted stability of possible mutations at position W15. In another example, the group of mutations W15R_E59D_F60D_K197W was designed to introduce a strong bidentate hydrogen bond interaction between W15R and F60D with additional intramolecular packing recovered by K197 in the uncomplexed state, resulting in a design that minimizes the detrimental impact of mutations at these positions to the predicted stability of uncomplexed p40 while also providing a large reduction to the predicted affinity between p40 and IL12Rβ1.

Using this approach as described in Example 1, preferred substitutions and groups of substitutions at p40-IL12R31 interface hotspots and neighboring residues were identified to produce a design library that spans a broad range of predicted affinities between p40 and IL12Rβ1 while having a positive or minimally negative impact on the predicted stability of the uncomplexed p40 domain. The preferred substitutions and groups of substitutions are provided in Table B.

TABLE B
Preferred p40 mutations identified by in silico design

| De- | | |
|---|---|---|
| sign | | |
| ID # | Precursor p40 mutation | Mature p40 mutation |
| 1 | W37H | W15H |
| 2 | W37K | W15K |
| 3 | W37R | W15R |
| 4 | D40G | D18G |
| 5 | E67K | E45K |
| 6 | E67R | E45R |
| 7 | K80H | K58H |
| 8 | K80S | K58S |
| 9 | K80W | K58W |
| 10 | E81D | E59D |
| 11 | E81G | E59G |
| 12 | E81R | E59R |
| 13 | E81S | E59S |
| 14 | F82D | F60D |
| 15 | F82E | F60E |
| 16 | F82K | F60K |
| 17 | F82R | F60R |
| 18 | F82V | F60V |
| 19 | D84H | D62H |
| 20 | D84I | D62I |
| 21 | D84N | D62N |
| 22 | K106E | K84E |
| 23 | K106I | K84I |
| 24 | K106L | K84L |
| 25 | K106V | K84V |
| 26 | K106W | K84W |
| 27 | K106Y | K84Y |
| 28 | E108L | E86L |
| 29 | E108R | E86R |
| 30 | E108S | E86S |
| 31 | E108W | E86W |
| 32 | D115E | D93E |
| 33 | D115H | D93H |
| 34 | D115R | D93R |
| 35 | D115W | D93W |
| 36 | D183R | D161R |
| 37 | D183S | D161S |
| 38 | K217D | K195D |
| 39 | K219D | K197D |
| 40 | K219E | K197E |
| 41 | K219Q | K197Q |
| 42 | K219T | K197T |
| 43 | K219W | K197W |
| 44 | W37H_K106L | W15H_K84L |
| 45 | K80H_K106I | K58H_K84I |
| 46 | E81D_K106W | E59D_K84W |
| 47 | E81G_K106W | E59G_K84W |
| 48 | E81R_K106E | E59R_K84E |
| 49 | E81R_K106W | E59R_K84W |
| 50 | E81R_E108W | E59R_E86W |
| 51 | E81D_D115H | E59D_D93H |
| 52 | E81R_D115R | E59R_D93R |
| 53 | E81R_K219E | E59R_K197E |
| 54 | E81R_K219W | E59R_K197W |
| 55 | F82E_K106W | F60E_K84W |
| 56 | F82R_K106Y | F60R_K84Y |
| 57 | F82K_K219W | F60K_K197W |
| 58 | F82R_K219W | F60R_K197W |
| 59 | K106I_E108R | K84I_E86R |
| 60 | K106E_D115H | K84E_D93H |
| 61 | K106I_D115H | K84I_D93H |
| 62 | K106V_D115H | K84V_D93H |
| 63 | K106W_D115W | K84W_D93W |
| 64 | K106I_D183R | K84I_D161R |
| 65 | K106W_D183R | K84W_D161R |
| 66 | K106W_K219E | K84W_K197E |
| 67 | K106W_K219Q | K84W_K197Q |
| 68 | K106W_K219W | K84W_K197W |
| 69 | E108W_D115E | E86W_D93E |
| 70 | E108R_K219D | E86R_K197D |
| 71 | E108W_K219W | E86W_K197W |
| 72 | W37H_K106L_K219Q | W15H_K84L_K197Q |
| 73 | K80S_E81S_K217D | K58S_E59S_K195D |
| 74 | K80H_E108R_K219D | K58H_E86R_K197D |
| 75 | E81D_K106W_K219W | E59D_K84W_K197W |
| 76 | F82R_K106E_K219W | F60R_K84E_K197W |
| 77 | K106I_E108R_D115H | K84I_E86R_D93H |
| 78 | W37R_E81D_F82D_K219W | W15R_E59D_F60D_K197W |
| 79 | E67R_K80S_E81S_K217D | E45R_K58S_E59S_K195D |

In various embodiments, described herein are modified p40 domains that have a reduced binding to the IL12Rβ1 subunit, compared to a p40 domain that does not comprise such amino acid substitution(s)). In some embodiments, a modified p40 domain comprises one or more amino acid substitution(s) selected from Table C.

TABLE C
p40 Domain Mutations Identified by the
Described In Silico Design Methods

| De- | | |
|---|---|---|
| sign | | Mature p40 mutation |
| ID # | Precursor p40 mutation | (based on SEQ ID NO: 10) |
| 1 | W37H | W15H |
| 2 | W37K | W15K |
| 3 | W37R | W15R |
| 4 | D40G | D18G |
| 5 | E67K | E45K |
| 7 | K80H | K58H |
| 9 | K80W | K58W |
| 10 | E81D | E59D |
| 11 | E81G | E59G |
| 12 | E81R | E59R |
| 14 | F82D | F60D |
| 15 | F82E | F60E |
| 16 | F82K | F60K |
| 17 | F82R | F60R |
| 18 | F82V | F60V |
| 19 | D84H | D62H |
| 20 | D84I | D62I |
| 21 | D84N | D62N |
| 22 | K106E | K84E |
| 23 | K106I | K84I |
| 24 | K106L | K84L |
| 25 | K106V | K84V |
| 26 | K106W | K84W |
| 27 | K106Y | K84Y |
| 28 | E108L | E86L |
| 29 | E108R | E86R |
| 30 | E108S | E86S |
| 31 | E108W | E86W |
| 32 | D115E | D93E |
| 33 | D115H | D93H |
| 34 | D115R | D93R |
| 35 | D115W | D93W |
| 36 | D183R | D161R |
| 37 | D183S | D161S |
| 39 | K219D | K197D |
| 40 | K219E | K197E |
| 41 | K219Q | K197Q |
| 42 | K219T | K197T |
| 43 | K219W | K197W |
| 44 | W37H_K106L | W15H_K84L |
| 45 | K80H_K106I | K58H_K84I |
| 46 | E81D_K106W | E59D_K84W |
| 47 | E81G_K106W | E59G_K84W |
| 48 | E81R_K106E | E59R_K84E |
| 49 | E81R_K106W | E59R_K84W |
| 50 | E81R_E108W | E59R_E86W |
| 51 | E81D_D115H | E59D_D93H |
| 52 | E81R_D115R | E59R_D93R |
| 53 | E81R_K219E | E59R_K197E |
| 54 | E81R_K219W | E59R_K197W |
| 55 | F82E_K106W | F60E_K84W |
| 56 | F82R_K106Y | F60R_K84Y |
| 57 | F82K_K219W | F60K_K197W |
| 58 | F82R_K219W | F60R_K197W |
| 59 | K106I_E108R | K84I_E86R |
| 60 | K106E_D115H | K84E_D93H |
| 61 | K106I_D115H | K84I_D93H |
| 62 | K106V_D115H | K84V_D93H |
| 63 | K106W_D115W | K84W_D93W |
| 64 | K106I_D183R | K84I_D161R |
| 65 | K106W_D183R | K84W_D161R |
| 66 | K106W_K219E | K84W_K197E |
| 67 | K106W_K219Q | K84W_K197Q |
| 68 | K106W_K219W | K84W_K197W |
| 69 | E108W_D115E | E86W_D93E |
| 70 | E108R_K219D | E86R_K197D |
| 71 | E108W_K219W | E86W_K197W |
| 72 | W37H_K106L_K219Q | W15H_K84L_K197Q |
| 73 | K80S_E81S_K217D | K58S_E59S_K195D |
| 74 | K80H_E108R_K219D | K58H_E86R_K197D |
| 75 | E81D_K106W_K219W | E59D_K84W_K197W |
| 76 | F82R_K106E_K219W | F60R_K84E_K197W |
| 77 | K106I_E108R_D115H | K84I_E86R_D93H |
| 78 | W37R_E81D_F82D_K219W | W15R_E59D_F60D_K197W |

In various embodiments, described herein are modified p40 domains that have a reduced binding affinity to the IL12Rβ1 subunit, compared to a p40 domain that does not comprise such amino acid substitution(s)), and wherein the modified p40 domains can contain at least one amino acid substitution or a set of amino acid substitutions. In some embodiments, such one or more amino acid substitution(s) or set(s) of substitutions can be (numbered according to the mature p40 sequence recited in SEQ ID NO: 10 and Table A): W15H, W15K, W15R, D18G, E45K, E45R, K58H, K58S, K58W, E59D, E59G, E59R, E595, F60D, F60E, F60K, F60R, F60V, D62H, D62I, D62N, K84E, K84I, K84L, K84V, K84W, K84Y, E86L, E86R, E86S, E86W, D93E, D93H, D93R, D93W, D161R, D161S, K195D, K197D, K197E, K197Q, K197T, K197W, W15H_K84L, K58H_K84I, E59D_K84W, E59G_K84W, E59R_K84E, E59R_K84W, E59R_E86W, E59D_D93H, E59R_D93R, E59R_K197E, E59R_K197W, F60E_K84W, F60R_K84Y, F60K_K197W, F60R_K197W, K84I_E86R, K84E_D93H, K84I_D93H, K84V_D93H, K84W_D93W, K84I_D161R, K84W_D161R, K84W_K197E, K84W_K197Q, K84W_K197W, E86W_D93E, E86R_K197D, E86W_K197W, W15H_K84L_K197Q, K58S_E59S_K195D, K58H_E86R_K197D, E59D_K84W_K197W, F60R_K84E_K197W, K84I_E86R_D93H, W15R_E59D_F60D_K197W and/or E45R_K58S_E59S_K195D (see, e.g., Table B).

In certain embodiments, described herein are modified p40 domains that have a reduced binding affinity to the IL12Rβ1 subunit, compared to a p40 domain that does not comprise such amino acid substitution(s)), and wherein such modified p40 domains comprise one or more of the following amino acid substitution(s) or set(s) of amino acid substitutions (numbered according to the mature p40 sequence recited in SEQ ID NO: 10 and Table A): W15H, W15K, W15R, D18G, E45K, K58H, K58W, E59D, E59G, E59R, F60D, F60E, F60K, F60R, F60V, D62H, D62I, D62N, K84E, K84I, K84L, K84V, K84W, K84Y, E86L, E86R, E86S, E86W, D93E, D93H, D93R, D93W, D161R, D161S, K197D, K197E, K197Q, K197T, K197W, W15H_K84L, K58H_K84I, E59D_K84W, E59G_K84W, E59R_K84E, E59R_K84W, E59R_E86W, E59D_D93H, E59R_D93R, E59R_K197E, E59R_K197W, F60E_K84W, F60R_K84Y, F60K_K197W, F60R_K197W, K84I_E86R, K84E_D93H, K84I_D93H, K84V_D93H, K84W_D93W, K84I_D161R, K84W_D161R, K84W_K197E, K84W_K197Q, K84W_K197W, E86W_D93E, E86R_K197D, E86W_K197W, W15H_K84L_K197Q, K58S_E59S_K195D, K58H_E86R_K197D, E59D_K84W_K197W, F60R_K84E_K197W, K84I_E86R_D93H and/or W15R_E59D_F60D_K197W (see, e.g., Table C).

In some embodiments, described herein are modified p40 domains that have a reduced binding affinity to the IL12Rβ1 subunit (e.g., compared to a p40 domain that does not comprise such amino acid substitution(s)) and that comprise one or more of the following amino acid substitution(s) or set(s) of substitutions (numbered according to the mature p40 sequence recited in SEQ ID NO:10): W15H, W15K, W15R, E45R, K58H, K58S, E59D, E59R, E595, F60D, F60K, F60R, K84E, K84I, K84L, K84W, K84Y, E86R, E86W, D93E, D93H, D93R, D161R, K195D, K197D, K197Q, K197T, K197W, W15H_K84L, E59D_D93H, E59R_D93R, F60K_K197W, F60R_K84Y, K84I_E86R, K84W_D161R, K84W_K197W, E86R_K197D, E86W_D93E, W15H_K84L_K197Q, K58H_E86R_K197D, K58S_E59S_K195D, E59D_K84W_K197W, F60R_K84E_K197W, K84I_E86R_D93H, W15R_E59D_F60D_K197W and/or E45R_K58S_E59S_K195D. The amino acid sequences of 46 modified p40 domains that each contains one of these 46 amino acid substitutions are provided in Table M herein and comprise or consist of the amino acid sequences set forth in SEQ ID NOS: 12-57, respectively, or a sequence having at least about 90%, 95%, 97%, or at least about 99% sequence identity thereto. In various embodiments, such amino acid sequences of the 46 modified p40 domains are encoded by the nucleic acid sequences provided in Table M herein and corresponding to SEQ ID NOS: 137-182, respectively.

In some embodiments, described herein are modified p40 domains that have a reduced binding affinity to the IL12Rβ1 subunit (e.g., compared to a p40 domain that does not comprise such amino acid substitution(s)) and that comprise one or more of the following amino acid substitution(s) or group(s) of substitutions (numbered according to the mature p40 sequence recited in SEQ ID NO:10): W15H, W15K, W15R, K58H, E59D, E59R, F60D, F60K, F60R, K84E, K84I, K84L, K84W, K84Y, E86R, E86W, D93E, D93H, D93R, D161R, K197D, K197Q, K197T, K197W, W15H_K84L, E59D_D93H, E59R_D93R, F60K_K197W, F60R_K84Y, K84I_E86R, K84W_D161R, K84W_K197W, E86R_K197D, E86W_D93E, W15H_K84L_K197Q, K58H_E86R_K197D, K58S_E59S_K195D, E59D_K84W_K197W, F60R_K84E_K197W, K84I_E86R_D93H, and/or W15R_E59D_F60D_K197W. The amino acid sequences of 41 modified p40 domains that each contains one of these 41 amino acid substitutions are described in Table M herein and comprise or consist of the amino acid sequences set forth in SEQ ID NOS: 12-14, 16, 18, 19, 21-34, 36-56, respectively, or a sequence having at least about 90%, 95%, 97%, or at least about 99% sequence identity thereto. In various embodiments, such amino acid sequences of the 41 modified p40 domains are encoded by the nucleic acid sequences described in Table M herein and corresponding to SEQ ID NOS: 137-139, 141, 143, 144, 146-159, 161-181, respectively.

In some embodiments, the present disclosure describes modified p40 domains that have a reduced binding affinity to the IL12Rβ1 subunit (e.g., compared to a p40 domain that does not comprise such amino acid substitution(s)), wherein the modified p40 domains comprise one or more of the following amino acid substitution(s) or group(s) of substitutions (numbered according to mature p40 sequence; see SEQ ID NO:10): W15H, W15K, E59R, E595, K84E, K84W, E86W, D161R, K197T, K197W, W15H_K84L, E59D_D93H, E59R_D93R, F60K_K197W, F60R_K84Y, K84I_E86R, K84W_D161R, K84W_K197W, E86R_K197D, E86W_D93E, W15H_K84L_K197Q, K58H_E86R_K197D, K58S_E59S_K195D, E59D_K84W_K197W, F60R_K84E_K197W, K84I_E86R_D93H, W15R_E59D_F60D_K197W and/or E45R_K58S_E59S_K195D. The amino acid sequences of modified p40 domains that each contain one of these amino acid substitutions are provided in Table M and can comprise or consist of the amino acid sequences set forth in SEQ ID NOS: 12, 13, 19, 20, 24, 27, 30, 34, 38, 39, 41, 47, 48, 49, 51, 52, 54, 55, 56, 57, 42, 44, 45, 46, 50, 53, 40 and 43, respectively, or a sequence having at least about 90%, 95%, 97%, or at least about 99% sequence identity thereto. In various embodiments, such amino acid sequences are encoded by nucleic acid sequences provided in Table M and corresponding to SEQ ID NOS: 137, 138, 144, 145, 149, 152, 155, 159, 163, 164, 166, 172, 173, 174, 176, 177, 179, 180, 181, 182, 167, 169, 170, 171, 175, 178, 165 and 168, respectively.

In some embodiments, the present disclosure describes modified p40 domains that have a reduced binding affinity to the IL12Rβ1 subunit (e.g., compared to a p40 domain that does not comprise such amino acid substitution(s)), wherein the modified p40 domains comprise one or more of the following amino acid substitution(s) or group(s) of substitutions (numbered according to mature p40 sequence; see SEQ ID NO:10): W15H, W15K, E59R, K84E, K84W, E86W, D161R, K197T, K197W, W15H_K84L, E59D_D93H, E59R_D93R, F60K_K197W, F60R_K84Y, K84I_E86R, K84W_D161R, K84W_K197W, E86R_K197D, E86W_D93E, W15H_K84L_K197Q, K58H_E86R_K197D, K58S_E59S_K195D, E59D_K84W_K197W, F60R_K84E_K197W, K84I_E86R_D93H and/or W15R_E59D_F60D_K197W. The amino acid sequences of modified p40 domains that each contain one of these amino acid substitutions are provided in Table M and can comprise or consist of the amino acid sequences set forth in SEQ ID NOS: 12, 13, 19, 24, 27, 30, 34, 38, 39, 41, 47, 48, 49, 51, 52, 54, 55, 56, 57, 42, 44, 45, 46, 50, 53 and 40, respectively, or a sequence having at least about 90%, 95%, 97%, or at least about 99% sequence identity thereto. In various embodiments, such amino acid sequences are encoded by nucleic acid sequences provided in Table M and corresponding to SEQ ID NOS: 137, 138, 144, 149, 152, 155, 159, 163, 164, 166, 172, 173, 174, 176, 177, 179, 180, 181, 182, 167, 169, 170, 171, 175, 178 and 165, respectively.

In some embodiments, described herein is a modified p40 domain having a reduced binding affinity to one or more of its cognate IL12 receptor(s) compared to an unmodified p40 domain, the modified p40 domain comprising one or more amino acid substitutions relative to the wild-type human mature IL12p40 domain sequence set forth in SEQ ID NO: 10, wherein the one or more amino acid substitutions are located at one or more positions of E45, D62 and/or D161, and wherein the numbering of the amino acid residues is based on the amino acid sequence set forth in SEQ ID NO: 10. In some embodiments, the one or more amino acid substitutions at the one or more positions are a K, H, I, N, R and/or S substitution. In certain embodiments, the one or more amino acid substitution are E45K, D62H, D62I, D62N, D161R and/or D161S.

In yet other embodiments, described herein is a modified p40 domain comprising one or more amino acid substitution(s) relative to the wild-type human mature IL12p40 domain sequence set forth in SEQ ID NO: 10, wherein the one or more amino acid substitution(s) are W15H, W15K, W15R, D18G, E45K, K58H, K58W, E59D, E59G, E59R, F60D, F60E, F60K, F60R, F60V, D62H, D62I, D62N, K84E, K84I, K84L, K84V, K84W, K84Y, E86L, E86R, E86S, E86W, D93E, D93H, D93R, D93W, D161R, D161S, K197D, K197E, K197Q, K197T, K197W, or any combination thereof, and wherein the numbering of the amino acid residues is based on the amino acid sequence set forth in SEQ ID NO: 10. In some embodiments, such modified p40 domain comprises one or more, two or more, or three or more amino acid substitutions. In certain embodiments, the one or more amino acid substitution(s) can be W15H, W15K, D18G, E45K, K58H, K58W, E59G, E59R, F60V, D62H, D62I, D62N, K84E, K84W, E86L, E86S, E86W, D93H, D93W, D161R, D161S, K197E, K197Q, K197T and/or K197W. In some of these embodiments, the one or more amino acid substitution can be W15H, W15K, E59R, K84E, K84W, E86W, D161R, K197T and/or K197W.

In embodiments in which a modified p40 domain comprises two or more amino acid substitutions relative to the wildtype p40 sequence set forth in SEQ ID NO: 10, the two or more amino acid substitutions are W15H_K84L, K58H_K84I, E59D_K84W, E59G_K84W, E59R_K84E, E59R_K84W, E59R_E86W, E59D_D93H, E59R_D93R, E59R_K197E, E59R_K197W, F60E_K84W, F60R_K84Y, F60K_K197W, F60R_K197W, K84I_E86R, K84E_D93H, K84I_D93H, K84V_D93H, K84W_D93W, K84I_D161R, K84W_D161R, K84W_K197E, K84W_K197Q, K84W_K197W, E86W_D93E, E86R_K197D, E86W_K197W, W15H_K84L_K197Q, K58H_E86R_K197D, E59D_K84W_K197W, F60R_K84E_KI97W, K84I_E86R_D93H and/or W15R_E59D_F60D_K197W.

In certain embodiments, the two or more amino acid substitutions are W15H_K84L, E59D_D93H, E59R_D93R, F60K_K197W, F60R_K84Y, K84I_E86R, K84W_D161R, K84W_K197W, E86R_K197D, E86W_D93E, W15H_K84L_K197Q, K58H_E86R_K197D, E59D_K84W_K197W, F60R_K84E_KI97W, K84I_E86R_D93H, and/or W15R_E59D_F60D_K197 W.

In other embodiments, the two or more amino acid substitutions are K58H_K84I, E59D_K84W, E59G_K84W, E59R_E86W, E59R_K197E, E59R_K197W, E59R_K84E, E59R_K84W, F60E_K84W, F60R_K197W, K84E_D93H, K84I_D161R, K84I_D93H, K84V_D93H, K84W_D93W, K84W_K197E, K84W_K197Q, and/or E86W_K197W.

In yet other embodiments, the two or more amino acid substitutions are W15H_K84L, K58H_K84I, E59D_K84W, E59G_K84W, E59R_K84E, E59R_K84W, E59R_E86W, E59D_D93H, E59R_D93R, E59R_K197E, E59R_K197W, F60E_K84W, F60R_K84Y, F60K_K197W, F60R_K197W, K84I_E86R, K84E_D93H, K84I_D93H, K84V_D93H, K84W_D93W, K84I_D161R, K84W_D161R, K84W_K197E, K84W_K197Q, K84W_K197W, E86W_D93E, E86R_K197D, and/or E86W_K197W.

In certain embodiments, the two or more amino acid substitutions are W15H_K84L, E59D_D93H, E59R_D93R, F60K_K197W, F60R_K84Y, K84I_E86R, K84W_D161R, K84W_K197W, E86R_K197D, and/or E86W_D93E.

In embodiments in which a modified p40 domain comprises three or more amino acid substitutions relative to the wildtype p40 sequence set forth in SEQ ID NO: 10, the three or more amino acid substitutions are W15H_K84L_K197Q, K58H_E86R_K197D, E59D_K84W_K197W, F60R_K84E_KI97W, K84I_E86R_D93H and/or W15R_E59D_F60D_K197 W.

In some embodiments, a modified p40 domain of the present disclosure comprises an amino acid substitution or a set of amino acid substitutions selected from one or more of the following: W15R_E59D_F60D_K197W, W15H_K84L_K197Q, E59D_K84W_K197W, K58H_E86R_K197D, W15H_K84L, F60R_K84E_K197W, K84W_K197W, F60K_K197W, E86R_K197D, K84I_E86R_D93H, E59R_D93R, K84I_E86R, W15K, K84W_D161R, E45R_K58S_E59S_K195D, E59D_D93H, F60R_K84Y, E86W_D93E, K58S_E59S_K195D, K84E, K197T, E59R, W15H, K84W, K197W, E59S, D161R and/or E86W. In some of these embodiments, the modified p40 domain comprises an amino acid substitution or a set of amino acid substitutions selected from one or more of the following: W15H_K84L_K197Q, E59D_K84W_K197W, K84W_K197W, F60K_K197W, E59R_D93R, W15K, and/or F60R_K84Y.

In various embodiments of the present disclosure, the one or more amino acid substitution(s) that a modified p40 domain can comprise is selected from Table C. In at least some of these embodiments, the modified p40 domain comprises or consists of an amino acid sequence having at least about 90%, 95%, 97%, 99%, or 100% sequence identity to the amino acid sequence set forth in any one of SEQ ID NOS: 12-14, 16, 18, 19, 21-34 and 36-56. In some of these embodiments, the modified p40 domain comprises or consists of an amino acid sequence having at least about 99% or 100% sequence identity to the amino acid sequence set forth in any one of SEQ ID NOS: 12-14, 16, 18, 19, 21-34 and 36-56. In some embodiments, the modified p40 domain can have a reduction in binding affinity for at least one of its cognate receptors (e.g., IL12Rβ1) of at least about 2-fold, 5-fold, 10-fold, 15-fold, 20-fold, 30-fold, 40-fold, 50-fold, 100-fold, 200-fold, 300-fold, 400-fold, 500-fold, or at least about 600-fold when compared to an unmodified p40 domain that does not contain the one or more amino acid modification disclosed herein. Such reduction in binding affinity can be measured by SPR, relative NK cell abundance, CD8+ T cell IFNγ release assay, a reporter gene assay, assays using cells transfected with one or more IL12 receptor types (e.g., IL12Rβ1), assays using stimulated T cells, or a combination of these assays.

In some embodiments, an IL12 fusion protein (e.g., an IL12 HetFc fusion protein) comprising an IL12 polypeptide comprising a modified p40 domain, as described herein, can have a reduction in binding affinity for at least one of the cognate IL12 receptors (e.g., IL12Rβ1) of at least about 2-fold, 5-fold, 10-fold, 15-fold, 20-fold, 30-fold, 40-fold, 50-fold, 100-fold, 200-fold, 300-fold, 400-fold, 500-fold, or at least about 600-fold when compared to a corresponding IL12 fusion protein that comprises an unmodified p40 domain not containing the one or more amino acid modification disclosed herein. Such reduction in binding affinity can be measured by SPR, relative NK cell abundance, CD8+ T cell IFNγ release assay, a reporter gene assay, assays using cells transfected with one or more IL12 receptor types (e.g., IL12Rβ1), assays using stimulated T cells, or a combination of these assays.

In some embodiments, a modified p40 domain of this disclosure can have a reduction in binding affinity for at least one of its cognate receptors (e.g., IL12R131) of at least about 5-fold to about 2000-fold, about 10-fold to about 1500-fold, about 15-fold to about 1000-fold, about 20-fold to about 800-fold, about 20-fold to about 600-fold, about 20-fold to about 400-fold, about 20-fold to about 200-fold, about 20-fold to about 100-fold, about 50-fold to about 100-fold, about 50-fold to about 2000-fold, about 100-fold to about 2000-fold, or of at least about 500-fold to about 2000-fold, compared to an unmodified p40 domain that does not contain the one or more amino acid modification disclosed herein.

In some embodiments, a modified p40 domain of this disclosure can have a reduction in binding affinity for at least one of its cognate receptors (e.g., IL12Rβ1) from about 5-fold to about 1000-fold, from about 5-fold to about 800-fold, from about 5-fold to about 600-fold, from about 10-fold to about 500-fold, from about 10-fold to about 300-fold, or from about 20-fold to about 200-fold, relative to the binding affinity of the unmodified wildtype p40 domain which sequence is set forth in SEQ ID NO: 10, and as determined in a reporter gene assay.

In some embodiments, a modified p40 domain of this disclosure can have a reduction in binding affinity for at least one of its cognate receptors (e.g., IL12R131) of at least about 20-fold to about 200-fold.

As used herein, and unless defined otherwise, the terms “reduced binding” and “reduction in binding affinity” in the context of modified p40 domains disclosed herein that comprise one or more amino acid modification(s), refers to a measurably reduced binding affinity of a modified p40 domain, or a fusion protein comprising such modified p40 domain, to at least one of its cognate receptors (e.g., IL12Rβ1), and relative to a wild type p40 domain that does not comprise such one or more amino acid modification(s), wherein the reduced binding determined using any of the analytical methods described herein. Such reduced binding or reduction in binding affinity can either be measured directly via p40-receptor interaction experiments, or indirectly analyzing changes in downstream processes that are affected by a reduced binding affinity of the modified p40 domain.

Functional IL12 activity can be measured, for example, in an NK cell relative abundance, in an assay measuring IFNγ production by immune, e.g., NK cells, or CD8+ T cell IFNγ release assay (as shown in, e.g., Example 4). In some embodiments, a modified p40 domain (or a fusion protein comprising such p40 domain as described herein) can show a complete elimination in binding affinity to IL12Rβ1, e.g., such that the binding activity of an IL12 polypeptide comprising the modified p40 domain is not detectable using established detection assays, such as SPR, an NK, CD8+ T cell or other cell-based assay.

In some embodiments, a modified p40 domain of the present disclosure and/or a fusion protein comprising such modified p40 domain can have a thermostability that is within ±5° C., ±4° C., ±3° C., ±2° C. or ±1° C. of that of the fusion protein containing an unmodified wildtype p40 domain that has the sequence set forth in SEQ ID NO: 10, and as determined by Differential Scanning Fluorimetry (DSF) or Differential Scanning Calorimetry (DSC) as further described herein for fusion proteins. Hence, in various embodiments, the one or more amino acid modifications (e.g., substitutions) may not significantly impact the thermostability of the p40 domains and/or fusion proteins comprising the modified p40 domain (e.g., at most about ±2° C., or less) compared to unmodified p40 domains or fusion proteins containing unmodified p40 domains, respectively.

### IL12 Fusion Proteins

One aspect of the present disclosure provides cytokine fusion proteins containing modified p40 domains and, in particular, provides IL12 and IL23 fusion proteins, also referred to herein as IL12 HetFc fusion proteins. The IL12 fusion proteins described herein comprise an IL12 polypeptide, an Fc domain comprising a first Fc polypeptide and a second Fc polypeptide; and optionally a masking moiety (MM) that reduces, inhibits or blocks IL12 activity. IL12 HetFc fusion proteins containing a MM are also referred to herein as masked IL12 fusion proteins, or as masked IL12 HetFc fusion proteins. Generally, the function of the IL12 fusion proteins with modified p40 domains and of the masked IL12 fusion proteins with modified p40 domains is to provide a biologically active IL12 protein that has reduced toxicity and a broader therapeutic window. In some embodiments, such modified p40 domain of an IL12 fusion protein can comprise one or more amino acid modification(s) listed in Table B herein. In certain embodiments, the modified p40 domain of an IL12 fusion protein can comprise one or more amino acid modification(s) listed in Table C herein.

In some embodiments, the function of an IL12 fusion protein comprising a modified p40 domain can be to provide a biologically active IL12 protein that has a reduced binding affinity for at least one of the cognate IL12 receptors, such as the IL12Rβ1 subunit, and which can in vivo provide a reduced toxicity and therefore broader therapeutic window when compared to an IL12 polypeptide that does not comprise the modified p40 domain. The above can be analogously envisioned for IL23 fusion proteins that comprise an IL23 polypeptide instead of an IL12 polypeptide, as further described herein.

In various embodiments, an IL12 HetFc fusion protein herein comprises: (i) an IL12 polypeptide comprising a modified p40 domain, (ii) a heterodimeric Fc domain (HetFc) comprising a first Fc polypeptide and a second Fc polypeptide, and, optionally, (iii) a masking moiety (MM) that inhibits, or at least reduces, IL12 activity compared to an unmasked IL12 polypeptide. In some embodiments, an IL12 HetFc fusion protein that comprises a MM can also be referred to herein as a masked IL12 fusion protein, or as a masked IL12 HetFc fusion protein.

In some embodiments, an IL12 fusion protein (e.g., an IL12 HetFc fusion protein) comprising a modified p40 domain of this disclosure can have a reduction in binding affinity for at least one of its cognate receptors (e.g., IL12Rβ1) of at least about 5-fold to about 2000-fold, about 10-fold to about 1500-fold, about 15-fold to about 1000-fold, about 20-fold to about 800-fold, about 20-fold to about 600-fold, about 20-fold to about 400-fold, about 20-fold to about 200-fold, about 20-fold to about 100-fold, about 50-fold to about 100-fold, about 50-fold to about 2000-fold, about 100-fold to about 2000-fold, or of at least about 500-fold to about 2000-fold, compared to an IL12 fusion protein comprising unmodified p40 domain that does not contain the one or more amino acid modification disclosed herein.

In some embodiments, an IL12 fusion protein (e.g., an IL12 HetFc fusion protein) comprising a modified p40 domain of this disclosure can have a reduction in binding affinity for at least one of its cognate receptors (e.g., IL12Rβ1) from about 5-fold to about 1000-fold, from about 5-fold to about 800-fold, from about 5-fold to about 600-fold, from about 10-fold to about 500-fold, from about 10-fold to about 300-fold, or from about 20-fold to about 200-fold, relative to the binding affinity of an IL12 fusion protein comprising an unmodified wildtype p40 domain which sequence is set forth in SEQ ID NO: 10, and as determined in a reporter gene assay.

In some embodiments, an IL12 fusion protein (e.g., an IL12 HetFc fusion protein) comprising a modified p40 domain can have a reduction in binding affinity for at least one of its cognate receptors (e.g., IL12Rβ1) of at least about 20-fold to about 200-fold, compared to an IL12 fusion protein comprising unmodified p40 domain that does not contain the one or more amino acid modification disclosed herein.

### Masked IL12 Fusion Protein Configurations

“Masked IL12 fusion protein” as used herein is specifically meant to include fusion proteins described herein comprising any cytokine from the IL12 family of heterodimeric cytokines and therefore, is meant specifically to include IL12 and IL23 masked fusion proteins.

In certain places, “masked cytokine fusion protein” may be used and is similarly meant to include masked IL12 or IL23 fusion proteins. Additionally, the masked IL12 fusion proteins may be referred to herein as “masked HetFc IL12 fusion proteins” as the fusion proteins are in some embodiments made with the modified Fc polypeptides described herein. The terminology “masked IL12 fusion protein” and “masked cytokine fusion protein” also are meant to include any masked HetFc IL12 fusion proteins.

It should be noted that the numbering of the linkers is for clarity only and the numbers are interchangeable. Any given linker may have a different number depending on the configuration or geometry. L1 in one geometry is not necessarily the same linker as L1 in a different geometry. Moreover, similar geometries may number the linkers differently.

In certain embodiments, an IL12 fusion protein or masked IL12 fusion protein containing a modified p40 domain as described herein demonstrates a complete reduction in potency of the IL12 polypeptide in that IL12 activity is undetected by, e.g., an NK or other cell-based assay. In this case, the “fold reduction in potency” cannot be calculated as activity is below the limit of detection.

Methods for measuring binding or functional IL12 activity are known in the art and described herein. In certain embodiments, binding activity can be measured using surface plasmon resonance (SPR). Functional IL12 activity can be measured, for example, in an NK cell relative abundance or CD8+ T cell IFNγ release assay (see also, e.g., Example 3).

Thus, in certain embodiments, provided herein are IL12 fusion proteins and masked IL12 fusion proteins containing modified p40 domains that exhibit at least 5-fold, 10-fold, 15-fold, 20-fold, 30-fold, 40-fold, 50-fold, 100-fold, 200-fold, 300-fold, 400-fold, 500-fold, 600-fold, 700-fold, 800-fold, 900-fold, 1000-fold, 1200-fold, 1500-fold, 2000-fold, 2500-fold, 3000-fold, or further reduced binding activity, functional IL12 activity, or potency as compared to an appropriate control, as measured by SPR, NK cell, CD8+ T cell IFNγ release, or other appropriate assay.

In some embodiments, the IL12 polypeptides used in the fusion proteins described herein can comprise a modified p40 domain as described herein, e.g., a p40 domain sequence comprising any one or more of the substitutions listed in Tables B and C. Such modified p40 domains can have a reduced binding to a receptor of IL12, compared to an unmodified (e.g., WT) p40 domain. In various embodiments, such modified p40 domain can comprise or consist of any one of the amino acid sequences set forth in SEQ ID NOS: 12-57, or a sequence having at least about 90%, 95%, 97%, or at least about 99% sequence identity thereto. In other embodiments, such modified p40 domain can comprise or consist of the amino acid sequences set forth in SEQ ID NOS: 12-14, 16, 18, 19, 21-34, 36-56, respectively, or a sequence having at least about 90%, 95%, 97%, or at least about 99% sequence identity thereto. In yet other embodiments, such modified p40 domain can comprise or consist of the amino acid sequences set forth in SEQ ID NOS: 12, 13, 19, 20, 24, 27, 30, 34, 38, 39, 41, 47, 48, 49, 51, 52, 54, 55, 56, 57, 42, 44, 45, 46, 50, 53, 40 and 43, respectively, or a sequence having at least about 90%, 95%, 97%, or at least about 99% sequence identity thereto.

### IL12 Family of Cytokines

The present disclosure provides IL12 fusion proteins. Interleukin 12 (IL12) was the first recognized member of a family of heterodimeric cytokines that includes IL12, IL23, IL27, IL35 and IL39. IL12 and IL23 are pro-inflammatory cytokines important for development of T helper 1 (Th-1) and T helper 17 (Th-17) T cell subsets, while IL27 and IL35 are potent inhibitory cytokines. IL39 is an important cytokine in regulating innate and/or adaptive immune response. IL12 can directly enhance the activity of effector CD4 and CD8 T cells as well as natural killer (NK) and NK T cells.

Interleukin-12 (IL12) is a heterodimeric molecule composed of an alpha chain (the p35 subunit) and a beta chain (the p40 subunit) covalently linked by a disulfide bridge to form the biologically active 70 kDa dimer. Exemplary amino acid sequences of mature p35 and p40 subunits of IL12 are provided in Table M. See SEQ ID NOS: 10 and 11 and variants thereof, such as, variants of the p40 subunit comprising amino acids substitutions to reduce affinity for IL12Rβ1 (SEQ ID NO: 12-57).

IL23 is a member of IL12 cytokine family and is also composed of two subunits: the p40 subunit that it shares with IL12 and p19. Exemplary amino acid sequence of the p19 subunit of IL23 is provided in Table M. See SEQ ID NOS: 127 and 128. The receptor for IL23 (IL23R) consists of an IL23R subunit and an IL12Rβ1 subunit, which is a common subunit for the IL12 receptor and interacts with Tyrosine kinase 2 (Tyk2). The IL23R is mainly expressed on immune cells, in particular T cells (e.g., Th17 and gamma delta T cells), macrophages, dendritic cells and NK cells (Duvallet et ah, 2011). It has been recently shown that non-activated neutrophils express a basal amount of IL23R and that IL23R expression is increased upon cell activation (Chen et al., 2016).

The term “a protein having the function of IL12” or “a protein having the function of IL23” encompasses mutants of a wild type IL12 or IL23 sequence, respectively, wherein the wild type sequence has been altered by one or more of addition, deletion, or substitution of amino acids. IL12 and IL23 sequences contemplated herein include IL12 and IL23 sequences from any animal, in particular any mammal, including human, mouse, dog, cat, pig, and non-human primate. By “wild type,” “wildtype” or “WT” herein is meant an amino acid sequence or a nucleotide sequence that is found in nature, including allelic variations. A WT protein has an amino acid sequence or a nucleotide sequence that has not been intentionally modified.

The bioactivities of IL12 are well known and include, without limitation, differentiation of naive T cells into Thl cells, stimulation of the growth and function of T cells, production of interferon-gamma (IFN-gamma) and tumor necrosis factor-alpha (TNF-a) from T and natural killer (NK) cells, reduction of IL4 mediated suppression of IFN-gamma, enhancement of the cytotoxic activity of NK cells and CD8⁺ cytotoxic T lymphocytes, stimulation of the expression of IL12Rβ1 and IL12Rβ2, facilitation of the presentation of tumor antigens through the upregulation of MHC I and II molecules, and anti-angiogenic activity. IL12 is produced primarily by antigen-presenting cells and drives cell-mediated immunity by binding to a two-chain receptor complex that is expressed on the surface of T cells or natural killer (NK) cells. The IL12 receptor beta-1 (IL12Rβ1) chain binds to the p40 subunit of IL12. IL12p35 ligation of the second receptor chain, IL12Rβ2, confers intracellular signaling (e.g. STAT4 phosphorylation) and activation of the receptor-bearing cell (Presky et al, 1996). Studies show equal cell-based affinity of IL12 for Rol and R132 individually, and higher affinity for the complex (J Immunol. 1998 Mar. 1; 160(5):2174-9). IL12 also acts on dendritic cells (DC), leading to increased maturation and antigen presentation, which can allow for the initiation of a T cell response to tumor specific antigens. It also drives the secretion of IL12 by DCs, creating a positive feedback mechanism to amplify the response.

Exemplary nucleic acid and amino acid sequences for the IL12, IL23 and the masked fusion proteins described herein are provided in Table M.

Variants of any of the nucleic acid and amino acid sequences provided herein are also contemplated for use in the fusion proteins as described herein in the section entitled “Polypeptides and Polynucleotides”. In certain embodiments, the IL12 fusion protein polypeptides described herein comprise a p35 amino acid sequence as set forth in SEQ ID NO: 11. In certain embodiments, the IL12 fusion proteins described herein comprise a p40 amino acid sequence as set forth in SEQ ID NO: 10. In another embodiment, the IL12 fusion polypeptides described herein comprise a p35 amino acid sequence as set forth in SEQ ID NO: 11 and a p40 amino acid sequence as set forth in SEQ ID NO: 10. In one embodiment, the IL12 fusion proteins described herein comprise a scIL12 having the configuration p40-L-p35, wherein L is a linker moiety, e.g., a peptide-based linker as described herein. In other embodiments, the IL12 polypeptides described herein may comprise a variant of the p35 and/or p40 sequence. In this regard, the variant may comprise a variant of the nucleic acid sequence encoding the p35 or p40 amino acid sequence where the variant encodes a protein that retains IL12 functional activity as compared to the wild type IL12, or other appropriate control. A variant nucleic acid sequence may comprise at least 50%, 55%, 60%, 65%, 70%, 75%, 80%, 85%, 86%, 87%, 88%, 89%, 90%, 91%, 92%, 93%, 94%, 95%, 96%, 97%, 98%, 99%, or higher % identity to the polynucleotide sequence encoding p35 and/or p40, such as the polynucleotide sequences set forth in SEQ ID NOS: 129 and 130. Illustrative variants of the IL12 polynucleotides include codon optimized polynucleotide sequences.

In certain embodiments, a variant may comprise a variant p35 and/or p40 polypeptide comprising at least 50%, 55%, 60%, 65%, 70%, 75%, 80%, 85%, 86%, 87%, 88%, 89%, 90%, 91%, 92%, 93%, 94%, 95%, 96%, 97%, 98%, 99%, or higher % identity to the amino acid sequence of IL12 p35 and/or p40 as set forth in SEQ ID NOS: 11 and 10, respectively, where such variant polypeptides retain IL12 functional activity as compared to an appropriate comparator molecule comprising a wild type IL12.

In other embodiments, the IL23 polypeptides described herein may comprise a variant of the p19 and/or p40 sequence. In this regard, the variant may comprise a variant of the nucleic acid sequence encoding the p19 or p40 amino acid sequence, where the variant encodes a protein that retains IL23 functional activity as compared to the wild type IL23. A variant nucleic acid sequence may comprise at least 50%, 55%, 60%, 65%, 70%, 75%, 80%, 85%, 86%, 87%, 88%, 89%, 90%, 91%, 92%, 93%, 94%, 95%, 96%, 97%, 98%, 99%, or higher % identity to the polynucleotide sequence encoding p19 and/or p40 as set forth in SEQ ID NOS: 131 and 130, respectively. Illustrative variants of the IL23 polynucleotides include codon optimized polynucleotide sequences.

By reduced or inhibited binding or activity it is meant that binding or functional IL12 activity is lower than the binding or functional IL12 activity of an appropriate control, such as wild type IL12, or a corresponding unmasked parental fusion protein. The reduced or inhibited binding or activity can be expressed as reduced potency. In certain embodiments, the potency of an IL12 fusion protein with a modified p40 domain is reduced by about 2-fold to about 2500-fold as compared to the IL12 activity of an appropriate control, such as IL12 fusion proteins with wild-type p40 domains. The potency of an IL12 fusion protein with a modified p40 domain as described herein is in certain embodiments reduced by about 5-fold to about 2000-fold, by about 10-fold to about 1500-fold, by about 15-fold to about 1000-fold, by about 20-fold to about 800-fold, by about 25-fold to about 600-fold, by about 25-fold to about 100-fold, by about 50-fold to about 100-fold, by about 50-fold to about 2000-fold, by about 100-fold to about 2000-fold, or by about 500-fold to about 2000-fold, as compared to an unmodified p40 domain. In some embodiments, the potency of an IL12 fusion protein with a modified p40 domain as described herein is reduced by about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2500, or 3000-fold, as compared to an unmodified p40 domain. In certain embodiments, potency is reduced by more than 3500, 4000, 4500, 5000, 5500, 6000, 7000, 8000, 9000 or 10,000-fold, as compared to an unmodified p40 domain. In some embodiments, a modified p40 domain comprising one or more amino acid modifications as described herein, or a fusion protein comprising such modified p40 domain, can have a reduced binding of at least about 2-fold to at least about 2500-fold, as compared to an unmodified p40 domain.

In certain embodiments, a variant may comprise a variant p19 and/or p40 polypeptide comprising at least 50%, 55%, 60%, 65%, 70%, 75%, 80%, 85%, 86%, 87%, 88%, 89%, 90%, 91%, 92%, 93%, 94%, 95%, 96%, 97%, 98%, 99%, or higher % identity to the amino acid sequence of IL23 p19 and/or p40 as set forth in SEQ ID NOS: 128 and 10, respectively, where such variant polypeptides retain IL23 functional activity as compared to the wild type IL23.

The variant cytokine polypeptides or fusion proteins comprising them as described herein, exhibit functional activity that is within 2 to 20-fold of the functional activity (e.g., IL12 or IL23) of an appropriate control, e.g., a relevant comparator fusion protein comprising a wild type cytokine (e.g., IL12 or IL23). In certain embodiments, cytokine variant polypeptides demonstrate equivalent potency as compared to wild type controls, e.g., as measured by relative abundance of NK cells, IFNγ release by CD8+ T cells, or cell signaling following receptor engagement. In other embodiments, cytokine variant polypeptides demonstrate a maximum attenuation of potency of between about 2-fold and about 20-fold, or between about 20-fold and about 200-fold. In certain embodiments, cytokine variant polypeptides or fusion proteins comprising them demonstrate attenuation of potency of between about 2-fold, 3, 4, 5, 6, 7, 8, 9, 10, 11, 12, 13, 14, 15, 16, 17, 18, 19 or about 20-fold. As noted elsewhere, IL12 is highly toxic. Accordingly, it may be desirable in certain embodiments to use a variant IL12 polypeptide having reduced potency. In certain embodiments, a variant may exhibit decreased functional activity or decreased potency as compared to the control, e.g., between about 2-fold and about 100-fold, or about 2-fold, 3-fold, 4-fold, 5-fold, 10-fold, 15-fold, 20-fold, 30-fold, 40-fold, 50-fold, 60-fold, 70-fold, 80-fold, 90-fold, 100-fold or 200-fold decreased activity or potency as compared to an appropriate control. Cytokine functional activity can be measured using assays known in the art and described herein such as a splenocyte, NK or CTLL-2 assay or IFNγ release by CD8+ T cells.

Methods of measuring the functional activity of IL12 family cytokines are known in the art. Such methods include assays known in the art, such as assays to determine cell responsiveness to IL12 or IL23, measuring cytokine production in response to incubating appropriate cells with IL12 or IL23, measuring receptor binding and signaling activation.

In certain embodiments, IL12 activity is determined by measuring cell proliferation of cells or cell lines that are sensitive to IL12. Illustrative cells that can be used to test IL12 activity include CTLL-2 or NK or CD8 cells. Such proliferation assays include assays as described, for example, by Khatri A, et al. 2007. J Immunol Methods 326(1-2):41-53; Puskas J, et al. 2011. Immunology 133(2):206-220; Hodge D L., et al. J Immunol. 2002 Jun. 15; 168(12):6090-8. Assays known in the art can be modified as desired to fit the particular cytokine being tested, such as IL12 or IL23.

In brief, a CTLL-2 assay for measuring IL12 functional activity may comprise serially diluting the recombinant proteins to be tested (e.g., a masked fusion protein as described herein) 1:5 in 50 μL of medium, then 4×10⁴ CTLL-2 cells in 100 μL of medium are added per well to a 96-well plate and incubated at 37° C. in 5% CO₂ for 18-22 h. At the end of this period, 75 μg/well of Thiazolyl Blue Tetrazolium Bromide (MTT; Sigma-Aldrich) is added and the plate is incubated for 8 h at 37° C. in 5% CO₂. Cells are lysed with 100 μL/well of 10% SDS (Gibco) acidified with HCl, incubated at 37° C. in 5% CO₂ overnight, and absorbance is read at 570 nm.

In brief, an NK assay for measuring IL12 functional activity can be carried out as follows: NK cells are cultured in growth medium without IL2 (assay media) for 12 hours, harvested and spun down to pellet cells. Cells are resuspended in assay media to 400 million cells/mL and 10,000 cells or 25 uL per well are added to assay plates. Variant test samples are titrated in triplicate at 1:5 dilution in 25 ul directly in 384-well black flat bottom assay plates. Recombinant cytokine (e.g., human IL12 (Peprotech, Rocky Hill, NJ)) is included as a positive control. Plates are incubated for 3 days at 37° C. and 5% carbon dioxide. Post incubation, 25 uL/well of supernatant is transferred to non-binding 384-well plates (Greiner-Bio-One, Kremsmunster, Austria) and stored at −80° C. After supernatant removal, CellTiter-Glo® Luminescent Cell Viability reagent (Promega, Madison, WI) or equivalent reagent can be added to plates at 25 uL/well and plates are incubated at room temperature away from light for 30 minutes. Following incubation, plate luminescence is scanned, such as on a BioTek synergy H1 plate reader (BioTek, Winooski, VT).

In one embodiment, IL12 activity can be determined by measuring cell signaling cascades triggered by IL12 interaction with its receptor (e.g., IL12Rβ2 and IL12Rβ1 interaction with IL12 p35-p40 heterodimers). In one embodiment, IL12 activity is determined by measuring STAT4 signaling activity using assays known in the art and commercially available for example, from Abeomics, San Diego, CA USA.

In one embodiment, IL12 activity can be determined by measuring IFNγ release from CD8+ T cells after stimulation with IL12 proteins, as described in Example 3.

### Masking Moieties

The masked IL12 or IL23 fusion proteins described herein comprise a masking moiety (MM) that blocks or reduces the binding of IL12 or IL23 to its native receptor(s) and/or blocks or reduces its functional activity. In some embodiments, a masked IL12 or IL23 fusion protein described herein comprises (i) a first fusion polypeptide comprising a first Fc polypeptide C-terminally fused to an IL12 polypeptide that comprises a modified p40 domain, and (ii) a second fusion polypeptide comprising a second Fc polypeptide C-terminally fused to the MM, wherein the two Fc polypeptides form the dimeric Fc domain complex (e.g., a heterodimeric Fc domain).

In certain embodiments, the MM specifically binds to the IL12. “Specifically binds”, “specific binding” or “selective binding” means that the binding is selective for the desired antigen (in the case of the present disclosure, the MM specifically binds IL12 or IL23) and can be discriminated from unwanted or non-specific interactions. The ability of a MM to bind to and block or reduce IL12/IL23 activity can be measured either through an enzyme-linked immunosorbent assay (ELISA) or other techniques familiar to one of skill in the art, e.g. surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of a MM to an unrelated protein is less than about 10% of the binding of the MM to IL12/IL23 as measured, e.g., by SPR. In certain embodiments, MM that binds to IL12/IL23 or a biologically active fragment thereof, has a dissociation constant (K_{d}) of <1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹³ M).

The MM of the present disclosure generally refers to an amino acid sequence present in the masked cytokine fusion protein and positioned such that it reduces the ability of the cytokine, within the context of the masked cytokine fusion protein, to specifically bind its target and/or to function. In some cases, the MM is coupled to the masked cytokine fusion protein by way of a linker.

When an IL12 fusion protein described herein comprises a MM and is in the presence of the target (e.g., an IL12 receptor), specific binding of the masked IL12 fusion protein to the IL12 receptor is reduced or inhibited as compared to specific binding of the non-masked parental IL12 fusion protein.

When an IL12 fusion protein described herein comprises a MM and is in the presence of the target (e.g., an IL12 receptor), the potency of the masked IL12 fusion protein is reduced or inhibited as compared to the non-masked parental IL12 fusion protein. Thus, the MM functions to block functional activity of the IL12.

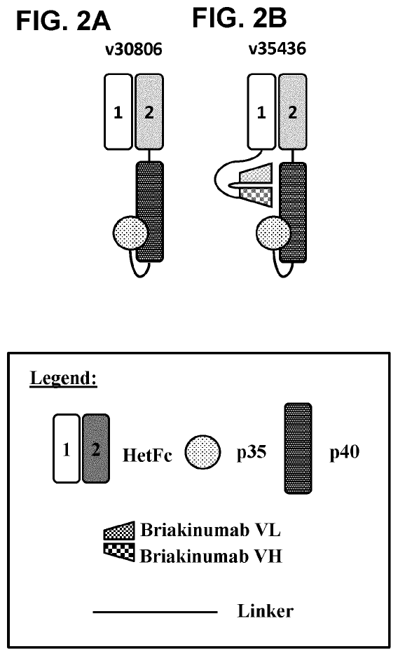

In certain embodiments of IL12 HetFc fusion proteins, as further described herein, the MM can be coupled to a first Fc polypeptide of a dimeric (e.g., heterodimeric) Fc domain, either directly or via a linker, and the IL12 polypeptide can be coupled to a second Fc polypeptide of the dimeric Fc domain, either directly or via a linker. In such configuration, e.g., as illustrated in FIG. 2B herein, the MM can specifically interact or bind to the IL12 moiety of the fusion protein. Such binding can comprise or consist of non-covalent binding. Interaction of the MM with the IL12 within the fusion protein can mask the IL12 by inhibiting or at least reducing its ability to interact with at least one of its cognate receptors, when compared to a corresponding “unmasked” IL12 polypeptide. Such masking activity of the MM can further inhibit or reduce any downstream events that are mediated by a receptor that was activated by IL12, and thus MM activity can be measured by various methods described herein and known in the art, e.g., enzyme-linked immunosorbent assays (ELISA) or other techniques familiar to one of skill in the art and described herein.

In certain embodiments, the dissociation constant (K_{d}) of the masked IL12 fusion proteins herein towards an IL12 receptor is generally greater than the K_{d} of the same IL12 fusion protein that does not contain a MM. Conversely, the binding affinity of the masked IL12 fusion proteins towards an IL12 receptor is generally lower than the binding affinity of the IL12 fusion protein not modified with a MM.

In certain embodiments, the K_{d} of the MM towards the IL12 polypeptide is generally greater than the K_{d} of the IL12 polypeptide towards an IL12 receptor. Conversely, in certain embodiments, the binding affinity of the MM towards the IL12 polypeptide is generally lower than the binding affinity of the IL12 polypeptide towards an IL12 receptor.

It should be noted that due to proximity (that is, when the MM is fused by a linker to the IL12 fusion protein), the apparent “affinity” of the MM for the IL12 polypeptide is greater than when the MM is not fused to the IL12 fusion protein.

The MM can inhibit the binding of the masked IL12 fusion protein to the IL12 receptor and thereby inhibit the IL12 functional activity of the fusion protein as compared to the IL12 polypeptide not modified by the MM. The MM can bind to the IL12 polypeptide and inhibit it from binding to its receptor. The MM can sterically inhibit the binding of the masked IL12 fusion protein to the IL12 receptor. The MM can allosterically inhibit the binding of the masked IL12 fusion protein to the IL12 receptor. In those embodiments when the masked IL12 fusion protein is in the presence of the IL12 receptor, there is no binding or substantially no binding of the masked IL12 fusion protein to the IL12 receptor, or no more than 0.001 percent, 0.01 percent, 0.1 percent, 1 percent, 2 percent, 3 percent, 4 percent, 5 percent, 6 percent, 7 percent, 8 percent, 9 percent, 10 percent, 15 percent, 20 percent, 25 percent, 30 percent, 35 percent, 40 percent, or 50 percent binding of the masked IL12 fusion protein to the target, as compared to the binding of the unmasked IL12 fusion protein, the binding of the parental IL12, for at least 2, 4, 6, 8, 12, 28, 24, 30, 36, 48, 60, 72, 84, 96 hours, or 5, 10, 15, 30, 45, 60, 90, 120, 150, 180 days, or 1, 2, 3, 4, 5, 6, 7, 8, 9, 10, 11, 12 months or greater when measured in vivo or by Surface Plasmon Resonance (SPR).

The MM can inhibit the binding of the masked IL12 fusion protein to the IL12 receptor and thereby inhibit the IL12 functional activity of the fusion protein as compared to the IL12 polypeptide not modified by the MM. The MM can bind to the IL12 polypeptide and inhibit it from binding to its receptor. The MM can sterically inhibit the binding of the masked IL12 fusion protein to the IL12 receptor. The MM can allosterically inhibit the binding of the masked IL12 fusion protein to the IL12 receptor. In those embodiments when the masked IL12 fusion protein is in the presence of the IL12 receptor, there is no binding or substantially no binding of the masked IL12 fusion protein to the IL12 receptor, or no more than 0.001 percent, 0.01 percent, 0.1 percent, 1 percent, 2 percent, 3 percent, 4 percent, 5 percent, 6 percent, 7 percent, 8 percent, 9 percent, 10 percent, 15 percent, 20 percent, 25 percent, 30 percent, 35 percent, 40 percent, or 50 percent binding of the masked IL12 fusion protein to the target, as compared to the binding of the unmasked IL12 fusion protein, the binding of the parental IL12, for at least about 2, 4, 6, 8, 12, 28, 24, 30, 36, 48, 60, 72, 84, 96 hours, or more, when measured in an in vitro assay such as a reporter gene assay (see, e.g., Example 4).

In certain embodiments the MM is not a natural binding partner of the IL12 polypeptide. The MM may be a modified binding partner for the IL12 polypeptide which contains amino acid changes that at least slightly decrease affinity and/or avidity of binding to the IL12 polypeptide. In some embodiments the MM contains no or substantially no homology to the IL12 receptor. In other embodiments the MM is no more than 5 percent, 10 percent, 15 percent, 20 percent, 25 percent, 30 percent, 35 percent, 40 percent, 45 percent, 50 percent, 55 percent, 60 percent, 65 percent, 70 percent, 75 percent, or 80 percent similar to an IL12 receptor.

When the IL12 fusion protein is in a ‘masked’ state, even in the presence of the IL12 receptor, the MM interferes with or inhibits the binding of the masked IL12 fusion protein to the receptor.

In various embodiments, the MM of a masked IL12 fusion protein herein interacts with the IL12 polypeptide in a non-covalent and reversible manner. Hence, in certain embodiments, described herein are masked IL12 fusion proteins which can comprise a masked IL12 polypeptide having an inhibited or reduced IL12 receptor binding affinity, and which can become unmasked under certain conditions in the in vitro or in vivo environment, e.g., pH, protease activity, etc., and as further described herein. Thus, in some embodiments, a masked IL12 fusion protein herein can be temporarily masked and become unmasked after a certain period of time, e.g., under certain conditions, such as pH or protease activity, that may be present in certain tissues or organs of a mammalian subject, e.g., a tumor microenvironment. The conditionally unmasked IL12 fusion protein can elicit its IL-12 activity which is generally comparable to the IL12 activity of an inherently unmasked IL12 fusion protein. However, since the IL12 polypeptides used in the fusion protein herein comprise a modified p40 domain, the IL12 functional activity of IL12 fusion protein that is unmasked under certain conditions may still be reduced, compared to a fusion protein that uses an unmasked wildtype IL12 polypeptide.

The structural properties of the MM will vary according to a variety of factors such as the minimum amino acid sequence required for interference with cytokine binding and/or activity, the cytokine-cytokine receptor protein binding pair of interest, the size of the cytokine and the fusion protein, the length of the protease cleavable linker (PCL), whether the PCL is positioned within the MM, between the Fc and the cytokine, between the Fc and mask, the presence or absence of additional linkers, etc.

The MM can be provided in a variety of different forms. In certain embodiments, the MM can be selected to be a known binding partner of the cytokine. In certain embodiments, the MM is one that masks the cytokine from target binding when the MM is covalently linked in the masked cytokine fusion protein but does not substantially or significantly interfere or compete for binding of the target with the cytokine polypeptide when the MM is not covalently linked in the cytokine fusion protein. In a specific embodiment, the MM do not contain the amino acid sequences of a naturally-occurring binding partner of the cytokine.

The efficiency of the MM to inhibit the binding or activity of the cytokine when coupled can be measured by SPR or a cell based assay as described herein and outlined in detail elsewhere (see e.g., NK, CTLL-2 or CD8+ T cell IFNγ release assays) and as described herein in the Examples section of the disclosure. Masking efficiency of MMs can be determined by at least two parameters: affinity of the MM for the cytokine or a fusion protein comprising the cytokine and the spatial relationship of the MM relative to the binding interface of the cytokine to its receptor.

Regarding affinity, by way of example, a MM may have high affinity but only partially inhibit the binding of the cytokine to its receptor, while another MM may have a lower affinity for the cytokine but fully inhibit target binding. For short time periods, the lower affinity MM may show sufficient masking; in contrast, over time, that same MM may be displaced by the target (due to insufficient affinity for the cytokine).

In a similar fashion, two MMs with the same affinity may show different extents of masking based on how well they promote inhibition of the cytokine from binding its receptor. In another example, a MM with high affinity may bind and change the structure of the cytokine or a fusion protein comprising the cytokine so that binding to its target is completely inhibited while another MM with high affinity may only partially inhibit binding. As a consequence, discovery of an effective MM is generally not based only on affinity but can include a measure of the potency of the masked cytokine fusion protein as compared to an appropriate control.

As described herein, in various embodiments, a MM of an IL12 fusion protein can comprise or consist of an antibody or antigen-binding fragment thereof, that specifically binds to IL12. Thus, in some embodiments, the MM may be a single-chain Fv (scFv) antibody fragment. Illustrative scFv MM comprise the VH and VL amino acid sequences provided in SEQ ID NOS: 2-3. In certain embodiments, illustrative MM comprise the VHCDR and VLCDR set forth in SEQ ID NOS:4-9.

### Antibodies and Antigen-Binding Fragments Thereof

In certain embodiments, the masking moieties used in the masked fusion proteins herein comprise an antibody or an antigen-binding fragment of an antibody. Antigen-binding fragments include but are not limited to variable or hypervariable regions of light and/or heavy chains of an antibody (V_{L}, VH), variable fragments (Fv), Fab′ fragments, F(ab′) 2 fragments, Fab fragments, single chain antibodies (scAb), single chain variable regions (scFv), complementarity determining regions (CDR), domain antibodies (dAbs), single domain heavy chain immunoglobulins, single domain light chain immunoglobulins, or other polypeptides known in the art containing an antigen-binding fragment capable of binding target proteins or epitopes on target proteins.

Illustrative antigen-binding domains are derived from antibodies that bind IL12 and/or IL23.

In one embodiment, the MM comprises an antibody or antigen-binding fragment thereof, that specifically binds to IL12. In one embodiment, the MM comprises an antibody or antigen-binding fragment thereof, that specifically binds to IL23. In certain embodiments, the MM comprises an scFv that specifically binds IL12 or IL23.

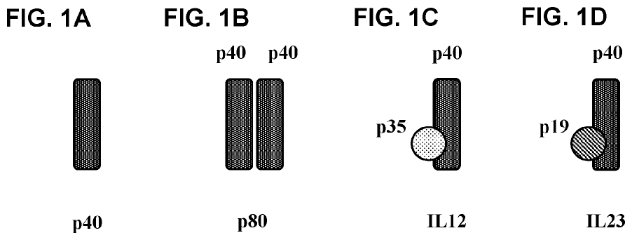

In some embodiments the MM can be identified through screening antibodies or antigen binding fragments thereof that bind to IL12 or IL23. The candidate MM can be fused in a variety of configurations in a cytokine fusion protein (see for example FIGS. 1A-B and the Examples herein) and screened for their ability to reduce cytokine binding, reduce IL12 potency and/or for recovery of cytokine activity after cleavage. Antibodies may be derived from antibodies known in the art that bind to IL12 and/or IL23. Such antibodies are known and available for example, from the literature or can be found in the TABS Therapeutic Antibody Database (see tabs(dot)craic(dot)com). Illustrative antibodies for use in the masked IL12 fusion proteins herein include Briakinumab (U.S. Pat. Nos. 6,914,128; 7,504,485; 8,168,760; 8,629,257; 9,035,030); ustekinumab (U.S. Pat. Nos. 6,902,734; 7,279,157; 8,080,247; 7,736,650; 8,420,081; 7,887,801; 8,361,474; 8,084,233; 9,676,848), AK101, PMA204 (see e.g., U.S. Pat. No. 8,563,697), 6F6 (see e.g., U.S. Pat. No. 8,563,697; Clarke A W et al., 2010 MAbs 2:539-49). The h6F6 antibody binds a different epitope on p40 than Briakinumab or Ustekinumab.

In one embodiment, the MM is derived from an antibody comprising an antigen binding domain that binds to human IL12 and human IL23. In another embodiment, the antibody binds human IL12p40 existing as a monomer (human IL12p40) and as a homodimer (human IL12p80) and the antibody inhibits the binding of human IL12 to human IL12Rβ2 and human IL23 to human IL23R but does not inhibit the binding of human IL12 or human IL23 or human IL12p40 or human IL12p80 to human IL12Rβ1.

Antibodies or antigen binding fragments thereof that bind to IL12 and/or IL23, can be further modified to increase or decrease affinity as needed and then further tested for ability to mask and reduce potency as described herein.

In certain embodiments, candidate peptides can be screened to identify a MM peptide capable of binding IL12 or IL23 using such methods as described for example in WO2010/081173 and U.S. Pat. No. 10,118,961. Such methods comprise, providing a library of peptide scaffolds, wherein each peptide scaffold comprises: a transmembrane protein (TM); and a candidate peptide; contacting an IL12 or IL23 with the library; identifying at least one candidate peptide capable of binding the IL12 or IL23 polypeptide; and determining whether the dissociation constant (K_{d}) of the candidate peptide towards the IL12 or IL23 is between 1-10 nM.

In various embodiments, a MM of an IL12 fusion protein comprises or consists of a single-chain variable fragment (scFv) of an antibody. In such embodiments, the scFv MM can comprise a heavy chain variable domain (V_{H}) comprising the complementarity determining region (CDR) sequences set forth in SEQ ID NOS:4-6, and a light chain variable domain (V_{L}) comprising the complementarity determining region (CDR) sequences set forth in SEQ ID NOS:7-9. Hence, in some embodiments, the scFv MM can comprise or consist of a V_{H} domain comprising or consisting of an amino acid sequence having at least about 90%, 95%, 97%, 99% or 100% sequence identity to the amino acid sequence set forth in SEQ ID NO: 2, and V_{L} domain comprising or consisting of an amino acid sequence having at least about 90%, 95%, 97%, 99% or 100% sequence identity to the amino acid sequence set forth in SEQ ID NO: 3.

### Linkers and Protease Cleavable Linkers

In certain embodiments of the fusion proteins of this disclosure, one or more different components or domains are fused directly one to the other with no linker. For example, in certain embodiments, an Fc domain may be fused directly to a MM or fused directly to a p35 or p40 polypeptide. However, in certain embodiments the masked cytokine fusion constructs comprise one or more linkers of varying lengths. Peptide linkers allow arrangement of the fusion protein to form a functional masking moiety as well as a cytokine that, when cleaved from the larger/full fusion protein, retains cytokine activity.

A “linker” is a peptide that joins or links other peptides or polypeptides, such as a linker of about 2 to about 150 amino acids. A peptide linker of the present disclosure can comprise or consist of an amino acid sequence of about 2 to about 150 amino acids, about 5 to about 100 amino acids, about 5 to about 75 amino acids, about 5 to about 50 amino acids, about 5 to about 40 amino acids, about 5 to about 30 amino acids, or about 5 to about 20 amino acids. In masked cytokine fusion proteins of this disclosure, a linker may be used to fuse any of the components of the fusion protein, such as an Fc polypeptide to a MM or a linker can join an Fc polypeptide to a cytokine polypeptide, e.g., p35 or p40 of IL12. In certain embodiments, a linker may be present within a MM such as where a MM is an scFv and a linker joins the VH and VL.

Exemplary linkers for use in the fusion proteins described herein include those belonging to the (GlyₙSer) family, such as (Gly₃Ser)ₙ(Gly₄Ser)₁, (Gly₃Ser)₁(Gly₄Ser)ₙ, (Gly₃Ser)ₙ(Gly₄Ser)ₙ, or (Gly₄Ser)ₙ, wherein n is an integer of 1 to 5. In certain embodiments, the peptide linkers suitable for connecting the different domains include sequences comprising glycine-serine linkers, for example, but not limited to, (GₘS)ₙ-GG, (SGn)m, (SEGn)m, wherein m and n are between 0-20.

In certain embodiments, a linker can be an amino acid sequence obtained, derived, or designed from an antibody hinge region sequence, a sequence linking a binding domain to a receptor, or a sequence linking a binding domain to a cell surface transmembrane region or membrane anchor. In some embodiments, a linker can have at least one cysteine capable of participating in at least one disulfide bond under physiological conditions or other standard peptide conditions (e.g., peptide purification conditions, conditions for peptide storage). In certain embodiments, a linker corresponding or similar to an immunoglobulin hinge peptide retains a cysteine that corresponds to the hinge cysteine disposed toward the amino-terminus of that hinge. In further embodiments, a linker is from an IgG1 hinge and has been modified to remove any cysteine residues or is an IgG1 hinge that has one cysteine or two cysteines corresponding to hinge cysteines.

In addition to providing a spacing function, a linker can provide flexibility or rigidity suitable for properly orienting the one or more domains of the unmasked or masked cytokine fusion proteins herein, both within the fusion protein and between or among the fusion proteins and their target(s). Further, a linker can support expression of a full-length fusion protein and stability of the purified protein both in vitro and in vivo following administration to a subject in need thereof, such as a human, and is preferably non-immunogenic or poorly immunogenic in those same subjects. In certain embodiments, a linker may comprise part or all of a human immunoglobulin hinge, a stalk region of C-type lectins, a family of type II membrane proteins. Linkers range in length from about 2 to about 100 amino acids, or about 5 to about 75 amino acids, or about 10 to about 50 amino acids, or about 2 to about 40 amino acids, or about 8 to about 20 amino acids, about 10 to about 60 amino acids, about 10 to about 30 amino acids, or about 15 to about 25 amino acids.

In certain embodiments, a linker for use herein may comprise an “altered wild type immunoglobulin hinge region” or “altered immunoglobulin hinge region”. Such altered hinge regions refers to (a) a wild type immunoglobulin hinge region with up to 30 percent amino acid changes (e.g., up to 25 percent, 20 percent, 15 percent, 10 percent, or 5 percent amino acid substitutions or deletions), (b) a portion of a wild type immunoglobulin hinge region that is at least 10 amino acids (e.g., at least 12, 13, 14 or 15 amino acids) in length with up to 30 percent amino acid changes (e.g., up to 25 percent, 20 percent, 15 percent, 10 percent, or 5 percent amino acid substitutions or deletions), or (c) a portion of a wild type immunoglobulin hinge region that comprises the core hinge region (which portion may be 4, 5, 6, 7, 8, 9, 10, 11, 12, 13, 14, or 15, or at least 4, 5, 6, 7, 8, 9, 10, 11, 12, 13, 14, or 15 amino acids in length). In certain embodiments, one or more cysteine residues in a wild type immunoglobulin hinge region, such as an IgG1 hinge comprising the upper and core regions, may be substituted by one or more other amino acid residues (e.g., one or more serine residues). An altered immunoglobulin hinge region may alternatively or additionally have a proline residue of a wild type immunoglobulin hinge region, such as an IgG1 hinge comprising the upper and core regions, substituted by another amino acid residue (e.g., a serine residue).

Alternative hinge and linker sequences that can be used as connecting regions may be crafted from portions of cell surface receptors that connect IgV-like or IgC-like domains. Regions between IgV-like domains where the cell surface receptor contains multiple IgV-like domains in tandem and between IgC-like domains where the cell surface receptor contains multiple tandem IgC-like regions could also be used as connecting regions or linker peptides. In certain embodiments, hinge and linker sequences are from 5 to 60 amino acids long, and may be primarily flexible, but may also provide more rigid characteristics, may contain primarily a helical structure with minimal beta sheet structure.

Certain illustrative linkers are provided in SEQ ID Nos: 132-135. Illustrative linkers are also provided within the context of various masked cytokine and parental non-masked fusion proteins herein as set forth in SEQ ID Nos: 58-89 (see also Table M).

Non-limiting examples of disease to be targeted with the masked cytokine fusion proteins herein include: all types of cancers, such as, but not limited to breast, including by way of non-limiting example, triple negative breast cancer, ER/PR+ breast cancer, and Her2+ breast cancer, lung cancer (e.g., non-small cell squamous and adenocarcinoma), colorectal cancer, gastric cancer, glioblastoma, ovarian cancer, endometrial cancer, renal cancer, sarcoma, skin cancer, cervical cancer, liver cancer, bladder cancer, cholangiocarcinoma, prostate cancer, melanomas, head and neck cancer (e.g. head and neck squamous cell carcinoma), esophageal, squamous cell cancer, basal cell carcinoma, pancreatic cancer, leukemias, including T-cell acute lymphoblastic leukemia (T-ALL), lymphoblastic diseases including multiple myeloma, and solid tumors. Indications also include bone disease or metastasis in cancer, regardless of primary tumor origin. Other illustrative diseases include rheumatoid arthritis, Crohn's disease, SLE, cardiovascular damage, and ischemia. In certain embodiments, the target disease is selected from the group consisting of colorectal cancer, pancreatic cancer, head and neck cancer, esophageal cancer, bladder cancer, cervical cancer, and lung cancer (e.g., non-small cell squamous and adenocarcinoma).

In one embodiment, the two heterologous polypeptides are selected from a cytokine polypeptide or functional fragment thereof, an antibody, an antigen-binding fragment of an antibody and an Fc domain. In another embodiment, the recombinant polypeptide comprises a cytokine polypeptide or a functional fragment thereof, a MM, and an Fc domain. In certain other embodiments, the MM is a single-chain Fv (scFv) antibody fragment that binds to the cytokine or a cytokine receptor polypeptide or a cytokine-binding fragment thereof. In a further embodiment, the recombinant polypeptide comprises an antibody or antigen binding fragment thereof that binds a target, and a MM that binds to the antibody or antigen binding fragment thereof and blocks binding of the antibody or antigen binding fragment thereof to the target.

In some embodiments, an IL12 fusion protein of the present disclosure comprises one or more peptide linkers that are cleavable. Such one or more cleavable linkers may be cleavable due to reactivity under certain conditions, e.g., such linkers may be protease-sensitive, acid-sensitive, or reduction-sensitive. In various embodiments, a cleavable linker herein comprises an amino acid sequence that is a cleavage recognition sequence for a protease, i.e., a protease cleavable linker. Many such cleavage recognition sequences are known in the art. In some embodiments, an amino acid sequence that is recognized and cleaved by a protease present in the extracellular matrix in the vicinity of a target cell, such as a cancer cell, can be employed. Examples of extracellular tumor-associated proteases include, for example, plasmin, matrix metalloproteases (MMPs), elastase and kallikrein-related peptidases.

### Fe Domains

In some embodiments, the masked IL12 fusion proteins described herein comprise an Fc, and in some embodiments, the Fc is a dimeric Fc. The dimeric Fc domain can be a heterodimeric Fc domain, as described herein. Such dimeric Fc domain of a fusion protein can comprise a first and a second Fc polypeptide, wherein the first Fc polypeptide can be coupled to a MM, and the second Fc polypeptide can be coupled to an IL12 polypeptide, e.g., as shown in FIG. 2B.

The term “Fc domain” or “Fc region” herein is used to define a C-terminal region of an immunoglobulin heavy chain that contains at least a portion of the constant region. The term includes native sequence Fc regions and variant Fc regions. Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, M D, 1991.

An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence.

An Fc domain comprises either a CH3 domain or a CH3 and a CH2 domain. The CH3 domain comprises two CH3 sequences, one from each of the two Fc polypeptides of the dimeric Fc. The CH2 domain comprises two CH2 sequences, one from each of the two Fc polypeptides of the dimeric Fc.

In some aspects, the Fc comprises at least one or two CH3 sequences. In some aspects, the Fc is coupled, with or without one or more linkers, to an IL12 polypeptide construct (e.g., C-terminally coupled to a first Fc polypeptide) and/or a masking moiety (MM) (e.g., C-terminally coupled to a second Fc polypeptide). In some aspects, the Fc is a human Fc. In some aspects, the Fc is a human IgG or IgG1 Fc. In some aspects, the Fc is a heterodimeric Fc. In some aspects, the Fc comprises at least one or two CH2 sequences.

In some aspects, the Fc comprises one or more modifications in at least one of the CH3 sequences. In some aspects, the Fc comprises one or more modifications in at least one of the CH2 sequences. In some aspects, an Fc is a single polypeptide. In some aspects, an Fc is multiple peptides, e.g., two polypeptides.

In some aspects, an Fc is an Fc described in patent applications PCT/CA2011/001238, filed Nov. 4, 2011 (WO2012058768; U.S. Pat. Nos. 9,562,109 and 10,875,931) or PCT/CA2012/050780, filed Nov. 2, 2012 (WO2013063702); U.S. Pat. Nos. 9,574,010; 9,732,155; 10,457,742 and US Pat. Application No.: US2020008741), all of which are herein incorporated by reference in their entirety.

### Modified CH3 Domains

In some aspects, the masked IL12 fusion proteins described herein comprises a heterodimeric Fc (“HetFc”) comprising a modified CH3 domain that has been asymmetrically modified. The heterodimeric Fc can comprise two heavy chain constant domain polypeptides: a first Fc polypeptide and a second Fc polypeptide, which can be used interchangeably provided that the Fc domain comprises one first Fc polypeptide and one second Fc polypeptide. Generally, the first Fc polypeptide comprises a first CH3 sequence and the second Fc polypeptide comprises a second CH3 sequence. In certain diagrams and elsewhere herein, a first Fc polypeptide and a second Fc polypeptide may be referred to as Fc polypeptide A and Fc polypeptide B (or chain A or chain B as shorthand), which similarly can be used interchangeably provided that the Fc domain or region comprises one Fc polypeptide A and one Fc polypeptide B. In some cases, the Fc domain which comprises one Fc polypeptide A and one Fc polypeptide B may be referred to as a variant and the variant may be referred to by a particular variant number to distinguish it from other Fc variants.

Two CH3 sequences that comprise one or more amino acid modifications introduced in an asymmetric fashion generally results in a heterodimeric Fc, rather than a homodimer, when the two CH3 sequences dimerize. As used herein, “asymmetric amino acid modifications” refers to any modification where an amino acid at a specific position on a first CH3 sequence is different from the amino acid on a second CH3 sequence at the same position, and the first and second CH3 sequence preferentially pair to form a heterodimer, rather than a homodimer. This heterodimerization can be a result of modification of only one of the two amino acids at the same respective amino acid position on each sequence; or modification of both amino acids on each sequence at the same respective position on each of the first and second CH3 sequences. The first and second CH3 sequence of a heterodimeric Fc can comprise one or more than one asymmetric amino acid modification.

Table D provides the amino acid sequence of the human IgG1 Fc sequence, corresponding to amino acids 231 to 447 of the full-length human IgG1 heavy chain. The CH3 sequence comprises amino acid 341-447 of the full-length human IgG1 heavy chain.

Typically, an Fc can include two contiguous heavy chain sequences (A and B) that are capable of dimerizing. In some aspects, one or both sequences of an Fc include one or more mutations or modifications at the following locations: L351, F405, Y407, T366, K392, T394, T350, S400, and/or N390, using EU numbering. In some aspects, an Fc includes a variant sequence shown in Table D. In some aspects, an Fc includes the mutations of Variant 1 A-B. In some aspects, an Fc includes the mutations of Variant 2 A-B. In some aspects, an Fc includes the mutations of Variant 3 A-B. In some aspects, an Fc includes the mutations of Variant 4 A B. In some aspects, an Fc includes the mutations of Variant 5 A-B.

TABLE D
IgG1 Fc sequences

| Human IgG1 | APELLGGPSVFLFPPKPKDTLMISRTP | |
|---|---|---|
| Fc sequence | EVTCVVVDVSHEDPEVKFNWYVDGVEV | |
| 231-447 | HNAKTKPREEQYNSTYRVVSVLTVLHQ | |
| (EU-numbering) | DWLNGKEYKCKVSNKALPAPIEKTISK | |
| | AKGQPREPQVYTLPPSRDELTKNQVSL | |
| | TCLVKGFYPSDIAVEWESNGQPENNYK | |
| | TTPPVLDSDGSFFLYSKLTVDKSRWQQ | |
| | GNVFSCSVMHEALHNHYTQKSLSLSPG | |
| | K (SEQ ID NO: 136) | |
| Variant IgG1 | | |
| Fc sequence | | |
| (231-447) | Chain | Mutations |
| 1 | A | L351Y_F405A_Y407V |
| 1 | B | T366L_K392M_T394W |
| 2 | A | L351Y_F405A_Y407V |
| 2 | B | T366L_K392L_T394W |
| 3 | A | T350V_L351Y_F405A_Y407V |
| 3 | B | T350V_T366L_K392L_T394W |
| 4 | A | T350V_L351Y_F405A_Y407V |
| 4 | B | T350V_T366L_K392M_T394W |
| 5 | A | T350V_L351Y_S400E_F405A_Y407V |
| 5 | B | T350V_T366L_N390R_K392M_T394W |

The first and second CH3 sequences can comprise amino acid mutations as described herein, with reference to amino acids 231 to 447 of the full-length human IgG1 heavy chain. In one embodiment, the heterodimeric Fc comprises a modified CH3 domain with a first CH3 sequence having amino acid modifications at positions F405 and Y407, and a second CH3 sequence having amino acid modifications at position T394. In one embodiment, the heterodimeric Fc comprises a modified CH3 domain with a first CH3 sequence having one or more amino acid modifications selected from L351Y, F405A, and Y407V, and the second CH3 sequence having one or more amino acid modifications selected from T366L, T366I, K392L, K392M, and T394W.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, and one of the first or second CH3 sequences further comprising amino acid modifications at position Q347, and the other CH3 sequence further comprising amino acid modification at position K360. In another embodiment, a heterodimeric Fc comprises a modified CH3 domain with a first CH3 sequence having amino acid modifications at positions L351, F405 and Y407, and a second CH3 sequence having amino acid modifications at position T366, K392, and T394, one of the first or second CH3 sequences further comprising amino acid modifications at position Q347, and the other CH3 sequence further comprising amino acid modification at position K360, and one or both of said CH3 sequences further comprise the amino acid modification T350V.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394 and one of said first and second CH3 sequences further comprising amino acid modification of D399R or D399K and the other CH3 sequence comprising one or more of T411E, T411D, K409E, K409D, K392E and K392D. In another embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, one of said first and second CH3 sequences further comprises amino acid modification of D399R or D399K and the other CH3 sequence comprising one or more of T411E, T411D, K409E, K409D, K392E and K392D, and one or both of said CH3 sequences further comprise the amino acid modification T350V.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, wherein one or both of said CH3 sequences further comprise the amino acid modification of T350V.

In one embodiment, a heterodimeric Fc comprises a modified CH3 domain comprising the following amino acid modifications, where “A” represents the amino acid modifications to the first CH3 sequence, and “B” represents the amino acid modifications to the second CH3 sequence: A:L351Y_F405A_Y407V, B:T366L_K392M_T394W, A: L351 Y_F405A_Y407V, B:T366L_K392L_T394W, A:T350V_L351Y_F405A_Y407V, B:T350V_T366L_K392L_T394W, A:T350V_L351Y_F405A_Y407V, B:T350V_T366L_K392M_T394W, A:T350V_L351 Y_S400E_F405A_Y407V, and/or B:T350V_T366L N390R_K392M_T394W.

The one or more asymmetric amino acid modifications can promote the formation of a heterodimeric Fc in which the heterodimeric CH3 domain has a stability that is comparable to a wild-type homodimeric CH3 domain. In an embodiment, the one or more asymmetric amino acid modifications promote the formation of a heterodimeric Fc domain in which the heterodimeric Fc domain has a stability that is comparable to a wild-type homodimeric Fc domain. In an embodiment, the one or more asymmetric amino acid modifications promote the formation of a heterodimeric Fc domain in which the heterodimeric Fc domain has a stability observed via the melting temperature (Tm) in a differential scanning calorimetry study, and where the melting temperature is within 4° C. of that observed for the corresponding symmetric wild-type homodimeric Fc domain. In some aspects, the Fc comprises one or more modifications in at least one of the CH3 sequences that promote the formation of a heterodimeric Fc with stability comparable to a wild-type homodimeric Fc.

### Modified CH2 Domains

In certain embodiments, an Fc domain contemplated for use herein is an Fc having a modified CH2 domain. In some embodiments, an Fc domain contemplated for use herein is an IgG Fc having a modified CH2 domain, wherein the modification of the CH2 domain results in altered binding to one or more Fc receptors (FcRs) such as receptors of the FcγRI, FcγRII and FcγRIII subclasses.

A number of amino acid modifications to the CH2 domain that selectively alter the affinity of the Fc for different Fcγ receptors are known in the art. Amino acid modifications that result in increased binding and amino acid modifications that result in decreased binding can both be useful in certain indications. For example, increasing binding affinity of an Fc for FcγRIIIa (an activating receptor) results in increased antibody dependent cell-mediated cytotoxicity (ADCC), which in turn results in increased lysis of the target cell. Decreased binding to FcγRIIb (an inhibitory receptor) likewise may be beneficial in some circumstances. In certain indications, a decrease in, or elimination of, ADCC and complement-mediated cytotoxicity (CDC) may be desirable. In such cases, modified CH2 domains comprising amino acid modifications that result in increased binding to FcγRIIb or amino acid modifications that decrease or eliminate binding of the Fc region to all of the Fcγ receptors (“knock-out” variants) may be useful.

Examples of amino acid modifications to the CH2 domain that alter binding of the Fc by Fcγ receptors include, but are not limited to, the following: S298A/E333A/K334A and S298A/E333A/K334A/K326A (increased affinity for FcγRIIIa) (Lu, et al., 2011, J Immunol Methods, 365(1-2):132-41); F243L/R292P/Y300L/V305I/P396L (increased affinity for FcγRIIIa) (Stavenhagen, et al., 2007, Cancer Res, 67(18):8882-90); F243L/R292P/Y300L/L235V/P396L (increased affinity for FcγRIIIa) (Nordstrom J L, et al., 2011, Breast Cancer Res, 13(6):R123); F243L (increased affinity for FcγRIIIa) (Stewart, et al., 2011, Protein Eng Des Sel., 24(9):671-8); S298A/E333A/K334A (increased affinity for FcγRIIIa) (Shields, et al., 2001, J Biol Chem, 276(9):6591-604); S239D/I332E/A330L and S239D/I332E (increased affinity for FcγRIIIa) (Lazar, et al., 2006, Proc Natl Acad Sci USA, 103(11):4005-10), and S239D/S267E and S267E/L328F (increased affinity for FcγRIIb) (Chu, et al., 2008, Mol Immunol, 45(15):3926-33).

Additional modifications that affect Fc binding to Fcγ receptors are described in Therapeutic Antibody Engineering (Strohl & Strohl, Woodhead Publishing series in Biomedicine No 11, ISBN 1 907568 37 9, October 2012, page 283).

In certain embodiments, a masked or nonmasked IL12 fusion protein comprises a scaffold based on an IgG Fc having a modified CH2 domain, in which the modified CH2 domain comprises one or more amino acid modifications that result in decreased or eliminated binding of the Fc region to all of the Fcγ receptors (i.e., a “knock-out” variant).

Various publications describe strategies that have been used to engineer antibodies to produce “knock-out” variants (see, for example, Strohl, 2009, Curr Opin Biotech 20:685-691, and Strohl & Strohl, “Antibody Fc engineering for optimal antibody performance” In Therapeutic Antibody Engineering, Cambridge: Woodhead Publishing, 2012, pp 225-249). These strategies include reduction of effector function through modification of glycosylation (described in more detail below), use of IgG2/IgG4 scaffolds, or the introduction of mutations in the hinge or CH2 domain of the Fc (see also, U.S. Patent Publication No. 2011/0212087, International Publication No. WO 2006/105338, U.S. Patent Publication No. 2012/0225058, U.S. Patent Publication No. 2012/0251531 and Strop et al., 2012, J. Mol. Biol., 420: 204-219).

Specific, non-limiting examples of known amino acid modifications to reduce FcγR and/or complement binding to the Fc include those identified in Table E.

TABLE E
Modifications to Reduce Fcγ Receptor
or Complement Binding to the Fc

| Company | Mutations |
|---|---|
| GSK | N297A |
| Ortho Biotech | L234A/L235A |
| Protein Design labs | IgG2 V234A/G237A |
| Wellcome Labs | IgG4 L235A/G237A/E318A |
| GSK | IgG4 S228P/L236E |
| Merck | IgG2 H268Q/V309L/A330S/A331S |
| Bristol-Myers | C220S/C226S/C229S/P238S |
| Seattle Genetics | C226S/C229S/E3233P/L235V/L235A |
| Medimmune | L234F/L235E/P331S |

Additional examples include Fc regions engineered to include the amino acid modifications L234A/L235A/D265S. In addition, asymmetric amino acid modifications in the CH2 domain that decrease binding of the Fc to all Fcγ receptors are described in International Publication No. WO 2014/190441.

In additional embodiments, certain amino acid substitutions are introduced into human IgG1 Fc for Fc domain of the present disclosure to ablate immune effector functions such as antibody-dependent cell cytotoxicity (ADCC) and complement-dependent cytotoxicity (CDC). Mutations in the CH2 region of the antibody heavy chains may include positions 234, 235, and 265 in EU numbering to reduce or eliminate immune effector functions.

### Targeting Domain

In certain embodiments, the IL12 fusion proteins described herein may comprise a “targeting domain” that targets the fusion proteins to a site of action (e.g. sites of inflammation, a particular anatomical site such as an organ, or to a tumor). As used herein, the “targeted antigen” is the antigen recognized and specifically bound by the targeting domain.

In some embodiments, the targeting domain is specific for (specifically binds) an antigen found on cells in a protease-rich environment such as the tumor microenvironment. In some embodiments, the encoded targeting domain is specific for (e.g., specifically binds or recognizes) regulatory T cells (Tregs), for example targeting the CCR4 or CD39 receptors. Other suitable targeting domains comprise those that have a cognate ligand that is overexpressed in inflamed tissues, e.g., the IL1 receptor, or the IL6 receptor. In other embodiments, a suitable targeting domain is one that has a cognate ligand present on an immune cell such as a dendritic cell (DC), a T cell, an NK cell, etc. In other embodiments, the suitable targeting domain comprise those that have a cognate ligand that is overexpressed in tumor tissue, e.g., a tumor-associated antigen (TAA).

TAAs contemplated herein for tumor targeting include but are not limited to EpCAM, EGFR, HER-2, HER-3, c-Met, FOLR1, and CEA. In certain embodiments, the masked fusion proteins comprise two targeting domains that bind to two different target antigens known to be expressed on a diseased cell or tissue. Exemplary pairs of antigen binding domains include but are not limited to EGFR/CEA, EpCAM/CEA, and HER-2/HER-3.

Suitable targeting domains include antigen-binding domains, such as antibodies and fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like. Other suitable antigen-binding domain include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of antigen-binding polypeptides include a ligand for a desired receptor, a ligand-binding portion of a receptor, a lectin, and peptides that binds to or associates with one or more target antigens.

In some embodiments, a targeting domain specifically binds to a cell surface molecule. In some embodiments, a targeting domain specifically binds to a tumor antigen. In some embodiments, the targeting domain specifically and independently binds to a tumor antigen selected from at least one of Fibroblast activation protein alpha (FAPa), Trophoblast glycoprotein (5T4), Tumor-associated calcium signal transducer 2 (Trop2), Fibronectin EDB (EDB-FN), fibronectin F.IIIB domain, CGS-2, EpCAM, EGER, HER-2, HER-3, cMet, CEA, and FOLR1. In some embodiments, the targeting polypeptides specifically and independently bind to two different antigens, wherein at least one of the antigens is a tumor antigen selected from EpCAM, EGFR, HER-2, HER-3, cMet, CEA, and FOLR1. The TAA targeted by the targeting domain can be a tumor antigen expressed on a tumor cell. Tumor antigens are well known in the art and include, for example, EpCAM, EGFR, HER-2, HER-3, c-Met, FOLR1, PSMA, CD38, BCMA, and CEA. 5T4, AFP, B7-H3, Cadherin-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Mucl, Mucl6, NaPi2b, Nectin-4, P-cadherin, NY-ESO-1, PRLR, PSCA, PTK7, ROR1, SLC44A4, SLTRK5, SLTRK6, STEAPI, TIM1, Trop2, FAP, or WT1.

In some embodiments, the targeted antigen is an immune checkpoint protein. Examples of immune checkpoint proteins include but are not limited to CD27, CD137, 2B4, TIGIT, CD155, ICOS, HVEM, CD40L, LIGHT, TIM-1, OX40, DNAM-1, PD-L1, PD1, PD-L2, CTLA-4, CD80, CD40, CEACAMI, CD48, CD70, A2AR, CD39, CD73, B7-H3, B7-H4, BTLA, IDOl, ID02, TDO, KIR, LAG-3, TIM-3, or VISTA. In certain embodiments, the targeting domain is an antibody or antigen-binding fragment thereof that specifically binds to an immune checkpoint protein, or the targeting domain is a ligand that binds to an immune checkpoint protein or is a binding fragment thereof.

The targeting domain can specifically bind to a cell surface molecule such as a protein, lipid or polysaccharide. In some embodiments, a targeted antigen is an antigen expressed on a tumor cell, virally infected cell, bacterially infected cell, damaged red blood cell, arterial plaque cell, inflamed or fibrotic tissue cell. The targeted antigen can comprise an immune response modulator. Examples of immune response modulator include but are not limited to granulocyte-macrophage colony stimulating factor (GM-CSF), macrophage colony stimulating factor (M-CSF), granulocyte colony stimulating factor (G-CSF), interleukin 2 (IL2), interleukin 3 (IL3), interleukin 12 (IL12), interleukin 15 (IL15), B7-1 (CD80), B7-2 (CD86), GITRL, CD3, or GITR.

In certain embodiments, the targeting domain specifically binds a cytokine receptor. Examples of cytokine receptors include, but are not limited to, Type I cytokine receptors, such as GM-CSF receptor, G-CSF receptor, Type I IL receptors, Epo receptor, LIF receptor, CNTF receptor, TPO receptor; Type II Cytokine receptors, such as IFN-alpha receptor (IFNAR1, IFNAR2), IFB-beta receptor, IFN-gamma receptor (IFNGR1, IFNGR2), Type II IF receptors; chemokine receptors, such as CC chemokine receptors, CXC chemokine receptors, CX3C chemokine receptors, XC chemokine receptors; tumor necrosis receptor superfamily receptors, such as TNFRSF5/CD40, TNFRSF8/CD30, TNFRSF7/CD27, TNFRSFIA/TNFR1/CD120a, TNFRSF1B/TNFR2/CD120b; TGF-beta receptors, such as TGF-beta receptor 1, TGF-beta receptor 2; Ig super family receptors, such as IF-1 receptors, CSF-1R, PDGFR (PDGFRA, PDGFRB), SCFR.

In some embodiments, the targeting domain is fused to the masked IL12 fusion protein via a linker or a PCL (also known as a protease cleavable linker). In certain embodiments, the linker fusing the targeting domain to the masked IL12 fusion protein is a PCL which is cleaved at the site of action (e.g., by inflammation or cancer specific proteases). In this regard, the PCL may be the same as or different from any other PCL that is present in the masked IL12 fusion protein, such as a PCL fusing a MM to an Fc polypeptide, a PCL present with the MM or a PCL that links an IL12 polypeptide to an Fc polypeptide. In certain embodiments, the PCL fusing the targeting domain is the same as a PCL fusing the MM to an Fc polypeptide and/or the PCL fusing the IL12 to an Fc polypeptide whereby, all of the cleavage sites are cleaved upon reaching the target. In some embodiments, the targeting domain is fused to the masked IL12 fusion protein via a linker which is not cleaved at the site of action (e.g., by inflammation or cancer specific proteases).

### Certain Embodiments Related to IL12 and Il23 Fusion Proteins

In certain embodiments, an IL12 fusion protein of the present disclosure can comprise: an IL12 polypeptide having (i) a modified p40 domain as described herein, coupled to (ii) a p35 domain, and an Fc domain comprising a first Fc polypeptide and a second Fc polypeptide. In some embodiments, the p35 domain comprises or consists of an amino acid sequence having at least about 90%, 95%, 97%, 99% or 100% sequence identity to the amino acid sequence set forth in SEQ ID NO: 11. In some embodiments, the N-terminus of the p35 domain is coupled to the C-terminus of the modified p40 domain either directly or via a first linker. The first linker can have the sequence of (G₄S)ₓ, wherein x is 1, 2, 3 or 4. In some embodiments, the IL12 fusion protein is an IL12 HetFc fusion protein that can further comprise a heterodimeric Fc domain comprising a first Fc polypeptide and a second Fc polypeptide. In such embodiments, the IL12 polypeptide can be coupled to the first Fc polypeptide either directly or via a second linker. In some embodiments, the IL12 polypeptide is coupled to the C-terminus of the first Fc polypeptide. Hence, in some embodiments, the IL12 polypeptide is coupled to the C-terminus of the first Fc polypeptide via the N-terminus of the modified p40 domain.

In some embodiments, the IL12 HetFc fusion protein can further comprise a masking moiety, wherein the masking moiety is capable of non-covalently interacting with the modified p40 domain, thereby masking the modified p40 domain and reducing the binding affinity (K_{D}) of the modified p40 domain for binding to at least one of its cognate receptors when compared to an unmasked modified p40 domain. In some embodiments, the masking moiety is coupled to the C-terminus of the second Fc polypeptide either directly or via a third linker. The third linker can be a protease-cleavable linker. The second and/or third linker(s) can each comprise or consist of an amino acid sequence spanning from 5 to about 50 amino acids. Hence, in some embodiments, the second linker comprises or consists of the amino acid sequence set forth in SEQ ID NO: 132, and the third linker comprises or consists of the amino acid sequence set forth in SEQ ID NO: 134. In some embodiments, the masking moiety comprises or consists of an scFv domain comprising a V_{H} domain coupled either directly or via a fourth linker to a V_{L} domain. In some embodiments, the fourth linker comprises or consists of the amino acid sequence set forth in SEQ ID NO: 135, the V_{H} domain comprises or consists of an amino acid sequence having about 95%, 97%, 99%, or 100% sequence identity to the sequence set forth in SEQ ID NO: 2, and the V_{L} domain comprises or consists of an amino acid sequence having about 95%, 97%, 99%, or 100% sequence identity to the sequence set forth in SEQ ID NO: 3.

In some embodiments, the masking moiety is capable of reducing the binding affinity of the modified p40 domain to the at least one cognate receptor by at least about 10-fold, 15-fold, 20-fold, 30-fold, 40-fold, 50-fold, 100-fold, 200-fold, or at least about 300-fold, compared to a corresponding fusion protein that does not comprise the masking moiety. The at least one cognate receptor can comprise or consist of IL12Rβ1.

In various embodiments, described herein is a fusion protein that is a masked IL12 HetFc fusion protein comprising: (i) an IL-12 polypeptide comprising the modified p40 domain according to any of the embodiments described herein, coupled via the linker (G₄S)₄ to the p35 domain comprising or consisting of the amino acid sequence set forth in SEQ ID NO: 11; (ii) a heterodimeric Fc domain comprising a first Fc polypeptide and a second Fc polypeptide; and (iii) a masking moiety (MM) comprising an anti-IL12 scFv domain, wherein: the IL12 polypeptide is coupled either directly or via a second linker to the C-terminus of the first Fc polypeptide, and the masking moiety is coupled either directly or via a third linker to the C-terminus of the second Fc polypeptide. In some of these embodiments, the fusion protein comprises or consists of two polypeptide chains, from N- to C-terminus, (i) an Fc-IL12 polypeptide chain and (ii) an Fc-MM polypeptide chain. In some embodiments, the Fc-IL12 polypeptide chain comprises or consists of an amino acid sequence having at least about 90%, 95%, 97%, 99%, or 100% sequence identity to any of the amino acid sequences set forth in SEQ ID NOs: 61-89. In some embodiments, the Fc-IL12 polypeptide chain comprises or consists of the amino acid sequence set forth in any one of SEQ ID NOs: 61-89. The Fc-MM polypeptide chain, which can pair via the CH3 domains with the Fc-IL12 polypeptide chain, can comprise or consist of an amino acid sequence having at least about 90%, 95%, 97%, 99%, or 100% sequence identity to the amino acid sequence set forth in SEQ ID NO: 60. In some embodiments, the Fc-MM polypeptide chain comprises or consists of the amino acid sequence set forth in SEQ ID NO: 60. Hence, in some embodiments, a masked IL12 HetFc fusion protein (i) the Fc-IL12 polypeptide chain (from N- to C-terminus) comprises a sequence that is selected from the group consisting of the sequences of: v28046, v28047, v28048, v28049, v28050, v28051, v28053, v28054, v28055, v28056, v28057, v28058, v28059, v28060, v28061, v28062, v28063, v28064, v28065, v28066, v28067, v28068, v28069, v28070, v28071, v28072, v28074 and v28075, and (ii) the Fc-MM polypeptide chain (from N- to C-terminus) comprises the amino acid sequence of v26503.

In some embodiments, also described herein is a fusion protein that is an unmasked IL12 HetFc fusion protein comprising: (i) an IL-12 polypeptide comprising the modified p40 domain according to any of the embodiments described herein, coupled via the linker (G4S)₄ to the p35 domain comprising or consisting of the amino acid sequence set forth in SEQ ID NO: 11; and (ii) a heterodimeric Fc domain comprising a first Fc polypeptide and a second Fc polypeptide, wherein the IL12 polypeptide is coupled either directly or via a second linker to the C-terminus of the first Fc polypeptide. In some embodiments, the fusion protein comprises or consists of two polypeptide chains, from N- to C-terminus, (i) an Fc-IL12 polypeptide chain and (ii) an Fc polypeptide chain. In some embodiments, the Fc-IL12 polypeptide chain comprises or consists of an amino acid sequence having at least about 90%, 95%, 97%, 99%, or 100% sequence identity to any of the amino acid sequences set forth in SEQ ID NOs: 61-89. In some embodiments, the Fc-IL12 polypeptide chain comprises or consists of the amino acid sequences set forth in SEQ ID NOs: 61-89. The Fc polypeptide chain, which can pair with the Fc-IL12 polypeptide chain, can comprise or consist of an amino acid sequence having at least about 90%, 95%, 97%, 99%, or 100% sequence identity to the amino acid sequence set forth in SEQ ID NO: 58. In some embodiments, the unmasked IL12 HetFc fusion protein comprises (i) the Fc-IL12 polypeptide chain (from N- to C-terminus) comprises an amino acid sequence selected from the group consisting of the sequences of: v28046, v28047, v28048, v28049, v28050, v28051, v28053, v28054, v28055, v28056, v28057, v28058, v28059, v28060, v28061, v28062, v28063, v28064, v28065, v28066, v28067, v28068, v28069, v28070, v28071, v28072, v28074 and v28075, and (ii) the Fc polypeptide chain comprising the amino acid sequence of v12153.

### Polypeptides and Polynucleotides

The cytokine (e.g., IL12 and other members of the IL12 family of cytokines) fusion proteins described herein comprise at least one polypeptide. Also described are polynucleotides encoding the polypeptides described herein. The masked cytokine fusion proteins are typically isolated.

As used herein, “isolated” means an agent (e.g., a polypeptide or polynucleotide) that has been identified and separated and/or recovered from a component of its natural cell culture environment. Contaminant components of its natural environment are materials that would interfere with diagnostic or therapeutic uses for the cytokine fusion proteins, and may include enzymes, hormones, and other proteinaceous or non-proteinaceous solutes. Isolated also refers to an agent that has been synthetically produced, e.g., via human intervention.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally encoded amino acid. As used herein, the terms encompass amino acid chains of any length, including full length proteins, wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, praline, serine, threonine, tryptophan, tyrosine, and valine) and pyrrolysine and selenocysteine. Amino acid analogs refers to compounds that have the same basic chemical structure as a naturally occurring amino acid, i.e., a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Reference to an amino acid includes, for example, naturally occurring proteogenic L-amino acids; D-amino acids, chemically modified amino acids such as amino acid variants and derivatives; naturally occurring non-proteogenic amino acids such as β-alanine, ornithine, etc.; and chemically synthesized compounds having properties known in the art to be characteristic of amino acids. Examples of non-naturally occurring amino acids include, but are not limited to, α-methyl amino acids (e.g. α-methyl alanine), D-amino acids, histidine-like amino acids (e.g., 2-amino-histidine, β-hydroxy-histidine, homohistidine), amino acids having an extra methylene in the side chain (“homo” amino acids), and amino acids in which a carboxylic acid functional group in the side chain is replaced with a sulfonic acid group (e.g., cysteic acid).

The incorporation of non-natural amino acids, including synthetic non-native amino acids, substituted amino acids, or one or more D-amino acids into the proteins described herein may be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore, less likely to induce humoral immune responses in the whole organism.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Also provided herein are polynucleotides encoding the masked cytokine fusion proteins. The term “polynucleotide” or “nucleotide sequence” is intended to indicate a consecutive stretch of two or more nucleotide molecules. The nucleotide sequence may be of genomic, cDNA, RNA, semisynthetic or synthetic origin, or any combination thereof.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

“Conservatively modified variants” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the deletion of an amino acid, addition of an amino acid, or substitution of an amino acid with a chemically similar amino acid. Conservative substitution tables providing functionally similar amino acids are known to those of ordinary skill in the art. Such conservatively modified variants are in addition to and do not exclude polymorphic variants, interspecies homologs, and alleles described herein.

Conservative substitution tables providing functionally similar amino acids are known to those of ordinary skill in the art. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and [0139] 8) Cysteine (C), Methionine (M).

The terms “identical” or percent “identity,” in the context of two or more nucleic acids or polypeptide sequences, refer to two or more sequences or subsequences that are the same. Sequences are “substantially identical” if they have a percentage of amino acid residues or nucleotides that are the same (i.e., about 60% identity, about 65%, about 70%, about 75%, about 80%, about 85%, about 90%, about 95%, about 96%, about 97%, about 98%, or about 99% identity over a specified region), when compared and aligned for maximum correspondence over a comparison window, or designated region as measured using one of the following sequence comparison algorithms (or other algorithms available to persons of ordinary skill in the art) or by manual alignment and visual inspection. This definition also refers to the complement of a test sequence. The identity can exist over a region that is at least about 50 amino acids or nucleotides in length, or over a region that is 75-100 amino acids or nucleotides in length, or, where not specified, across the entire sequence of a polynucleotide or polypeptide. A polynucleotide encoding a polypeptide described herein, including homologs from species other than human, may be obtained by a process comprising the steps of screening a library under stringent hybridization conditions with a labeled probe having a polynucleotide sequence described herein or a fragment thereof, and isolating full-length cDNA and genomic clones containing said polynucleotide sequence. Such hybridization techniques are well known to the skilled artisan.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are known to those of ordinary skill in the art. Optimal alignment of sequences for comparison can be conducted, including but not limited to, by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

One example of an algorithm that is suitable for determining percent sequence identity and sequence similarity are the BLAST and BLAST 2.0 algorithms, which are described in Altschul et al. (1997) Nuc. Acids Res. 25:3389-3402, and Altschul et al. (1990) J. Mol. Biol. 215:403-410, respectively. Software for performing BLAST analyses is publicly available through the National Center for Biotechnology Information available at the World Wide Web at ncbi.nlm.nih.gov. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1992) Proc. Natl. Acad. Sci. USA 89:10915) alignments (B) of 50, expectation (E) of 10, M=5, N=−4, and a comparison of both strands. The BLAST algorithm is typically performed with the “low complexity” filter turned off.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, or less than about 0.01, or less than about 0.001.

The phrase “selectively (or specifically) hybridizes to” refers to the binding, duplexing, or hybridizing of a molecule only to a particular nucleotide sequence under stringent hybridization conditions when that sequence is present in a complex mixture (including but not limited to, total cellular or library DNA or RNA).

The phrase “stringent hybridization conditions” refers to hybridization of sequences of DNA, RNA, or other nucleic acids, or combinations thereof under conditions of low ionic strength and high temperature as is known in the art. Typically, under stringent conditions a probe will hybridize to its target subsequence in a complex mixture of nucleic acid (including but not limited to, total cellular or library DNA or RNA) but does not hybridize to other sequences in the complex mixture. Stringent conditions are sequence-dependent and will be different in different circumstances. Longer sequences hybridize specifically at higher temperatures. An extensive guide to the hybridization of nucleic acids is found in Tijssen, Laboratory Techniques in Biochemistry and Molecular Biology—Hybridization with Nucleic Probes, “Overview of principles of hybridization and the strategy of nucleic acid assays” (1993).

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches. The engineered proteins are expressed and produced by standard molecular biology techniques.

By “isolated nucleic acid molecule or polynucleotide” is intended a nucleic acid molecule, DNA or RNA, which has been removed from its native environment. For example, a recombinant polynucleotide encoding a polypeptide contained in a vector is considered isolated. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extra-chromosomally or at a chromosomal location that is different from its natural chromosomal location. Isolated RNA molecules include in vivo or in vitro RNA transcripts, as well as positive and negative strand forms, and double-stranded forms. Isolated polynucleotides or nucleic acids described herein, further include such molecules produced synthetically, e.g., via PCR or chemical synthesis. In addition, a polynucleotide or a nucleic acid, in certain embodiments, include a regulatory element such as a promoter, ribosome binding site, or a transcription terminator.

The term “polymerase chain reaction” or “PCR” generally refers to a method for amplification of a desired nucleotide sequence in vitro, as described, for example, in U.S. Pat. No. 4,683,195. In general, the PCR method involves repeated cycles of primer extension synthesis, using oligonucleotide primers capable of hybridising preferentially to a template nucleic acid.

By a nucleic acid or polynucleotide having a nucleotide sequence at least, for example, 95% “identical” to a reference nucleotide sequence of the present disclosure, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present disclosure can be determined conventionally using known computer programs, such as the ones discussed above for polypeptides (e.g., ALIGN-2).

A derivative, or a variant of a polypeptide is said to share “homology” or be “homologous” with the peptide if the amino acid sequences of the derivative or variant has at least 50% identity with a 100 amino acid sequence from the original peptide. In certain embodiments, the derivative or variant is at least 75% the same as that of either the peptide or a fragment of the peptide having the same number of amino acid residues as the derivative. In certain embodiments, the derivative or variant is at least 85% the same as that of either the peptide or a fragment of the peptide having the same number of amino acid residues as the derivative. In certain embodiments, the amino acid sequence of the derivative is at least 90% the same as the peptide or a fragment of the peptide having the same number of amino acid residues as the derivative. In some embodiments, the amino acid sequence of the derivative is at least 95%, 96%, 97%, or 98% the same as the peptide or a fragment of the peptide having the same number of amino acid residues as the derivative. In certain embodiments, the derivative or variant is at least 99% the same as that of either the peptide or a fragment of the peptide having the same number of amino acid residues as the derivative.

The term “modified,” as used herein refers to any changes made to a given polypeptide, such as changes to the length of the polypeptide, the amino acid sequence, chemical structure, co-translational modification, or post-translational modification of a polypeptide. The form “(modified)” term means that the polypeptides being discussed are optionally modified, that is, the polypeptides under discussion can be modified or unmodified.

In some aspects, a cytokine fusion protein construct comprises an amino acid sequence that is at least 80, 85, 90, 91, 92, 93, 94, 95, 96, 97, 98, 99, or 100% identical to a relevant amino acid sequence or fragment thereof set forth in the Table(s) or accession number(s) disclosed herein. In some aspects, a cytokine fusion protein comprises an amino acid sequence encoded by a polynucleotide that is at least 80, 85, 90, 91, 92, 93, 94, 95, 96, 97, 98, 99, or 100% identical to a relevant nucleotide sequence or fragment thereof set forth in the Table(s) or accession number(s) disclosed herein.

In some embodiments, the one or more amino acid substitutions are one or more non-conservative substitutions. In other embodiments, the one or more amino acid substitutions are one or more conservative substitutions. In general, a “conservative substitution,” as used herein, is considered to be a substitution of one amino acid with another amino acid having similar physical, chemical and/or structural properties. Common conservative substitutions are listed under Column 1 of Table F. One skilled in the art will appreciate that the main factors in determining what constitutes a conservative substitution are usually the size of the amino acid side chain and its physical/chemical properties, but that certain environments allow for substitution of a given amino acid with a broader range of amino acids than those listed in Column 1 of Table F. These additional amino acids tend to either have similar properties to the amino acid being substituted but to vary more widely in size or be of similar size but vary more widely in physical/chemical properties. This broader range of conservative substitutions is listed under Column 2 of Table F. The skilled person can readily ascertain the most appropriate group of substituents to select from in view of the particular protein environment in which the amino acid substitution is being made.

TABLE F
Conservative Amino Acid Substitutions

| Original | | |
|---|---|---|
| Amino | | |
| Acid | Column 1 | Column 2 |
| Ala (A) | Gly, Ile, Leu, Met, | Cys, Gly, Ile, Leu, Met, Norleucine, |
| | Norleucine, Val | Phe, Trp, Tyr, Val |
| Arg (R) | His, Lys | His, Lys |
| Asn (N) | Cys, Gln, Ser, Thr | Asp, Cys, Gln, Glu, Ser, Thr |
| Asp (D) | Glu | Asn, Cys, Gln, Glu, Ser, Thr |
| Cys (C) | Asn, Gln, Ser, Thr | Asn, Asp, Gln, Glu, Ser, Thr |
| Gln (Q) | Asn, Cys, Ser, Thr | Asn, Asp, Cys, Glu, Ser, Thr |
| Glu (E) | Asp | Asp, Asn, Cys, Gln, Ser, Thr |
| Gly (G) | Pro | Ala, Ile, Leu, Met, Norleucine, Pro, Val |
| His (H) | Arg, Lys | Arg, Lys, Phe, Trp, Tyr |
| Ile (I) | Ala, Gly, Leu, Met, | Ala, Cys, Gly, Leu, Met, Norleucine, |
| | Norleucine, Val | Phe, Trp, Tyr, Val |
| Leu (L) | Ala, Gly, Ile, Met, | Ala, Cys, Gly, Ile, Met, Norleucine, |
| | Norleucine, Val | Phe, Trp, Tyr, Val |
| Lys (K) | Arg, His | Arg, His |
| Met (M) | Ala, Gly, Ile, Leu, | Ala, Cys, Gly, Ile, Leu, Norleucine, |
| | Norleucine, Val | Phe, Trp, Tyr, Val |
| Phe (F) | Tyr, Trp | Ala, Cys, Gly, His, Ile, Leu, Met, |
| | | Norleucine, Trp, Tyr, Val |
| Pro (P) | Gly | Gly |
| Ser (S) | Asn, Cys, Gln, Thr | Asp, Asn, Cys, Gln, Glu, Thr |
| Thr (T) | Asn, Cys, Gln, Ser | Asp, Asn, Cys, Gln, Glu, Ser |
| Trp (W) | Phe, Tyr | Ala, Cys, Gly, His, Ile, Leu, Met, |
| | | Norleucine, Phe, Tyr, Val |
| Tyr (Y) | Phe, Trp | Ala, Cys, Gly, His, Ile, Leu, Met, |
| | | Norleucine, Phe, Trp, Val |
| Val (V) | Ala, Gly, Ile, Leu, | Ala, Cys, Gly, Ile, Leu, Met, |
| | Met, Norleucine | Norleucine, Phe, Trp, Tyr |

### Methods of Preparing IL12 Fusion Proteins/Recombinant Proteins

The IL12 fusion proteins or other recombinant proteins (e.g., recombinant proteins comprising a modified p40 domain) described herein may be produced using standard recombinant methods known in the art (see, e.g., U.S. Pat. No. 4,816,567 and “Antibodies: A Laboratory Manual,” 2nd Edition, Ed. Greenfield, Cold Spring Harbor Laboratory Press, New York, 2014) and as further outlined herein.

Typically, for recombinant production of a IL12 fusion proteins or other recombinant proteins, nucleic acid encoding the IL12 fusion proteins or other recombinant proteins is isolated and inserted into one or more vectors for further cloning and/or expression in a host cell. Such nucleic acid may be readily isolated and sequenced using conventional procedures (e.g. by using oligonucleotide probes that are capable of binding specifically to genes IL12 fusion proteins or other recombinant proteins).

Suitable host cells for cloning or expression of IL12 fusion proteins or other recombinant proteins encoding vectors include prokaryotic or eukaryotic cells described herein.

A “recombinant host cell” or “host cell” refers to a cell that includes an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome.

As used herein, the term “eukaryote” refers to organisms belonging to the phylogenetic domain Eucarya such as animals (including but not limited to, mammals, insects, reptiles and birds), ciliates, plants (including but not limited to, monocots, dicots and algae), fungi, yeasts, flagellates, microsporidia, protists, and the like.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, and the like) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, and the like) phylogenetic domain.

For example, a IL12 fusion protein construct or other recombinant protein comprising modified p40 domain construct described herein may be produced in bacteria, in particular when glycosylation and Fc effector function are not needed. For expression of polypeptides in bacteria, see, for example, U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the IL12 fusion protein or other recombinant protein as described herein may be isolated from the bacterial cell paste in a soluble fraction and can be further purified.

In addition to prokaryotes, eukaryotic microbes such as filamentous fungi or yeast are suitable cloning or expression hosts for IL12 fusion protein-encoding vectors, including fungi and yeast strains whose glycosylation pathways have been “humanized,” resulting in the production of an IL12 fusion protein with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing recombinant proteins in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol., 36:59 (1977)); baby hamster kidney cells (BHK); mouse sertoli cells (TM4 cells as described, e.g., in Mather, Biol Reprod, 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumour (MMT 060562); TRI cells, as described, e.g., in Mather et al., Annals N.Y. Acad Sci, 383:44-68 (1982); MRC 5 cells; and FS4 cells. Other useful mammalian host cell lines include Chinese hamster ovary (CHO) cells, including DHFR-CHO cells (Urlaub et al., Proc Natl Acad Sci USA, 77:4216 (1980)); and myeloma cell lines such as YO, NSO and Sp2/0. For a review of certain mammalian host cell lines suitable for fusion protein production, see, e.g., Yazaki & Wu, Methods in Molecular Biology, Vol. 248 (B. K. C. Lo, ed., Humana Press, Totowa, N.J.), pp. 255-268 (2003).

In some embodiments, the IL12 fusion proteins or other recombinant proteins described herein are produced in stable mammalian cells by a method comprising transfecting at least one stable mammalian cell with nucleic acid encoding the IL12 fusion protein or other recombinant protein described herein, in a predetermined ratio, and expressing the nucleic acid in the at least one mammalian cell. In some embodiments, the predetermined ratio of nucleic acid is determined in transient transfection experiments to determine the relative ratio of input nucleic acids that results in the highest percentage of the fusion proteins in the expressed product (see also Example section for Protocols 3 and 4 and Example 3).

In some embodiments, in the method of producing a IL12 fusion protein or other recombinant protein described herein, in stable mammalian cells, the expression product of the stable mammalian cell comprises a larger percentage of the desired HetFc IL12 fusion protein as compared to the monomeric fusion protein. In certain embodiments, the fusion proteins herein are glycosylated.

In some embodiments, in the method of producing a fusion protein in stable mammalian cells, the method further comprises identifying and purifying the desired fusion protein. In some embodiments, identification is by one or both of liquid chromatography and mass spectrometry (see also the Examples herein).

If required, the IL12 fusion proteins or other recombinant proteins can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can be used for purification of IL12 fusion protein. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, Ni+2 affinity chromatography if a His-tag is employed or immobilized anti-flag antibody if a flag-tag is used. For general guidance in suitable purification techniques, see, e.g., Protein Purification: Principles and Practice, 3rd Ed., Scopes, Springer-Verlag, NY (1994). The degree of purification necessary will vary depending on the use of the IL12 fusion protein. In some instances, no purification may be necessary.

In certain embodiments, the IL12 fusion proteins or other recombinant proteins may be purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q or DEAE columns, or their equivalents or comparables.

In some embodiments, the IL12 fusion proteins or other recombinant proteins may be purified using Cation Exchange Chromatography including, but not limited to, chromatography on SP-sepharose, CM sepharose, poros HS, poros CM, Toyopearl SP, Toyopearl CM, Resource/Source S or CM, or Fractogel S or CM columns, or their equivalents or comparables.

In certain embodiments, the IL12 fusion proteins or other recombinant proteins herein are substantially pure. The term “substantially pure” (or “substantially purified”) refers to a construct described herein, or variant thereof, that may be substantially or essentially free of components that normally accompany or interact with the protein as found in its naturally occurring environment, i.e., a native cell, or host cell in the case of recombinantly produced construct. In certain embodiments, a construct that is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein. When the construct is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the construct is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less.

In certain embodiments, the term “substantially purified” as applied to a HetFc IL12 fusion protein comprising a heterodimeric Fc as described herein means that the heterodimeric Fc has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, size-exclusion chromatography (SEC) and capillary electrophoresis.

The IL12 fusion proteins and other recombinant proteins may also be chemically synthesized using techniques known in the art (see, e.g., Creighton, Proteins: Structures and Molecular Principles, W. H. Freeman & Co., N.Y. (1983), and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, alpha-amino isobutyric acid, 4aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as α-methyl amino acids, C α-methyl amino acids, N α-methyl amino acids, and amino acid analogs in general. Furthermore, the amino acid can be D (dextrorotary) or L (levorotary)

Certain embodiments of the present disclosure relate to isolated nucleic acid encoding a masked or nonmasked HetFc IL12 fusion protein or other recombinant protein described herein. Such nucleic acid may encode an amino acid sequence comprising the VL and/or an amino acid sequence comprising the VH of the MM, or a modified IL12 polypeptide, etc.

Certain embodiments relate to vectors (e.g. expression vectors) comprising nucleic acid encoding a HetFc IL12 fusion protein or other recombinant protein described herein. The nucleic acid may be comprised by a single vector, or it may be comprised by more than one vector. In some embodiments, the nucleic acid is comprised by a multicistronic vector.

Certain embodiments relate to host cells comprising such nucleic acid or one or more vectors comprising the nucleic acid. In some embodiments, a host cell comprises (e.g., has been transformed with) a vector comprising a nucleic acid that encodes an amino acid sequence comprising a first fusion protein as described herein (e.g., a first Fc polypeptide fused to a MM etc.) and an amino acid sequence comprising a second fusion protein as described herein (e.g., a second Fc polypeptide fused to an IL12 or IL23 polypeptide). In some embodiments, a host cell comprises (e.g., has been transformed with) a first vector comprising a nucleic acid that encodes an amino acid sequence comprising a first fusion protein as described herein (e.g., a first Fc polypeptide fused to a MM) and a second vector comprising a nucleic acid that encodes an amino acid sequence comprising a second fusion protein as described herein (e.g., a second Fc polypeptide fused to an IL12 or IL23 polypeptide). In some embodiments, the host cell is eukaryotic, e.g., a Chinese Hamster Ovary (CHO) cell, or human embryonic kidney (HEK) cell, or lymphoid cell (e.g. YO, NSO, Sp20 cell).

Certain embodiments relate to a method of making a IL12 fusion protein by culturing a host cell into which nucleic acid encoding the fusion protein has been introduced, under conditions suitable for expression of the IL12 fusion protein, and optionally recovering the IL12 fusion protein from the host cell (or host cell culture medium).

### Post-Translational Modifications

In certain embodiments, the IL12 fusion proteins described herein may be differentially modified during or after translation.

The term “modified,” as used herein, refers to any changes made to a given polypeptide, such as changes to the length of the polypeptide, the amino acid sequence, chemical structure, co-translational modification, or post-translational modification of a polypeptide.

The term “post-translationally modified” refers to any modification of a natural or non-natural amino acid that occurs to such an amino acid after it has been incorporated into a polypeptide chain. The term encompasses, by way of example only, co-translational in vivo modifications, co-translational in vitro modifications (such as in a cell-free translation system), post-translational in vivo modifications, and post-translational in vitro modifications.

In some embodiments, the IL12 fusion proteins may comprise a modification such as glycosylation, acetylation, phosphorylation, amidation, derivatization by known protectingiblocking groups, proteolytic cleavage or linkage to an antibody molecule or antigen-binding construct or other cellular ligand, or a combination of these modifications. In some embodiments, the IL12 fusion proteins may be chemically modified by known techniques including, but not limited to, specific chemical cleavage by cyanogen bromide, trypsin, chymotrypsin, papain, V8 protease or NaBH4; acetylation; formylation; oxidation; reduction or metabolic synthesis in the presence of tunicamycin.

Additional optional post-translational modifications of IL12 fusion proteins or portions thereof, terminal ends, attachment of chemical moieties to the amino acid backbone, chemical modifications of N-linked or O-linked carbohydrate chains, and addition or deletion of an N-terminal methionine residue as a result of prokaryotic host cell expression. The IL12 fusion proteins described herein may optionally be modified with a detectable label, such as an enzymatic, fluorescent, isotopic or affinity label to allow for detection and isolation of the protein. Examples of suitable enzyme labels include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin or aequorin; and examples of suitable radioactive materials include iodine, carbon, sulfur, tritium, indium, technetium, thallium, gallium, palladium, molybdenum, xenon or fluorine.

In some embodiments, the IL12 fusion proteins described herein may be attached to macrocyclic chelators that associate with radiometal ions.

In those embodiments in which the IL12 fusion proteins are modified, either by natural processes, such as post-translational processing, or by chemical modification techniques, the same type of modification may optionally be present in the same or varying degrees at several sites in a given polypeptide. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination (see, e.g., Proteins-Structure and Molecular Properties, 2nd Ed., T. E. Creighton, W. H. Freeman and Company, New York (1993); Post-Translational Covalent Modification of Proteins, B. C. Johnson, Ed., Academic Press, New York, pgs. 1-12 (1983); Seifter et al., Meth. Enzymol. 182:626-646 (1990); Rattan et al., Ann. N.Y. Acad. Sci. 663:48-62 (1992)).

In certain embodiments, the IL12 fusion proteins may be attached to a solid support, which may be particularly useful for immunoassays or purification of polypeptides that are bound by, or bind to, or associate with proteins described herein. Such solid supports include, but are not limited to, glass, cellulose, polyacrylamide, nylon, polystyrene, polyvinyl chloride or polypropylene.

### Pharmaceutical Compositions

Also provided herein are pharmaceutical compositions comprising a IL12 fusion protein described herein. Pharmaceutical compositions comprise the IL12 fusion protein and a pharmaceutically acceptable carrier.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. In some aspects, the carrier is a man-made carrier not found in nature. Water can be used as a carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the IL12 fusion protein, preferably in purified form, together with a suitable amount of carrier so as to provide the form for proper administration to the patient. The formulation should suit the mode of administration.

In certain embodiments, the composition comprising a IL12 fusion protein is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, the compositions described herein are formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxide isopropylamine, triethylamine, 2-ethylamino ethanol, histidine, procaine, etc.

### Methods of Use

The present disclosure provides methods of using the IL12 fusion proteins and other recombinant fusion proteins comprising the modified p40 domains described herein.

In particular, further provided herein are methods of treating a subject with or at risk of developing cancer, autoimmune disease, inflammatory disorders or an infectious disease. Further provided herein are methods of treating a subject with or at risk of developing a disease selected from the group consisting of: all types of cancers, such as, but not limited to breast, including by way of non-limiting example, triple negative breast cancer, ER/PR+ breast cancer, and Her2+ breast cancer, lung cancer (e.g., non-small cell squamous and adenocarcinoma), colorectal cancer, gastric cancer, glioblastoma, ovarian cancer, endometrial cancer, renal cancer, sarcoma, skin cancer, cervical cancer, liver cancer, bladder cancer, cholangiocarcinoma, prostate cancer, melanomas, head and neck cancer (e.g. head and neck squamous cell carcinoma), esophageal, squamous cell cancer, basal cell carcinoma, pancreatic cancer, leukemias, including T-cell acute lymphoblastic leukemia (T-ALL), lymphoblastic diseases including multiple myeloma, solid tumors, bone disease or metastasis in cancer, regardless of primary tumor origin. Further provided are methods of treating a subject with or at risk of developing rheumatoid arthritis, Crohn's disease, SLE, cardiovascular damage, or ischemia.

In certain embodiments, the present disclosure provides methods of treating a disease in a subject by administering to the subject a therapeutically effective amount of a cytokine fusion protein disclosed herein where the disease is selected from the group consisting of colorectal cancer, pancreatic cancer, head and neck cancer, esophageal cancer, bladder cancer, cervical cancer, and lung cancer (e.g., non-small cell squamous and adenocarcinoma).

The methods comprise administering to the subject in need thereof an effective amount of a IL12 fusion protein or other recombinant fusion protein as described herein (e.g., comprising a modified p40 domain) (a fusion protein) as disclosed herein that is typically administered as a pharmaceutical composition. In some embodiments, the method further comprises selecting a subject with or at risk of developing cancer. In some embodiments, the pharmaceutical composition comprises a L12 fusion protein, or a fragment thereof that is activated at a tumor site. In one embodiment, the tumor is a solid tumor.

In certain embodiments, provided is a method of treating a cancer comprising administering to a subject in which such treatment or amelioration is desired, a IL12 fusion protein described herein, in an amount effective to treat or ameliorate the cancer. In other embodiments, there is provided a method of using the IL12 fusion protein described herein in the preparation of a medicament for the treatment or amelioration of cancer in a subject.

The term “subject” refers to an animal, in some embodiments a mammal, which is the object of treatment, observation or experiment. An animal may be a human, a non-human primate, a companion animal (e.g., dogs, cats, and the like), farm animal (e.g., cows, sheep, pigs, horses, and the like) or a laboratory animal (e.g., rats, mice, guinea pigs, and the like).

The term “mammal” as used herein includes but is not limited to humans, non-human primates, canines, felines, murines, bovines, equines, and porcines.

“Treatment” refers to clinical intervention in an attempt to alter the natural course of the individual or cell being treated and can be performed during the course of clinical pathology. Desirable effects of treatment include preventing recurrence of disease, alleviation of symptoms, diminishing of any direct or indirect pathological consequences of the disease, preventing metastasis, decreasing the rate of disease progression, amelioration or palliation of the disease state, and remission or improved prognosis. In some embodiments, IL12 fusion protein described herein are used to delay development of a disease or disorder. In one embodiment, IL12 fusion protein described herein, and methods described herein effect tumor regression. In one embodiment, IL12 fusion protein described herein, and methods described herein effect inhibition of tumor/cancer growth.

Desirable effects of treatment include, but are not limited to, one or more of preventing recurrence of disease, alleviation of symptoms, diminishment of any direct or indirect pathological consequences of the disease, preventing metastasis, decreasing the rate of disease progression, amelioration or palliation of the disease state, improved survival, and remission or improved prognosis. In some embodiments, IL12 fusion protein described herein are used to delay development of a disease or to slow the progression of a disease.

In some embodiments, the IL12 fusion proteins of the present disclosure can be used to prevent occurrence of a disease, e.g., cancer.

The term “effective amount” as used herein refers to that amount of a IL12 fusion protein described herein or a composition comprising a IL12 fusion protein described herein being administered, which will accomplish the goal of the recited method, e.g., relieve to some extent one or more of the symptoms of the disease, condition or disorder being treated. The amount of the composition described herein which will be effective in the treatment and inhibition of a disease or disorder associated with aberrant expression and/or activity of a therapeutic protein can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses are extrapolated from dose-response curves derived from in vitro or animal model test systems.

The IL12 fusion protein described herein is administered to a subject. Various delivery systems are known and can be used to administer an IL12 fusion protein formulation described herein, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intra-tumoral, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, in certain embodiments, it is desirable to introduce the IL12 fusion protein compositions described herein into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it is desirable to administer the IL12 fusion proteins described herein, or compositions described herein, locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including a IL12 fusion protein described herein, care must be taken to use materials to which the protein does not absorb.

In another embodiment, the IL12 fusion proteins described herein or composition comprising same can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, a IL12 fusion protein described herein or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, e.g., the brain, thus requiring only a fraction of the systemic dose (see, e.g., Goodson, in Medical Applications of Controlled Release, vol. 2, pp. 115-138 (1984)).

In a specific embodiment comprising a nucleic acid encoding a IL12 fusion protein described herein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The IL12 fusion proteins described herein may be administered alone or in combination with other types of treatments (e.g., radiation therapy, chemotherapy, hormonal therapy, immunotherapy, immune checkpoint inhibitors, and anti-tumor agents). Generally, administration of products of a species origin or species reactivity (in the case of antibodies) that is the same species as that of the patient is preferred.

The IL12 fusion proteins described herein may be used in the treatment of cancer. In some embodiments, the IL12 fusion proteins described herein may be used in the treatment of a patient who has undergone one or more alternate forms of anti-cancer therapy. In some embodiments, the patient has relapsed or failed to respond to one or more alternate forms of anti-cancer therapy. In other embodiments, a IL12 fusion protein is administered to a patient in combination with one or more alternate forms of anti-cancer therapy. In other embodiments, the IL12 fusion protein is administered to a patient that has become refractory to treatment with one or more alternate forms of anti-cancer therapy.

### Kits and Articles of Manufacture

Also described herein are kits comprising one or more fusion protein(s) or another recombinant protein described herein. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the masked IL12 fusion proteins.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilized form and the kit can additionally contain a suitable solvent for reconstitution of the lyophilized components. Irrespective of the number or type of containers, the kits described herein also may comprise an instrument for assisting with the administration of the composition to a patient. Such an instrument may be an inhalant, nasal spray device, syringe, pipette, forceps, measured spoon, eye dropper or similar medically approved delivery vehicle.

Certain embodiments relate to an article of manufacture containing materials useful for treatment of a patient as described herein. The article of manufacture comprises a container and a label or package insert on or associated with the container. Suitable containers include, for example, bottles, vials, syringes, intravenous solution bags, etc. The containers may be formed from a variety of materials such as glass or plastic. The container holds a composition comprising the IL12 fusion protein which is by itself or combined with another composition effective for treating the patient and may have a sterile access port (for example the container may be an intravenous solution bag or a vial having a stopper pierceable by a hypodermic injection needle). The label or package insert indicates that the composition is used for treating the condition of choice. In some embodiments, the article of manufacture may comprise (a) a first container with a composition contained therein, wherein the composition comprises a IL12 fusion protein described herein; and (b) a second container with a composition contained therein, wherein the composition in the second container comprises a further cytotoxic or otherwise therapeutic agent. In such embodiments, the article of manufacture may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. The article of manufacture may optionally further include other materials desirable from a commercial and user standpoint, including other buffers, diluents, filters, needles, and syringes.

### EXEMPLARY EMBODIMENTS

Further particular embodiments of the present disclosure are described as follows.

These embodiments are intended to illustrate the compositions and methods described in the present disclosure and are not intended to limit the scope of the present disclosure. Furthermore, the terms “p40 domain” and “p40 subunit” can be used interchangeably herein.

Embodiment 1. A modified p40 subunit of interleukin 12 (IL12) or IL23 protein, comprising a human IL12 p40 subunit polypeptide, wherein the polypeptide comprises at least one amino acid substitution relative to the wild-type human IL-12 p40 of SEQ ID NO: 10 at one or more positions corresponding to residues 15, 18, 45, 58, 59, 60, 62, 84, 86, 93, 161, 195, and 197.

Embodiment 2. The modified IL12 or IL23 p40 subunit polypeptide of embodiment 1, wherein at least one amino acid substitution is a substitution selected from the group consisting of W15H, W15K, W15R, D18G, E45K, E45R, K58H, K58S, K58W, E59D, E59G, E59R, E59S, F60D, F60E, F60K, F60R, F60V, D62H, D62I, D62N, K84E, K84I, K84L, K84V, K84W, K84Y, E86L, E86R, E865, E86W, D93E, D93H, D93R, D93W, D161R, D161S, K195D, K197D, K197E, K197Q, K197T, and K197W.

Embodiment 3. The modified IL12 or IL23 p40 subunit polypeptide of embodiment 2, wherein at least one amino acid substation is a substitution selected from the group consisting of W15H, W15K, D18G, E45K, E45R, K58H, K58S, K58W, E59G, E59R, E59S, F60V, D62H, D62I, D62N, K84E, K84W, E86L, E86S, E86W, D93H, D93W, D161R, D161S, K195D, K197E, K197Q, K197T, and K197W.

Embodiment 4. The modified IL12 or IL23 p40 subunit polypeptide of embodiments 2 or 3, wherein at least one amino acid substitution comprises a substitution selected from the group consisting of W15H, W15K, E59R, E59S, K84E, K84W, E86W, D161R, K197T, and K197W.

Embodiment 5. The modified IL12 or IL 23 p40 subunit polypeptide of embodiment 2, wherein at least one amino acid substitution comprises a substitution selected from the group consisting of W15H, W15R, E45R, K58H, K58S, E59D, E59G, E59R, E59S, F60D, F60E, F60K, F60R, K84E, K84I, K84L, K84V, K84W, K84Y, E86R, E86W, D93E, D93H, D93R, D93W, D161R, K195D, K197D, K197E, K197Q, and K197W.

Embodiment 6. The modified IL12 or IL23 p40 subunit polypeptide of embodiments 2 or 5 comprising: W15H_K84L, K58H_K84I, E59D_K84W, E59G_K84W, E59R_K84E, E59R_K84W, E59R_E86W, E59D_D93H, E59R_D93R, E59R_K197E, E59R_K197W, F60E_K84W, F60R_K84Y, F60K_K197W, F60R_K197W, K84I_E86R, K84E_D93H, K84I_D93H, K84V_D93H, K84W_D93W, K84I_D161R, K84W_D161R, K84W_K197E, K84W_K197Q, K84W_K197W, E86W_D93E, E86R_K197D, E86W_K197W, W15H_K84L_K197Q, K58S_E59S_K195D, K58H_E86R_K197D, E59D_K84W_K197W, F60R_K84E_K197W, K84I_E86R_D93H, W15R_E59D_F60D_K197W, and E45R_K58S_E59S_K195D.

Embodiment 7. The modified IL12 or IL 23 p40 subunit polypeptide of embodiment 6 comprising: W15H_K84L, E59D_D93H, E59R_D93R, F60K_K197W, F60R_K84Y, K84I_E86R, K84W_D161R, K84W_K197W, E86R_K197D, E86W_D93E, W15H_K84L_K197Q, K58H_E86R_K197D, K58S_E59S_K195D, E59D_K84W_K197W, F60R_K84E_K197W, K84I_E86R_D93H, W15R_E59D_F60D_K197W, and E45R_K58S_E59S_K195D.

Embodiment 8. The modified IL12 or IL23 p40 subunit polypeptide of embodiment 6 comprising: K58H_K84I, E59D_K84W, E59G_K84W, E59R_E86W, E59R_K197E, E59R_K197W, E59R_K84E, E59R_K84W, F60E_K84W, F60R_K197W, K84E_D93H, K84I_D161R, K84I_D93H, K84V_D93H, K84W_D93W, K84W_K197E, K84W_K197Q, and E86W_K197W.

Embodiment 9. The modified IL12 or IL23 p40 subunit polypeptide of embodiments 6-8 comprising: W15H_K84L, K58H_K84I, E59D_K84W, E59G_K84W, E59R_K84E, E59R_K84W, E59R_E86W, E59D_D93H, E59R_D93R, E59R_K197E, E59R_K197W, F60E_K84W, F60R_K84Y, F60K_K197W, F60R_K197W, K84I_E86R, K84E_D93H, K84I_D93H, K84V_D93H, K84W_D93W, K84I_D161R, K84W_D161R, K84W_K197E, K84W_K197Q, K84W_K197W, E86W_D93E, E86R_K197D, and E86W_K197W.

Embodiment 10. The modified IL12 or IL23 p40 subunit polypeptide of embodiments 6-9 comprising: W15H_K84L, E59D_D93H, E59R_D93R, F60K_K197W, F60R_K84Y, K84I_E86R, K84W_D161R, K84W_K197W, E86R_K197D, and E86W_D93E.

Embodiment 11. The modified IL12 or IL 23 p40 subunit polypeptide of embodiment 7-8 comprising: W15H_K84L_K197Q, K58H_E86R_K197D, K58S_E59S_K195D, E59D_K84W_K197W, F60R_K84E_K197W, K84I_E86R_D93H, W15R_E59D_F60D_K197W, and E45R_K58S_E59S_K195D.

Embodiment 12. The modified IL12 or IL 23 p40 subunit polypeptide of any one of embodiments 1-11, comprising a single amino acid substitution relative to human IL12 p40 subunit of SEQ ID NO: 10.

Embodiment 13. An IL12 protein containing the modified IL12 p40 subunit polypeptide of any one of embodiments 1-12. 14. An IL23 protein containing a modified IL23 p40 subunit polypeptide of any one of embodiment 1-12.

Embodiment 14. A fusion protein comprising the modified IL12 or IL 23 p40 subunit polypeptide of any one of embodiments 1-14 and a heterologous polypeptide.

Embodiment 15. The fusion protein of embodiment 14, wherein the heterologous polypeptide is an antibody or fragment thereof.

Embodiment 16. The fusion protein of embodiment 15, wherein the heterologous polypeptide is a heterodimeric Fc protein.

Embodiment 17. A composition comprising the modified IL12 or IL23 p 40 subunit polypeptides of any one of embodiments 1-14 or the fusion protein of any one of embodiments 15-16, and a pharmaceutically acceptable carrier.

Embodiment 18. A composition of embodiment 17 for use in treating cancer in a subject.

Embodiment 19. A nucleic acid molecule encoding the modified IL12 or IL23 p40 subunit polypeptide of any one of embodiments 1-14 or the fusion protein of any one of embodiments 15-16.

Embodiment 20. A vector comprising the nucleic acid molecule of embodiment 19.

Embodiment 21. A composition comprising the nucleic acid molecule of embodiment 19 or the vector of embodiment 20, and a pharmaceutically acceptable carrier.

Embodiment 22. A modified p40 domain, the modified p40 domain comprising one or more amino acid substitutions relative to the wild-type human mature IL12 p40 domain sequence set forth in SEQ ID NO: 10, wherein the one or more amino acid substitutions are located at one or more positions of E45, D62 and D161, and wherein the numbering of the amino acid residues is based on the amino acid sequence set forth in SEQ ID NO: 10.

Embodiment 23. The modified p40 domain of embodiment 22, wherein the one or more amino acid substitutions at the one or more positions are a K, H, I, N, R or an S substitution, or a combination thereof.

Embodiment 24. The modified p40 domain of any one of embodiments 22-23, wherein the one or more amino acid substitutions comprise E45K.

Embodiment 25. The modified p40 domain of any one of embodiments 22-24, wherein the one or more amino acid substitutions comprise D62H.

Embodiment 26. The modified p40 domain of any one of embodiments 22-24, wherein the one or more amino acid substitutions comprise D62I.

Embodiment 27. The modified p40 domain of any one of embodiments 22-24, wherein the one or more amino acid substitutions comprise D62N.

Embodiment 28. The modified p40 domain of any one of embodiments 22-27, wherein the one or more amino acid substitutions comprise D161R.

Embodiment 29. The modified p40 domain of any one of embodiments 22-27, wherein the one or more amino acid substitutions comprise D161S.

Embodiment 30. The modified p40 domain of any one of embodiments 22-29, wherein the one or more amino acid substitutions comprise a combination of two or more substitutions of E45K, D62H, D62I, D62N, D161R or D161S.

Embodiment 31. A modified p40 domain, the modified p40 domain comprising one or more amino acid substitutions relative to the wild-type human mature IL12 p40 domain sequence set forth in SEQ ID NO: 10, wherein the one or more amino acid substitutions are W15H, W15K, W15R, D18G, E45K, K58H, K58W, E59D, E59G, E59R, F60D, F60E, F60K, F60R, F60V, D62H, D62I, D62N, K84E, K84I, K84L, K84V, K84W, K84Y, E86L, E86R, E86S, E86W, D93E, D93H, D93R, D93W, D161R, D161S, K197D, K197E, K197Q, K197T, or K197W, or a combination thereof, and wherein the numbering of the amino acid residues is based on the amino acid sequence set forth in SEQ ID NO: 10.

Embodiment 32. The modified p40 domain of embodiment 31, wherein the modified p40 domain comprises one or more, two or more, or three or more amino acid substitutions.

Embodiment 33. The modified p40 domain of any one of embodiments 31-32, wherein the one or more amino acid substitutions are W15H, W15K, D18G, E45K, K58H, K58W, E59G, E59R, F60V, D62H, D62I, D62N, K84E, K84W, E86L, E86S, E86W, D93H, D93W, D161R, D161S, K197E, K197Q, K197T, or K197W, or a combination thereof.

Embodiment 34. The modified p40 domain of any one of embodiments 31-33, wherein the one or more amino acid substitutions are W15H, W15K, E59R, K84E, K84W, E86W, D161R, K197T, or K197W, or a combination thereof.

Embodiment 35. The modified p40 domain of any one of embodiments 31-32, wherein the two or more amino acid substitutions are W15H_K84L, K58H_K84I, E59D_K84W, E59G_K84W, E59R_K84E, E59R_K84W, E59R_E86W, E59D_D93H, E59R_D93R, E59R_K197E, E59R_K197W, F60E_K84W, F60R_K84Y, F60K_K197W, F60R_K197W, K84I_E86R, K84E_D93H, K84I_D93H, K84V_D93H, K84W_D93W, K84I_D161R, K84W_D161R, K84W_K197E, K84W_K197Q, K84W_K197W, E86W_D93E, E86R_K197D, E86W_K197W, W15H_K84L_K197Q, K58H_E86R_K197D, E59D_K84W_K197W, F60R_K84E_K197W, K84I_E86R_D93H, or W15R_E59D_F60D_K197W, or a combination thereof.

Embodiment 36. The modified p40 domain of embodiment 35, wherein the two or more amino acid substitutions are W15H_K84L, E59D_D93H, E59R_D93R, F60K_K197W, F60R_K84Y, K84I_E86R, K84W_D161R, K84W_K197W, E86R_K197D, E86W_D93E, W15H_K84L_K197Q, K58H_E86R_K197D, E59D_K84W_K197W, F60R_K84E_K197W, K84I_E86R_D93H, or W15R_E59D_F60D_K197W, or a combination thereof.

Embodiment 37. The modified p40 domain of embodiment 35, wherein the two or more amino acid substitutions are K58H_K84I, E59D_K84W, E59G_K84W, E59R_E86W, E59R_K197E, E59R_K197W, E59R_K84E, E59R_K84W, F60E_K84W, F60R_K197W, K84E_D93H, K84I_D161R, K84I_D93H, K84V_D93H, K84W_D93W, K84W_K197E, K84W_K197Q, or E86W_K197W, or a combination thereof.

Embodiment 38. The modified p40 domain of embodiment 35, wherein the two or more amino acid substitutions are W15H_K84L, K58H_K84I, E59D_K84W, E59G_K84W, E59R_K84E, E59R_K84W, E59R_E86W, E59D_D93H, E59R_D93R, E59R_K197E, E59R_K197W, F60E_K84W, F60R_K84Y, F60K_K197W, F60R_K197W, K84I_E86R, K84E_D93H, K84I_D93H, K84V_D93H, K84W_D93W, K84I_D161R, K84W_D161R, K84W_K197E, K84W_K197Q, K84W_K197W, E86W_D93E, E86R_K197D, or E86W_K197W, or a combination thereof.

Embodiment 39. The modified p40 domain of embodiment 35, wherein the two or more amino acid substitutions are W15H_K84L, E59D_D93H, E59R_D93R, F60K_K197W, F60R_K84Y, K84I_E86R, K84W_D161R, K84W_K197W, E86R_K197D, or E86W_D93E, or a combination thereof.

Embodiment 40. The modified p40 domain of any one of embodiments 31-32, wherein the three or more amino acid substitutions are W15H_K84L_K197Q, K58H_E86R_K197D, E59D_K84W_K197W, F60R_K84E_K197W, K84I_E86R_D93H or W15R_E59D_F60D_K197W, or a combination thereof.

Embodiment 41. The modified p40 domain of any one of embodiments 31-32, wherein the one or more amino acid substitutions are selected from Table C.

Embodiment 42. The modified p40 domain of any one of embodiments 31-32, wherein the modified p40 domain comprises or consists of an amino acid sequence having at least about 90%, 95%, 97%, 99%, or 100% sequence identity to the amino acid sequence set forth in any one of SEQ ID NOS: 12-14, 16, 18, 19, 21-34 and 36-56.

Embodiment 43. The modified p40 domain of any one of embodiments 31-32, wherein the modified p40 domain comprises an amino acid substitution or a set of amino acid substitutions selected from: W15R_E59D_F60D_K197W, W15H_K84L_K197Q, E59D_K84W_K197W, K58H_E86R_K197D, W15H_K84L, F60R_K84E_K197W, K84W_K197W, F60K_K197W, E86R_K197D, K84I_E86R_D93H, E59R_D93R, K84I_E86R, W15K, K84W_D161R, E45R_K58S_E59S_K195D, E59D_D93H, F60R_K84Y, E86W_D93E, K58S_E59S_K195D, K84E, K197T, E59R, W15H, K84W, K197W, E595, D161R or E86W, or a combination thereof.

Embodiment 44. The modified p40 domain of embodiment 43, wherein the modified p40 domain comprises an amino acid substitution or a set of amino acid substitutions selected from: W15H_K84L_K197Q, E59D_K84W_K197W, K84W_K197W, F60K_K197W, E59R_D93R, W15K, or F60R_K84Y, or a combination thereof.

Embodiment 45. The modified p40 domain of any one of embodiments 22-44, wherein the modified p40 domain has a binding affinity to at least one of its cognate receptors that is reduced from about 5-fold to about 1000-fold, from about 5-fold to about 800-fold, from about 5-fold to about 600-fold, from about 10-fold to about 500-fold, from about 10-fold to about 300-fold, or from about 20-fold to about 200-fold, relative to the binding affinity of the unmodified wildtype p40 domain having the sequence set forth in SEQ ID NO: 10, and as determined in a reporter gene assay (RGA).

Embodiment 46. The modified p40 domain of embodiment 45, wherein the modified p40 domain has a binding affinity to at least one of its cognate receptors that is reduced from about 20-fold to about 200-fold.

Embodiment 47. The modified p40 domain of any one of embodiments 45-46, wherein the at least one cognate receptor comprises IL12Rβ1.

Embodiment 48. The modified p40 domain of any one of embodiments 22-47, wherein the modified p40 domain has a thermostability as measured by a melting temperature that is within ±5° C., ±4° C., ±3° C., ±2° C. or ±1° C. of that of the unmodified wildtype p40 domain that has the sequence set forth in SEQ ID NO: 10, and wherein the melting temperature is determined by Differential Scanning Fluorimetry (DSF) or Differential Scanning Calorimetry (DSC).

Embodiment 49. An IL12 fusion protein comprising the modified p40 domain of any one of embodiments 22-48.

Embodiment 50. An IL12 fusion protein comprising an IL12 polypeptide, wherein the IL12 polypeptide comprises the modified p40 domain of any one of embodiments 22-48; coupled to a p35 domain.

Embodiment 51. The IL12 fusion protein of embodiment 50, wherein the p35 domain comprises or consists of an amino acid sequence having at least about 90%, 95%, 97%, 99% or 100% sequence identity to the amino acid sequence set forth in SEQ ID NO: 11.

Embodiment 52. The IL12 fusion protein of any one of embodiments 50-51, wherein the N-terminus of the p35 domain is coupled to the C-terminus of the modified p40 domain either directly or via a first linker.

Embodiment 53. The IL12 fusion protein of embodiment 52, wherein the first linker has the sequence of (G4S)x, and wherein xis 1, 2, 3 or 4.

Embodiment 54. The IL12 fusion protein of any one of embodiments 50-53, further comprising a heterodimeric Fc (HetFc) domain comprising a first Fc polypeptide and a second Fc polypeptide, thereby forming an IL12 HetFc fusion protein.

Embodiment 55. The IL12 HetFc fusion protein of embodiment 54, wherein the IL12 polypeptide is coupled to the first Fc polypeptide either directly or via a second linker.

Embodiment 56. The IL12 HetFc fusion protein of embodiment 55, wherein the IL12 polypeptide is coupled to the C-terminus of the first Fc polypeptide.

Embodiment 57. The IL12 HetFc fusion protein of any one of embodiments 55-56, wherein the IL12 polypeptide is coupled to the C-terminus of the first Fc polypeptide via the N-terminus of the modified p40 domain.

Embodiment 58. The IL12 HetFc fusion protein of any one of embodiments 55-57, further comprising a masking moiety, thereby forming a masked IL12 HetFc fusion protein, wherein the masking moiety is capable of non-covalently interacting with the modified p40 domain, thereby masking the modified p40 domain and reducing the binding affinity (K_{D}) of the modified p40 domain for binding to at least one of its cognate receptors when compared to an unmasked modified p40 domain.

Embodiment 59. The masked IL12 HetFc fusion protein of embodiment 58, wherein the masking moiety is coupled to the C-terminus of the second Fc polypeptide either directly or via a third linker.

Embodiment 60. The masked IL12 HetFc fusion protein of embodiment 59, wherein the third linker is a protease-cleavable linker.

Embodiment 61. The masked IL12 HetFc fusion protein of any one of embodiments 58-60, wherein the second linker and the third linker each comprise or consist of an amino acid sequence spanning from 5 to about 50 amino acids.

Embodiment 62. The masked IL12 HetFc fusion protein of embodiment 61, wherein the second linker comprises or consists of the amino acid sequence set forth in SEQ ID NO: 132, and the third linker comprises or consists of the amino acid sequence set forth in SEQ ID NO: 134.

Embodiment 63. The masked IL12 HetFc fusion protein of any one of embodiments 58-62, wherein the masking moiety comprises or consists of an scFv domain comprising a V_{H} domain coupled either directly or via a fourth linker to a V_{L} domain.

Embodiment 64. The masked IL12 HetFc fusion protein of embodiment 63, wherein the fourth linker comprises or consists of the amino acid sequence set forth in SEQ ID NO: 135.

Embodiment 65. The masked IL12 HetFc fusion protein of any one of embodiments 63-64, wherein the V_{H} domain comprises or consists of an amino acid sequence having about 95%, 97%, 99%, or 100% sequence identity to the sequence set forth in SEQ ID NO: 2, and the V_{L} domain comprises or consists of an amino acid sequence having about 95%, 97%, 99%, or 100% sequence identity to the sequence set forth in SEQ ID NO: 3.

Embodiment 66. The masked IL12 HetFc fusion protein of any one of embodiments 58-65, wherein the masking moiety is capable of reducing the binding affinity of the modified p40 domain to the at least one cognate receptor by at least about 10-fold, 15-fold, 20-fold, 30-fold, 40-fold, 50-fold, 100-fold, 200-fold, or at least about 300-fold, compared to a corresponding polypeptide construct that does not comprise the masking moiety.

Embodiment 67. The masked IL12 HetFc fusion protein of embodiment 66, wherein the at least one cognate receptor comprises IL12Rβ1.

Embodiment 68. A masked IL12 HetFc fusion protein comprising:
(i) an IL-12 polypeptide comprising the modified p40 domain according to any one of embodiments 22-48 coupled via the linker (G4S)₄ to a p35 domain comprising or consisting of the amino acid sequence set forth in SEQ ID NO: 11;
(ii) a heterodimeric Fc domain comprising a first Fc polypeptide and a second Fc polypeptide; and
(iii) a masking moiety (MM) comprising an anti-IL12 scFv domain, wherein:
   the IL12 polypeptide is coupled either directly or via a second linker to the C-terminus of the first Fc polypeptide, and
   the masking moiety is coupled either directly or via a third linker to the C-terminus of the second Fc polypeptide and is capable of non-covalently interacting with the IL12 polypeptide, thereby reducing the binding affinity of the IL12 polypeptide to at least one of its cognate receptors.

Embodiment 69. The masked IL12 HetFc fusion protein of embodiment 68, comprising or consisting of two polypeptide chains, from N- to C-terminus, (i) an Fc-IL12 polypeptide chain and (ii) an Fc-MM polypeptide chain.

Embodiment 70. The masked IL12 HetFc fusion protein of embodiment 69, wherein the Fc-IL12 polypeptide chain comprises or consists of an amino acid sequence having at least about 90%, 95%, 97%, 99%, or 100% sequence identity to any of the amino acid sequences set forth in SEQ ID NOs: 61-89.

Embodiment 71. The masked IL12 HetFc fusion protein of any one of embodiments 69-70, wherein the Fc-IL12 polypeptide chain comprises or consists of the amino acid sequence set forth in any one of SEQ ID NOs: 61-89.

Embodiment 72. The masked IL12 HetFc fusion protein of any one of embodiments 69-71, wherein the Fc-MM polypeptide chain comprises or consists of an amino acid sequence having at least about 90%, 95%, 97%, 99%, or 100% sequence identity to the amino acid sequence set forth in SEQ ID NO: 60.

Embodiment 73. The masked IL12 HetFc fusion protein of any one of embodiments 69-72, wherein the Fc-MM polypeptide chain comprises or consists of the amino acid sequence set forth in SEQ ID NO: 60.

Embodiment 74. The masked IL12 HetFc fusion protein of any one of embodiments 69-73, wherein (i) the Fc-IL12 polypeptide chain amino acid sequence is selected from the group consisting of the sequences of: v28046, v28047, v28048, v28049, v28050, v28051, v28053, v28054, v28055, v28056, v28057, v28058, v28059, v28060, v28061, v28062, v28063, v28064, v28065, v28066, v28067, v28068, v28069, v28070, v28071, v28072, v28074, v28075, and (ii) the Fc-MM polypeptide chain comprises or consists of the amino acid sequence of v26503.

Embodiment 75. An unmasked IL12 HetFc fusion protein comprising: (i) an IL-12 polypeptide comprising the modified p40 domain according to any one of embodiments 22-48 coupled via the linker (G4S)₄ to the p35 domain comprising or consisting of the amino acid sequence set forth in SEQ ID NO: 11; and (ii) a heterodimeric Fc domain comprising a first Fc polypeptide and a second Fc polypeptide, wherein the IL12 polypeptide is coupled either directly or via a second linker to the C-terminus of the first Fc polypeptide.

Embodiment 76. The unmasked IL12 HetFc fusion protein of embodiment 75, comprising or consisting of two polypeptide chains, from N- to C-terminus, (i) an Fc-IL12 polypeptide chain and (ii) an Fc polypeptide chain.

Embodiment 77. The unmasked IL12 HetFc fusion protein of embodiment 76, wherein the Fc-IL12 polypeptide chain comprises or consists of an amino acid sequence having at least about 90%, 95%, 97%, 99%, or 100% sequence identity to any of the amino acid sequences set forth in SEQ ID NOs: 61-89.

Embodiment 78. The unmasked IL12 HetFc fusion protein of any one of embodiments 76-77, wherein the Fc-IL12 polypeptide chain comprises or consists of the amino acid sequence set forth in any one of SEQ ID NOs: 61-89.

Embodiment 79. The unmasked IL12 HetFc fusion protein of any one of embodiments 76-78, wherein the Fc polypeptide chain comprises or consists of an amino acid sequence having at least about 90%, 95%, 97%, 99%, or 100% sequence identity to the amino acid sequence set forth in SEQ ID NO: 58.

Embodiment 80. The unmasked IL12 HetFc fusion protein of any one of embodiments 76-79, wherein (i) the IL12 polypeptide chain amino acid sequence is selected from the group consisting of the sequences of: v28046, v28047, v28048, v28049, v28050, v28051, v28053, v28054, v28055, v28056, v28057, v28058, v28059, v28060, v28061, v28062, v28063, v28064, v28065, v28066, v28067, v28068, v28069, v28070, v28071, v28072, v28074, v28075, and (ii) the Fc polypeptide chain comprises or consists of the amino acid sequence of v12153.

Embodiment 81. A pharmaceutical composition comprising (i) the modified p40 domain of any one of embodiments 22-48, (ii) the masked IL12 HetFc fusion protein of any one of embodiments 58-74, and/or (iii) the unmasked IL12 HetFc fusion protein of embodiments 75-80, and a pharmaceutically acceptable carrier.

Embodiment 82. A nucleic acid molecule or a set of nucleic acid molecules encoding (i) the modified p40 domain of any one of embodiments 22-48, (ii) the masked IL12 HetFc fusion protein of any one of embodiments 58-74, and/or (iii) the unmasked IL12 HetFc fusion protein of embodiments 75-80.

Embodiment 83. A vector or a set of vectors comprising the nucleic acid molecule or the set of nucleic acid molecules of embodiment 82.

Embodiment 84. A method of identifying one or more amino acid substitutions in a p40 domain amino acid sequence to produce a modified p40 domain, the method comprising performing molecular dynamics and mutagenesis simulations, thereby identifying the one or more amino acid substitutions listed in Table C, wherein the one or more amino acid substitutions in the modified p40 domain amino acid sequence are relative to the sequence set forth in SEQ ID NO: 1, and wherein the one or more amino acid substitutions reduce the binding affinity (K_{D}) of the modified p40 domain to at least one of its cognate receptors, and relative to an unmodified p40 domain that does not comprise the one or more amino acid substitutions.

Embodiment 85. The method of embodiment 84, wherein the at least one cognate receptor comprises IL12Rβ1.

Embodiment 86. The method of any one of embodiments 84-85, wherein the binding affinity of the modified p40 domain is reduced by at least about 2-fold, 5-fold, 10-fold, 15-fold, 20-fold, 30-fold, 40-fold, 50-fold, 100-fold, 200-fold, 300-fold, 400-fold, 500-fold, or at least about 600-fold, compared to an unmodified p40 domain and as determined in a reporter gene assay.

Embodiment 87. A method of treating a disease in a subject in need thereof, the method comprising administering to the subject a therapeutically effective amount of a pharmaceutical composition comprising (i) the modified p40 domain of any one of embodiments 22-48, (ii) the masked IL12 HetFc fusion protein of any one of embodiments 58-74, and/or (iii) the unmasked IL12 HetFc fusion protein of embodiments 75-80, thereby treating the disease in the subject.

Embodiment 88. The method of embodiment 87, wherein the polypeptide construct is the masked IL12 HetFc fusion protein of any one of embodiments 58-74.

Embodiment 89. The method of embodiment 88, wherein the masking moiety of the masked IL12 HetFc fusion protein is cleaved from the fusion protein in a diseased tissue or organ.

Embodiment 90. The method of any one of embodiments 87-89, wherein the disease is a cancer.

Embodiment 91. A modified p40 domain of any one of embodiments 22-48, a masked IL12 HetFc fusion protein of any one of embodiments 58-74, or an unmasked IL12 HetFc fusion protein of any one of embodiments 75-80, for use in therapy.

Embodiment 92. A modified p40 domain of any one of embodiments 22-48, a masked IL12 HetFc fusion protein of any one of embodiments 58-74, or an unmasked IL12 HetFc fusion protein of any one of embodiments 75-80, for use in the treatment of cancer.

Embodiment 93. Use of a modified p40 domain of any one of embodiments 22-48, a masked IL12 HetFc fusion protein of any one of embodiments 58-74, or an unmasked IL12 HetFc fusion protein of any one of embodiments 75-80, in the manufacture of a medicament for the treatment of cancer.

Unless defined otherwise, all technical and scientific terms used herein have the same meaning as is commonly understood by one of skill in the art to which the claimed subject matter belongs. In the event that there is a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information. Terms understood by those in the art of antibody technology are each given the meaning acquired in the art, unless expressly defined differently herein.

It is to be understood that the general description and following detailed description are exemplary and explanatory only and are not restrictive of any subject matter claimed.

In this application, the use of the singular includes the plural unless specifically stated otherwise.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” means±10% of the indicated range, value, sequence, or structure, unless otherwise indicated. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives. As used herein, the terms “include” and “comprise” are used synonymously. In addition, it should be understood that the individual single chain polypeptides or immunoglobulin constructs derived from various combinations of the structures and substituents described herein are disclosed by the present application to the same extent as if each single chain polypeptide or heterodimer were set forth individually. Thus, selection of particular components to form individual single chain polypeptides or heterodimers is within the scope of the present disclosure.

The section headings used herein are for organizational purposes only and are not to be construed as limiting the subject matter described.

It is to be understood that the methods and compositions described herein are not limited to the particular methodology, protocols, cell lines, constructs, and reagents described herein and as such may vary. It is also to be understood that the terminology used herein is for the purpose of describing particular embodiments only and is not intended to limit the scope of the methods and compositions described herein, which will be limited only by the appended claims.

### EXAMPLES

### Example 1: Design of Modified P40 Domains with Reduced Affinity for IL12RB1 by Molecular Modeling and Computer-Guided Engineering

The clinical utility of IL12 as an anti-tumour agent is limited by high toxicity driven by its immunostimulatory effects. To reduce the immunostimulatory potency of IL12, the affinity of IL12 for its receptors IL12Rβ1 and/or IL12Rβ2 may be reduced through engineering.

This example describes the in-silico design by molecular modeling and computer-guided engineering of a library of modified p40 domains with reduced affinity for IL12Rβ1.

### Methods

### Structure Preparation and Analysis

As a starting point for engineering, the structural coordinates of the p40 domain in complex with IL12Rβ1 were extracted from the crystal structure of IL12 in complex with IL12Rβ1 (PDB ID 6wdq). Structural models were optimized using proprietary in silico modelling tools. This procedure rebuilds missing atoms and corrects poorly modelled regions in the structure, including amino acid sidechain and backbone conformations that may be influenced by crystal packing restrictions.

The optimized structure was analyzed using several proprietary molecular modeling tools (e.g., the ResidueContacts™ tool and AffinityDecomposition™ tool) and visually to identify hotspot positions, i.e. key residues contributing to stability and affinity, at the p40-IL12Rβ1 interface.

The same procedure was used to prepare and assess structures of the p40 domain in complex with the antibody antigen-binding fragments (Fab) of the antibodies Briakinumab (from PDB ID 5njd) and Ustekinumab (from PDB ID 3hmx).

A comparison of multiple published crystal structures containing the p40 domain (e.g. 1f45, 3duh, 3hmx, 3qrw, 4grw, 5mxa, 5mzv, 5njd, 6wdq, etc.) revealed that it possesses a significant degree of conformational flexibility. To evaluate the impact of potential mutations on the stability of uncomplexed p40, it was desirable to obtain structures of the uncomplexed p40 domain representative of the most common conformations in the vicinity of the IL12Rβ1 binding site where mutations would be designed. Explicit molecular dynamics simulations of an isolated p40 domain structure were performed and trajectory coordinates were joined and clustered using an alignment of only the immunoglobulin domain and the first fibronectin type-III domain of p40 to generate structures representing five conformational clusters.

### Mutation Design Using Computer-Guided Engineering

Potential mutations at select positions within p40 were simulated by in silico mutagenesis and modeling with the ZymeCAD® platform. The ZymeCAD® platform is a software suite that, given an input structure and a set of mutations, will alter the residue types in the input structure according to the supplied mutations and generate new structures that are approximations to the physical structure of the mutant protein. Additionally, the ZymeCAD® platform evaluates the properties of the mutant protein relative to the parent non-mutated protein by computing a variety of quantitative physics-based and knowledge-based metrics. These metrics include, for example, measures of steric and electrostatic complementarity computed on the basis of energy factors such as van der Waals packing, cavitation effects, close contact of hydrophobic groups, coulombic interactions between charges, hydrogen bonds, desolvation effects, etc., which may correlate with the stability, binding affinity, or binding specificity of the mutant protein(s).

This procedure was performed using the prepared structure of p40 in complex with IL12Rβ1, and with the prepared structures of p40 in complex the antibody antigen-binding fragments (Fab) of the antibodies Briakinumab and Ustekinumab, to assess the impact of the potential mutations on the affinity and stability of each complex.

Besides their impact on the stability and affinity of the above complexes, potential p40 mutations were also assessed for their impact on the stability of the isolated, uncomplexed p40 domain. This was accomplished by using the ZymeCAD® platform to simulate the potential mutations on representative structures for each of the three top conformational clusters of the uncomplexed p40 domain generated by molecular dynamics simulations and assessing their effects on predicted stability.

Potential mutations were designed in an iterative process beginning with simulated mutations of individual hotspot residues and evaluation with the ZymeCAD™ platform, selection of promising mutations based on stability and affinity metrics and visual inspection, and subsequent simulation, evaluation, and selection of additional mutation sets comprising the parental selected mutation(s) and additional mutations of nearby residues or alternative hotspots that may provide synergistic or otherwise desirable impacts on the predicted stability and/or affinity metrics of modified p40 domains containing the resulting mutation sets.

Potential mutations and mutation sets were assessed further for their impact on the predicted T-Cell epitope content of the resultant modified p40 domains using a proprietary in silico tool that computes per-residue and composite T-cell epitope scores of protein sequences based on MHC Class II peptide binding affinity predictions for representative MHC allele sets from the Immune Epitope Database (IEDB; https://www.iedb.org/)

### Results

Potential mutations and sets of mutations generated by the iterative process of in silico simulated mutagenesis and structure evaluation with the ZymeCAD® platform were filtered based on a number of in silico metrics and by visual inspection to obtain a library of mutations and mutation sets that are predicted to provide a broad range of affinities between the modified p40 domains and IL 12Rβ1, have a positive or minimally negative impact on the stability of the uncomplexed modified p40 domains, have a minimal impact on the affinity between the modified p40 domains and Briakinumab, and have a minimal impact on the predicted T-Cell epitope content of the modified p40 domains.

The resulting library is provided in Table G, along with select in silico metrics generated by the ZymeCAD® platform that may correlate with the affinity between the modified p40 domains and IL12Rβ1 (“ΔA Rβ1” and “ΔS Rβ1” for affinity and stability physics-based metrics, respectively; calculated as deltas between the unmodified and modified p40 domains in complex with IL12Rβ1; a negative ΔA value indicates improved or strengthened affinity, while a positive ΔA value indicates worsened or weakened affinity; a negative ΔS value indicates improved or increased stability, while a positive ΔS value indicates worsened or decreased stability), the stability of the uncomplexed modified p40 domains (“ΔS Apo” for the delta stability score between the uncomplexed unmodified and modified p40 domains), the affinity between the modified p40 domains and Briakinumab and Ustekinumab (“ΔA Bria” and “ΔA Uste”, for the delta affinity scores between the unmodified and modified p40 domains in complex with Briakinumab and Ustekinumab, respectively), and the predicted T-Cell epitope content of the modified p40 domains (“TCES” for the composite T-Cell Epitope Score of the modified p40 domains; a positive TCES value indicates an increase in predicted T-Cell epitope content relative to unmodified p40).

TABLE G
p40 mutations and in silico metrics

| P40 mutations ^{a} | ΔA Rβ1 | ΔS Rβ1 | ΔS Apo | ΔA Bria | ΔA Uste | TCES | SEQ ID |
|---|---|---|---|---|---|---|---|
| W15R_E59D_F60D_ | 92.6 | 143.8 | 37.2 | 11.4 | 21.3 | 5.47 | 40 |
| K197W | | | | | | | |
| W15H_K84L_K197Q | 78.5 | 133 | 36.4 | −11.1 | 10.5 | 9.83 | 42 |
| E59D_K84W_K197W | 65.7 | 104.5 | −39.4 | −21.6 | 12.3 | 7.52 | 46 |
| K58H_E86R_K197D | 65.2 | 100.8 | 18.4 | −1.4 | −0.7 | 6.32 | 44 |
| W15H_K84L | 62 | 111.9 | 32 | −3.3 | 11.1 | 3.64 | 41 |
| F60R_K84E_K197W | 58.6 | 145.5 | 4.1 | −11.1 | 3.4 | 8.52 | 50 |
| K84W_K197W | 54.6 | 95.9 | −37.5 | −21 | 6.4 | 7.52 | 55 |
| F60K_K197W | 53.9 | 81.7 | −3.1 | −1.8 | 9.6 | 6.57 | 49 |
| E86R_K197D | 50.8 | 83.1 | 13.7 | 0.6 | 2.1 | 6.32 | 56 |
| K84I_E86R_D93H | 50.6 | 89.1 | −6.8 | 4.4 | 1.9 | 7.79 | 53 |
| E59R_D93R | 49 | 133.4 | 22.3 | −1.8 | 93.9 | 2.74 | 48 |
| K84I_E86R | 43.1 | 49.9 | −4.6 | 2.6 | 1.3 | 6.73 | 52 |
| W15K | 41.9 | 89.4 | 53.7 | 16.9 | 16 | 0 | 13 |
| K84W_D161R | 36.5 | 46.2 | −25 | −15.2 | 7.9 | 3.19 | 54 |
| E45R_K58S_E59S_ | 36 | 49.3 | 26.5 | −2.7 | 54 | 19.05 | 43 |
| K195D | | | | | | | |
| E59D_D93H | 31.1 | 58.3 | −0.7 | 3.5 | 4.2 | 0.85 | 47 |
| F60R_K84Y | 30 | 128.2 | 9.5 | −6.4 | 10.9 | 4.44 | 51 |
| E86W_D93E | 29.3 | 41.5 | −11.3 | −9.7 | 1.2 | 3.77 | 57 |
| K58S_E59S_K195D | 27.4 | 41.2 | 21.8 | −4.4 | 9.1 | 9.64 | 45 |
| K84E | 27.3 | 60 | 7.8 | −7.2 | 2.3 | 1.86 | 24 |
| K197T | 23.2 | 34.1 | 9.1 | −2 | −0.1 | 5.06 | 38 |
| E59R | 22.8 | 62.4 | 8.6 | 2 | 90.3 | 1.55 | 19 |
| W15H | 20.9 | 51.1 | 35.5 | 4.4 | 10.2 | 0 | 12 |
| K84W | 19.6 | 18.3 | −28.2 | −18.5 | 6.6 | 2.05 | 27 |
| K197W | 16.8 | 62.8 | −6.9 | −6.3 | −4.4 | 5.47 | 39 |
| E59S | 16.1 | 25.1 | 6.1 | 0.7 | 19.2 | 1.01 | 20 |
| D161R | 14.5 | 19.7 | 4.3 | 2.5 | 1.3 | 1.13 | 34 |
| E86W | 12.1 | 8.5 | −8.2 | −14.2 | 0.6 | 3.77 | 30 |

^{a} Mutations are numbered according to the mature p40 sequence without signal peptide (SP) (SEQ ID NO: 10). The same mutations applied to the p40 precursor sequence including SP are numbered as follows (see also, e.g., Tables B and C): W37R_E81D_F82D_K219W, W37H_K106L_K219Q, E81D_K106W_K219W, K80H_E108R_K219D, W37H_K106L, F82R_K106E_K219W, K106W_K219W, F82K_K219W, E108R_K219D, K106I_E108R_D115H, E81R_D115R, K106I_E108R, W37K, K106W_D183R, E67R_K80S_E81S_K217D, E81D_D115H, F82R_K106Y, E108W_D115E, K80S_E81S_K217D, K106E, K219T, E81R, W37H, K106W, K219W, E81S, D183R, E108W

### Example 2: Production, Purification, and Biophysical Characterization of IL12 HetFc Fusion Proteins with P40 Domains Modified for Reduced Affinity for IL12RB1

The biophysical and functional impact of modified p40 domains engineered for reduced affinity to IL12Rβ1 may be assessed in the context of any p40 domain-containing fusion protein, by comparing the properties of fusion proteins containing modified p40 domains to corresponding fusion proteins with non-modified p40 domains. This example describes the expression, purification, and biophysical characterization of IL12 HetFc fusion proteins and masked IL12 HetFc fusion proteins containing modified and non-modified p40 domains.

### Methods

### Design of IL12 HetFc and Masked IL12 HetFc Fusion Proteins:

IL12 fusion proteins to a heterodimeric Fc (‘HetFc’) were constructed with the geometry of variant 30806 as depicted in FIGS. 2A-B. Briefly, fusion proteins were designed using single-chain IL12 (‘scIL12’) consisting of a p35 domain fused C-terminally to a p40 domain by a peptide linker, and scIL12 fused C-terminally to one of two chains of the HetFc by a polypeptide linker. Masked IL12 fusion proteins were constructed in the same fashion, with the addition of a Briakinumab-derived scFv fused C-terminally to the second HetFc chain by a peptide linker, as depicted for variant v35436 in FIG. 2B. IL12 HetFc and masked IL12 HetFc fusion proteins were constructed with mutated and non-mutated p40 domains as outlined in Table H.

### Cloning:

The polypeptide sequences of the designed IL12 HetFc fusion proteins were reverse translated to DNA, codon optimized for mammalian cell expression, and gene synthesized. All sequences were preceded by an artificially designed signal peptide of sequence MRPTWAWWLFLVLLLALWAPARG (SEQ ID NO: 1) (Barash S et al., Biochem and Biophys Res. Comm. 2002; 294, 835-842). For all sequences, vector inserts consisting of a 5′-EcoR1 restriction site, the signal peptide described above, the codon-optimized DNA sequence corresponding to clones presented in this example, a TGA or TAA stop codon, and a BamH1 restriction site-3′, were ligated into pTT5 vectors to produce expression vectors (Durocher Y et al., Nucl. Acids Res. 2002; 30, No. 2 e9). The resulting expression vectors were sequenced to confirm correct reading frame and sequence of the coding DNA.

### Mammalian Cell Transient Transfection and Protein Expression:

To produce recombinant IL12 HetFc fusion proteins, HEK293-6E cells at a density of 1.5-2.2×10⁶ cells/ml are cultured at 37° C. in FreeStyle™ F17 medium (GIBCO Cat #A13835-01) supplemented with G418 (Wisentbioproducts cat #400-130-IG), 4 mM glutamine, and 0.1% Pluronic F-68 (Gibco Cat #24040-032). Cells are transfected with 1 μg DNA per 1 mL of cells (DNA comprised of Variant expression vector DNA mixtures and GFP/AKT/stuffer DNA in a 1:1 w/w ratio) using PEI-max (Polyscience, Philadelphia, PA) at a DNA:PEI ratio of 1:2.5 (w/w). Twenty-four hours after the addition of the DNA-PEI mixture, 0.5 mM Valproic acid (final concentration) and 0.5% w/v Tryptone Ni (final concentration) are added to the cells, which are then transferred to 37° C. and incubated for 7 days prior to harvesting.

### Protein Purification by Protein-A Affinity Chromatography:

Supernatants from transient transfections are applied to slurries containing 50% mAb Select SuRe™ resin (GE Healthcare, Chicago, IL) and incubated overnight at 2-8° C. on an orbital shaker at 150 rpm. The slurries are transferred into chromatography columns and flow-throughs are collected. The resins are then washed with 5 Bed Volumes (BV) of resin Equilibration buffer (PBS). To elute the targeted proteins, 5.5 BV of acidic Elution Buffer (100 mM sodium citrate buffer pH 3.5) is added to the columns and collected in fractions. Elution fractions are then neutralized by adding 10% (v/v) 1 M Tris pH 9 to reach a final pH of 6-7. The protein content of each elution fraction is determined by 280 nm absorbance measurement using a Nanodrop™ or with a relative colorimetric protein assay. The most concentrated fractions are pooled, which correspond to at least 80% of the total eluted protein.

### Protein Purification by Preparative Size-Exclusion Chromatography (Prep-SEC):

Samples are loaded onto a Superdex 200 Increase 10/300 column (#28-9909-44, GE Healthcare Life Sciences, Marlborough, MA) on an Akta pure 25 chromatography system (GE Healthcare Life Sciences, Marlborough, MA) in PBS with a flow rate of 0.8 mL/min. Fractions of eluted protein were collected based on A280 nm and their purity were analyzed by non-reducing CE-SDS with LabChip™ GXII Touch (Perkin Elmer, Waltham, MA). Protein containing fractions of high purity were pooled and protein in final pools was quantitated based on A280 nm (Nanodrop™) post SEC.

### Purity Assessment by Capillary Electrophoresis (CE) Using LabChip™

The purity of fusion protein samples is assessed by non-reducing and reducing LabChip™ CE-SDS. LabChip™ GXII Touch (Perkin Elmer, Waltham, MA). Analysis is carried out according to Protein Express Assay User Guide (PerkinElmer, Waltham, MA), with the following modifications. Samples at a concentration range of 5-2000 ng/μl are added to separate wells in 96 well plates (#MSP9631, BioRad, Hercules, CA) along with 7 μl of HT Protein Express Sample Buffer (#CLS920003, Perkin Elmer) and denatured at 90° C. for 5 mins. The LabChip™ instrument is operated using the LabChip™ HT Protein Express Chip (Perkin Elmer #760528) with HT Protein Express 200 assay setting.

### Purity Assessment by UPLC-SEC

The fusion protein samples are assessed by UPLC-SEC to determine their percentage of high molecular weight species. UPLC-SEC is performed using a Waters Acquity BEH200 SEC column (2.5 mL, 4.6×150 mm, stainless steel, 1.7 μm particles) (Waters LTD, Mississauga, ON) set to 30° C. and mounted on an Agilent Technologies 1260 infinity II system with a PDA detector. Run times consist of 7 min and a total volume per injection of 2.8 mL with a running buffer of either 150 mM Sodium Phosphate pH 6.95, DPBS+0.02% Tween 20, or 200 mM KPO4, 200 mM KCl, pH7, or 200 mM KPO4, 200 mM KCl, 0.02% Tween 20, pH7, at 0.4 mL/min. Elution is monitored by UV absorbance in the range 210-500 nm, and chromatograms are extracted at 280 nm. Peak integration is performed using OpenLAB™ CDS ChemStation™ software.

### Stability Assessment by Differential Scanning Fluorimetry (DSF)

The stability of fusion protein samples is assessed by DSF using a CFX Connect real-time PCR detection system (BioRad). 25 μl of buffer, 5 μl of 40×SYPRO Orange dye, and 10 μl of variant are added to wells of a 96-well PCR plate. The plate is sealed with and optical PCR plate seal, centrifuged at 1000 rpm for 1 min, placed in the thermocycler and read over a 25° C. to 95° C. gradient at 0.5° C./min or 1° C./min.

### Results

It is expected that samples of IL12 HetFc and masked IL12 HetFc fusion proteins containing modified and non-modified p40 domains may be expressed from transiently transfected mammalian cells and purified to homogeneity by a two-step purification process consisting of Protein A affinity chromatography followed by Prep-SEC or IEX. It is expected that expression titers, purification yields, and stability of IL12 HetFc and masked IL12 HetFc fusion proteins containing modified p40 domains will differ from those of IL12 HetFc and masked IL12 HetFc fusion proteins containing non-modified p40 domains in a trend that correlates with the stability of modified p40 domains predicted in silico as described in Example 1.

TABLE H
p40 mutations and clone and variant numbers for IL12 HetFc fusion
proteins containing corresponding modified p40 domains

| | | | IL12 HetFc | Masked IL12 HetFc |
|---|---|---|---|---|
| | HetFc-scIL12 | SEQ | fusion protein | fusion protein |
| P40 mutations ^{a} | clone number | ID NO | variant number ^{b} | variant number ^{c} |
| None | 22279 | 61 | 30806 | 35436 |
| E59R_D93R | 28046 | 62 | 37154 | 37467 |
| F60R_K84Y | 28047 | 63 | 37155 | 37468 |
| E86R_K197D | 28048 | 64 | 37156 | 37469 |
| K84W_K197W | 28049 | 65 | 37157 | 37470 |
| F60K_K197W | 28050 | 66 | 37158 | 37471 |
| K84I_E86R | 28051 | 67 | 37159 | 37472 |
| E86W_D93E | 28053 | 68 | 37161 | 37474 |
| E59D_D93H | 28054 | 69 | 37162 | 37475 |
| K84W_D161R | 28055 | 70 | 37163 | 37476 |
| K84E | 28056 | 71 | 37164 | 37477 |
| E59R | 28057 | 72 | 37165 | 37478 |
| K197T | 28058 | 73 | 37166 | 37479 |
| D161R | 28059 | 74 | 37167 | 37480 |
| E59S | 28060 | 75 | 37168 | 37481 |
| K84W | 28061 | 76 | 37169 | 37482 |
| E86W | 28062 | 77 | 37170 | 37483 |
| K197W | 28063 | 78 | 37171 | 37484 |
| W15R_E59D_F60D_K197W | 28064 | 79 | 37172 | 37485 |
| F60R_K84E_K197W | 28065 | 80 | 37173 | 37486 |
| W15H_K84L_K197Q | 28066 | 81 | 37174 | 37487 |
| E59D_K84W_K197W | 28067 | 82 | 37175 | 37488 |
| K58H_E86R_K197D | 28068 | 83 | 37176 | 37489 |
| K84I_E86R_D93H | 28069 | 84 | 37177 | 37490 |
| W15H_K84L | 28070 | 85 | 37178 | 37491 |
| W15K | 28071 | 86 | 37179 | 37492 |
| W15H | 28072 | 87 | 37180 | 37493 |
| E45R_K58S_E59S_K195D | 28074 | 88 | 37181 | 37494 |
| K58S_E59S_K195D | 28075 | 89 | 37182 | 37495 |

^{a} Mutations are numbered according to the mature p40 sequence without SP. The same mutations applied to the p40 precursor sequence including SP are numbered as follows: E81R_D115R, F82R_K106Y, E108R_K219D, K106W_K219W, F82K_K219W, K106I_E108R, E108W_D115E, E81D_D115H, K106W_D183R, K106E, E81R, K219T, D183R, E81S, K106W, E108W, K219W, W37R_E81D_F82D_K219W, F82R_K106E_K219W, W37H_K106L_K219Q, E81D_K106W_K219W, K80H_E108R_K219D, K106I_E108R_D115H, W37H_K106L, W37K, W37H, E67R_K80S_E81S_K217D, K80S_E81S_K217D
^{b} Each IL12 HetFc fusion protein variant contains a HetFc-scIL12 clone as listed in Table H, plus the HetFc clone 12153 (SEQ ID NO: 58)
^{c} Each masked IL12 HetFc fusion protein variant contains a HetFc-scIL12 clone as listed in Table H, plus the HetFc-scFv clone 26503 (SEQ ID NO: 60)

### Example 3: Production, Purification, and Biophysical Characterization of IL12 HetFc Fusion Proteins with P40 Domains Modified for Reduced Affinity for IL12RB1

The biophysical and functional impact of modified p40 domains engineered for reduced affinity to IL12Rβ1 were assessed in the context of any p40 domain-containing fusion protein, by comparing the properties of fusion proteins containing modified p40 domains to corresponding fusion proteins with non-modified p40 domains. This example describes the expression, purification, and biophysical characterization of IL12 HetFc fusion proteins and masked IL12 HetFc fusion proteins containing modified and non-modified p40 domains.

### Methods

### Design of IL12 HetFc and Masked IL12 HetFc Fusion Proteins:

IL12 fusion proteins to a heterodimeric Fe (‘HetFc’) were constructed with the geometry of variant 30806 as depicted in FIGS. 2A-B. Briefly, fusion proteins were designed using single-chain IL12 (‘scIL12’) consisting of a p35 domain fused C-terminally to a p40 domain by a peptide linker, and scIL12 fused C-terminally to one of two chains of the HetFc by a polypeptide linker. Masked IL12 fusion proteins were constructed in the same fashion, with the addition of a Briakinumab-derived scFv fused C-terminally to the second HetFc chain by a peptide linker, as depicted for variant v35436 in FIG. 2B. IL12 HetFc and masked IL12 HetFc fusion proteins were constructed with mutated and non-mutated p40 domains as outlined in Table I.

### Cloning:

The polypeptide sequences of the designed IL12 HetFc fusion proteins were reverse translated to DNA, codon optimized for mammalian cell expression, and gene synthesized. All sequences were preceded by an artificially designed signal peptide of sequence MRPTWAWWLFLVLLLALWAPARG (SEQ ID NO: 1) (Barash S et al., Biochem and Biophys Res. Comm. 2002; 294, 835-842). For all sequences, vector inserts consisting of a 5′-EcoR1 restriction site, the signal peptide described above, the codon-optimized DNA sequence corresponding to clones presented in this example, a TGA or TAA stop codon, and a BamH1 restriction site-3′, were ligated into pTT5 vectors to produce expression vectors (Durocher Y et al., Nucl. Acids Res. 2002; 30, No. 2 e9). The resulting expression vectors were sequenced to confirm correct reading frame and sequence of the coding DNA.

### Mammalian Cell Transient Transfection and Protein Expression:

To produce recombinant IL12 HetFc fusion proteins, HEK293-6E cells at a density of 1.5-2.2×10⁶ cells/ml were cultured at 37° C. in FreeStyle™ F17 medium (GIBCO Cat #A13835-01) supplemented with G418 (Wisentbioproducts cat #400-130-IG), 4 mM glutamine, and 0.1% Pluronic F-68 (Gibco Cat #24040-032). Cells were transfected with 1 μg DNA per 1 mL of cells (DNA comprised of Variant expression vector DNA mixtures and GFP/AKT/stuffer DNA in a 1:1 w/w ratio) using PEI-max (Polyscience, Philadelphia, PA) at a DNA:PEI ratio of 1:2.5 (w/w). Twenty-four hours after the addition of the DNA-PEI mixture, 0.5 mM Valproic acid (final concentration) and 0.5% w/v Tryptone Ni (final concentration) were added to the cells, which were then transferred to 37° C. and incubated for 7 days prior to harvesting.

### Protein Purification by Protein-A Affinity Chromatography:

Supernatants from transient transfections were applied to slurries containing 50% mAb Select SuRe™ resin (GE Healthcare, Chicago, IL) and incubated overnight at 2-8° C. on an orbital shaker at 150 rpm. The slurries were transferred into chromatography columns and flow-throughs were collected. The resins were then washed with 5 Bed Volumes (BV) of resin Equilibration buffer (PBS). To elute the targeted proteins, 5.5 BV of acidic Elution Buffer (100 mM sodium citrate buffer pH 3.5) was added to the columns and collected in fractions. Elution fractions were then neutralized by adding 10% (v/v) 1 M Tris pH 9 to reach a final pH of 6-7. The protein content of each elution fraction was determined by 280 nm absorbance measurement using a Nanodrop or with a relative colorimetric protein assay. The most concentrated fractions were pooled, which correspond to at least 80% of the total eluted protein.

### Protein Purification by Preparative Size-Exclusion Chromatography (Prep-SEC):

Samples were loaded onto a Superdex 200 Increase 10/300 column (#28-9909-44, GE Healthcare Life Sciences, Marlborough, MA) on an Akta pure 25 chromatography system (GE Healthcare Life Sciences, Marlborough, MA) in PBS with a flow rate of 0.8 mL/min. Fractions of eluted protein were collected based on A280 nm and their purity were analyzed by non-reducing CE-SDS with LabChip™ GXII Touch (Perkin Elmer, Waltham, MA). Protein containing fractions of high purity were pooled and protein in final pools was quantitated based on A280 nm (Nanodrop™) post SEC.

Purity assessment by Capillary Electrophoresis (CE) using LabChip™ The purity of fusion protein samples was assessed by non-reducing and reducing LabChip™ CE-SDS. LabChip™ GXII Touch (Perkin Elmer, Waltham, MA). Analysis was carried out according to Protein Express Assay User Guide (PerkinElmer, Waltham, MA), with the following modifications. Samples at a concentration range of 5-2000 ng/μl were added to separate wells in 96 well plates (#MSP9631, BioRad, Hercules, CA) along with 7 μl of HT Protein Express Sample Buffer (#CLS920003, Perkin Elmer) and denatured at 90° C. for 5 mins. The LabChip™ instrument was operated using the LabChip™ HT Protein Express Chip (Perkin Elmer #760528) with HT Protein Express 200 assay setting.

### Purity Assessment by UPLC-SEC

The fusion protein samples were assessed by UPLC-SEC to determine their percentage of high molecular weight species. UPLC-SEC was performed using a Waters Acquity BEH200 SEC column (2.5 mL, 4.6×150 mm, stainless steel, 1.7 μm particles) (Waters LTD, Mississauga, ON) set to 30° C. and mounted on an Agilent Technologies 1260 infinity II system with a PDA detector. Run times consisted of 7 min and a total volume per injection of 2.8 mL with a running buffer of either 150 mM Sodium Phosphate pH 6.95, DPBS+0.02% Tween 20, or 200 mM KPO4, 200 mM KCl, pH7, or 200 mM KPO4, 200 mM KCl, 0.02% Tween 20, pH7, at 0.4 mL/min. Elution was monitored by UV absorbance in the range 210-500 nm, and chromatograms were extracted at 280 nm. Peak integration was performed using OpenLAB™ CDS ChemStation™ software.

### Stability Assessment by Differential Scanning Fluorimetry (DSF)

The stability of fusion protein samples was assessed by DSF using a CFX Connect real-time PCR detection system (BioRad). 25 μl of buffer, 5 μl of 40×SYPRO Orange dye, and 10 μl of variant were added to wells of a 96-well PCR plate. The plate was sealed with an optical PCR plate seal, centrifuged at 1000 rpm for 1 min, placed in the thermocycler and read over a 25° C. to 95° C. gradient at 0.5° C./min or 1° C./min.

### Results

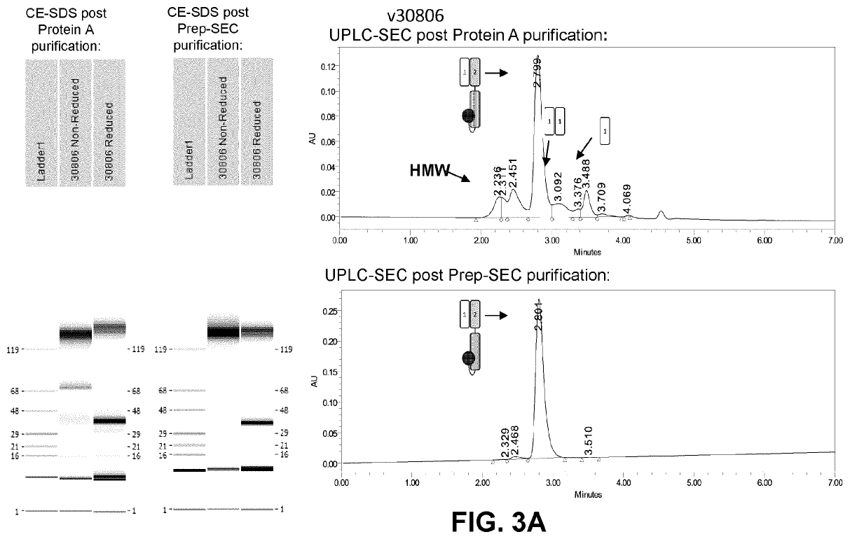

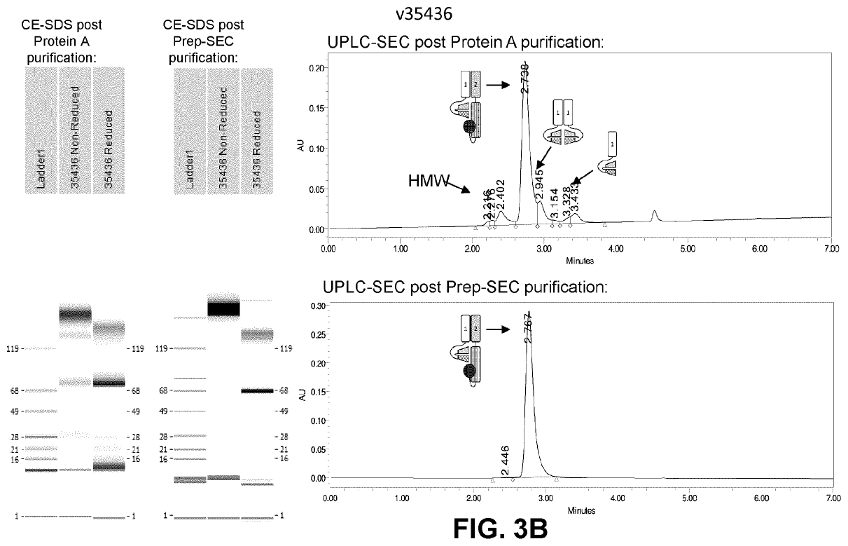

IL12 HetFc and masked IL12 HetFc fusion proteins containing modified and non-modified p40 domains were expressed from transiently transfected mammalian cells and purified to homogeneity by a two-step purification process consisting of Protein A affinity chromatography followed by Prep-SEC. After Protein A affinity purification, the yields of IL12 HetFc and masked IL12 HetFc fusion proteins containing modified p40 domains were between 50% and 138% of the corresponding fusion proteins with non-modified p40 domains (Tables J-K). All fusion proteins containing modified p40 domains displayed similar UPLC-SEC and CE-SDS profiles after Protein A affinity purification as compared to the corresponding fusion proteins with non-modified p40 domains (FIGS. 3A and 3B), with contaminants being dimer/trimer high molecular weight (HMW) species, and low molecular weight (LMW) species consisting of single chain or homodimer of the non-fused HetFc chain for non-masked IL12 HetFc fusion proteins or the scFv-fused HetFc chain for masked IL12 HetFc fusion proteins. Monodispersity of desired heterodimeric IL12 HetFc fusion protein species after Protein A purification was between 44% and 75% (Tables J-K). Following Prep-SEC purification, all samples displayed over 93% monodispersity.

The thermostability, as measured by the melting temperature using DSF as described herein, of the tested fusion proteins containing a modified p40 domain that are listed in Table I were nearly identical to that of fusion proteins containing wildtype p40 domains, i.e., the p40 domain substitutions did not materially affect the stability of the fusion proteins described herein.

TABLE I
p40 mutations and clone and variant numbers for IL12 HetFc Fusion
Proteins containing corresponding modified p40 domains

| | | | IL12 HetFc | Masked IL12 HetFc |
|---|---|---|---|---|
| | HetFc-scIL12 | SEQ | fusion protein | fusion protein |
| P40 mutations ^{a} | clone number | ID NO | variant number ^{b} | variant number ^{c} |
| None | 22279 | 61 | 30806 | 35436 |
| E59R_D93R | 28046 | 62 | 37154 | 37467 |
| F60R_K84Y | 28047 | 63 | 37155 | 37468 |
| E86R_K197D | 28048 | 64 | 37156 | 37469 |
| K84W_K197W | 28049 | 65 | 37157 | 37470 |
| F60K_K197W | 28050 | 66 | 37158 | 37471 |
| K84I_E86R | 28051 | 67 | 37159 | 37472 |
| E86W_D93E | 28053 | 68 | 37161 | 37474 |
| E59D_D93H | 28054 | 69 | 37162 | 37475 |
| K84W_D161R | 28055 | 70 | 37163 | 37476 |
| K84E | 28056 | 71 | 37164 | 37477 |
| E59R | 28057 | 72 | 37165 | 37478 |
| K197T | 28058 | 73 | 37166 | 37479 |
| D161R | 28059 | 74 | 37167 | 37480 |
| E59S | 28060 | 75 | 37168 | 37481 |
| K84W | 28061 | 76 | 37169 | 37482 |
| E86W | 28062 | 77 | 37170 | 37483 |
| K197W | 28063 | 78 | 37171 | 37484 |
| W15R_E59D_F60D_K197W | 28064 | 79 | 37172 | 37485 |
| F60R_K84E_K197W | 28065 | 80 | 37173 | 37486 |
| W15H_K84L_K197Q | 28066 | 81 | 37174 | 37487 |
| E59D_K84W_K197W | 28067 | 82 | 37175 | 37488 |
| K58H_E86R_K197D | 28068 | 83 | 37176 | 37489 |
| K84I_E86R_D93H | 28069 | 84 | 37177 | 37490 |
| W15H_K84L | 28070 | 85 | 37178 | 37491 |
| W15K | 28071 | 86 | 37179 | 37492 |
| W15H | 28072 | 87 | 37180 | 37493 |
| E45R_K58S_E59S_K195D | 28074 | 88 | 37181 | 37494 |
| K58S_E59S_K195D | 28075 | 89 | 37182 | 37495 |

^{a} Mutations are numbered according to the mature p40 sequence without SP. The same mutations applied to the p40 precursor sequence including SP are numbered as follows: E81R_D115R, F82R_K106Y, E108R_K219D, K106W_K219W, F82K_K219W, K106I_E108R, E108W_D115E, E81D_D115H, K106W_D183R, K106E, E81R, K219T, D183R, E81S, K106W, E108W, K219W, W37R_E81D_F82D_K219W, F82R_K106E_K219W, W37H_K106L_K219Q, E81D_K106W_K219W, K80H_E108R_K219D, K106I_E108R_D115H, W37H_K106L, W37K, W37H, E67R_K80S_E81S_K217D, K80S_E81S_K217D
^{b} Each IL12 HetFc fusion protein variant contains a HetFc-scIL12 clone as listed in Table I, plus the HetFc clone 12153 (SEQ ID NO: 58)
^{c} Each masked IL12 HetFc fusion protein variant contains a HetFc-scIL12 clone as listed in Table I, plus the HetFc-scFv clone 26503 (SEO ID NO: 60)

TABLE J
Production and Purification of Unmasked IL12 HetFc Fusion Proteins

| | | | | | | Post-prep-SEC | |
|---|---|---|---|---|---|---|---|
| | | | | | | Purification | |
| | IL12 HetFc | | | | | | UPLC- |
| | Fusion | Post-pA Purification | | | | | SEC % |
| | Protein | | UPLC-SEC peak % | | | | area |
| | Variant | Yield | area | | | Yield | main |
| P40 mutations ^{a} | Number | (mg) ^{b} | HMW ^{c} | Main | LMW ^{c} | (mg) ^{b} | peak |
| None | 30806 | 66.9 | 23.7 | 57.0 | 19.3 | 23.9 | 98.1 |
| E59R_D93R | 37154 | 39.8 | 17.7 | 51.6 | 30.7 | 8.7 | 93.3 |
| F60R_K84Y | 37155 | 54.2 | 29.6 | 52.6 | 17.8 | 13.6 | 97.8 |
| E86R_K197D | 37156 | 64.1 | 26.9 | 49.5 | 23.7 | 16.2 | 98.4 |
| K84W_K197W | 37157 | 33.5 | 18.1 | 50.1 | 31.9 | 6.6 | 97.8 |
| F60K_K197W | 37158 | 61.5 | 8.7 | 61.5 | 29.8 | 17.5 | 98.9 |
| K84I_E86R | 37159 | 80.0 | 19.6 | 61.1 | 19.3 | 27.2 | 99.2 |
| E86W_D93E | 37161 | 60.9 | 31.7 | 43.7 | 24.6 | 13.4 | 98.1 |
| E59D_D93H | 37162 | 71.9 | 28.4 | 47.7 | 24.0 | 19.3 | 98.0 |
| K84W_D161R | 37163 | 68.7 | 27.8 | 46.2 | 26.0 | 14.4 | 98.8 |
| K84E | 37164 | 60.9 | 27.0 | 51.7 | 21.3 | 14.5 | 97.6 |
| E59R | 37165 | 49.6 | 28.8 | 52.9 | 18.3 | 14.0 | 98.6 |
| K197T | 37166 | 83.6 | 28.2 | 51.5 | 20.3 | 21.7 | 98.7 |
| D161R | 37167 | 77.8 | 35.9 | 46.9 | 17.2 | 16.4 | 98.2 |
| E59S | 37168 | 84.9 | 23.0 | 56.7 | 20.3 | 25.7 | 99.0 |
| K84W | 37169 | 63.1 | 28.8 | 49.3 | 21.9 | 14.8 | 98.5 |
| E86W | 37170 | 61.2 | 28.6 | 47.0 | 24.4 | 14.6 | 98.6 |
| K197W | 37171 | 77.9 | 40.0 | 51.9 | 8.1 | 19.0 | 98.8 |
| W15R_E59D_F60D_ | 37172 | 75.0 | 21.0 | 59.7 | 19.3 | 25.4 | 99.1 |
| K197W | | | | | | | |
| F60R_K84E_K197W | 37173 | 42.1 | 23.8 | 46.4 | 29.9 | 10.1 | 97.8 |
| W15H_K84L_K197Q | 37174 | 80.6 | 20.4 | 59.7 | 19.8 | 25.2 | 99.0 |
| E59D_K84W_K197W | 37175 | 49.4 | 20.0 | 49.2 | 30.8 | 10.3 | 97.0 |
| K58H_E86R_K197D | 37176 | 54.2 | 25.9 | 53.5 | 20.6 | 16.4 | 98.4 |
| K84I_E86R_D93H | 37177 | 61.8 | 20.7 | 53.2 | 26.1 | 21.7 | 98.8 |
| W15H_K84L | 37178 | 91.4 | 18.8 | 60.2 | 21.1 | 37.1 | 99.1 |
| W15K | 37179 | 77.3 | 26.6 | 55.7 | 17.7 | 23.0 | 98.7 |
| W15H | 37180 | 70.2 | 24.1 | 57.6 | 18.3 | 25.5 | 98.8 |
| E45R_K58S_E59S_ | 37181 | 68.5 | 23.2 | 55.9 | 21.0 | 19.6 | 98.8 |
| K195D | | | | | | | |
| K58S_E59S_K195D | 37182 | 67.9 | 21.5 | 58.8 | 19.7 | 18.4 | 98.7 |

^{a} Mutations are numbered according to the mature p40 sequence without SP.
^{b} Yield normalized to mg per L of expression culture.
^{c} HMW: High Molecular Weight; LMW: Low Molecular Weight.

TABLE K
Production and Purification of Masked IL12 HetFc Fusion Proteins

| | | | | | | Post-prep-SEC | |
|---|---|---|---|---|---|---|---|
| | Masked | | | | | Purification | |
| | IL12 HetFc | | | | | | UPLC- |
| | Fusion | Post-pA Purification | | | | | SEC % |
| | Protein | | UPLC-SEC peak % | | | | area |
| | Variant | Yield | area | | | Yield | main |
| P40 mutations ^{a} | Number | (mg) ^{b} | HMW ^{c} | Main | LMW ^{c} | (mg) ^{b} | peak |
| None | 35436 | 42.8 | 9.8 | 72.8 | 17.4 | 11.5 | 99.1 |
| E59R_D93R | 37467 | 42.1 | 11.4 | 68.7 | 19.9 | 9.7 | 99.2 |
| F60R_K84Y | 37468 | 45.2 | 9.1 | 74.1 | 16.8 | 12.8 | 99.0 |
| E86R_K197D | 37469 | 51.6 | 11.8 | 69.6 | 18.6 | 13.3 | 99.2 |
| K84W_K197W | 37470 | 39.7 | 10.3 | 73.3 | 16.4 | 12.3 | 99.0 |
| F60K_K197W | 37471 | 45.4 | 10.9 | 73.4 | 15.8 | 14.9 | 98.8 |
| K84I_E86R | 37472 | 51.2 | 11.4 | 71.4 | 17.2 | 14.3 | 99.0 |
| E86W_D93E | 37474 | 46.2 | 12.9 | 65.9 | 21.3 | 11.2 | 99.1 |
| E59D_D93H | 37475 | 48.4 | 10.3 | 71.7 | 18.0 | 11.5 | 98.7 |
| K84W_D161R | 37476 | 46.0 | 12.0 | 67.4 | 20.7 | 8.3 | 99.5 |
| K84E | 37477 | 40.4 | 9.2 | 75.3 | 15.6 | 10.6 | 99.3 |
| E59R | 37478 | 50.1 | 12.5 | 71.7 | 15.8 | 12.8 | 99.1 |
| K197T | 37479 | 47.0 | 11.6 | 72.5 | 16.0 | 12.8 | 99.1 |
| D161R | 37480 | 37.8 | 11.9 | 71.0 | 17.1 | 8.5 | 99.2 |
| E59S | 37481 | 46.2 | 10.7 | 73.5 | 15.9 | 11.9 | 98.7 |
| K84W | 37482 | 47.3 | 9.4 | 75.3 | 15.3 | 13.3 | 99.0 |
| E86W | 37483 | 38.7 | 11.4 | 65.6 | 23.0 | 6.9 | 99.1 |
| K197W | 37484 | 49.7 | 11.3 | 71.4 | 17.3 | 10.7 | 99.0 |
| W15R_E59D_F60D_K197W | 37485 | 43.4 | 9.3 | 72.5 | 18.2 | 11.7 | 99.3 |
| F60R_K84E_K197W | 37486 | 43.4 | 9.7 | 73.8 | 16.5 | 14.3 | 99.3 |
| W15H_K84L_K197Q | 37487 | 48.7 | 8.8 | 74.8 | 16.4 | 13.3 | 99.1 |
| E59D_K84W_K197W | 37488 | 46.5 | 10.5 | 72.0 | 17.5 | 12.5 | 99.4 |
| K58H_E86R_K197D | 37489 | 46.9 | 12.8 | 68.0 | 19.2 | 10.3 | 99.1 |
| K84I_E86R_D93H | 37490 | 42.3 | 10.8 | 70.7 | 18.5 | 10.1 | 99.4 |
| W15H_K84L | 37491 | 55.0 | 9.6 | 73.2 | 17.2 | 11.6 | 99.2 |
| W15K | 37492 | 53.0 | 10.5 | 67.0 | 22.5 | 9.2 | 99.4 |
| W15H | 37493 | 46.7 | 10.3 | 70.3 | 19.4 | 8.3 | 99.1 |
| E45R_K58S_E59S_K195D | 37494 | 44.7 | 11.1 | 70.7 | 18.2 | 8.5 | 99.2 |
| K58S_E59S_K195D | 37495 | 59.1 | 11.9 | 69.6 | 18.5 | 10.5 | 99.0 |

^{a} Mutations are numbered according to the mature p40 sequence without SP.
^{b} Yield normalized to mg per L of expression culture.
^{c} HMW: High Molecular Weight; LMW: Low Molecular Weight.

### Example 4: Cellular Activity of IL12 HetFc Fusion Proteins

CD8+ T cells are an important target population for IL12. The potency of select variants on CD8+ T cells may be assessed by IFNγ release.

### Methods

### CD8+ T Cell IFNγ Release Assay

CD8+ T cells are thawed, stimulated with anti-CD3/CD28 dynabeads (ThermoFisher, Waltham, MA) at a cell to bead ratio of 10:1, and plated in 384-well black flat bottom assay plates (ThermoFisher, Watham, MA) at 30,000 cells/well in 30 ul RPMI1640 (Gibco)+10% FBS (ThermoFisher)+1% Pen-Strep (Gibco). Plates are incubated overnight at 37° C. and 5% carbon dioxide. The following day, samples are prepared as below and 30 ul are added to CD8+ T cells. Plates are incubated for 3 days at 37° C. and 5% carbon dioxide. Post incubation, 30 uL/well of supernatant is transferred to non-binding 384-well plates (Greiner-Bio-One, Kremsmunster, Austria) and stored at −80° C.

Aliquots of variant or control samples are thawed from −80° C. storage the day of the assay. Samples are titrated in quadruplicate at 1:10 dilution in 100 ul in non-binding 384-well plates (Greiner-Bio-One, Kremsmünster, Austria). Recombinant human IL12 (Peprotech, Rocky Hill, NJ) is included as a positive control. 30 ul of titrated variants are then transferred to simulated CD8+ T cells as above.

IFNγ is quantified using MSD (Mesoscale Discovery, Piscataway, NJ). MSD plates are blocked and coated in capture antibodies according to the manufacturers' instructions. Plates are washed 3× in PBS-T and 5 ul of assay diluent is added to each plate. IFNγ standard is titrated from 500 ng/mL down to 0.5 pg/mL. 5 uL of supernatants are transferred to MSD plates and incubated for 1h at room temperature. Plates are washed 3× in PBS-T and 10 ul of detection antibody at appropriate dilution is added to each sample and standard well. The plates are sealed with and adhesive plate seal for one hour. Plates are washed 3× in PBS-T and 40 uL MSD Gold read buffer B is added to each well. Plates are read on the MESO SECTOR 6000 and cytokine concentration is determined using MSD software. Data from a standard curve and samples are used to perform a nonlinear curve-fit with x-interpolation to obtain IFNγ concentrations in pg/mL. Three independent experiments are conducted and data from each is analyzed in a nonlinear mixed effect model to generate curve fit and 95% confidence intervals.

### Results

It is expected that a control IL12 HetFc fusion protein containing a non-modified p40 domain will stimulate a similar level (within ˜10-fold) of IFNγ release from CD8+ T Cells in the CD8+ T Cell IFNγ release assay as recombinant IL12.

It is expected that a control masked IL12 HetFc fusion protein containing a non-modified p40 domain will stimulate a significantly reduced level (>10-fold reduction) of IFNγ release from CD8+ T Cells as compared to a control IL12 HetFc fusion protein containing a non-modified p40 domain.

It is expected that IL12 HetFc fusion proteins and masked IL12 HetFc fusion proteins containing modified p40 domains will stimulate reduced levels of IFNγ release from CD8+ T Cells as compared to the corresponding control IL12 HetFc and masked IL12 HetFc fusion proteins containing non-modified p40 domains. It is expected that the extent by which IFNγ release from CD8+ T Cells is reduced will correlate with the affinity of modified p40 domains for IL12Rβ1 predicted in silico as described in Example 1.

It is expected that the reduced activity of modified p40 domains may be synergistic with that of Masked IL12 HetFc fusion proteins, such that the fold-reduction in the level of IFNγ release stimulated by Masked IL12 HetFc fusion proteins containing modified p40 domains compared to a control Masked IL12 HetFc fusion proteins containing a non-modified p40 domain will be greater than the fold-reduction in the level of IFNγ release stimulated by the corresponding IL12 HetFc fusion proteins containing modified p40 domains compared to a control IL12 HetFc fusion protein containing a non-modified p40 domain.

### Example 5: Cellular Activity of IL12 Fusion Proteins

As described herein, IL12 can exert its biological activity by binding to the IL12 receptor heterodimer, which can trigger a signalling pathway resulting in activation of STAT4. This example describes the characterization of IL12 fusion proteins and masked IL12 fusion proteins containing a heterodimeric Fc domain (HetFc) as well as modified or non-modified p40 domains using an IL12-responsive colorimetric Reporter Gene Assay (RGA), in which HEK cells stably transfected with genes for the IL12 receptor, IL12 signalling pathway, and a STAT4-inducible reporter, responded to IL12 by secreting a phosphatase that was detected in a colorimetric assay.

Reporter cells transfected with IL12 receptor suspended in assay buffer (DMEM 4.5 g/l glucose, 2 mM L-glutamine, 10% heat-inactivated FBS, Pen-Strep (100 U/ml-100 μg/ml)) were plated in 384-well white flat-bottom plates (Thermo Scientific Nunc Cat #164610) at a concentration of 10,000 cells in 30 ul per well. IL12 HetFc fusion protein variants were separately serially diluted in assay buffer from 8 nM to 0.14 pM (for variant 30806) or from 100 nM to 1.7 pM (all other variants) in 96-well V-bottom plates (Sarstedt 82.1583001). 30 ul of variants were transferred and mixed with the 30 ul of IL12R transfected reporter cells in 384-well flat-bottom plates, and plates were transferred to an incubator at 37C with 5% CO₂ and humidity for 18-24 hrs. 90 ul of Quanti-Blue solution was added per well and 10 ul of supernatant was transferred to new 384-well clear flat-bottom plates, then plates were incubated at 37C with 5% CO₂ for 1 hr and read on a Synergy H1 at 620 nm.

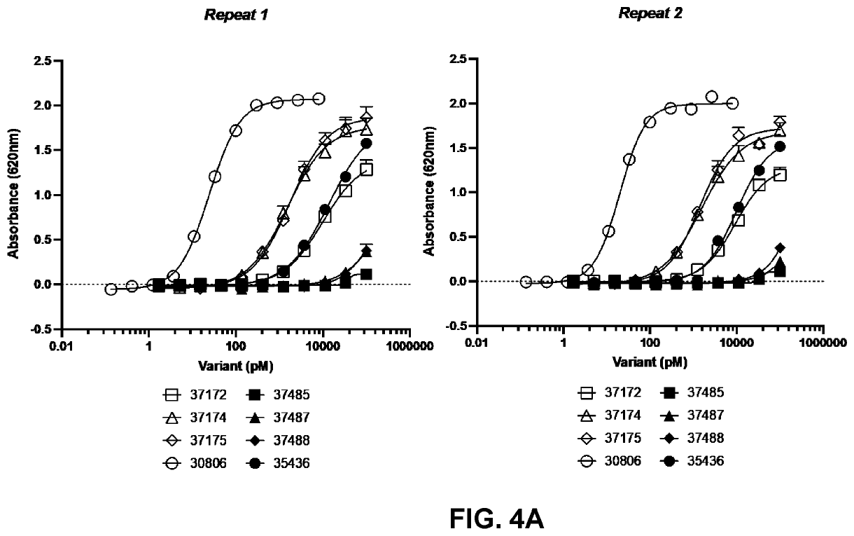

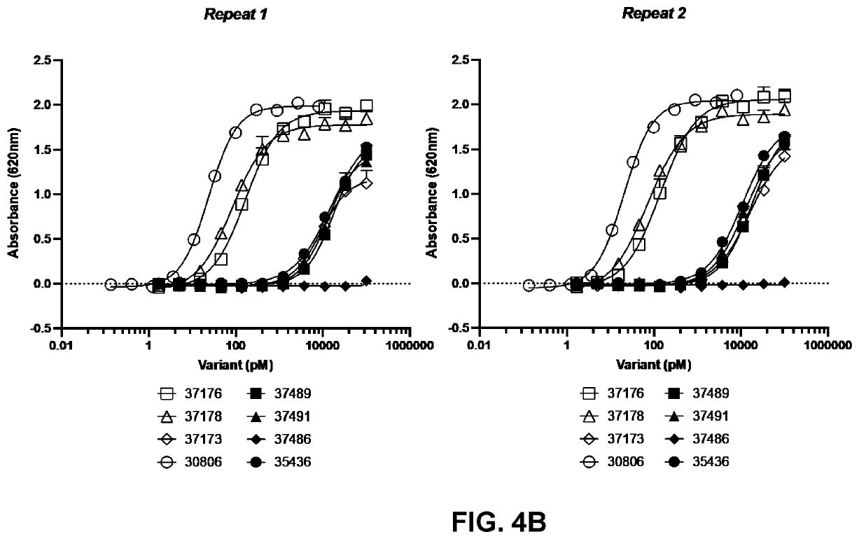

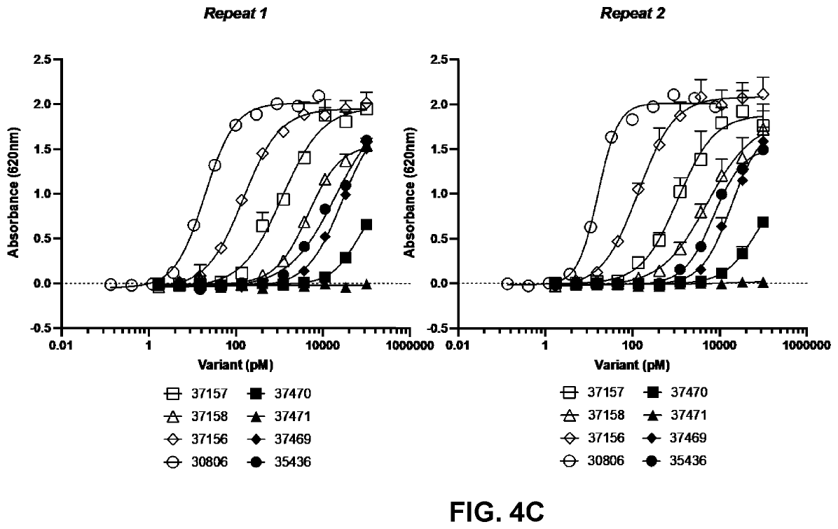

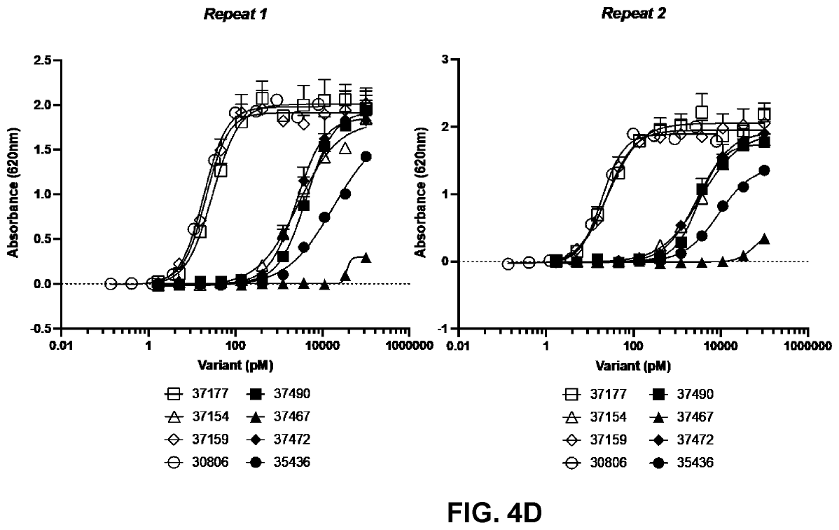

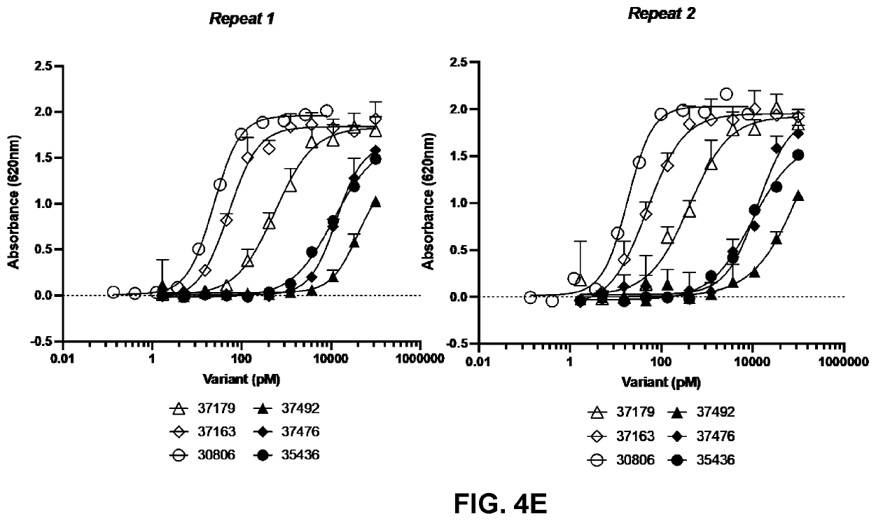

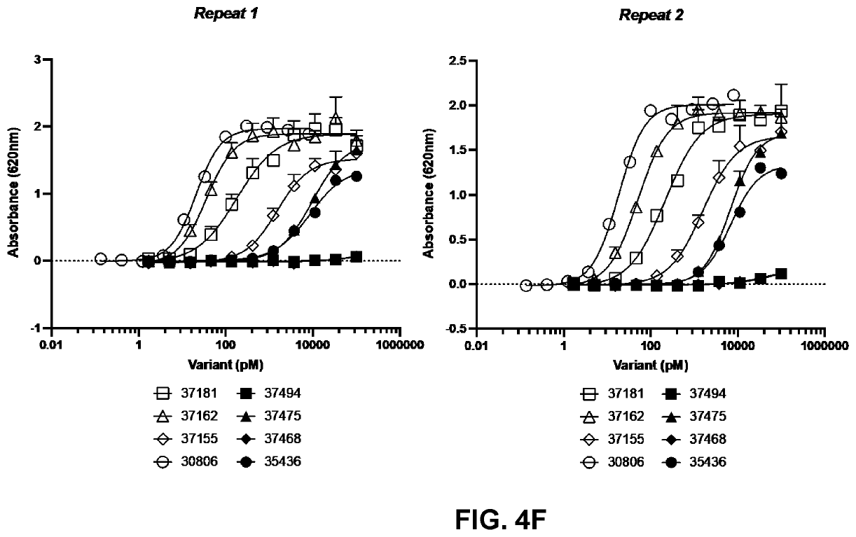

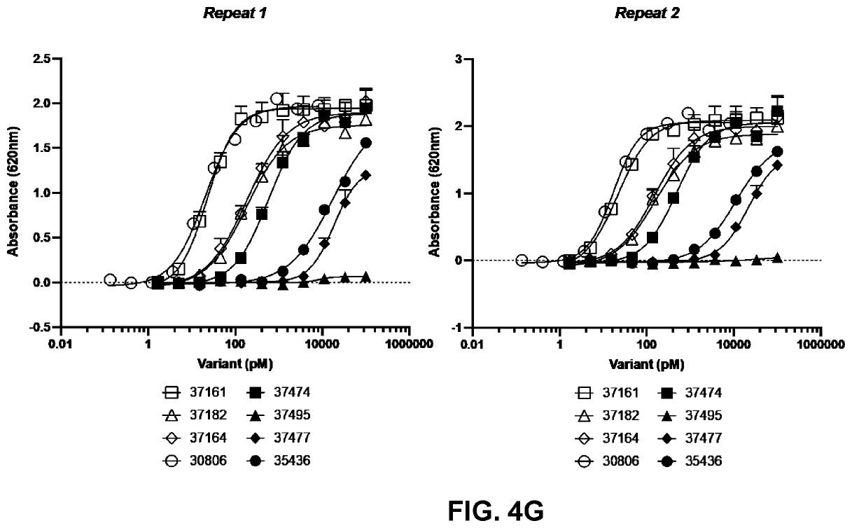

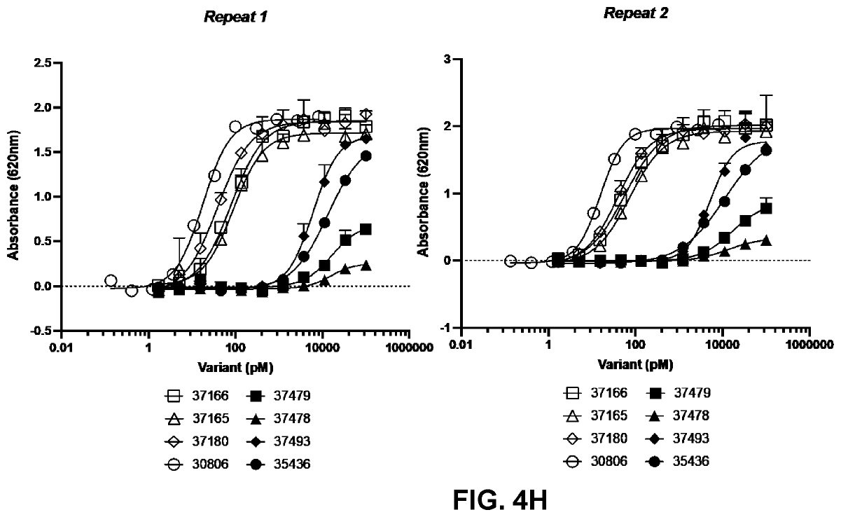

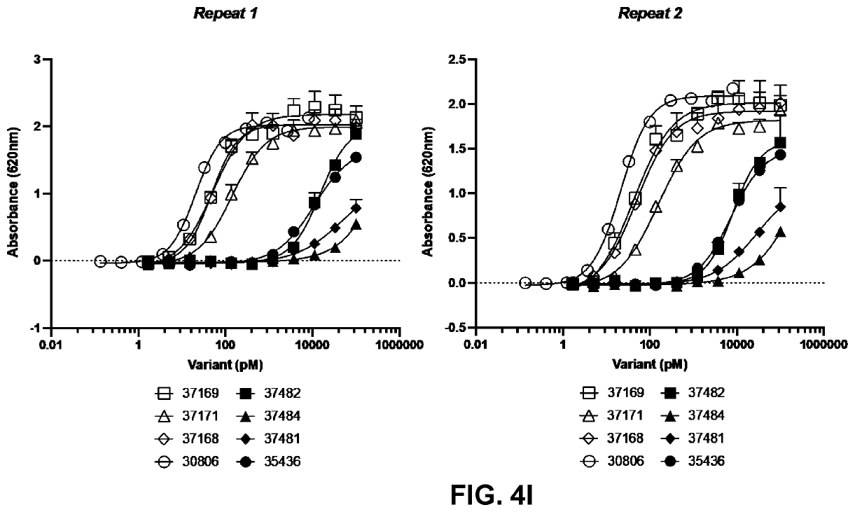

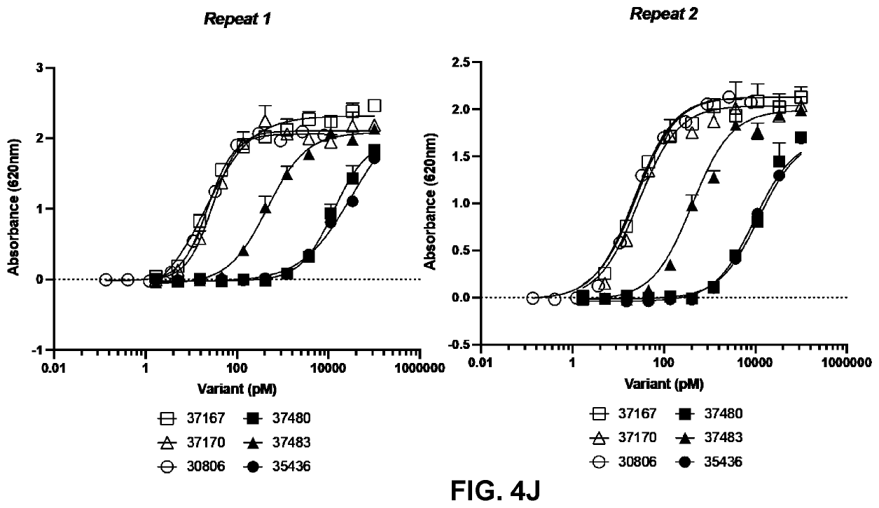

IL12 HetFc fusion proteins containing modified p40 domains (e.g., any one variants 37467-37495) display reduced potency compared to a control IL12 HetFc fusion protein containing a non-modified p40 domain (e.g., variant 30806) when assessed by an IL12 RGA (Table L, FIGS. 4A-4J), with EC50 values increasing up to approximately 570-fold for variant 37173 (avg. of n=2 repeats) and relative to variant 30806.

Masked IL12 HetFc fusion proteins containing modified p40 domains display reduced potency compared to the corresponding non-masked IL12 HetFc fusion proteins (Table L, FIGS. 4A-4J). Potency of Masked IL12 HetFc fusion proteins range from unchanged compared to a control Masked IL12 HetFc fusion protein containing a non-modified p40 domain (variant 35436) up to a complete loss of detectable activity (e.g., variants 37468, 37471, 37475, 37486).

TABLE L
RGA Activity of Unmasked and Masked IL12 HetFc Fusion Proteins
Containing Modified p40 Domains

| | | | | | | Masked Variants | | |
|---|---|---|---|---|---|---|---|---|
| | Unmasked Variants | | | | | | Repeat | Repeat |
| | | Repeat 1 | | Repeat 2 | | | 1 | 2 |
| p40 mu- | Variant | EC₅₀ | Fold- | EC₅₀ | Fold- | Variant | EC₅₀ | EC₅₀ |
| tations ^{a} | number | (pM) | EC₅₀ ^{b} | (pM) | EC₅₀ ^{b} | number | (pM) | (pM) |

Plate group 1-FIG. 4A

| | | | | | | | | |
|---|---|---|---|---|---|---|---|---|
| W15R_ | 37172 | 9841 | 401.0 | 9167 | 461.1 | 37485 | ND | ND |
| E59D_ | | | | | | | | |
| F60D_ | | | | | | | | |
| K197W | | | | | | | | |
| W15H_ | 37174 | 1527 | 62.2 | 1564 | 78.7 | 37487 | ND | ND |
| K84L_ | | | | | | | | |
| K197Q | | | | | | | | |
| E59D_ | 37175 | 1754 | 71.5 | 1431 | 72.0 | 37488 | ND | ND |
| K84W_ | | | | | | | | |
| K197W | | | | | | | | |
| None | 30806 | 25 | 1.0 | 20 | 1.0 | 35436 | 13572 | 10005 |

Plate group 2-FIG. 4B

| | | | | | | | | |
|---|---|---|---|---|---|---|---|---|
| K58H_ | 37176 | 167 | 6.9 | 141 | 6.9 | 37489 | 17432 | 17949 |
| E86R_ | | | | | | | | |
| K197D | | | | | | | | |
| W15H_ | 37178 | 83 | 3.5 | 73 | 3.6 | 37491 | 12848 | 12649 |
| K84L | | | | | | | | |
| F60R_ | 37173 | 8935 | 371.4 | 15834 | 771.6 | 37486 | ND | ND |
| K84E_ | | | | | | | | |
| K197W | | | | | | | | |
| None | 30806 | 24 | 1.0 | 21 | 1.0 | 35436 | 15694 | 10927 |

Plate group 3-FIG. 4C

| | | | | | | | | |
|---|---|---|---|---|---|---|---|---|
| K84W_ | 37157 | 1153 | 59.2 | 1103 | 68.3 | 37470 | 63187 | 68556 |
| K197W | | | | | | | | |
| F60K_ | 37158 | 4539 | 232.9 | 5176 | 320.7 | 37471 | ND | ND |
| K197W | | | | | | | | |
| E86R_ | 37156 | 149 | 7.6 | 137 | 8.5 | 37469 | 27824 | 18672 |
| K197D | | | | | | | | |
| None | 30806 | 19 | 1.0 | 16 | 1.0 | 35436 | 19472 | 7243 |

Plate group 4-FIG. 4D

| | | | | | | | | |
|---|---|---|---|---|---|---|---|---|
| K84I_ | 37177 | 30 | 1.6 | 26 | 1.4 | 37490 | 4283 | 3072 |
| E86R_ | | | | | | | | |
| D93H | | | | | | | | |
| E59R_ | 37154 | 2775 | 149.2 | 3471 | 182.6 | 37467 | ND | ND |
| D93R | | | | | | | | |
| K84I_ | 37159 | 21 | 1.1 | 24 | 1.3 | 37472 | 2540 | 3011 |
| E86R | | | | | | | | |
| None | 30806 | 19 | 1.0 | 19 | 1.0 | 35436 | 17498 | 8922 |

Plate group 5-FIG. 4E

| | | | | | | | | |
|---|---|---|---|---|---|---|---|---|
| W15K | 37179 | 562 | 23.7 | 412 | 22.6 | 37492 | 38713 | 121642 |
| K84W_ | 37163 | 51 | 2.2 | 49 | 2.7 | 37476 | 13253 | 12965 |
| D161R | | | | | | | | |
| None | 30806 | 24 | 1.0 | 18 | 1.0 | 35436 | 11318 | 9776 |

Plate group 6-FIG. 4F

| | | | | | | | | |
|---|---|---|---|---|---|---|---|---|
| E45R_ | 37181 | 180 | 8.7 | 215 | 11.8 | 37494 | ND | ND |
| K58S_ | | | | | | | | |
| E59S_ | | | | | | | | |
| K195D | | | | | | | | |
| E59D_ | 37162 | 36 | 1.7 | 51 | 2.8 | 37475 | 9506 | 6789 |
| D93H | | | | | | | | |
| F60R_ | 37155 | 1512 | 73.5 | 1582 | 87.1 | 37468 | ND | ND |
| K84Y | | | | | | | | |
| None | 30806 | 21 | 1.0 | 18 | 1.0 | 35436 | 8440 | 6859 |

Plate group 7-FIG. 4G

| | | | | | | | | |
|---|---|---|---|---|---|---|---|---|
| E86W_ | 37161 | 23 | 1.1 | 22 | 1.3 | 37474 | 585 | 482 |
| D93E | | | | | | | | |
| K58S_ | 37182 | 185 | 8.9 | 159 | 9.4 | 37495 | ND | ND |
| E59S_ | | | | | | | | |
| K195D | | | | | | | | |
| K84E | 37164 | 187 | 9.0 | 153 | 9.0 | 37477 | 18497 | 21767 |
| None | 30806 | 21 | 1.0 | 17 | 1.0 | 35436 | 16904 | 10973 |

Plate group 8-FIG. 4H

| | | | | | | | | |
|---|---|---|---|---|---|---|---|---|
| K197T | 37166 | 79 | 4.4 | 57 | 3.7 | 37479 | 16422 | 16381 |
| E59R | 37165 | 89 | 5.0 | 73 | 4.7 | 37478 | 15146 | 13120 |
| W15H | 37180 | 38 | 2.2 | 40 | 2.6 | 37493 | 6049 | 5122 |
| None | 30806 | 18 | 1.0 | 15 | 1.0 | 35436 | 13108 | 11064 |

Plate group 9-FIG. 4I

| | | | | | | | | |
|---|---|---|---|---|---|---|---|---|
| K84W | 37169 | 52 | 2.5 | 42 | 2.0 | 37482 | 14867 | 8147 |
| K197W | 37171 | 143 | 7.0 | 156 | 7.4 | 37484 | ND | ND |
| E59S | 37168 | 48 | 2.3 | 49 | 2.3 | 37481 | 53085 | 29188 |
| None | 30806 | 21 | 1.0 | 21 | 1.0 | 35436 | 10838 | 7349 |

Plate group 10-FIG. 4J

| | | | | | | | | |
|---|---|---|---|---|---|---|---|---|
| D161R | 37167 | 20 | 0.9 | 19 | 0.8 | 37480 | 12975 | 12869 |
| E86W | 37170 | 29 | 1.3 | 24 | 1.0 | 37483 | 457 | 474 |
| None | 30806 | 23 | 1.0 | 23 | 1.0 | 35436 | 32005 | 12943 |

^{a} Mutations are numbered according to the mature p40 sequence without signal peptide (SP). See, e.g., Tables B and/or C for p40 precursor sequence numbering.
^{b} Fold-change EC₅₀ compared to variant 30806 (IL12 HetFc fusion protein with a non-modified p40 domain) on the same experimental plate.
^{c} ND = Not Determined due to incomplete curve.

These data demonstrate that at least some of the one or more amino acid substitutions determined using in silico methods as described in EXAMPLE 1 successfully generated modified p40 domains with reduced affinity to its cognate IL12 receptor.

### SEQUENCE TABLE

TABLE M
Sequences

| SEQ | | | |
|---|---|---|---|
| ID NO | Clone # | Description | Sequence (amino acid or nucleic acid) |
| 1 | | Signal peptide | MRPTWAWWLFLVLLLALWAPARG |
| 2 | | Bria VH | QVQLVESGGGVVQPGRSLRLSCAASGFTFSSYGMHWVRQAP |
| | | | GKGLEWVAFIRYDGSNKYYADSVKGRFTISRDNSKNTLYLQ |
| | | | MNSLRAEDTAVYYCKTHGSHDNWGQGTMVTVSS |
| 3 | | Bria VL | QSVLTQPPSVSGAPGQRVTISCSGSRSNIGSNTVKWYQQLPGT |
| | | | APKLLIYYNDQRPSGVPDRFSGSKSGTSASLAITGLQAEDEAD |
| | | | YYCQSYDRYTHPALLFGTGTKVTVL |
| 4 | | Bria VHCDR1 | SYGMH |
| 5 | | Bria VHCDR2 | FIRYDGSNKYYADSVKG |
| 6 | | Bria VHCDR3 | HGSHDN |
| 7 | | Bria VLCDR1 | SGSRSNIGSNTVK |
| 8 | | Bria VLCDR2 | YNDQRPS |
| 9 | | Bria VLCDR3 | QSYDRYTHPALL |
| 10 | | p40 (mature) | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 11 | | p35 (mature) | RNLPVATPDPGMFPCLHHSQNLLRAVSNMLQKARQTLEFYP |
| | | | CTSEEIDHEDITKDKTSTVEACLPLELTKNESCLNSRETSFITN |
| | | | GSCLASRKTSFMMALCLSSIYEDLKMYQVEFKTMNAKLLMD |
| | | | PKRQIFLDQNMLAVIDELMQALNFNSETVPQKSSLEEPDFYK |
| | | | TKIKLCILLHAFRIRAVTIDRVMSYLNAS |
| 12 | | Mutated p40 | IWELKKDVYVVELDHYPDAPGEMVVLTCDTPEEDGITWTLD |
| | | | QSSEVLGSGKTLTIQVKEFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 13 | | Mutated p40 | IWELKKDVYVVELDKYPDAPGEMVVLTCDTPEEDGITWTLD |
| | | | QSSEVLGSGKTLTIQVKEFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 14 | | Mutated p40 | IWELKKDVYVVELDRYPDAPGEMVVLTCDTPEEDGITWTLD |
| | | | QSSEVLGSGKTLTIQVKEFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 15 | | Mutated p40 | IWELKKDVYVVELDWYPDAPGEMVVLTCDTPEEDGITWTLD |
| | | | QSSRVLGSGKTLTIQVKEFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 16 | | Mutated p40 | IWELKKDVYVVELDWYPDAPGEMVVLTCDTPEEDGITWTLD |
| | | | QSSEVLGSGKTLTIQVHEFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 17 | | Mutated p40 | IWELKKDVYVVELDWYPDAPGEMVVLTCDTPEEDGITWTLD |
| | | | QSSEVLGSGKTLTIQVSEFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 18 | | Mutated p40 | IWELKKDVYVVELDWYPDAPGEMVVLTCDTPEEDGITWTLD |
| | | | QSSEVLGSGKTLTIQVKDFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 19 | | Mutated p40 | IWELKKDVYVVELDWYPDAPGEMVVLTCDTPEEDGITWTLD |
| | | | QSSEVLGSGKTLTIQVKRFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 20 | | Mutated p40 | IWELKKDVYVVELDWYPDAPGEMVVLTCDTPEEDGITWTLD |
| | | | QSSEVLGSGKTLTIQVKSFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 21 | | Mutated p40 | IWELKKDVYVVELDWYPDAPGEMVVLTCDTPEEDGITWTLD |
| | | | QSSEVLGSGKTLTIQVKEDGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 22 | | Mutated p40 | IWELKKDVYVVELDWYPDAPGEMVVLTCDTPEEDGITWTLD |
| | | | QSSEVLGSGKTLTIQVKEKGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 23 | | Mutated p40 | IWELKKDVYVVELDWYPDAPGEMVVLTCDTPEEDGITWTLD |
| | | | QSSEVLGSGKTLTIQVKERGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 24 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | EKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 25 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLHI |
| | | | KEDGIWSTDILKDQKEPKNKTFLRCEAKNYSGRFTCWWLTTI |
| | | | STDLTFSVKSSRGSSDPQGVTCGAATLSAERVRGDNKEYEYS |
| | | | VECQEDSACPAAEESLPIEVMVDAVHKLKYENYTSSFFIRDII |
| | | | KPDPPKNLQLKPLKNSRQVEVSWEYPDTWSTPHSYFSLTFCV |
| | | | QVQGKSKREKKDRVFTDKTSATVICRKNASISVRAQDRYYSS |
| | | | SWSEWASVPCS |
| 26 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | LKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 27 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | WKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 28 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | YKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 29 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | KKRDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 30 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | KKWDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 31 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | KKEDGIWSTEILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 32 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | KKEDGIWSTHILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 33 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | KKEDGIWSTRILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 34 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGRNKEYEY |
| | | | SVECQEDSACPAAEESLPIEVMVDAVHKLKYENYTSSFFIRDII |
| | | | KPDPPKNLQLKPLKNSRQVEVSWEYPDTWSTPHSYFSLTFCV |
| | | | QVQGKSKREKKDRVFTDKTSATVICRKNASISVRAQDRYYSS |
| | | | SWSEWASVPCS |
| 35 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHDLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 36 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLDYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 37 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLQYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 38 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLTYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 39 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLWYENYTSSFFIR |
| | | | DIIKPDPPKNLQLKPLKNSRQVEVSWEYPDTWSTPHSYFSLTF |
| | | | CVQVQGKSKREKKDRVFTDKTSATVICRKNASISVRAQDRY |
| | | | YSSSWSEWASVPCS |
| 40 | | Mutated p40 | IWELKKDVYVVELDRYPDAPGEMVVLTCDTPEEDGITWTLD |
| | | | QSSEVLGSGKTLTIQVKDDGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLWYENYTSSFFIR |
| | | | DIIKPDPPKNLQLKPLKNSRQVEVSWEYPDTWSTPHSYFSLTF |
| | | | CVQVQGKSKREKKDRVFTDKTSATVICRKNASISVRAQDRY |
| | | | YSSSWSEWASVPCS |
| 41 | | Mutated p40 | IWELKKDVYVVELDHYPDAPGEMVVLTCDTPEEDGITWTLD |
| | | | QSSEVLGSGKTLTIQVKEFGDAGQYTCHKGGEVLSHSLLLLH |
| | | | LKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 42 | | Mutated p40 | IWELKKDVYVVELDHYPDAPGEMVVLTCDTPEEDGITWTLD |
| | | | QSSEVLGSGKTLTIQVKEFGDAGQYTCHKGGEVLSHSLLLLH |
| | | | LKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLQYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 43 | | Mutated p40 | IWELKKDVYVVELDWYPDAPGEMVVLTCDTPEEDGITWTLD |
| | | | QSSRVLGSGKTLTIQVSSFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHDLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 44 | | Mutated p40 | IWELKKDVYVVELDWYPDAPGEMVVLTCDTPEEDGITWTLD |
| | | | QSSEVLGSGKTLTIQVHEFGDAGQYTCHKGGEVLSHSLLLLH |
| | | | KKRDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLDYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 45 | | Mutated p40 | IWELKKDVYVVELDWYPDAPGEMVVLTCDTPEEDGITWTLD |
| | | | QSSEVLGSGKTLTIQVSSFGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHDLKYENYTSSFFIRD |
| | | | TIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 46 | | Mutated p40 | IWELKKDVYVVELDWYPDAPGEMVVLTCDTPEEDGITWTLD |
| | | | QSSEVLGSGKTLTIQVKDFGDAGQYTCHKGGEVLSHSLLLLH |
| | | | WKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLWYENYTSSFFIR |
| | | | DIIKPDPPKNLQLKPLKNSRQVEVSWEYPDTWSTPHSYFSLTF |
| | | | CVQVQGKSKREKKDRVFTDKTSATVICRKNASISVRAQDRY |
| | | | YSSSWSEWASVPCS |
| 47 | | Mutated p40 | IWELKKDVYVVELDWYPDAPGEMVVLTCDTPEEDGITWTLD |
| | | | QSSEVLGSGKTLTIQVKDFGDAGQYTCHKGGEVLSHSLLLLH |
| | | | KKEDGIWSTHILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 48 | | Mutated p40 | IWELKKDVYVVELDWYPDAPGEMVVLTCDTPEEDGITWTLD |
| | | | QSSEVLGSGKTLTIQVKRFGDAGQYTCHKGGEVLSHSLLLLH |
| | | | KKEDGIWSTRILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 49 | | Mutated p40 | IWELKKDVYVVELDWYPDAPGEMVVLTCDTPEEDGITWTLD |
| | | | QSSEVLGSGKTLTIQVKEKGDAGQYTCHKGGEVLSHSLLLLH |
| | | | KKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLWYENYTSSFFIR |
| | | | DIIKPDPPKNLQLKPLKNSRQVEVSWEYPDTWSTPHSYFSLTF |
| | | | CVQVQGKSKREKKDRVFTDKTSATVICRKNASISVRAQDRY |
| | | | YSSSWSEWASVPCS |
| 50 | | Mutated p40 | IWELKKDVYVVELDWYPDAPGEMVVLTCDTPEEDGITWTLD |
| | | | QSSEVLGSGKTLTIQVKERGDAGQYTCHKGGEVLSHSLLLLH |
| | | | EKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLWYENYTSSFFIR |
| | | | DIIKPDPPKNLQLKPLKNSRQVEVSWEYPDTWSTPHSYFSLTF |
| | | | CVQVQGKSKREKKDRVFTDKTSATVICRKNASISVRAQDRY |
| | | | YSSSWSEWASVPCS |
| 51 | | Mutated p40 | IWELKKDVYVVELDWYPDAPGEMVVLTCDTPEEDGITWTLD |
| | | | QSSEVLGSGKTLTIQVKERGDAGQYTCHKGGEVLSHSLLLLH |
| | | | YKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 52 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLHI |
| | | | KRDGIWSTDILKDQKEPKNKTFLRCEAKNYSGRFTCWWLTTI |
| | | | STDLTFSVKSSRGSSDPQGVTCGAATLSAERVRGDNKEYEYS |
| | | | VECQEDSACPAAEESLPIEVMVDAVHKLKYENYTSSFFIRDII |
| | | | KPDPPKNLQLKPLKNSRQVEVSWEYPDTWSTPHSYFSLTFCV |
| | | | QVQGKSKREKKDRVFTDKTSATVICRKNASISVRAQDRYYSS |
| | | | SWSEWASVPCS |
| 53 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLHI |
| | | | KRDGIWSTHILKDQKEPKNKTFLRCEAKNYSGRFTCWWLTTI |
| | | | STDLTFSVKSSRGSSDPQGVTCGAATLSAERVRGDNKEYEYS |
| | | | VECQEDSACPAAEESLPIEVMVDAVHKLKYENYTSSFFIRDII |
| | | | KPDPPKNLQLKPLKNSRQVEVSWEYPDTWSTPHSYFSLTFCV |
| | | | QVQGKSKREKKDRVFTDKTSATVICRKNASISVRAQDRYYSS |
| | | | SWSEWASVPCS |
| 54 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | WKEDGIWSTDILKDQKEPKNKTFLRCEAKNYSGRFTCWWLT |
| | | | TISTDLTFSVKSSRGSSDPQGVTCGAATLSAERVRGRNKEYEY |
| | | | SVECQEDSACPAAEESLPIEVMVDAVHKLKYENYTSSFFIRDII |
| | | | KPDPPKNLQLKPLKNSRQVEVSWEYPDTWSTPHSYFSLTFCV |
| | | | QVQGKSKREKKDRVFTDKTSATVICRKNASISVRAQDRYYSS |
| | | | SWSEWASVPCS |
| 55 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | WKEDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLWYENYTSSFFIR |
| | | | DIIKPDPPKNLQLKPLKNSRQVEVSWEYPDTWSTPHSYFSLTF |
| | | | CVQVQGKSKREKKDRVFTDKTSATVICRKNASISVRAQDRY |
| | | | YSSSWSEWASVPCS |
| 56 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | KKRDGIWSTDILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLDYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 57 | | Mutated p40 | IWELKKDVYVVELDWYPDAPGEMVVLTCDTPEEDGITWTLD |
| | | | QSSEVLGSGKTLTIQVKEFGDAGQYTCHKGGEVLSHSLLLLH |
| | | | KKWDGIWSTEILKDQKEPKNKTFLRCEAKNYSGRFTCWWLT |
| | | | TISTDLTFSVKSSRGSSDPQGVTCGAATLSAERVRGDNKEYE |
| | | | YSVECQEDSACPAAEESLPIEVMVDAVHKLKYENYTSSFFIRD |
| | | | IIKPDPPKNLQLKPLKNSRQVEVSWEYPDTWSTPHSYFSLTFC |
| | | | VQVQGKSKREKKDRVFTDKTSATVICRKNASISVRAQDRYYS |
| | | | SSWSEWASVPCS |
| 58 | CL_#12153 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVLPPSRDELTKNQVSLLCLVKGFYPSDIAVEWE |
| | | | SNGQPENNYLTWPPVLDSDGSFFLYSKLTVDKSRWQQGNVF |
| | | | SCSVMHEALHNHYTQKSLSLSPG |
| 59 | CL_#12155 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPG |
| 60 | CL_#26503 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVLPPSRDELTKNQVSLLCLVKGFYPSDIAVEWE |
| | | | SNGQPENNYLTWPPVLDSDGSFFLYSKLTVDKSRWQQGNVF |
| | | | SCSVMHEALHNHYTQKSLSLSPGGGGGSGGGGSGGGSGGGS |
| | | | GGGGSGGGGSQVQLVESGGGVVQPGRSLRLSCAASGFTFSSY |
| | | | GMHWVRQAPGKGLEWVAFIRYDGSNKYYADSVKGRFTISR |
| | | | DNSKNTLYLQMNSLRAEDTAVYYCKTHGSHDNWGQGTMV |
| | | | TVSSGGSGGGSGGGSGGGSGGGSGQSVLTQPPSVSGAPGQRV |
| | | | TISCSGSRSNIGSNTVKWYQQLPGTAPKLLIYYNDQRPSGVPD |
| | | | RFSGSKSGTSASLAITGLQAEDEADYYCQSYDRYTHPALLFG |
| | | | TGTKVTVL |
| 61 | CL_#22279 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKEFGDAGQYTCHKGGEVLSHSLLLLHKKEDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLKYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 62 | CL_#28046 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKRFGDAGQYTCHKGGEVLSHSLLLLHKKEDGIWSTRIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLKYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 63 | CL_#28047 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKERGDAGQYTCHKGGEVLSHSLLLLHYKEDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLKYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 64 | CL_#28048 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKEFGDAGQYTCHKGGEVLSHSLLLLHKKRDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLDYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 65 | CL_#28049 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKEFGDAGQYTCHKGGEVLSHSLLLLHWKEDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLWYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 66 | CL_#28050 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKEKGDAGQYTCHKGGEVLSHSLLLLHKKEDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLWYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 67 | CL_#28051 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKEFGDAGQYTCHKGGEVLSHSLLLLHIKRDGIWSTDILK |
| | | | DQKEPKNKTFLRCEAKNYSGRFTCWWLTTISTDLTFSVKSSR |
| | | | GSSDPQGVTCGAATLSAERVRGDNKEYEYSVECQEDSACPA |
| | | | AEESLPIEVMVDAVHKLKYENYTSSFFIRDIIKPDPPKNLQLKP |
| | | | LKNSRQVEVSWEYPDTWSTPHSYFSLTFCVQVQGKSKREKK |
| | | | DRVFTDKTSATVICRKNASISVRAQDRYYSSSWSEWASVPCS |
| | | | GGGGSGGGGSGGGGSNLPVATPDPGMFPCLHHSQNLLRAVS |
| | | | NMLQKARQTLEFYPCTSEEIDHEDITKDKTSTVEACLPLELTK |
| | | | NESCLNSRETSFITNGSCLASRKTSFMMALCLSSIYEDLKMYQ |
| | | | VEFKTMNAKLLMDPKRQIFLDQNMLAVIDELMQALNFNSET |
| | | | VPQKSSLEEPDFYKTKIKLCILLHAFRIRAVTIDRVMSYLNAS |
| 68 | CL_#28053 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKEFGDAGQYTCHKGGEVLSHSLLLLHKKWDGIWSTEIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLKYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 69 | CL_#28054 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKDFGDAGQYTCHKGGEVLSHSLLLLHKKEDGIWSTHIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLKYENYTSSFFIRDIIKPDPPKNLOL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 70 | CL_#28055 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKEFGDAGQYTCHKGGEVLSHSLLLLHWKEDGIWSTDIL |
| | | | KDQKEPKNKTFLRCEAKNYSGRFTCWWLTTISTDLTFSVKSS |
| | | | RGSSDPQGVTCGAATLSAERVRGRNKEYEYSVECQEDSACP |
| | | | AAEESLPIEVMVDAVHKLKYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 71 | CL_#28056 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKEFGDAGQYTCHKGGEVLSHSLLLLHEKEDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLKYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 72 | CL_#28057 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKRFGDAGQYTCHKGGEVLSHSLLLLHKKEDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLKYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 73 | CL_#28058 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKEFGDAGQYTCHKGGEVLSHSLLLLHKKEDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLTYENYTSSFFIRDIIKPDPPKNLQLK |
| | | | PLKNSRQVEVSWEYPDTWSTPHSYFSLTFCVQVQGKSKREK |
| | | | KDRVFTDKTSATVICRKNASISVRAQDRYYSSSWSEWASVPC |
| | | | SGGGGSGGGGSGGGGSNLPVATPDPGMFPCLHHSQNLLRAV |
| | | | SNMLQKARQTLEFYPCTSEEIDHEDITKDKTSTVEACLPLELT |
| | | | KNESCLNSRETSFITNGSCLASRKTSFMMALCLSSIYEDLKMY |
| | | | QVEFKTMNAKLLMDPKRQIFLDQNMLAVIDELMQALNFNSE |
| | | | TVPQKSSLEEPDFYKTKIKLCILLHAFRIRAVTIDRVMSYLNAS |
| 74 | CL_#28059 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKEFGDAGQYTCHKGGEVLSHSLLLLHKKEDGIWSTDIL |
| | | | KDQKEPKNKTFLRCEAKNYSGRFTCWWLTTISTDLTFSVKSS |
| | | | RGSSDPQGVTCGAATLSAERVRGRNKEYEYSVECQEDSACP |
| | | | AAEESLPIEVMVDAVHKLKYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 75 | CL_#28060 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKSFGDAGQYTCHKGGEVLSHSLLLLHKKEDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLKYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 76 | CL_#28061 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKEFGDAGQYTCHKGGEVLSHSLLLLHWKEDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLKYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 77 | CL_#28062 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKEFGDAGQYTCHKGGEVLSHSLLLLHKKWDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLKYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 78 | CL_#28063 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKEFGDAGQYTCHKGGEVLSHSLLLLHKKEDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLWYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 79 | CL_#28064 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDRYPDAPGEMVVLTCDTPEEDGITWTLDQSSEVLGSGKTLT |
| | | | IQVKDDGDAGQYTCHKGGEVLSHSLLLLHKKEDGIWSTDILK |
| | | | DQKEPKNKTFLRCEAKNYSGRFTCWWLTTISTDLTFSVKSSR |
| | | | GSSDPQGVTCGAATLSAERVRGDNKEYEYSVECQEDSACPA |
| | | | AEESLPIEVMVDAVHKLWYENYTSSFFIRDIIKPDPPKNLQLK |
| | | | PLKNSRQVEVSWEYPDTWSTPHSYFSLTFCVQVQGKSKREK |
| | | | KDRVFTDKTSATVICRKNASISVRAQDRYYSSSWSEWASVPC |
| | | | SGGGGSGGGGSGGGGSNLPVATPDPGMFPCLHHSQNLLRAV |
| | | | SNMLQKARQTLEFYPCTSEEIDHEDITKDKTSTVEACLPLELT |
| | | | KNESCLNSRETSFITNGSCLASRKTSFMMALCLSSIYEDLKMY |
| | | | QVEFKTMNAKLLMDPKRQIFLDQNMLAVIDELMQALNFNSE |
| | | | TVPQKSSLEEPDFYKTKIKLCILLHAFRIRAVTIDRVMSYLNAS |
| 80 | CL_#28065 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKERGDAGQYTCHKGGEVLSHSLLLLHEKEDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLWYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 81 | CL_#28066 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDHYPDAPGEMVVLTCDTPEEDGITWTLDQSSEVLGSGKTLT |
| | | | IQVKEFGDAGQYTCHKGGEVLSHSLLLLHLKEDGIWSTDILK |
| | | | DQKEPKNKTFLRCEAKNYSGRFTCWWLTTISTDLTFSVKSSR |
| | | | GSSDPQGVTCGAATLSAERVRGDNKEYEYSVECQEDSACPA |
| | | | AEESLPIEVMVDAVHKLQYENYTSSFFIRDIIKPDPPKNLQLKP |
| | | | LKNSRQVEVSWEYPDTWSTPHSYFSLTFCVQVQGKSKREKK |
| | | | DRVFTDKTSATVICRKNASISVRAQDRYYSSSWSEWASVPCS |
| | | | GGGGSGGGGSGGGGSNLPVATPDPGMFPCLHHSQNLLRAVS |
| | | | NMLQKARQTLEFYPCTSEEIDHEDITKDKTSTVEACLPLELTK |
| | | | NESCLNSRETSFITNGSCLASRKTSFMMALCLSSIYEDLKMYQ |
| | | | VEFKTMNAKLLMDPKRQIFLDQNMLAVIDELMQALNFNSET |
| | | | VPQKSSLEEPDFYKTKIKLCILLHAFRIRAVTIDRVMSYLNAS |
| 82 | CL_#28067 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKDFGDAGQYTCHKGGEVLSHSLLLLHWKEDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLWYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 83 | CL_#28068 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVHEFGDAGQYTCHKGGEVLSHSLLLLHKKRDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHKLDYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 84 | CL_#28069 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVKEFGDAGQYTCHKGGEVLSHSLLLLHIKRDGIWSTHILK |
| | | | DQKEPKNKTFLRCEAKNYSGRFTCWWLTTISTDLTFSVKSSR |
| | | | GSSDPQGVTCGAATLSAERVRGDNKEYEYSVECQEDSACPA |
| | | | AEESLPIEVMVDAVHKLKYENYTSSFFIRDIIKPDPPKNLQLKP |
| | | | LKNSRQVEVSWEYPDTWSTPHSYFSLTFCVQVQGKSKREKK |
| | | | DRVFTDKTSATVICRKNASISVRAQDRYYSSSWSEWASVPCS |
| | | | GGGGSGGGGSGGGGSNLPVATPDPGMFPCLHHSQNLLRAVS |
| | | | NMLQKARQTLEFYPCTSEEIDHEDITKDKTSTVEACLPLELTK |
| | | | NESCLNSRETSFITNGSCLASRKTSFMMALCLSSIYEDLKMYQ |
| | | | VEFKTMNAKLLMDPKRQIFLDQNMLAVIDELMQALNFNSET |
| | | | VPQKSSLEEPDFYKTKIKLCILLHAFRIRAVTIDRVMSYLNAS |
| 85 | CL_#28070 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDHYPDAPGEMVVLTCDTPEEDGITWTLDQSSEVLGSGKTLT |
| | | | IQVKEFGDAGQYTCHKGGEVLSHSLLLLHLKEDGIWSTDILK |
| | | | DQKEPKNKTFLRCEAKNYSGRFTCWWLTTISTDLTFSVKSSR |
| | | | GSSDPQGVTCGAATLSAERVRGDNKEYEYSVECQEDSACPA |
| | | | AEESLPIEVMVDAVHKLKYENYTSSFFIRDIIKPDPPKNLQLKP |
| | | | LKNSRQVEVSWEYPDTWSTPHSYFSLTFCVQVQGKSKREKK |
| | | | DRVFTDKTSATVICRKNASISVRAQDRYYSSSWSEWASVPCS |
| | | | GGGGSGGGGSGGGGSNLPVATPDPGMFPCLHHSQNLLRAVS |
| | | | NMLQKARQTLEFYPCTSEEIDHEDITKDKTSTVEACLPLELTK |
| | | | NESCLNSRETSFITNGSCLASRKTSFMMALCLSSIYEDLKMYQ |
| | | | VEFKTMNAKLLMDPKRQIFLDQNMLAVIDELMQALNFNSET |
| | | | VPQKSSLEEPDFYKTKIKLCILLHAFRIRAVTIDRVMSYLNAS |
| 86 | CL_#28071 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDKYPDAPGEMVVLTCDTPEEDGITWTLDQSSEVLGSGKTLT |
| | | | IQVKEFGDAGQYTCHKGGEVLSHSLLLLHKKEDGIWSTDILK |
| | | | DQKEPKNKTFLRCEAKNYSGRFTCWWLTTISTDLTFSVKSSR |
| | | | GSSDPQGVTCGAATLSAERVRGDNKEYEYSVECQEDSACPA |
| | | | AEESLPIEVMVDAVHKLKYENYTSSFFIRDIIKPDPPKNLQLKP |
| | | | LKNSRQVEVSWEYPDTWSTPHSYFSLTFCVQVQGKSKREKK |
| | | | DRVFTDKTSATVICRKNASISVRAQDRYYSSSWSEWASVPCS |
| | | | GGGGSGGGGSGGGGSNLPVATPDPGMFPCLHHSQNLLRAVS |
| | | | NMLQKARQTLEFYPCTSEEIDHEDITKDKTSTVEACLPLELTK |
| | | | NESCLNSRETSFITNGSCLASRKTSFMMALCLSSIYEDLKMYQ |
| | | | VEFKTMNAKLLMDPKRQIFLDQNMLAVIDELMQALNFNSET |
| | | | VPQKSSLEEPDFYKTKIKLCILLHAFRIRAVTIDRVMSYLNAS |
| 87 | CL_#28072 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDHYPDAPGEMVVLTCDTPEEDGITWTLDQSSEVLGSGKTLT |
| | | | IQVKEFGDAGQYTCHKGGEVLSHSLLLLHKKEDGIWSTDILK |
| | | | DQKEPKNKTFLRCEAKNYSGRFTCWWLTTISTDLTFSVKSSR |
| | | | GSSDPQGVTCGAATLSAERVRGDNKEYEYSVECQEDSACPA |
| | | | AEESLPIEVMVDAVHKLKYENYTSSFFIRDIIKPDPPKNLQLKP |
| | | | LKNSRQVEVSWEYPDTWSTPHSYFSLTFCVQVQGKSKREKK |
| | | | DRVFTDKTSATVICRKNASISVRAQDRYYSSSWSEWASVPCS |
| | | | GGGGSGGGGSGGGGSNLPVATPDPGMFPCLHHSQNLLRAVS |
| | | | NMLQKARQTLEFYPCTSEEIDHEDITKDKTSTVEACLPLELTK |
| | | | NESCLNSRETSFITNGSCLASRKTSFMMALCLSSIYEDLKMYQ |
| | | | VEFKTMNAKLLMDPKRQIFLDQNMLAVIDELMQALNFNSET |
| | | | VPQKSSLEEPDFYKTKIKLCILLHAFRIRAVTIDRVMSYLNAS |
| 88 | CL_#28074 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSRVLGSGKTL |
| | | | TIQVSSFGDAGQYTCHKGGEVLSHSLLLLHKKEDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHDLKYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 89 | CL_#28075 | Full AA | EPKSSDKTHTCPPCPAPEAAGGPSVFLFPPKPKDTLMISRTPEV |
| | | | TCVVVSVSHEDPEVKFNWYVDGVEVHNAKTKPREEQYNST |
| | | | YRVVSVLTVLHQDWLNGKEYKCKVSNKALPAPIEKTISKAK |
| | | | GQPREPQVYVYPPSRDELTKNQVSLTCLVKGFYPSDIAVEWE |
| | | | SNGQPENNYKTTPPVLDSDGSFALVSKLTVDKSRWQQGNVF |
| | | | SCSVMHEALHNHYTQKSLSLSPGGSADGGIWELKKDVYVVE |
| | | | LDWYPDAPGEMVVLTCDTPEEDGITWTLDQSSEVLGSGKTL |
| | | | TIQVSSFGDAGQYTCHKGGEVLSHSLLLLHKKEDGIWSTDIL |
| | | | KDQKEPKNKTFLRCEAKNYSGRFTCWWLTTISTDLTFSVKSS |
| | | | RGSSDPQGVTCGAATLSAERVRGDNKEYEYSVECQEDSACP |
| | | | AAEESLPIEVMVDAVHDLKYENYTSSFFIRDIIKPDPPKNLQL |
| | | | KPLKNSRQVEVSWEYPDTWSTPHSYFSLTFCVQVQGKSKRE |
| | | | KKDRVFTDKTSATVICRKNASISVRAQDRYYSSSWSEWASVP |
| | | | CSGGGGSGGGGSGGGGSNLPVATPDPGMFPCLHHSQNLLRA |
| | | | VSNMLQKARQTLEFYPCTSEEIDHEDITKDKTSTVEACLPLEL |
| | | | TKNESCLNSRETSFITNGSCLASRKTSFMMALCLSSIYEDLKM |
| | | | YQVEFKTMNAKLLMDPKRQIFLDQNMLAVIDELMQALNFNS |
| | | | ETVPQKSSLEEPDFYKTKIKLCILLHAFRIRAVTIDRVMSYLNA |
| | | | S |
| 90 | CL_#12153 | Full nt | GAGCCAAAGAGCTCCGACAAGACCCACACATGCCCCCCTT |
| | | | GTCCGGCGCCAGAGGCAGCAGGAGGACCAAGCGTGTTCCT |
| | | | GTTTCCACCCAAGCCCAAAGACACCCTGATGATTAGCCGA |
| | | | ACCCCTGAAGTCACATGCGTGGTCGTGTCCGTGTCTCACGA |
| | | | GGACCCAGAAGTCAAGTTCAACTGGTACGTGGATGGCGTC |
| | | | GAGGTGCATAATGCCAAGACAAAACCCCGGGAGGAACAG |
| | | | TACAACAGCACCTATAGAGTCGTGTCCGTCCTGACAGTGC |
| | | | TGCACCAGGATTGGCTGAACGGCAAGGAATATAAGTGCAA |
| | | | AGTGTCCAATAAGGCCCTGCCCGCTCCTATCGAGAAAACC |
| | | | ATTTCTAAGGCAAAAGGCCAGCCTCGCGAACCACAGGTCT |
| | | | ACGTGCTGCCTCCATCCCGGGACGAGCTGACAAAGAACCA |
| | | | GGTCTCTCTGCTGTGCCTGGTGAAAGGCTTCTATCCATCAG |
| | | | ATATTGCTGTGGAGTGGGAAAGCAATGGGCAGCCCGAGAA |
| | | | CAATTACCTGACTTGGCCCCCTGTGCTGGACTCTGATGGGA |
| | | | GTTTCTTTCTGTATTCTAAGCTGACCGTGGATAAAAGTAGG |
| | | | TGGCAGCAGGGAAATGTCTTTAGTTGTTCAGTGATGCATG |
| | | | AAGCCCTGCATAACCACTACACCCAGAAAAGCCTGTCCCT |
| | | | GTCCCCCGGA |
| 91 | CL_#12155 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCACAGGTC |
| | | | TACGTGTATCCTCCAAGCCGGGACGAGCTGACAAAGAACC |
| | | | AGGTCTCCCTGACTTGTCTGGTGAAAGGGTTTTACCCTAGT |
| | | | GATATCGCTGTGGAGTGGGAATCAAATGGACAGCCAGAGA |
| | | | ACAATTATAAGACTACCCCCCCTGTGCTGGACAGTGATGG |
| | | | GTCATTCGCACTGGTCTCCAAGCTGACAGTGGACAAATCT |
| | | | CGGTGGCAGCAGGGAAATGTCTTTTCATGTAGCGTGATGC |
| | | | ATGAAGCACTGCACAACCATTACACCCAGAAGTCACTGTC |
| | | | ACTGTCACCAGGA |
| 92 | CL_#26503 | Full nt | GAGCCAAAGAGCTCCGACAAGACCCACACATGCCCCCCTT |
| | | | GTCCGGCGCCAGAGGCAGCAGGAGGACCAAGCGTGTTCCT |
| | | | GTTTCCACCCAAGCCCAAAGACACCCTGATGATTAGCCGA |
| | | | ACCCCTGAAGTCACATGCGTGGTCGTGTCCGTGTCTCACGA |
| | | | GGACCCAGAAGTCAAGTTCAACTGGTACGTGGATGGCGTC |
| | | | GAGGTGCATAATGCCAAGACAAAACCCCGGGAGGAACAG |
| | | | TACAACAGCACCTATAGAGTCGTGTCCGTCCTGACAGTGC |
| | | | TGCACCAGGATTGGCTGAACGGCAAGGAATATAAGTGCAA |
| | | | AGTGTCCAATAAGGCCCTGCCCGCTCCTATCGAGAAAACC |
| | | | ATTTCTAAGGCAAAAGGCCAGCCTCGCGAGCCTCAGGTGT |
| | | | ACGTGCTGCCACCTTCCCGCGACGAGCTGACCAAGAATCA |
| | | | GGTGTCTCTGCTGTGCCTGGTGAAGGGCTTCTATCCAAGCG |
| | | | ATATCGCAGTGGAGTGGGAGTCCAACGGACAGCCCGAGA |
| | | | ACAATTACCTGACCTGGCCACCCGTGCTGGACAGCGATGG |
| | | | CTCCTTCTTTCTGTATTCCAAGCTGACAGTGGACAAGTCTA |
| | | | GATGGCAGCAGGGCAACGTGTTCAGCTGTTCCGTGATGCA |
| | | | CGAGGCCCTGCACAACCACTACACCCAGAAGTCTCTGAGC |
| | | | TTAAGCCCTGGAGGAGGAGGAGGCTCCGGCGGAGGAGGC |
| | | | TCCGGCGGCGGGAGCGGCGGCGGCAGCGGGGGCGGCGGC |
| | | | TCCGGCGGCGGCGGCTCTCAGGTGCAGCTGGTGGAGAGCG |
| | | | GAGGAGGAGTGGTGCAGCCCGGCAGAAGCCTGCGGCTGA |
| | | | GCTGCGCAGCCTCTGGCTTCACCTTTAGCTCCTACGGCATG |
| | | | CACTGGGTGCGGCAGGCCCCTGGCAAGGGACTGGAGTGGG |
| | | | TGGCCTTCATCAGATATGACGGCAGCAATAAGTACTATGC |
| | | | CGATTCCGTGAAGGGCAGGTTTACCATCAGCCGCGACAAC |
| | | | TCCAAGAATACACTGTACCTGCAGATGAACAGCCTGAGGG |
| | | | CCGAGGACACCGCCGTGTACTATTGTAAGACACACGGCTC |
| | | | CCACGATAATTGGGGCCAGGGCACCATGGTGACAGTGTCT |
| | | | AGCGGCGGCTCTGGCGGCGGCAGCGGGGGTGGCTCTGGAG |
| | | | GAGGCAGCGGAGGAGGCTCCGGCCAGTCTGTGCTGACCCA |
| | | | GCCACCTTCTGTGAGCGGAGCCCCCGGACAGAGGGTGACA |
| | | | ATCTCCTGCTCTGGCAGCCGCTCCAACATCGGCAGCAATA |
| | | | CCGTGAAGTGGTATCAGCAGCTGCCAGGCACAGCCCCCAA |
| | | | GCTGCTGATCTACTATAATGACCAGCGGCCTTCCGGCGTGC |
| | | | CAGATAGATTCTCTGGCAGCAAGTCCGGCACCTCTGCCAG |
| | | | CCTGGCCATCACAGGCCTGCAGGCAGAGGACGAGGCAGAT |
| | | | TACTATTGTCAGTCCTACGATAGATATACCCACCCCGCCCT |
| | | | GCTGTTTGGCACCGGCACAAAGGTGACAGTGCTG |
| 93 | CL_#22279 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCAC |
| | | | AAGAAGGAGGACGGCATCTGGAGCACAGACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGAAGTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 94 | CL_#28046 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGAGATTCGGCGACGCCGGACAGTACACCTGTCACAA |
| | | | GGGCGGCGAGGTGCTGTCCCACTCTCTGCTGCTGCTGCAC |
| | | | AAGAAGGAGGACGGCATCTGGAGCACAAGAATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGAAGTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 95 | CL_#28047 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGAGAGGCGACGCCGGACAGTACACCTGTCACA |
| | | | AGGGCGGCGAGGTGCTGTCCCACTCTCTGCTGCTGCTGCA |
| | | | CTACAAGGAGGACGGCATCTGGAGCACAGACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGAAGTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 96 | CL_#28048 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCAC |
| | | | AAGAAGAGAGACGGCATCTGGAGCACAGACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGGACTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 97 | CL_#28049 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCACT |
| | | | GGAAGGAGGACGGCATCTGGAGCACAGACATCCTGAAGG |
| | | | ATCAGAAGGAGCCCAAGAACAAGACCTTCCTGAGGTGCGA |
| | | | GGCCAAGAATTATAGCGGCAGATTCACCTGTTGGTGGCTG |
| | | | ACCACAATCTCCACCGATCTGACATTTTCTGTGAAGTCTAG |
| | | | CAGGGGCTCCTCTGACCCTCAGGGAGTGACATGCGGAGCC |
| | | | GCCACCCTGAGCGCCGAGCGGGTGAGAGGCGATAACAAG |
| | | | GAGTACGAGTATTCCGTGGAGTGCCAGGAGGACTCTGCCT |
| | | | GTCCCGCCGCCGAGGAGTCCCTGCCTATCGAAGTGATGGT |
| | | | GGATGCCGTGCACAAGCTGTGGTACGAGAATTATACAAGC |
| | | | TCCTTCTTTATCAGGGACATCATCAAGCCCGATCCCCCTAA |
| | | | GAACCTGCAGCTGAAGCCTCTGAAGAATAGCCGCCAGGTG |
| | | | GAGGTGTCCTGGGAGTACCCTGACACCTGGAGCACACCAC |
| | | | ACTCCTATTTCTCTCTGACCTTTTGCGTGCAGGTGCAGGGC |
| | | | AAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 98 | CL_#28050 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGAAGGGCGACGCCGGACAGTACACCTGTCACA |
| | | | AGGGCGGCGAGGTGCTGTCCCACTCTCTGCTGCTGCTGCA |
| | | | CAAGAAGGAGGACGGCATCTGGAGCACAGACATCCTGAA |
| | | | GGATCAGAAGGAGCCCAAGAACAAGACCTTCCTGAGGTGC |
| | | | GAGGCCAAGAATTATAGCGGCAGATTCACCTGTTGGTGGC |
| | | | TGACCACAATCTCCACCGATCTGACATTTTCTGTGAAGTCT |
| | | | AGCAGGGGCTCCTCTGACCCTCAGGGAGTGACATGCGGAG |
| | | | CCGCCACCCTGAGCGCCGAGCGGGTGAGAGGCGATAACA |
| | | | AGGAGTACGAGTATTCCGTGGAGTGCCAGGAGGACTCTGC |
| | | | CTGTCCCGCCGCCGAGGAGTCCCTGCCTATCGAAGTGATG |
| | | | GTGGATGCCGTGCACAAGCTGTGGTACGAGAATTATACAA |
| | | | GCTCCTTCTTTATCAGGGACATCATCAAGCCCGATCCCCCT |
| | | | AAGAACCTGCAGCTGAAGCCTCTGAAGAATAGCCGCCAGG |
| | | | TGGAGGTGTCCTGGGAGTACCCTGACACCTGGAGCACACC |
| | | | ACACTCCTATTTCTCTCTGACCTTTTGCGTGCAGGTGCAGG |
| | | | GCAAGTCTAAGAGGGAGAAGAAGGACCGCGTGTTCACCG |
| | | | ATAAGACAAGCGCCACCGTGATCTGTCGGAAGAACGCCAG |
| | | | CATCTCCGTGCGGGCCCAGGATAGATACTATTCTAGCTCCT |
| | | | GGAGCGAGTGGGCCTCCGTGCCATGTTCTGGAGGAGGAGG |
| | | | CAGCGGCGGAGGAGGCTCCGGCGGCGGCGGCTCTAATCTG |
| | | | CCAGTGGCCACCCCTGACCCAGGCATGTTCCCCTGCCTGCA |
| | | | CCACTCTCAGAACCTGCTGCGGGCCGTGAGCAATATGCTG |
| | | | CAGAAGGCCAGACAGACACTGGAGTTTTACCCATGTACCT |
| | | | CCGAGGAGATCGACCACGAGGATATCACAAAGGATAAGA |
| | | | CCTCTACAGTGGAGGCATGCCTGCCACTGGAGCTGACCAA |
| | | | GAACGAGAGCTGTCTGAACAGCCGGGAGACATCTTTCATC |
| | | | ACCAACGGCAGCTGCCTGGCCTCCAGAAAGACATCTTTTA |
| | | | TGATGGCCCTGTGCCTGTCTAGCATCTACGAGGACCTGAA |
| | | | GATGTATCAGGTGGAGTTCAAGACCATGAACGCCAAGCTG |
| | | | CTGATGGACCCTAAGAGGCAGATCTTTCTGGATCAGAATA |
| | | | TGCTGGCCGTGATCGACGAGCTGATGCAGGCCCTGAACTT |
| | | | CAATAGCGAGACAGTGCCACAGAAGTCCTCTCTGGAGGAG |
| | | | CCCGATTTCTACAAGACCAAGATCAAGCTGTGCATCCTGCT |
| | | | GCACGCCTTCCGGATCAGAGCCGTGACCATCGACCGCGTG |
| | | | ATGAGCTACCTGAACGCCAGC |
| 99 | CL_#28051 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCAC |
| | | | ATCAAGAGAGACGGCATCTGGAGCACAGACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGAAGTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 100 | CL_#28053 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCAC |
| | | | AAGAAGTGGGACGGCATCTGGAGCACAGAGATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGAAGTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 101 | CL_#28054 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGACTTCGGCGACGCCGGACAGTACACCTGTCACAA |
| | | | GGGCGGCGAGGTGCTGTCCCACTCTCTGCTGCTGCTGCAC |
| | | | AAGAAGGAGGACGGCATCTGGAGCACACACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGAAGTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 102 | CL_#28055 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCACT |
| | | | GGAAGGAGGACGGCATCTGGAGCACAGACATCCTGAAGG |
| | | | ATCAGAAGGAGCCCAAGAACAAGACCTTCCTGAGGTGCGA |
| | | | GGCCAAGAATTATAGCGGCAGATTCACCTGTTGGTGGCTG |
| | | | ACCACAATCTCCACCGATCTGACATTTTCTGTGAAGTCTAG |
| | | | CAGGGGCTCCTCTGACCCTCAGGGAGTGACATGCGGAGCC |
| | | | GCCACCCTGAGCGCCGAGCGGGTGAGAGGCAGAAACAAG |
| | | | GAGTACGAGTATTCCGTGGAGTGCCAGGAGGACTCTGCCT |
| | | | GTCCCGCCGCCGAGGAGTCCCTGCCTATCGAAGTGATGGT |
| | | | GGATGCCGTGCACAAGCTGAAGTACGAGAATTATACAAGC |
| | | | TCCTTCTTTATCAGGGACATCATCAAGCCCGATCCCCCTAA |
| | | | GAACCTGCAGCTGAAGCCTCTGAAGAATAGCCGCCAGGTG |
| | | | GAGGTGTCCTGGGAGTACCCTGACACCTGGAGCACACCAC |
| | | | ACTCCTATTTCTCTCTGACCTTTTGCGTGCAGGTGCAGGGC |
| | | | AAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 103 | CL_#28056 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCAC |
| | | | GAGAAGGAGGACGGCATCTGGAGCACAGACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGAAGTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 104 | CL_#28057 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGAGATTCGGCGACGCCGGACAGTACACCTGTCACAA |
| | | | GGGCGGCGAGGTGCTGTCCCACTCTCTGCTGCTGCTGCAC |
| | | | AAGAAGGAGGACGGCATCTGGAGCACAGACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGAAGTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 105 | CL_#28058 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCAC |
| | | | AAGAAGGAGGACGGCATCTGGAGCACAGACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGACCTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 106 | CL_#28059 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCAC |
| | | | AAGAAGGAGGACGGCATCTGGAGCACAGACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCAGAAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGAAGTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 107 | CL_#28060 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGAGCTTCGGCGACGCCGGACAGTACACCTGTCACAA |
| | | | GGGCGGCGAGGTGCTGTCCCACTCTCTGCTGCTGCTGCAC |
| | | | AAGAAGGAGGACGGCATCTGGAGCACAGACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGAAGTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 108 | CL_#28061 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCACT |
| | | | GGAAGGAGGACGGCATCTGGAGCACAGACATCCTGAAGG |
| | | | ATCAGAAGGAGCCCAAGAACAAGACCTTCCTGAGGTGCGA |
| | | | GGCCAAGAATTATAGCGGCAGATTCACCTGTTGGTGGCTG |
| | | | ACCACAATCTCCACCGATCTGACATTTTCTGTGAAGTCTAG |
| | | | CAGGGGCTCCTCTGACCCTCAGGGAGTGACATGCGGAGCC |
| | | | GCCACCCTGAGCGCCGAGCGGGTGAGAGGCGATAACAAG |
| | | | GAGTACGAGTATTCCGTGGAGTGCCAGGAGGACTCTGCCT |
| | | | GTCCCGCCGCCGAGGAGTCCCTGCCTATCGAAGTGATGGT |
| | | | GGATGCCGTGCACAAGCTGAAGTACGAGAATTATACAAGC |
| | | | TCCTTCTTTATCAGGGACATCATCAAGCCCGATCCCCCTAA |
| | | | GAACCTGCAGCTGAAGCCTCTGAAGAATAGCCGCCAGGTG |
| | | | GAGGTGTCCTGGGAGTACCCTGACACCTGGAGCACACCAC |
| | | | ACTCCTATTTCTCTCTGACCTTTTGCGTGCAGGTGCAGGGC |
| | | | AAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 109 | CL_#28062 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCAC |
| | | | AAGAAGTGGGACGGCATCTGGAGCACAGACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGAAGTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 110 | CL_#28063 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCAC |
| | | | AAGAAGGAGGACGGCATCTGGAGCACAGACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGTGGTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 111 | CL_#28064 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACAGATATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGACGACGGCGACGCCGGACAGTACACCTGTCACAA |
| | | | GGGCGGCGAGGTGCTGTCCCACTCTCTGCTGCTGCTGCAC |
| | | | AAGAAGGAGGACGGCATCTGGAGCACAGACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGTGGTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 112 | CL_#28065 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGAGAGGCGACGCCGGACAGTACACCTGTCACA |
| | | | AGGGCGGCGAGGTGCTGTCCCACTCTCTGCTGCTGCTGCA |
| | | | CGAGAAGGAGGACGGCATCTGGAGCACAGACATCCTGAA |
| | | | GGATCAGAAGGAGCCCAAGAACAAGACCTTCCTGAGGTGC |
| | | | GAGGCCAAGAATTATAGCGGCAGATTCACCTGTTGGTGGC |
| | | | TGACCACAATCTCCACCGATCTGACATTTTCTGTGAAGTCT |
| | | | AGCAGGGGCTCCTCTGACCCTCAGGGAGTGACATGCGGAG |
| | | | CCGCCACCCTGAGCGCCGAGCGGGTGAGAGGCGATAACA |
| | | | AGGAGTACGAGTATTCCGTGGAGTGCCAGGAGGACTCTGC |
| | | | CTGTCCCGCCGCCGAGGAGTCCCTGCCTATCGAAGTGATG |
| | | | GTGGATGCCGTGCACAAGCTGTGGTACGAGAATTATACAA |
| | | | GCTCCTTCTTTATCAGGGACATCATCAAGCCCGATCCCCCT |
| | | | AAGAACCTGCAGCTGAAGCCTCTGAAGAATAGCCGCCAGG |
| | | | TGGAGGTGTCCTGGGAGTACCCTGACACCTGGAGCACACC |
| | | | ACACTCCTATTTCTCTCTGACCTTTTGCGTGCAGGTGCAGG |
| | | | GCAAGTCTAAGAGGGAGAAGAAGGACCGCGTGTTCACCG |
| | | | ATAAGACAAGCGCCACCGTGATCTGTCGGAAGAACGCCAG |
| | | | CATCTCCGTGCGGGCCCAGGATAGATACTATTCTAGCTCCT |
| | | | GGAGCGAGTGGGCCTCCGTGCCATGTTCTGGAGGAGGAGG |
| | | | CAGCGGCGGAGGAGGCTCCGGCGGCGGCGGCTCTAATCTG |
| | | | CCAGTGGCCACCCCTGACCCAGGCATGTTCCCCTGCCTGCA |
| | | | CCACTCTCAGAACCTGCTGCGGGCCGTGAGCAATATGCTG |
| | | | CAGAAGGCCAGACAGACACTGGAGTTTTACCCATGTACCT |
| | | | CCGAGGAGATCGACCACGAGGATATCACAAAGGATAAGA |
| | | | CCTCTACAGTGGAGGCATGCCTGCCACTGGAGCTGACCAA |
| | | | GAACGAGAGCTGTCTGAACAGCCGGGAGACATCTTTCATC |
| | | | ACCAACGGCAGCTGCCTGGCCTCCAGAAAGACATCTTTTA |
| | | | TGATGGCCCTGTGCCTGTCTAGCATCTACGAGGACCTGAA |
| | | | GATGTATCAGGTGGAGTTCAAGACCATGAACGCCAAGCTG |
| | | | CTGATGGACCCTAAGAGGCAGATCTTTCTGGATCAGAATA |
| | | | TGCTGGCCGTGATCGACGAGCTGATGCAGGCCCTGAACTT |
| | | | CAATAGCGAGACAGTGCCACAGAAGTCCTCTCTGGAGGAG |
| | | | CCCGATTTCTACAAGACCAAGATCAAGCTGTGCATCCTGCT |
| | | | GCACGCCTTCCGGATCAGAGCCGTGACCATCGACCGCGTG |
| | | | ATGAGCTACCTGAACGCCAGC |
| 113 | CL_#28066 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACCACTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCACC |
| | | | TGAAGGAGGACGGCATCTGGAGCACAGACATCCTGAAGG |
| | | | ATCAGAAGGAGCCCAAGAACAAGACCTTCCTGAGGTGCGA |
| | | | GGCCAAGAATTATAGCGGCAGATTCACCTGTTGGTGGCTG |
| | | | ACCACAATCTCCACCGATCTGACATTTTCTGTGAAGTCTAG |
| | | | CAGGGGCTCCTCTGACCCTCAGGGAGTGACATGCGGAGCC |
| | | | GCCACCCTGAGCGCCGAGCGGGTGAGAGGCGATAACAAG |
| | | | GAGTACGAGTATTCCGTGGAGTGCCAGGAGGACTCTGCCT |
| | | | GTCCCGCCGCCGAGGAGTCCCTGCCTATCGAAGTGATGGT |
| | | | GGATGCCGTGCACAAGCTGCAGTACGAGAATTATACAAGC |
| | | | TCCTTCTTTATCAGGGACATCATCAAGCCCGATCCCCCTAA |
| | | | GAACCTGCAGCTGAAGCCTCTGAAGAATAGCCGCCAGGTG |
| | | | GAGGTGTCCTGGGAGTACCCTGACACCTGGAGCACACCAC |
| | | | ACTCCTATTTCTCTCTGACCTTTTGCGTGCAGGTGCAGGGC |
| | | | AAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 114 | CL_#28067 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGACTTCGGCGACGCCGGACAGTACACCTGTCACAA |
| | | | GGGCGGCGAGGTGCTGTCCCACTCTCTGCTGCTGCTGCACT |
| | | | GGAAGGAGGACGGCATCTGGAGCACAGACATCCTGAAGG |
| | | | ATCAGAAGGAGCCCAAGAACAAGACCTTCCTGAGGTGCGA |
| | | | GGCCAAGAATTATAGCGGCAGATTCACCTGTTGGTGGCTG |
| | | | ACCACAATCTCCACCGATCTGACATTTTCTGTGAAGTCTAG |
| | | | CAGGGGCTCCTCTGACCCTCAGGGAGTGACATGCGGAGCC |
| | | | GCCACCCTGAGCGCCGAGCGGGTGAGAGGCGATAACAAG |
| | | | GAGTACGAGTATTCCGTGGAGTGCCAGGAGGACTCTGCCT |
| | | | GTCCCGCCGCCGAGGAGTCCCTGCCTATCGAAGTGATGGT |
| | | | GGATGCCGTGCACAAGCTGTGGTACGAGAATTATACAAGC |
| | | | TCCTTCTTTATCAGGGACATCATCAAGCCCGATCCCCCTAA |
| | | | GAACCTGCAGCTGAAGCCTCTGAAGAATAGCCGCCAGGTG |
| | | | GAGGTGTCCTGGGAGTACCCTGACACCTGGAGCACACCAC |
| | | | ACTCCTATTTCTCTCTGACCTTTTGCGTGCAGGTGCAGGGC |
| | | | AAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 115 | CL_#28068 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGCACGAGTTCGGCGACGCCGGACAGTACACCTGTCACAA |
| | | | GGGCGGCGAGGTGCTGTCCCACTCTCTGCTGCTGCTGCAC |
| | | | AAGAAGAGAGACGGCATCTGGAGCACAGACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGGACTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 116 | CL_#28069 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCAC |
| | | | ATCAAGAGAGACGGCATCTGGAGCACACACATCCTGAAGG |
| | | | ATCAGAAGGAGCCCAAGAACAAGACCTTCCTGAGGTGCGA |
| | | | GGCCAAGAATTATAGCGGCAGATTCACCTGTTGGTGGCTG |
| | | | ACCACAATCTCCACCGATCTGACATTTTCTGTGAAGTCTAG |
| | | | CAGGGGCTCCTCTGACCCTCAGGGAGTGACATGCGGAGCC |
| | | | GCCACCCTGAGCGCCGAGCGGGTGAGAGGCGATAACAAG |
| | | | GAGTACGAGTATTCCGTGGAGTGCCAGGAGGACTCTGCCT |
| | | | GTCCCGCCGCCGAGGAGTCCCTGCCTATCGAAGTGATGGT |
| | | | GGATGCCGTGCACAAGCTGAAGTACGAGAATTATACAAGC |
| | | | TCCTTCTTTATCAGGGACATCATCAAGCCCGATCCCCCTAA |
| | | | GAACCTGCAGCTGAAGCCTCTGAAGAATAGCCGCCAGGTG |
| | | | GAGGTGTCCTGGGAGTACCCTGACACCTGGAGCACACCAC |
| | | | ACTCCTATTTCTCTCTGACCTTTTGCGTGCAGGTGCAGGGC |
| | | | AAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 117 | CL_#28070 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACCACTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCACC |
| | | | TGAAGGAGGACGGCATCTGGAGCACAGACATCCTGAAGG |
| | | | ATCAGAAGGAGCCCAAGAACAAGACCTTCCTGAGGTGCGA |
| | | | GGCCAAGAATTATAGCGGCAGATTCACCTGTTGGTGGCTG |
| | | | ACCACAATCTCCACCGATCTGACATTTTCTGTGAAGTCTAG |
| | | | CAGGGGCTCCTCTGACCCTCAGGGAGTGACATGCGGAGCC |
| | | | GCCACCCTGAGCGCCGAGCGGGTGAGAGGCGATAACAAG |
| | | | GAGTACGAGTATTCCGTGGAGTGCCAGGAGGACTCTGCCT |
| | | | GTCCCGCCGCCGAGGAGTCCCTGCCTATCGAAGTGATGGT |
| | | | GGATGCCGTGCACAAGCTGAAGTACGAGAATTATACAAGC |
| | | | TCCTTCTTTATCAGGGACATCATCAAGCCCGATCCCCCTAA |
| | | | GAACCTGCAGCTGAAGCCTCTGAAGAATAGCCGCCAGGTG |
| | | | GAGGTGTCCTGGGAGTACCCTGACACCTGGAGCACACCAC |
| | | | ACTCCTATTTCTCTCTGACCTTTTGCGTGCAGGTGCAGGGC |
| | | | AAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 118 | CL_#28071 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACAAGTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCAC |
| | | | AAGAAGGAGGACGGCATCTGGAGCACAGACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGAAGTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 119 | CL_#28072 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACCACTATC |
| | | | CAGATGCACCAGGAGAGATGGTGGTGCTGACCTGCGACAC |
| | | | ACCAGAGGAGGATGGCATCACCTGGACACTGGATCAGAGC |
| | | | TCCGAGGTGCTGGGCTCCGGCAAGACCCTGACAATCCAGG |
| | | | TGAAGGAGTTCGGCGACGCCGGACAGTACACCTGTCACAA |
| | | | GGGCGGCGAGGTGCTGTCCCACTCTCTGCTGCTGCTGCAC |
| | | | AAGAAGGAGGACGGCATCTGGAGCACAGACATCCTGAAG |
| | | | GATCAGAAGGAGCCCAAGAACAAGACCTTCCTGAGGTGCG |
| | | | AGGCCAAGAATTATAGCGGCAGATTCACCTGTTGGTGGCT |
| | | | GACCACAATCTCCACCGATCTGACATTTTCTGTGAAGTCTA |
| | | | GCAGGGGCTCCTCTGACCCTCAGGGAGTGACATGCGGAGC |
| | | | CGCCACCCTGAGCGCCGAGCGGGTGAGAGGCGATAACAA |
| | | | GGAGTACGAGTATTCCGTGGAGTGCCAGGAGGACTCTGCC |
| | | | TGTCCCGCCGCCGAGGAGTCCCTGCCTATCGAAGTGATGG |
| | | | TGGATGCCGTGCACAAGCTGAAGTACGAGAATTATACAAG |
| | | | CTCCTTCTTTATCAGGGACATCATCAAGCCCGATCCCCCTA |
| | | | AGAACCTGCAGCTGAAGCCTCTGAAGAATAGCCGCCAGGT |
| | | | GGAGGTGTCCTGGGAGTACCCTGACACCTGGAGCACACCA |
| | | | CACTCCTATTTCTCTCTGACCTTTTGCGTGCAGGTGCAGGG |
| | | | CAAGTCTAAGAGGGAGAAGAAGGACCGCGTGTTCACCGAT |
| | | | AAGACAAGCGCCACCGTGATCTGTCGGAAGAACGCCAGCA |
| | | | TCTCCGTGCGGGCCCAGGATAGATACTATTCTAGCTCCTGG |
| | | | AGCGAGTGGGCCTCCGTGCCATGTTCTGGAGGAGGAGGCA |
| | | | GCGGCGGAGGAGGCTCCGGCGGCGGCGGCTCTAATCTGCC |
| | | | AGTGGCCACCCCTGACCCAGGCATGTTCCCCTGCCTGCACC |
| | | | ACTCTCAGAACCTGCTGCGGGCCGTGAGCAATATGCTGCA |
| | | | GAAGGCCAGACAGACACTGGAGTTTTACCCATGTACCTCC |
| | | | GAGGAGATCGACCACGAGGATATCACAAAGGATAAGACC |
| | | | TCTACAGTGGAGGCATGCCTGCCACTGGAGCTGACCAAGA |
| | | | ACGAGAGCTGTCTGAACAGCCGGGAGACATCTTTCATCAC |
| | | | CAACGGCAGCTGCCTGGCCTCCAGAAAGACATCTTTTATG |
| | | | ATGGCCCTGTGCCTGTCTAGCATCTACGAGGACCTGAAGA |
| | | | TGTATCAGGTGGAGTTCAAGACCATGAACGCCAAGCTGCT |
| | | | GATGGACCCTAAGAGGCAGATCTTTCTGGATCAGAATATG |
| | | | CTGGCCGTGATCGACGAGCTGATGCAGGCCCTGAACTTCA |
| | | | ATAGCGAGACAGTGCCACAGAAGTCCTCTCTGGAGGAGCC |
| | | | CGATTTCTACAAGACCAAGATCAAGCTGTGCATCCTGCTG |
| | | | CACGCCTTCCGGATCAGAGCCGTGACCATCGACCGCGTGA |
| | | | TGAGCTACCTGAACGCCAGC |
| 120 | CL_#28074 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGACCAGAG |
| | | | CTCCAGAGTGCTGGGCTCCGGCAAGACCCTGACAATCCAG |
| | | | GTGAGCAGCTTCGGCGACGCCGGACAGTACACCTGTCACA |
| | | | AGGGCGGCGAGGTGCTGTCCCACTCTCTGCTGCTGCTGCA |
| | | | CAAGAAGGAGGACGGCATCTGGAGCACAGACATCCTGAA |
| | | | GGATCAGAAGGAGCCCAAGAACAAGACCTTCCTGAGGTGC |
| | | | GAGGCCAAGAATTATAGCGGCAGATTCACCTGTTGGTGGC |
| | | | TGACCACAATCTCCACCGATCTGACATTTTCTGTGAAGTCT |
| | | | AGCAGGGGCTCCTCTGACCCTCAGGGAGTGACATGCGGAG |
| | | | CCGCCACCCTGAGCGCCGAGCGGGTGAGAGGCGATAACA |
| | | | AGGAGTACGAGTATTCCGTGGAGTGCCAGGAGGACTCTGC |
| | | | CTGTCCCGCCGCCGAGGAGTCCCTGCCTATCGAAGTGATG |
| | | | GTGGATGCCGTGCACGACCTGAAGTACGAGAATTATACAA |
| | | | GCTCCTTCTTTATCAGGGACATCATCAAGCCCGATCCCCCT |
| | | | AAGAACCTGCAGCTGAAGCCTCTGAAGAATAGCCGCCAGG |
| | | | TGGAGGTGTCCTGGGAGTACCCTGACACCTGGAGCACACC |
| | | | ACACTCCTATTTCTCTCTGACCTTTTGCGTGCAGGTGCAGG |
| | | | GCAAGTCTAAGAGGGAGAAGAAGGACCGCGTGTTCACCG |
| | | | ATAAGACAAGCGCCACCGTGATCTGTCGGAAGAACGCCAG |
| | | | CATCTCCGTGCGGGCCCAGGATAGATACTATTCTAGCTCCT |
| | | | GGAGCGAGTGGGCCTCCGTGCCATGTTCTGGAGGAGGAGG |
| | | | CAGCGGCGGAGGAGGCTCCGGCGGCGGCGGCTCTAATCTG |
| | | | CCAGTGGCCACCCCTGACCCAGGCATGTTCCCCTGCCTGCA |
| | | | CCACTCTCAGAACCTGCTGCGGGCCGTGAGCAATATGCTG |
| | | | CAGAAGGCCAGACAGACACTGGAGTTTTACCCATGTACCT |
| | | | CCGAGGAGATCGACCACGAGGATATCACAAAGGATAAGA |
| | | | CCTCTACAGTGGAGGCATGCCTGCCACTGGAGCTGACCAA |
| | | | GAACGAGAGCTGTCTGAACAGCCGGGAGACATCTTTCATC |
| | | | ACCAACGGCAGCTGCCTGGCCTCCAGAAAGACATCTTTTA |
| | | | TGATGGCCCTGTGCCTGTCTAGCATCTACGAGGACCTGAA |
| | | | GATGTATCAGGTGGAGTTCAAGACCATGAACGCCAAGCTG |
| | | | CTGATGGACCCTAAGAGGCAGATCTTTCTGGATCAGAATA |
| | | | TGCTGGCCGTGATCGACGAGCTGATGCAGGCCCTGAACTT |
| | | | CAATAGCGAGACAGTGCCACAGAAGTCCTCTCTGGAGGAG |
| | | | CCCGATTTCTACAAGACCAAGATCAAGCTGTGCATCCTGCT |
| | | | GCACGCCTTCCGGATCAGAGCCGTGACCATCGACCGCGTG |
| | | | ATGAGCTACCTGAACGCCAGC |
| 121 | CL_#28075 | Full nt | GAGCCAAAGAGCTCCGACAAGACCCACACATGCCCCCCTT |
| | | | GTCCGGCGCCAGAGGCTGCAGGAGGACCAAGCGTGTTCCT |
| | | | GTTTCCACCCAAGCCTAAAGACACACTGATGATTTCCCGA |
| | | | ACCCCCGAAGTCACATGCGTGGTCGTGTCTGTGAGTCACG |
| | | | AGGACCCTGAAGTCAAGTTCAACTGGTACGTGGATGGCGT |
| | | | CGAGGTGCATAATGCCAAGACTAAACCTAGGGAGGAACA |
| | | | GTACAACTCAACCTATCGCGTCGTGAGCGTCCTGACAGTG |
| | | | CTGCACCAGGATTGGCTGAACGGCAAAGAATATAAGTGCA |
| | | | AAGTGAGCAATAAGGCCCTGCCCGCTCCTATCGAGAAAAC |
| | | | CATTTCCAAGGCTAAAGGGCAGCCTCGCGAACCTCAGGTG |
| | | | TACGTGTATCCACCCTCCCGCGATGAGCTGACAAAGAACC |
| | | | AGGTGTCTCTGACCTGTCTGGTGAAGGGCTTTTACCCTAGC |
| | | | GACATCGCCGTGGAGTGGGAGTCCAATGGCCAGCCAGAGA |
| | | | ACAATTATAAGACCACACCTCCAGTGCTGGACTCTGATGG |
| | | | CAGCTTCGCCCTGGTGTCTAAGCTGACAGTGGACAAGAGC |
| | | | AGATGGCAGCAGGGCAACGTGTTTTCTTGTAGCGTGATGC |
| | | | ACGAGGCCCTGCACAATCACTACACCCAGAAGTCCCTGAG |
| | | | CTTAAGCCCTGGAGGCAGCGCCGATGGAGGAATCTGGGAG |
| | | | CTGAAGAAGGACGTGTACGTGGTGGAGCTGGACTGGTATC |
| | | | CAGATGCACCAGGAGAGATGGTGGTGCTGACCTGCGACAC |
| | | | ACCAGAGGAGGATGGCATCACCTGGACACTGGACCAGAG |
| | | | CTCCGAGGTGCTGGGCTCCGGCAAGACCCTGACAATCCAG |
| | | | GTGAGCAGCTTCGGCGACGCCGGACAGTACACCTGTCACA |
| | | | AGGGCGGCGAGGTGCTGTCCCACTCTCTGCTGCTGCTGCA |
| | | | CAAGAAGGAGGACGGCATCTGGAGCACAGACATCCTGAA |
| | | | GGATCAGAAGGAGCCCAAGAACAAGACCTTCCTGAGGTGC |
| | | | GAGGCCAAGAATTATAGCGGCAGATTCACCTGTTGGTGGC |
| | | | TGACCACAATCTCCACCGATCTGACATTTTCTGTGAAGTCT |
| | | | AGCAGGGGCTCCTCTGACCCTCAGGGAGTGACATGCGGAG |
| | | | CCGCCACCCTGAGCGCCGAGCGGGTGAGAGGCGATAACA |
| | | | AGGAGTACGAGTATTCCGTGGAGTGCCAGGAGGACTCTGC |
| | | | CTGTCCCGCCGCCGAGGAGTCCCTGCCTATCGAAGTGATG |
| | | | GTGGATGCCGTGCACGACCTGAAGTACGAGAATTATACAA |
| | | | GCTCCTTCTTTATCAGGGACATCATCAAGCCCGATCCCCCT |
| | | | AAGAACCTGCAGCTGAAGCCTCTGAAGAATAGCCGCCAGG |
| | | | TGGAGGTGTCCTGGGAGTACCCTGACACCTGGAGCACACC |
| | | | ACACTCCTATTTCTCTCTGACCTTTTGCGTGCAGGTGCAGG |
| | | | GCAAGTCTAAGAGGGAGAAGAAGGACCGCGTGTTCACCG |
| | | | ATAAGACAAGCGCCACCGTGATCTGTCGGAAGAACGCCAG |
| | | | CATCTCCGTGCGGGCCCAGGATAGATACTATTCTAGCTCCT |
| | | | GGAGCGAGTGGGCCTCCGTGCCATGTTCTGGAGGAGGAGG |
| | | | CAGCGGCGGAGGAGGCTCCGGCGGCGGCGGCTCTAATCTG |
| | | | CCAGTGGCCACCCCTGACCCAGGCATGTTCCCCTGCCTGCA |
| | | | CCACTCTCAGAACCTGCTGCGGGCCGTGAGCAATATGCTG |
| | | | CAGAAGGCCAGACAGACACTGGAGTTTTACCCATGTACCT |
| | | | CCGAGGAGATCGACCACGAGGATATCACAAAGGATAAGA |
| | | | CCTCTACAGTGGAGGCATGCCTGCCACTGGAGCTGACCAA |
| | | | GAACGAGAGCTGTCTGAACAGCCGGGAGACATCTTTCATC |
| | | | ACCAACGGCAGCTGCCTGGCCTCCAGAAAGACATCTTTTA |
| | | | TGATGGCCCTGTGCCTGTCTAGCATCTACGAGGACCTGAA |
| | | | GATGTATCAGGTGGAGTTCAAGACCATGAACGCCAAGCTG |
| | | | CTGATGGACCCTAAGAGGCAGATCTTTCTGGATCAGAATA |
| | | | TGCTGGCCGTGATCGACGAGCTGATGCAGGCCCTGAACTT |
| | | | CAATAGCGAGACAGTGCCACAGAAGTCCTCTCTGGAGGAG |
| | | | CCCGATTTCTACAAGACCAAGATCAAGCTGTGCATCCTGCT |
| | | | GCACGCCTTCCGGATCAGAGCCGTGACCATCGACCGCGTG |
| | | | ATGAGCTACCTGAACGCCAGC |
| 122 | | p40 precursor | MCHQQLVISWFSLVFLASPLVA |
| | | SP | |
| 123 | | p40 precursor | MCHQQLVISWFSLVFLASPLVAIWELKKDVYVVELDWYPDA |
| | | | PGEMVVLTCDTPEEDGITWTLDQSSEVLGSGKTLTIQVKEFG |
| | | | DAGQYTCHKGGEVLSHSLLLLHKKEDGIWSTDILKDQKEPK |
| | | | NKTFLRCEAKNYSGRFTCWWLTTISTDLTFSVKSSRGSSDPQ |
| | | | GVTCGAATLSAERVRGDNKEYEYSVECQEDSACPAAEESLPI |
| | | | EVMVDAVHKLKYENYTSSFFIRDIIKPDPPKNLQLKPLKNSRQ |
| | | | VEVSWEYPDTWSTPHSYFSLTFCVQVQGKSKREKKDRVFTD |
| | | | KTSATVICRKNASISVRAQDRYYSSSWSEWASVPCS |
| 124 | | p35 precursor | MCPARSLLLVATLVLLDHLSLA |
| | | SP | |
| 125 | | p35 precursor | MCPARSLLLVATLVLLDHLSLARNLPVATPDPGMFPCLHHSQ |
| | | | NLLRAVSNMLQKARQTLEFYPCTSEEIDHEDITKDKTSTVEA |
| | | | CLPLELTKNESCLNSRETSFITNGSCLASRKTSFMMALCLSSIY |
| | | | EDLKMYQVEFKTMNAKLLMDPKRQIFLDQNMLAVIDELMQ |
| | | | ALNFNSETVPQKSSLEEPDFYKTKIKLCILLHAFRIRAVTIDRV |
| | | | MSYLNAS |
| 126 | | p19 precursor | MLGSRAVMLLLLLPWTAQG |
| | | SP | |
| 127 | | p19 precursor | MLGSRAVMLLLLLPWTAQGRAVPGGSSPAWTQCQQLSQKL |
| | | | CTLAWSAHPLVGHMDLREEGDEETTNDVPHIQCGDGCDPQG |
| | | | LRDNSQFCLQRIHQGLIFYEKLLGSDIFTGEPSLLPDSPVGQLH |
| | | | ASLLGLSQLLQPEGHHWETQQIPSLSPSQPWQRLLLRFKILRS |
| | | | LQAFVAVAARVFAHGAATLSP |
| 128 | | p19 mature | RAVPGGSSPAWTQCQQLSQKLCTLAWSAHPLVGHMDLREE |
| | | | GDEETTNDVPHIQCGDGCDPQGLRDNSQFCLQRIHQGLIFYE |
| | | | KLLGSDIFTGEPSLLPDSPVGQLHASLLGLSQLLQPEGHHWET |
| | | | QQIPSLSPSQPWQRLLLRFKILRSLQAFVAVAARVFAHGAATL |
| | | | SP |
| 129 | 17872 | p35 nucleotide | AGAAACCTGCCCGTGGCCACACCCGATCCTGGCATGTTTC |
| | | | CCTGCCTGCACCACAGCCAGAACCTGCTGCGGGCCGTGTC |
| | | | CAATATGCTGCAGAAGGCCAGACAGACCCTGGAGTTCTAC |
| | | | CCCTGTACATCTGAGGAGATCGACCACGAGGATATCACCA |
| | | | AGGACAAGACCTCCACAGTGGAGGCCTGCCTGCCTCTGGA |
| | | | GCTGACAAAGAACGAGTCCTGTCTGAACAGCCGGGAGACC |
| | | | AGCTTCATCACAAATGGCTCCTGCCTGGCCTCTCGCAAGAC |
| | | | CAGCTTTATGATGGCCCTGTGCCTGAGCTCCATCTACGAGG |
| | | | ATCTGAAGATGTATCAGGTGGAGTTCAAGACAATGAACGC |
| | | | CAAGCTGCTGATGGACCCTAAGCGGCAGATCTTTCTGGAT |
| | | | CAGAATATGCTGGCCGTGATCGACGAGCTGATGCAGGCCC |
| | | | TGAACTTTAATAGCGAGACCGTGCCTCAGAAGTCTAGCCT |
| | | | GGAGGAGCCAGATTTCTACAAGACAAAGATCAAGCTGTGC |
| | | | ATCCTGCTGCACGCCTTCCGGATCAGAGCCGTGACCATCG |
| | | | ACAGAGTGATGTCCTATCTGAACGCCTCT |
| 130 | 17871 | p40 nucleotide | ATCTGGGAGCTGAAGAAGGACGTGTACGTGGTGGAGCTGG |
| | | | ACTGGTATCCAGATGCACCAGGAGAGATGGTGGTGCTGAC |
| | | | CTGCGACACACCAGAGGAGGATGGCATCACCTGGACACTG |
| | | | GACCAGAGCTCCGAGGTGCTGGGCAGCGGCAAGACCCTGA |
| | | | CAATCCAGGTGAAGGAGTTCGGCGATGCCGGACAGTACAC |
| | | | ATGTCACAAGGGCGGCGAGGTGCTGTCTCACAGCCTGCTG |
| | | | CTGCTGCACAAGAAGGAGGATGGCATCTGGTCCACAGACA |
| | | | TCCTGAAGGATCAGAAGGAGCCCAAGAACAAGACCTTCCT |
| | | | GAGGTGCGAGGCCAAGAATTATTCTGGCCGCTTTACCTGTT |
| | | | GGTGGCTGACCACAATCTCTACCGACCTGACCTTCAGCGT |
| | | | GAAGTCTAGCCGGGGCTCCTCTGATCCTCAGGGAGTGACA |
| | | | TGCGGAGCCGCCACCCTGAGCGCCGAGCGGGTGAGAGGC |
| | | | GACAACAAGGAGTACGAGTATAGCGTGGAGTGCCAGGAG |
| | | | GATTCCGCCTGTCCCGCCGCCGAGGAGTCCCTGCCTATCGA |
| | | | AGTGATGGTGGACGCCGTGCACAAGCTGAAGTACGAGAAT |
| | | | TATACAAGCTCCTTCTTTATCAGGGACATCATCAAGCCAGA |
| | | | TCCCCCTAAGAACCTGCAGCTGAAGCCCCTGAAGAACAGC |
| | | | AGACAGGTGGAGGTGTCTTGGGAGTACCCTGATACCTGGT |
| | | | CCACACCACACAGCTATTTCTCCCTGACCTTTTGCGTGCAG |
| | | | GTGCAGGGCAAGTCTAAGCGGGAGAAGAAGGACAGAGTG |
| | | | TTTACCGATAAGACAAGCGCCACCGTGATCTGTAGAAAGA |
| | | | ACGCCTCCATCTCTGTGAGGGCCCAGGACCGCTACTATTCT |
| | | | AGCTCCTGGTCTGAGTGGGCCAGCGTGCCTTGTTCC |
| 131 | 17908 | p19 nucleotide | AGAGCCGTGCCTGGCGGCAGCTCCCCAGCCTGGACCCAGT |
| | | | GCCAGCAGCTGAGCCAGAAGCTGTGCACACTGGCCTGGTC |
| | | | CGCCCACCCACTGGTGGGACACATGGACCTGCGGGAGGAG |
| | | | GGCGATGAGGAGACCACAAACGACGTGCCACACATCCAGT |
| | | | GCGGCGACGGATGTGATCCTCAGGGCCTGCGGGATAATTC |
| | | | TCAGTTCTGCCTGCAGAGAATCCACCAGGGCCTGATCTTCT |
| | | | ACGAGAAGCTGCTGGGCAGCGATATCTTTACCGGAGAGCC |
| | | | TTCTCTGCTGCCAGACAGCCCTGTGGGACAGCTGCACGCCT |
| | | | CCCTGCTGGGCCTGTCTCAGCTGCTGCAGCCAGAGGGCCA |
| | | | CCACTGGGAGACACAGCAGATCCCCTCTCTGAGCCCATCC |
| | | | CAGCCATGGCAGAGGCTGCTGCTGCGCTTTAAGATCCTGA |
| | | | GGTCCCTGCAGGCCTTCGTGGCAGTGGCAGCACGCGTGTT |
| | | | TGCCCACGGAGCCGCCACACTGTCTCCC |
| 132 | | Linker | GSADGG |
| 133 | | Linker | GGGGSGGGGSGGGGS |
| 134 | | Linker | GGGGSGGGGSGGGSGGGSGGGGSGGGGS |
| 135 | | Linker | GGSGGGSGGGSGGGSGGGSG |
| 136 | | Human IgG1 | APELLGGPSVFLFPPKPKDTLMISRTPEVTCVVVDVSHEDPEV |
| | | Fc 231-447 | KFNWYVDGVEVHNAKTKPREEQYNSTYRVVSVLTVLHQDW |
| | | (EU- | LNGKEYKCKVSNKALPAPIEKTISKAKGQPREPQVYTLPPSRD |
| | | numbering) | ELTKNQVSLTCLVKGFYPSDIAVEWESNGQPENNYKTTPPVL |
| | | | DSDGSFFLYSKLTVDKSRWQQGNVFSCSVMHEALHNHYTQK |
| | | | SLSLSPGK |
| 183 | | Human IgG1 | APELLGGPSVFLFPPKPKDTLMISRTPEVTCVVVDVSHEDPEV |
| | | CH2 domain | KFNWYVDGVEVHNAKTKPREEQYNSTYRVVSVLTVLHQDW |
| | | | LNGKEYKCKVSNKALPAPIEKTISKAK |
| 184 | | Human IgG1 | GQPREPQVYTLPPSRDELTKNQVSLTCLVKGFYPSDIAVEWE |
| | | CH3 domain | SNGQPENNYKTTPPVLDSDGSFFLYSKLTVDKSRWQQGNVFS |
| | | | CSVMHEALHNHYTQKSLSLSPGK |
| 185 | | Modified IgG1 | APEAAGGPSVFLFPPKPKDTLMISRTPEVTCVVVSVSHEDPEV |
| | | CH2 domain | KFNWYVDGVEVHNAKTKPREEQYNSTYRVVSVLTVLHQDW |
| | | | LNGKEYKCKVSNKALPAPIEKTISKAK |
| 186 | | Modified IgG1 | GQPREPQVYVYPPSRDELTKNQVSLTCLVKGFYPSDIAVEWE |
| | | CH3 domain | SNGQPENNYKTTPPVLDSDGSFALVSKLTVDKSRWQQGNVF |
| | | | SCSVMHEALHNHYTQKSLSLSPG |
| 187 | | Modified IgG1 | GQPREPQVYVLPPSRDELTKNQVSLLCLVKGFYPSDIAVEWE |
| | | CH3 domain | SNGQPENNYLTWPPVLDSDGSFFLYSKLTVDKSRWQQGNVF |
| | | | SCSVMHEALHNHYTQKSLSLSPG |
| 137 | | Mutated p40 | ATCTGGGAGCTGAAGAAGGACGTGTACGTGGTGGAGCTGG |
| | | nucleotide | ACCACTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 138 | | Mutated p40 | ATCTGGGAGCTGAAGAAGGACGTGTACGTGGTGGAGCTGG |
| | | nucleotide | ACAAGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 139 | | Mutated p40 | ATCTGGGAGCTGAAGAAGGACGTGTACGTGGTGGAGCTGG |
| | | nucleotide | ACAGATATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 140 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCAGAGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 141 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGCACGAGTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 142 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAGCGAGTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 143 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGACTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 144 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGAGATTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 145 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGAGCTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 146 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGGACGGCGACGCCGGACAGTACA |
| | | | CCTGTCACAAGGGCGGCGAGGTGCTGTCCCACTCTCTGCT |
| | | | GCTGCTGCACAAGAAGGAGGACGGCATCTGGAGCACAGA |
| | | | CATCCTGAAGGATCAGAAGGAGCCCAAGAACAAGACCTTC |
| | | | CTGAGGTGCGAGGCCAAGAATTATAGCGGCAGATTCACCT |
| | | | GTTGGTGGCTGACCACAATCTCCACCGATCTGACATTTTCT |
| | | | GTGAAGTCTAGCAGGGGCTCCTCTGACCCTCAGGGAGTGA |
| | | | CATGCGGAGCCGCCACCCTGAGCGCCGAGCGGGTGAGAG |
| | | | GCGATAACAAGGAGTACGAGTATTCCGTGGAGTGCCAGGA |
| | | | GGACTCTGCCTGTCCCGCCGCCGAGGAGTCCCTGCCTATCG |
| | | | AAGTGATGGTGGATGCCGTGCACAAGCTGAAGTACGAGAA |
| | | | TTATACAAGCTCCTTCTTTATCAGGGACATCATCAAGCCCG |
| | | | ATCCCCCTAAGAACCTGCAGCTGAAGCCTCTGAAGAATAG |
| | | | CCGCCAGGTGGAGGTGTCCTGGGAGTACCCTGACACCTGG |
| | | | AGCACACCACACTCCTATTTCTCTCTGACCTTTTGCGTGCA |
| | | | GGTGCAGGGCAAGTCTAAGAGGGAGAAGAAGGACCGCGT |
| | | | GTTCACCGATAAGACAAGCGCCACCGTGATCTGTCGGAAG |
| | | | AACGCCAGCATCTCCGTGCGGGCCCAGGATAGATACTATT |
| | | | CTAGCTCCTGGAGCGAGTGGGCCTCCGTGCCATGTTCT |
| 147 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGAAGGGCGACGCCGGACAGTACA |
| | | | CCTGTCACAAGGGCGGCGAGGTGCTGTCCCACTCTCTGCT |
| | | | GCTGCTGCACAAGAAGGAGGACGGCATCTGGAGCACAGA |
| | | | CATCCTGAAGGATCAGAAGGAGCCCAAGAACAAGACCTTC |
| | | | CTGAGGTGCGAGGCCAAGAATTATAGCGGCAGATTCACCT |
| | | | GTTGGTGGCTGACCACAATCTCCACCGATCTGACATTTTCT |
| | | | GTGAAGTCTAGCAGGGGCTCCTCTGACCCTCAGGGAGTGA |
| | | | CATGCGGAGCCGCCACCCTGAGCGCCGAGCGGGTGAGAG |
| | | | GCGATAACAAGGAGTACGAGTATTCCGTGGAGTGCCAGGA |
| | | | GGACTCTGCCTGTCCCGCCGCCGAGGAGTCCCTGCCTATCG |
| | | | AAGTGATGGTGGATGCCGTGCACAAGCTGAAGTACGAGAA |
| | | | TTATACAAGCTCCTTCTTTATCAGGGACATCATCAAGCCCG |
| | | | ATCCCCCTAAGAACCTGCAGCTGAAGCCTCTGAAGAATAG |
| | | | CCGCCAGGTGGAGGTGTCCTGGGAGTACCCTGACACCTGG |
| | | | AGCACACCACACTCCTATTTCTCTCTGACCTTTTGCGTGCA |
| | | | GGTGCAGGGCAAGTCTAAGAGGGAGAAGAAGGACCGCGT |
| | | | GTTCACCGATAAGACAAGCGCCACCGTGATCTGTCGGAAG |
| | | | AACGCCAGCATCTCCGTGCGGGCCCAGGATAGATACTATT |
| | | | CTAGCTCCTGGAGCGAGTGGGCCTCCGTGCCATGTTCT |
| 148 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGAGAGGCGACGCCGGACAGTACA |
| | | | CCTGTCACAAGGGCGGCGAGGTGCTGTCCCACTCTCTGCT |
| | | | GCTGCTGCACAAGAAGGAGGACGGCATCTGGAGCACAGA |
| | | | CATCCTGAAGGATCAGAAGGAGCCCAAGAACAAGACCTTC |
| | | | CTGAGGTGCGAGGCCAAGAATTATAGCGGCAGATTCACCT |
| | | | GTTGGTGGCTGACCACAATCTCCACCGATCTGACATTTTCT |
| | | | GTGAAGTCTAGCAGGGGCTCCTCTGACCCTCAGGGAGTGA |
| | | | CATGCGGAGCCGCCACCCTGAGCGCCGAGCGGGTGAGAG |
| | | | GCGATAACAAGGAGTACGAGTATTCCGTGGAGTGCCAGGA |
| | | | GGACTCTGCCTGTCCCGCCGCCGAGGAGTCCCTGCCTATCG |
| | | | AAGTGATGGTGGATGCCGTGCACAAGCTGAAGTACGAGAA |
| | | | TTATACAAGCTCCTTCTTTATCAGGGACATCATCAAGCCCG |
| | | | ATCCCCCTAAGAACCTGCAGCTGAAGCCTCTGAAGAATAG |
| | | | CCGCCAGGTGGAGGTGTCCTGGGAGTACCCTGACACCTGG |
| | | | AGCACACCACACTCCTATTTCTCTCTGACCTTTTGCGTGCA |
| | | | GGTGCAGGGCAAGTCTAAGAGGGAGAAGAAGGACCGCGT |
| | | | GTTCACCGATAAGACAAGCGCCACCGTGATCTGTCGGAAG |
| | | | AACGCCAGCATCTCCGTGCGGGCCCAGGATAGATACTATT |
| | | | CTAGCTCCTGGAGCGAGTGGGCCTCCGTGCCATGTTCT |
| 149 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACGAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 150 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACATCAAGGAGGACGGCATCTGGAGCACAGACA |
| | | | TCCTGAAGGATCAGAAGGAGCCCAAGAACAAGACCTTCCT |
| | | | GAGGTGCGAGGCCAAGAATTATAGCGGCAGATTCACCTGT |
| | | | TGGTGGCTGACCACAATCTCCACCGATCTGACATTTTCTGT |
| | | | GAAGTCTAGCAGGGGCTCCTCTGACCCTCAGGGAGTGACA |
| | | | TGCGGAGCCGCCACCCTGAGCGCCGAGCGGGTGAGAGGC |
| | | | GATAACAAGGAGTACGAGTATTCCGTGGAGTGCCAGGAGG |
| | | | ACTCTGCCTGTCCCGCCGCCGAGGAGTCCCTGCCTATCGAA |
| | | | GTGATGGTGGATGCCGTGCACAAGCTGAAGTACGAGAATT |
| | | | ATACAAGCTCCTTCTTTATCAGGGACATCATCAAGCCCGAT |
| | | | CCCCCTAAGAACCTGCAGCTGAAGCCTCTGAAGAATAGCC |
| | | | GCCAGGTGGAGGTGTCCTGGGAGTACCCTGACACCTGGAG |
| | | | CACACCACACTCCTATTTCTCTCTGACCTTTTGCGTGCAGG |
| | | | TGCAGGGCAAGTCTAAGAGGGAGAAGAAGGACCGCGTGT |
| | | | TCACCGATAAGACAAGCGCCACCGTGATCTGTCGGAAGAA |
| | | | CGCCAGCATCTCCGTGCGGGCCCAGGATAGATACTATTCT |
| | | | AGCTCCTGGAGCGAGTGGGCCTCCGTGCCATGTTCT |
| 151 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACCTGAAGGAGGACGGCATCTGGAGCACAGACA |
| | | | TCCTGAAGGATCAGAAGGAGCCCAAGAACAAGACCTTCCT |
| | | | GAGGTGCGAGGCCAAGAATTATAGCGGCAGATTCACCTGT |
| | | | TGGTGGCTGACCACAATCTCCACCGATCTGACATTTTCTGT |
| | | | GAAGTCTAGCAGGGGCTCCTCTGACCCTCAGGGAGTGACA |
| | | | TGCGGAGCCGCCACCCTGAGCGCCGAGCGGGTGAGAGGC |
| | | | GATAACAAGGAGTACGAGTATTCCGTGGAGTGCCAGGAGG |
| | | | ACTCTGCCTGTCCCGCCGCCGAGGAGTCCCTGCCTATCGAA |
| | | | GTGATGGTGGATGCCGTGCACAAGCTGAAGTACGAGAATT |
| | | | ATACAAGCTCCTTCTTTATCAGGGACATCATCAAGCCCGAT |
| | | | CCCCCTAAGAACCTGCAGCTGAAGCCTCTGAAGAATAGCC |
| | | | GCCAGGTGGAGGTGTCCTGGGAGTACCCTGACACCTGGAG |
| | | | CACACCACACTCCTATTTCTCTCTGACCTTTTGCGTGCAGG |
| | | | TGCAGGGCAAGTCTAAGAGGGAGAAGAAGGACCGCGTGT |
| | | | TCACCGATAAGACAAGCGCCACCGTGATCTGTCGGAAGAA |
| | | | CGCCAGCATCTCCGTGCGGGCCCAGGATAGATACTATTCT |
| | | | AGCTCCTGGAGCGAGTGGGCCTCCGTGCCATGTTCT |
| 152 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACTGGAAGGAGGACGGCATCTGGAGCACAGACA |
| | | | TCCTGAAGGATCAGAAGGAGCCCAAGAACAAGACCTTCCT |
| | | | GAGGTGCGAGGCCAAGAATTATAGCGGCAGATTCACCTGT |
| | | | TGGTGGCTGACCACAATCTCCACCGATCTGACATTTTCTGT |
| | | | GAAGTCTAGCAGGGGCTCCTCTGACCCTCAGGGAGTGACA |
| | | | TGCGGAGCCGCCACCCTGAGCGCCGAGCGGGTGAGAGGC |
| | | | GATAACAAGGAGTACGAGTATTCCGTGGAGTGCCAGGAGG |
| | | | ACTCTGCCTGTCCCGCCGCCGAGGAGTCCCTGCCTATCGAA |
| | | | GTGATGGTGGATGCCGTGCACAAGCTGAAGTACGAGAATT |
| | | | ATACAAGCTCCTTCTTTATCAGGGACATCATCAAGCCCGAT |
| | | | CCCCCTAAGAACCTGCAGCTGAAGCCTCTGAAGAATAGCC |
| | | | GCCAGGTGGAGGTGTCCTGGGAGTACCCTGACACCTGGAG |
| | | | CACACCACACTCCTATTTCTCTCTGACCTTTTGCGTGCAGG |
| | | | TGCAGGGCAAGTCTAAGAGGGAGAAGAAGGACCGCGTGT |
| | | | TCACCGATAAGACAAGCGCCACCGTGATCTGTCGGAAGAA |
| | | | CGCCAGCATCTCCGTGCGGGCCCAGGATAGATACTATTCT |
| | | | AGCTCCTGGAGCGAGTGGGCCTCCGTGCCATGTTCT |
| 153 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACTACAAGGAGGACGGCATCTGGAGCACAGACA |
| | | | TCCTGAAGGATCAGAAGGAGCCCAAGAACAAGACCTTCCT |
| | | | GAGGTGCGAGGCCAAGAATTATAGCGGCAGATTCACCTGT |
| | | | TGGTGGCTGACCACAATCTCCACCGATCTGACATTTTCTGT |
| | | | GAAGTCTAGCAGGGGCTCCTCTGACCCTCAGGGAGTGACA |
| | | | TGCGGAGCCGCCACCCTGAGCGCCGAGCGGGTGAGAGGC |
| | | | GATAACAAGGAGTACGAGTATTCCGTGGAGTGCCAGGAGG |
| | | | ACTCTGCCTGTCCCGCCGCCGAGGAGTCCCTGCCTATCGAA |
| | | | GTGATGGTGGATGCCGTGCACAAGCTGAAGTACGAGAATT |
| | | | ATACAAGCTCCTTCTTTATCAGGGACATCATCAAGCCCGAT |
| | | | CCCCCTAAGAACCTGCAGCTGAAGCCTCTGAAGAATAGCC |
| | | | GCCAGGTGGAGGTGTCCTGGGAGTACCCTGACACCTGGAG |
| | | | CACACCACACTCCTATTTCTCTCTGACCTTTTGCGTGCAGG |
| | | | TGCAGGGCAAGTCTAAGAGGGAGAAGAAGGACCGCGTGT |
| | | | TCACCGATAAGACAAGCGCCACCGTGATCTGTCGGAAGAA |
| | | | CGCCAGCATCTCCGTGCGGGCCCAGGATAGATACTATTCT |
| | | | AGCTCCTGGAGCGAGTGGGCCTCCGTGCCATGTTCT |
| 154 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGAGAGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 155 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGTGGGACGGCATCTGGAGCACAGACA |
| | | | TCCTGAAGGATCAGAAGGAGCCCAAGAACAAGACCTTCCT |
| | | | GAGGTGCGAGGCCAAGAATTATAGCGGCAGATTCACCTGT |
| | | | TGGTGGCTGACCACAATCTCCACCGATCTGACATTTTCTGT |
| | | | GAAGTCTAGCAGGGGCTCCTCTGACCCTCAGGGAGTGACA |
| | | | TGCGGAGCCGCCACCCTGAGCGCCGAGCGGGTGAGAGGC |
| | | | GATAACAAGGAGTACGAGTATTCCGTGGAGTGCCAGGAGG |
| | | | ACTCTGCCTGTCCCGCCGCCGAGGAGTCCCTGCCTATCGAA |
| | | | GTGATGGTGGATGCCGTGCACAAGCTGAAGTACGAGAATT |
| | | | ATACAAGCTCCTTCTTTATCAGGGACATCATCAAGCCCGAT |
| | | | CCCCCTAAGAACCTGCAGCTGAAGCCTCTGAAGAATAGCC |
| | | | GCCAGGTGGAGGTGTCCTGGGAGTACCCTGACACCTGGAG |
| | | | CACACCACACTCCTATTTCTCTCTGACCTTTTGCGTGCAGG |
| | | | TGCAGGGCAAGTCTAAGAGGGAGAAGAAGGACCGCGTGT |
| | | | TCACCGATAAGACAAGCGCCACCGTGATCTGTCGGAAGAA |
| | | | CGCCAGCATCTCCGTGCGGGCCCAGGATAGATACTATTCT |
| | | | AGCTCCTGGAGCGAGTGGGCCTCCGTGCCATGTTCT |
| 156 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAG |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 157 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACACAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 158 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAAGA |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 159 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CAGAAACAAGGAGTACGAGTATTCCGTGGAGTGCCAGGA |
| | | | GGACTCTGCCTGTCCCGCCGCCGAGGAGTCCCTGCCTATCG |
| | | | AAGTGATGGTGGATGCCGTGCACAAGCTGAAGTACGAGAA |
| | | | TTATACAAGCTCCTTCTTTATCAGGGACATCATCAAGCCCG |
| | | | ATCCCCCTAAGAACCTGCAGCTGAAGCCTCTGAAGAATAG |
| | | | CCGCCAGGTGGAGGTGTCCTGGGAGTACCCTGACACCTGG |
| | | | AGCACACCACACTCCTATTTCTCTCTGACCTTTTGCGTGCA |
| | | | GGTGCAGGGCAAGTCTAAGAGGGAGAAGAAGGACCGCGT |
| | | | GTTCACCGATAAGACAAGCGCCACCGTGATCTGTCGGAAG |
| | | | AACGCCAGCATCTCCGTGCGGGCCCAGGATAGATACTATT |
| | | | CTAGCTCCTGGAGCGAGTGGGCCTCCGTGCCATGTTCT |
| 160 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACGACCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 161 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGGACTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 162 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGCAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 163 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGACCTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 164 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGTGGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 165 | | Mutated p40 | ATCTGGGAGCTGAAGAAGGACGTGTACGTGGTGGAGCTGG |
| | | nucleotide | ACAGATATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGACGACGGCGACGCCGGACAGTACA |
| | | | CCTGTCACAAGGGCGGCGAGGTGCTGTCCCACTCTCTGCT |
| | | | GCTGCTGCACAAGAAGGAGGACGGCATCTGGAGCACAGA |
| | | | CATCCTGAAGGATCAGAAGGAGCCCAAGAACAAGACCTTC |
| | | | CTGAGGTGCGAGGCCAAGAATTATAGCGGCAGATTCACCT |
| | | | GTTGGTGGCTGACCACAATCTCCACCGATCTGACATTTTCT |
| | | | GTGAAGTCTAGCAGGGGCTCCTCTGACCCTCAGGGAGTGA |
| | | | CATGCGGAGCCGCCACCCTGAGCGCCGAGCGGGTGAGAG |
| | | | GCGATAACAAGGAGTACGAGTATTCCGTGGAGTGCCAGGA |
| | | | GGACTCTGCCTGTCCCGCCGCCGAGGAGTCCCTGCCTATCG |
| | | | AAGTGATGGTGGATGCCGTGCACAAGCTGTGGTACGAGAA |
| | | | TTATACAAGCTCCTTCTTTATCAGGGACATCATCAAGCCCG |
| | | | ATCCCCCTAAGAACCTGCAGCTGAAGCCTCTGAAGAATAG |
| | | | CCGCCAGGTGGAGGTGTCCTGGGAGTACCCTGACACCTGG |
| | | | AGCACACCACACTCCTATTTCTCTCTGACCTTTTGCGTGCA |
| | | | GGTGCAGGGCAAGTCTAAGAGGGAGAAGAAGGACCGCGT |
| | | | GTTCACCGATAAGACAAGCGCCACCGTGATCTGTCGGAAG |
| | | | AACGCCAGCATCTCCGTGCGGGCCCAGGATAGATACTATT |
| | | | CTAGCTCCTGGAGCGAGTGGGCCTCCGTGCCATGTTCT |
| 166 | | Mutated p40 | ATCTGGGAGCTGAAGAAGGACGTGTACGTGGTGGAGCTGG |
| | | nucleotide | ACCACTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACCTGAAGGAGGACGGCATCTGGAGCACAGACA |
| | | | TCCTGAAGGATCAGAAGGAGCCCAAGAACAAGACCTTCCT |
| | | | GAGGTGCGAGGCCAAGAATTATAGCGGCAGATTCACCTGT |
| | | | TGGTGGCTGACCACAATCTCCACCGATCTGACATTTTCTGT |
| | | | GAAGTCTAGCAGGGGCTCCTCTGACCCTCAGGGAGTGACA |
| | | | TGCGGAGCCGCCACCCTGAGCGCCGAGCGGGTGAGAGGC |
| | | | GATAACAAGGAGTACGAGTATTCCGTGGAGTGCCAGGAGG |
| | | | ACTCTGCCTGTCCCGCCGCCGAGGAGTCCCTGCCTATCGAA |
| | | | GTGATGGTGGATGCCGTGCACAAGCTGAAGTACGAGAATT |
| | | | ATACAAGCTCCTTCTTTATCAGGGACATCATCAAGCCCGAT |
| | | | CCCCCTAAGAACCTGCAGCTGAAGCCTCTGAAGAATAGCC |
| | | | GCCAGGTGGAGGTGTCCTGGGAGTACCCTGACACCTGGAG |
| | | | CACACCACACTCCTATTTCTCTCTGACCTTTTGCGTGCAGG |
| | | | TGCAGGGCAAGTCTAAGAGGGAGAAGAAGGACCGCGTGT |
| | | | TCACCGATAAGACAAGCGCCACCGTGATCTGTCGGAAGAA |
| | | | CGCCAGCATCTCCGTGCGGGCCCAGGATAGATACTATTCT |
| | | | AGCTCCTGGAGCGAGTGGGCCTCCGTGCCATGTTCT |
| 167 | | Mutated p40 | ATCTGGGAGCTGAAGAAGGACGTGTACGTGGTGGAGCTGG |
| | | nucleotide | ACCACTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACCTGAAGGAGGACGGCATCTGGAGCACAGACA |
| | | | TCCTGAAGGATCAGAAGGAGCCCAAGAACAAGACCTTCCT |
| | | | GAGGTGCGAGGCCAAGAATTATAGCGGCAGATTCACCTGT |
| | | | TGGTGGCTGACCACAATCTCCACCGATCTGACATTTTCTGT |
| | | | GAAGTCTAGCAGGGGCTCCTCTGACCCTCAGGGAGTGACA |
| | | | TGCGGAGCCGCCACCCTGAGCGCCGAGCGGGTGAGAGGC |
| | | | GATAACAAGGAGTACGAGTATTCCGTGGAGTGCCAGGAGG |
| | | | ACTCTGCCTGTCCCGCCGCCGAGGAGTCCCTGCCTATCGAA |
| | | | GTGATGGTGGATGCCGTGCACAAGCTGCAGTACGAGAATT |
| | | | ATACAAGCTCCTTCTTTATCAGGGACATCATCAAGCCCGAT |
| | | | CCCCCTAAGAACCTGCAGCTGAAGCCTCTGAAGAATAGCC |
| | | | GCCAGGTGGAGGTGTCCTGGGAGTACCCTGACACCTGGAG |
| | | | CACACCACACTCCTATTTCTCTCTGACCTTTTGCGTGCAGG |
| | | | TGCAGGGCAAGTCTAAGAGGGAGAAGAAGGACCGCGTGT |
| | | | TCACCGATAAGACAAGCGCCACCGTGATCTGTCGGAAGAA |
| | | | CGCCAGCATCTCCGTGCGGGCCCAGGATAGATACTATTCT |
| | | | AGCTCCTGGAGCGAGTGGGCCTCCGTGCCATGTTCT |
| 168 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GACCAGAGCTCCAGAGTGCTGGGCTCCGGCAAGACCCTGA |
| | | | CAATCCAGGTGAGCAGCTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACGACCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 169 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGCACGAGTTCGGCGACGCCGGACAGTACAC |
| | | | CTGTCACAAGGGCGGCGAGGTGCTGTCCCACTCTCTGCTG |
| | | | CTGCTGCACAAGAAGAGAGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGGACTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 170 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GACCAGAGCTCCGAGGTGCTGGGCTCCGGCAAGACCCTGA |
| | | | CAATCCAGGTGAGCAGCTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACGACCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 171 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGACTTCGGCGACGCCGGACAGTACAC |
| | | | CTGTCACAAGGGCGGCGAGGTGCTGTCCCACTCTCTGCTG |
| | | | CTGCTGCACTGGAAGGAGGACGGCATCTGGAGCACAGACA |
| | | | TCCTGAAGGATCAGAAGGAGCCCAAGAACAAGACCTTCCT |
| | | | GAGGTGCGAGGCCAAGAATTATAGCGGCAGATTCACCTGT |
| | | | TGGTGGCTGACCACAATCTCCACCGATCTGACATTTTCTGT |
| | | | GAAGTCTAGCAGGGGCTCCTCTGACCCTCAGGGAGTGACA |
| | | | TGCGGAGCCGCCACCCTGAGCGCCGAGCGGGTGAGAGGC |
| | | | GATAACAAGGAGTACGAGTATTCCGTGGAGTGCCAGGAGG |
| | | | ACTCTGCCTGTCCCGCCGCCGAGGAGTCCCTGCCTATCGAA |
| | | | GTGATGGTGGATGCCGTGCACAAGCTGTGGTACGAGAATT |
| | | | ATACAAGCTCCTTCTTTATCAGGGACATCATCAAGCCCGAT |
| | | | CCCCCTAAGAACCTGCAGCTGAAGCCTCTGAAGAATAGCC |
| | | | GCCAGGTGGAGGTGTCCTGGGAGTACCCTGACACCTGGAG |
| | | | CACACCACACTCCTATTTCTCTCTGACCTTTTGCGTGCAGG |
| | | | TGCAGGGCAAGTCTAAGAGGGAGAAGAAGGACCGCGTGT |
| | | | TCACCGATAAGACAAGCGCCACCGTGATCTGTCGGAAGAA |
| | | | CGCCAGCATCTCCGTGCGGGCCCAGGATAGATACTATTCT |
| | | | AGCTCCTGGAGCGAGTGGGCCTCCGTGCCATGTTCT |
| 172 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGACTTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACACAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 173 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGAGATTCGGCGACGCCGGACAGTACAC |
| | | | CTGTCACAAGGGCGGCGAGGTGCTGTCCCACTCTCTGCTG |
| | | | CTGCTGCACAAGAAGGAGGACGGCATCTGGAGCACAAGA |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 174 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGAAGGGCGACGCCGGACAGTACA |
| | | | CCTGTCACAAGGGCGGCGAGGTGCTGTCCCACTCTCTGCT |
| | | | GCTGCTGCACAAGAAGGAGGACGGCATCTGGAGCACAGA |
| | | | CATCCTGAAGGATCAGAAGGAGCCCAAGAACAAGACCTTC |
| | | | CTGAGGTGCGAGGCCAAGAATTATAGCGGCAGATTCACCT |
| | | | GTTGGTGGCTGACCACAATCTCCACCGATCTGACATTTTCT |
| | | | GTGAAGTCTAGCAGGGGCTCCTCTGACCCTCAGGGAGTGA |
| | | | CATGCGGAGCCGCCACCCTGAGCGCCGAGCGGGTGAGAG |
| | | | GCGATAACAAGGAGTACGAGTATTCCGTGGAGTGCCAGGA |
| | | | GGACTCTGCCTGTCCCGCCGCCGAGGAGTCCCTGCCTATCG |
| | | | AAGTGATGGTGGATGCCGTGCACAAGCTGTGGTACGAGAA |
| | | | TTATACAAGCTCCTTCTTTATCAGGGACATCATCAAGCCCG |
| | | | ATCCCCCTAAGAACCTGCAGCTGAAGCCTCTGAAGAATAG |
| | | | CCGCCAGGTGGAGGTGTCCTGGGAGTACCCTGACACCTGG |
| | | | AGCACACCACACTCCTATTTCTCTCTGACCTTTTGCGTGCA |
| | | | GGTGCAGGGCAAGTCTAAGAGGGAGAAGAAGGACCGCGT |
| | | | GTTCACCGATAAGACAAGCGCCACCGTGATCTGTCGGAAG |
| | | | AACGCCAGCATCTCCGTGCGGGCCCAGGATAGATACTATT |
| | | | CTAGCTCCTGGAGCGAGTGGGCCTCCGTGCCATGTTCT |
| 175 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGAGAGGCGACGCCGGACAGTACA |
| | | | CCTGTCACAAGGGCGGCGAGGTGCTGTCCCACTCTCTGCT |
| | | | GCTGCTGCACGAGAAGGAGGACGGCATCTGGAGCACAGA |
| | | | CATCCTGAAGGATCAGAAGGAGCCCAAGAACAAGACCTTC |
| | | | CTGAGGTGCGAGGCCAAGAATTATAGCGGCAGATTCACCT |
| | | | GTTGGTGGCTGACCACAATCTCCACCGATCTGACATTTTCT |
| | | | GTGAAGTCTAGCAGGGGCTCCTCTGACCCTCAGGGAGTGA |
| | | | CATGCGGAGCCGCCACCCTGAGCGCCGAGCGGGTGAGAG |
| | | | GCGATAACAAGGAGTACGAGTATTCCGTGGAGTGCCAGGA |
| | | | GGACTCTGCCTGTCCCGCCGCCGAGGAGTCCCTGCCTATCG |
| | | | AAGTGATGGTGGATGCCGTGCACAAGCTGTGGTACGAGAA |
| | | | TTATACAAGCTCCTTCTTTATCAGGGACATCATCAAGCCCG |
| | | | ATCCCCCTAAGAACCTGCAGCTGAAGCCTCTGAAGAATAG |
| | | | CCGCCAGGTGGAGGTGTCCTGGGAGTACCCTGACACCTGG |
| | | | AGCACACCACACTCCTATTTCTCTCTGACCTTTTGCGTGCA |
| | | | GGTGCAGGGCAAGTCTAAGAGGGAGAAGAAGGACCGCGT |
| | | | GTTCACCGATAAGACAAGCGCCACCGTGATCTGTCGGAAG |
| | | | AACGCCAGCATCTCCGTGCGGGCCCAGGATAGATACTATT |
| | | | CTAGCTCCTGGAGCGAGTGGGCCTCCGTGCCATGTTCT |
| 176 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGAGAGGCGACGCCGGACAGTACA |
| | | | CCTGTCACAAGGGCGGCGAGGTGCTGTCCCACTCTCTGCT |
| | | | GCTGCTGCACTACAAGGAGGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 177 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACATCAAGAGAGACGGCATCTGGAGCACAGACA |
| | | | TCCTGAAGGATCAGAAGGAGCCCAAGAACAAGACCTTCCT |
| | | | GAGGTGCGAGGCCAAGAATTATAGCGGCAGATTCACCTGT |
| | | | TGGTGGCTGACCACAATCTCCACCGATCTGACATTTTCTGT |
| | | | GAAGTCTAGCAGGGGCTCCTCTGACCCTCAGGGAGTGACA |
| | | | TGCGGAGCCGCCACCCTGAGCGCCGAGCGGGTGAGAGGC |
| | | | GATAACAAGGAGTACGAGTATTCCGTGGAGTGCCAGGAGG |
| | | | ACTCTGCCTGTCCCGCCGCCGAGGAGTCCCTGCCTATCGAA |
| | | | GTGATGGTGGATGCCGTGCACAAGCTGAAGTACGAGAATT |
| | | | ATACAAGCTCCTTCTTTATCAGGGACATCATCAAGCCCGAT |
| | | | CCCCCTAAGAACCTGCAGCTGAAGCCTCTGAAGAATAGCC |
| | | | GCCAGGTGGAGGTGTCCTGGGAGTACCCTGACACCTGGAG |
| | | | CACACCACACTCCTATTTCTCTCTGACCTTTTGCGTGCAGG |
| | | | TGCAGGGCAAGTCTAAGAGGGAGAAGAAGGACCGCGTGT |
| | | | TCACCGATAAGACAAGCGCCACCGTGATCTGTCGGAAGAA |
| | | | CGCCAGCATCTCCGTGCGGGCCCAGGATAGATACTATTCT |
| | | | AGCTCCTGGAGCGAGTGGGCCTCCGTGCCATGTTCT |
| 178 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACATCAAGAGAGACGGCATCTGGAGCACACACA |
| | | | TCCTGAAGGATCAGAAGGAGCCCAAGAACAAGACCTTCCT |
| | | | GAGGTGCGAGGCCAAGAATTATAGCGGCAGATTCACCTGT |
| | | | TGGTGGCTGACCACAATCTCCACCGATCTGACATTTTCTGT |
| | | | GAAGTCTAGCAGGGGCTCCTCTGACCCTCAGGGAGTGACA |
| | | | TGCGGAGCCGCCACCCTGAGCGCCGAGCGGGTGAGAGGC |
| | | | GATAACAAGGAGTACGAGTATTCCGTGGAGTGCCAGGAGG |
| | | | ACTCTGCCTGTCCCGCCGCCGAGGAGTCCCTGCCTATCGAA |
| | | | GTGATGGTGGATGCCGTGCACAAGCTGAAGTACGAGAATT |
| | | | ATACAAGCTCCTTCTTTATCAGGGACATCATCAAGCCCGAT |
| | | | CCCCCTAAGAACCTGCAGCTGAAGCCTCTGAAGAATAGCC |
| | | | GCCAGGTGGAGGTGTCCTGGGAGTACCCTGACACCTGGAG |
| | | | CACACCACACTCCTATTTCTCTCTGACCTTTTGCGTGCAGG |
| | | | TGCAGGGCAAGTCTAAGAGGGAGAAGAAGGACCGCGTGT |
| | | | TCACCGATAAGACAAGCGCCACCGTGATCTGTCGGAAGAA |
| | | | CGCCAGCATCTCCGTGCGGGCCCAGGATAGATACTATTCT |
| | | | AGCTCCTGGAGCGAGTGGGCCTCCGTGCCATGTTCT |
| 179 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACTGGAAGGAGGACGGCATCTGGAGCACAGACA |
| | | | TCCTGAAGGATCAGAAGGAGCCCAAGAACAAGACCTTCCT |
| | | | GAGGTGCGAGGCCAAGAATTATAGCGGCAGATTCACCTGT |
| | | | TGGTGGCTGACCACAATCTCCACCGATCTGACATTTTCTGT |
| | | | GAAGTCTAGCAGGGGCTCCTCTGACCCTCAGGGAGTGACA |
| | | | TGCGGAGCCGCCACCCTGAGCGCCGAGCGGGTGAGAGGC |
| | | | AGAAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 180 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACTGGAAGGAGGACGGCATCTGGAGCACAGACA |
| | | | TCCTGAAGGATCAGAAGGAGCCCAAGAACAAGACCTTCCT |
| | | | GAGGTGCGAGGCCAAGAATTATAGCGGCAGATTCACCTGT |
| | | | TGGTGGCTGACCACAATCTCCACCGATCTGACATTTTCTGT |
| | | | GAAGTCTAGCAGGGGCTCCTCTGACCCTCAGGGAGTGACA |
| | | | TGCGGAGCCGCCACCCTGAGCGCCGAGCGGGTGAGAGGC |
| | | | GATAACAAGGAGTACGAGTATTCCGTGGAGTGCCAGGAGG |
| | | | ACTCTGCCTGTCCCGCCGCCGAGGAGTCCCTGCCTATCGAA |
| | | | GTGATGGTGGATGCCGTGCACAAGCTGTGGTACGAGAATT |
| | | | ATACAAGCTCCTTCTTTATCAGGGACATCATCAAGCCCGAT |
| | | | CCCCCTAAGAACCTGCAGCTGAAGCCTCTGAAGAATAGCC |
| | | | GCCAGGTGGAGGTGTCCTGGGAGTACCCTGACACCTGGAG |
| | | | CACACCACACTCCTATTTCTCTCTGACCTTTTGCGTGCAGG |
| | | | TGCAGGGCAAGTCTAAGAGGGAGAAGAAGGACCGCGTGT |
| | | | TCACCGATAAGACAAGCGCCACCGTGATCTGTCGGAAGAA |
| | | | CGCCAGCATCTCCGTGCGGGCCCAGGATAGATACTATTCT |
| | | | AGCTCCTGGAGCGAGTGGGCCTCCGTGCCATGTTCT |
| 181 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGAGAGACGGCATCTGGAGCACAGAC |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGGACTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |
| 182 | | Mutated p40 | ATCTGGGAGCTGAAGAAGGACGTGTACGTGGTGGAGCTGG |
| | | nucleotide | ACTGGTATCCAGATGCACCAGGAGAGATGGTGGTGCTGAC |
| | | | CTGCGACACACCAGAGGAGGATGGCATCACCTGGACACTG |
| | | | GATCAGAGCTCCGAGGTGCTGGGCTCCGGCAAGACCCTGA |
| | | | CAATCCAGGTGAAGGAGTTCGGCGACGCCGGACAGTACAC |
| | | | CTGTCACAAGGGCGGCGAGGTGCTGTCCCACTCTCTGCTG |
| | | | CTGCTGCACAAGAAGTGGGACGGCATCTGGAGCACAGAG |
| | | | ATCCTGAAGGATCAGAAGGAGCCCAAGAACAAGACCTTCC |
| | | | TGAGGTGCGAGGCCAAGAATTATAGCGGCAGATTCACCTG |
| | | | TTGGTGGCTGACCACAATCTCCACCGATCTGACATTTTCTG |
| | | | TGAAGTCTAGCAGGGGCTCCTCTGACCCTCAGGGAGTGAC |
| | | | ATGCGGAGCCGCCACCCTGAGCGCCGAGCGGGTGAGAGG |
| | | | CGATAACAAGGAGTACGAGTATTCCGTGGAGTGCCAGGAG |
| | | | GACTCTGCCTGTCCCGCCGCCGAGGAGTCCCTGCCTATCGA |
| | | | AGTGATGGTGGATGCCGTGCACAAGCTGAAGTACGAGAAT |
| | | | TATACAAGCTCCTTCTTTATCAGGGACATCATCAAGCCCGA |
| | | | TCCCCCTAAGAACCTGCAGCTGAAGCCTCTGAAGAATAGC |
| | | | CGCCAGGTGGAGGTGTCCTGGGAGTACCCTGACACCTGGA |
| | | | GCACACCACACTCCTATTTCTCTCTGACCTTTTGCGTGCAG |
| | | | GTGCAGGGCAAGTCTAAGAGGGAGAAGAAGGACCGCGTG |
| | | | TTCACCGATAAGACAAGCGCCACCGTGATCTGTCGGAAGA |
| | | | ACGCCAGCATCTCCGTGCGGGCCCAGGATAGATACTATTC |
| | | | TAGCTCCTGGAGCGAGTGGGCCTCCGTGCCATGTTCT |

What is claimed is:

1. A modified p40 domain, the modified p40 domain comprising one or more amino acid substitutions relative to the wild-type human mature IL12 p40 domain sequence set forth in SEQ ID NO: 10, wherein the one or more amino acid substitutions are located at one or more positions of E45, D62 and D161, and wherein the numbering of the amino acid residues is based on the amino acid sequence set forth in SEQ ID NO: 10.

2. The modified p40 domain of claim 1, wherein the one or more amino acid substitutions at the one or more positions are a K, H, I, N, R or an S substitution, or a combination thereof.

3. The modified p40 domain of any one of claims 1-2, wherein the one or more amino acid substitutions comprise E45K.

4. The modified p40 domain of any one of claims 1-3, wherein the one or more amino acid substitutions comprise D62H.

5. The modified p40 domain of any one of claims 1-3, wherein the one or more amino acid substitutions comprise D62I.

6. The modified p40 domain of any one of claims 1-3, wherein the one or more amino acid substitutions comprise D62N.

7. The modified p40 domain of any one of claims 1-6, wherein the one or more amino acid substitutions comprise D161R.

8. The modified p40 domain of any one of claims 1-6, wherein the one or more amino acid substitutions comprise D161S.

9. The modified p40 domain of any one of claims 1-8, wherein the one or more amino acid substitutions comprise a combination of two or more substitutions of E45K, D62H, D62I, D62N, D161R or D161S.

10. A modified p40 domain, the modified p40 domain comprising one or more amino acid substitutions relative to the wild-type human mature IL12 p40 domain sequence set forth in SEQ ID NO: 10, wherein the one or more amino acid substitutions are W15H, W15K, W15R, D18G, E45K, K58H, K58W, E59D, E59G, E59R, F60D, F60E, F60K, F60R, F60V, D62H, D62I, D62N, K84E, K84I, K84L, K84V, K84W, K84Y, E86L, E86R, E865, E86W, D93E, D93H, D93R, D93W, D161R, D161S, K197D, K197E, K197Q, K197T, or K197W, or a combination thereof, and wherein the numbering of the amino acid residues is based on the amino acid sequence set forth in SEQ ID NO: 10.

11. The modified p40 domain of claim 10, wherein the modified p40 domain comprises one or more, two or more, or three or more amino acid substitutions.

12. The modified p40 domain of any one of claims 10-11, wherein the one or more amino acid substitutions are W15H, W15K, D18G, E45K, K58H, K58W, E59G, E59R, F60V, D62H, D62I, D62N, K84E, K84W, E86L, E86S, E86W, D93H, D93W, D161R, D161S, K197E, K197Q, K197T, or K197W, or a combination thereof.

13. The modified p40 domain of any one of claims 10-12, wherein the one or more amino acid substitutions are W15H, W15K, E59R, K84E, K84W, E86W, D161R, K197T, or K197W, or a combination thereof.

14. The modified p40 domain of any one of claims 10-11, wherein the two or more amino acid substitutions are W15H_K84L, K58H_K84I, E59D_K84W, E59G_K84W, E59R_K84E, E59R_K84W, E59R_E86W, E59D_D93H, E59R_D93R, E59R_K197E, E59R_K197W, F60E_K84W, F60R_K84Y, F60K_K197W, F60R_K197W, K84I_E86R, K84E_D93H, K84I_D93H, K84V_D93H, K84W_D93W, K84I_D161R, K84W_D161R, K84W_K197E, K84W_K197Q, K84W_K197W, E86W_D93E, E86R_K197D, E86W_K197W, W15H_K84L_K197Q, K58H_E86R_K197D, E59D_K84W_K197W, F60R_K84E_K197W, K84I_E86R_D93H, or W15R_E59D_F60D_K197W, or a combination thereof.

15. The modified p40 domain of claim 14, wherein the two or more amino acid substitutions are W15H_K84L, E59D_D93H, E59R_D93R, F60K_K197W, F60R_K84Y, K84I_E86R, K84W_D161R, K84W_K197W, E86R_K197D, E86W_D93E, W15H_K84L_K197Q, K58H_E86R_K197D, E59D_K84W_K197W, F60R_K84E_K197W, K84I_E86R_D93H, or W15R_E59D_F60D_K197W, or a combination thereof.

16. The modified p40 domain of claim 14, wherein the two or more amino acid substitutions are K58H_K84I, E59D_K84W, E59G_K84W, E59R_E86W, E59R_K197E, E59R_K197W, E59R_K84E, E59R_K84W, F60E_K84W, F60R_K197W, K84E_D93H, K84I_D161R, K84I_D93H, K84V_D93H, K84W_D93W, K84W_K197E, K84W_K197Q, or E86W_K197W, or a combination thereof.

17. The modified p40 domain of claim 14, wherein the two or more amino acid substitutions are W15H_K84L, K58H_K84I, E59D_K84W, E59G_K84W, E59R_K84E, E59R_K84W, E59R_E86W, E59D_D93H, E59R_D93R, E59R_K197E, E59R_K197W, F60E_K84W, F60R_K84Y, F60K_K197W, F60R_K197W, K84I_E86R, K84E_D93H, K84I_D93H, K84V_D93H, K84W_D93W, K84I_D161R, K84W_D161R, K84W_K197E, K84W_K197Q, K84W_K197W, E86W_D93E, E86R_K197D, or E86W_K197W, or a combination thereof.

18. The modified p40 domain of claim 14, wherein the two or more amino acid substitutions are W15H_K84L, E59D_D93H, E59R_D93R, F60K_K197W, F60R_K84Y, K84I_E86R, K84W_D161R, K84W_K197W, E86R_K197D, or E86W_D93E, or a combination thereof.

19. The modified p40 domain of any one of claims 10-11, wherein the three or more amino acid substitutions are W15H_K84L_K197Q, K58H_E86R_K197D, E59D_K84W_K197W, F60R_K84E_K197W, K84I_E86R_D93H or W15R_E59D_F60D_K197W, or a combination thereof.

20. The modified p40 domain of any one of claims 10-11, wherein the one or more amino acid substitutions are selected from Table C.

21. The modified p40 domain of any one of claims 10-11, wherein the modified p40 domain comprises or consists of an amino acid sequence having at least about 90%, 95%, 97%, 99%, or 100% sequence identity to the amino acid sequence set forth in any one of SEQ ID NOS: 12-14, 16, 18, 19, 21-34 and 36-56.

22. The modified p40 domain of any one of claims 10-11, wherein the modified p40 domain comprises an amino acid substitution or a set of amino acid substitutions selected from: W15R_E59D_F60D_K197W, W15H_K84L_K197Q, E59D_K84W_K197W, K58H_E86R_K197D, W15H_K84L, F60R_K84E_K197W, K84W_K197W, F60K_K197W, E86R_K197D, K84I_E86R_D93H, E59R_D93R, K84I_E86R, W15K, K84W_D161R, E45R_K58S_E59S_K195D, E59D_D93H, F60R_K84Y, E86W_D93E, K58S_E59S_K195D, K84E, K197T, E59R, W15H, K84W, K197W, E595, D161R or E86W, or a combination thereof.

23. The modified p40 domain of claim 22, wherein the modified p40 domain comprises an amino acid substitution or a set of amino acid substitutions selected from: W15H_K84L_K197Q, E59D_K84W_K197W, K84W_K197W, F60K_K197W, E59R_D93R, W15K, or F60R_K84Y, or a combination thereof.

24. The modified p40 domain of any one of claims 1-23, wherein the modified p40 domain has a binding affinity to at least one of its cognate receptors that is reduced from about 5-fold to about 1000-fold, from about 5-fold to about 800-fold, from about 5-fold to about 600-fold, from about 10-fold to about 500-fold, from about 10-fold to about 300-fold, or from about 20-fold to about 200-fold, relative to the binding affinity of the unmodified wildtype p40 domain having the sequence set forth in SEQ ID NO: 10, and as determined in a reporter gene assay (RGA).

25. The modified p40 domain of claim 24, wherein the modified p40 domain has a binding affinity to at least one of its cognate receptors that is reduced from about 20-fold to about 200-fold.

26. The modified p40 domain of any one of claims 24-25, wherein the at least one cognate receptor comprises IL12R131.

27. The modified p40 domain of any one of claims 1-26, wherein the modified p40 domain has a thermostability as measured by a melting temperature that is within ±5° C., ±4° C., ±3° C., ±2° C. or ±1° C. of that of the unmodified wildtype p40 domain that has the sequence set forth in SEQ ID NO: 10, and wherein the melting temperature is determined by Differential Scanning Fluorimetry (DSF) or Differential Scanning Calorimetry (DSC).

28. An IL12 fusion protein comprising the modified p40 domain of any one of claims 1-27.

29. An IL12 fusion protein comprising an IL12 polypeptide, wherein the IL12 polypeptide comprises the modified p40 domain of any one of claims 1-27; coupled to a p35 domain.

30. The IL12 fusion protein of claim 29, wherein the p35 domain comprises or consists of an amino acid sequence having at least about 90%, 95%, 97%, 99% or 100% sequence identity to the amino acid sequence set forth in SEQ ID NO: 11.

31. The IL12 fusion protein of claim 30, wherein the N-terminus of the p35 domain is coupled to the C-terminus of the modified p40 domain either directly or via a first linker.

32. The IL12 fusion protein of claim 31, wherein the first linker has the sequence of (G4S)x, and wherein x is 1, 2, 3 or 4.

33. The IL12 fusion protein of any one of claims 29-32, further comprising a heterodimeric Fc (HetFc) domain comprising a first Fc polypeptide and a second Fc polypeptide, thereby forming an IL12 HetFc fusion protein.

34. The IL12 HetFc fusion protein of claim 33, wherein the IL12 polypeptide is coupled to the first Fc polypeptide either directly or via a second linker.

35. The IL12 HetFc fusion protein of claim 34, wherein the IL12 polypeptide is coupled to the C-terminus of the first Fc polypeptide.

36. The IL12 HetFc fusion protein of any one of claims 34-35, wherein the IL12 polypeptide is coupled to the C-terminus of the first Fc polypeptide via the N-terminus of the modified p40 domain.

37. The IL12 HetFc fusion protein of any one of claims 33-36, further comprising a masking moiety, thereby forming a masked IL12 HetFc fusion protein, wherein the masking moiety is capable of non-covalently interacting with the modified p40 domain, thereby masking the modified p40 domain and reducing the binding affinity (K_{D}) of the modified p40 domain for binding to at least one of its cognate receptors when compared to an unmasked modified p40 domain.

38. The masked IL12 HetFc fusion protein of claim 37, wherein the masking moiety is coupled to the C-terminus of the second Fc polypeptide either directly or via a third linker.

39. The masked IL12 HetFc fusion protein of claim 38, wherein the third linker is a protease-cleavable linker.

40. The masked IL12 HetFc fusion protein of any one of claims 34-39, wherein the second linker and the third linker each comprise or consist of an amino acid sequence spanning from 5 to about 50 amino acids.

41. The masked IL12 HetFc fusion protein of claim 40, wherein the second linker comprises or consists of the amino acid sequence set forth in SEQ ID NO: 132, and the third linker comprises or consists of the amino acid sequence set forth in SEQ ID NO: 134.

42. The masked IL12 HetFc fusion protein of any one of claims 37-41, wherein the masking moiety comprises or consists of an scFv domain comprising a V_{H} domain coupled either directly or via a fourth linker to a V_{L} domain.

43. The masked IL12 HetFc fusion protein of claim 42, wherein the fourth linker comprises or consists of the amino acid sequence set forth in SEQ ID NO: 135.

44. The masked IL12 HetFc fusion protein of any one of claims 42-43, wherein the V_{H} domain comprises or consists of an amino acid sequence having about 95%, 97%, 99%, or 100% sequence identity to the sequence set forth in SEQ ID NO: 2, and the V_{L} domain comprises or consists of an amino acid sequence having about 95%, 97%, 99%, or 100% sequence identity to the sequence set forth in SEQ ID NO: 3.

45. The masked IL12 HetFc fusion protein of any one of claims 37-44, wherein the masking moiety is capable of reducing the binding affinity of the modified p40 domain to the at least one cognate receptor by at least about 10-fold, 15-fold, 20-fold, 30-fold, 40-fold, 50-fold, 100-fold, 200-fold, or at least about 300-fold, compared to a corresponding polypeptide construct that does not comprise the masking moiety.

46. The masked IL12 HetFc fusion protein of claim 45, wherein the at least one cognate receptor comprises IL12R131.

47. A masked IL12 HetFc fusion protein comprising:
(i) an IL-12 polypeptide comprising the modified p40 domain according to any one of claims 1-27 coupled via the linker (G4S)₄ to a p35 domain comprising or consisting of the amino acid sequence set forth in SEQ ID NO: 11;
(ii) a heterodimeric Fc domain comprising a first Fc polypeptide and a second Fc polypeptide; and
(iii) a masking moiety (MM) comprising an anti-IL12 scFv domain,
wherein:
the IL12 polypeptide is coupled either directly or via a second linker to the C-terminus of the first Fc polypeptide, and
the masking moiety is coupled either directly or via a third linker to the C-terminus of the second Fc polypeptide and is capable of non-covalently interacting with the IL12 polypeptide, thereby reducing the binding affinity of the IL12 polypeptide to at least one of its cognate receptors.

48. The masked IL12 HetFc fusion protein of claim 47, comprising or consisting of two polypeptide chains, from N- to C-terminus, (i) an Fc-IL12 polypeptide chain and (ii) an Fc-MM polypeptide chain.

49. The masked IL12 HetFc fusion protein of claim 48, wherein the Fc-IL12 polypeptide chain comprises or consists of an amino acid sequence having at least about 90%, 95%, 97%, 99%, or 100% sequence identity to any of the amino acid sequences set forth in SEQ ID NOs: 61-89.

50. The masked IL12 HetFc fusion protein of any one of claims 48-49, wherein the Fc-IL12 polypeptide chain comprises or consists of the amino acid sequence set forth in any one of SEQ ID NOs: 61-89.

51. The masked IL12 HetFc fusion protein of any one of claims 48-50, wherein the Fc-MM polypeptide chain comprises or consists of an amino acid sequence having at least about 90%, 95%, 97%, 99%, or 100% sequence identity to the amino acid sequence set forth in SEQ ID NO: 60.

52. The masked IL12 HetFc fusion protein of any one of claims 48-51, wherein the Fc-MM polypeptide chain comprises or consists of the amino acid sequence set forth in SEQ ID NO: 60.

53. The masked IL12 HetFc fusion protein of any one of claims 48-52, wherein (i) the Fc-IL12 polypeptide chain amino acid sequence is selected from the group consisting of the sequences of: v28046, v28047, v28048, v28049, v28050, v28051, v28053, v28054, v28055, v28056, v28057, v28058, v28059, v28060, v28061, v28062, v28063, v28064, v28065, v28066, v28067, v28068, v28069, v28070, v28071, v28072, v28074, v28075, and (ii) the Fc-MM polypeptide chain comprises or consists of the amino acid sequence of v26503.

54. An unmasked IL12 HetFc fusion protein comprising:
(i) an IL-12 polypeptide comprising the modified p40 domain according to any one of claims 1-27 coupled via the linker (G4S)₄ to the p35 domain comprising or consisting of the amino acid sequence set forth in SEQ ID NO: 11; and
(ii) a heterodimeric Fc domain comprising a first Fc polypeptide and a second Fc polypeptide,
wherein the IL12 polypeptide is coupled either directly or via a second linker to the C-terminus of the first Fc polypeptide.

55. The unmasked IL12 HetFc fusion protein of claim 54, comprising or consisting of two polypeptide chains, from N- to C-terminus, (i) an Fc-IL12 polypeptide chain and (ii) an Fc polypeptide chain.

56. The unmasked IL12 HetFc fusion protein of claim 55, wherein the Fc-IL12 polypeptide chain comprises or consists of an amino acid sequence having at least about 90%, 95%, 97%, 99%, or 100% sequence identity to any of the amino acid sequences set forth in SEQ ID NOs: 61-89.

57. The unmasked IL12 HetFc fusion protein of any one of claims 55-56, wherein the Fc-IL12 polypeptide chain comprises or consists of the amino acid sequence set forth in any one of SEQ ID NOs: 61-89.

58. The unmasked IL12 HetFc fusion protein of any one of claims 55-57, wherein the Fc polypeptide chain comprises or consists of an amino acid sequence having at least about 90%, 95%, 97%, 99%, or 100% sequence identity to the amino acid sequence set forth in SEQ ID NO: 58.

59. The unmasked IL12 HetFc fusion protein of any one of claims 55-58, wherein (i) the IL12 polypeptide chain amino acid sequence is selected from the group consisting of the sequences of: v28046, v28047, v28048, v28049, v28050, v28051, v28053, v28054, v28055, v28056, v28057, v28058, v28059, v28060, v28061, v28062, v28063, v28064, v28065, v28066, v28067, v28068, v28069, v28070, v28071, v28072, v28074, v28075, and (ii) the Fc polypeptide chain comprises or consists of the amino acid sequence of v12153.

60. A pharmaceutical composition comprising (i) the modified p40 domain of any one of claims 1-27, (ii) the masked IL12 HetFc fusion protein of any one of claims 38-53, and/or (iii) the unmasked IL12 HetFc fusion protein of claims 54-59, and a pharmaceutically acceptable carrier.

61. A nucleic acid molecule or a set of nucleic acid molecules encoding (i) the modified p40 domain of any one of claims 1-27, (ii) the masked IL12 HetFc fusion protein of any one of claims 37-53, and/or (iii) the unmasked IL12 HetFc fusion protein of claims of any one of claims 54-59.

62. A vector or a set of vectors comprising the nucleic acid molecule or the set of nucleic acid molecules of claim 61.

63. A method of identifying one or more amino acid substitutions in a p40 domain amino acid sequence to produce a modified p40 domain, the method comprising performing molecular dynamics and mutagenesis simulations, thereby identifying the one or more amino acid substitutions listed in Table C, wherein the one or more amino acid substitutions in the modified p40 domain amino acid sequence are relative to the sequence set forth in SEQ ID NO: 1, and wherein the one or more amino acid substitutions reduce the binding affinity (K_{D}) of the modified p40 domain to at least one of its cognate receptors, and relative to an unmodified p40 domain that does not comprise the one or more amino acid substitutions.

64. The method of claim 63, wherein the at least one cognate receptor comprises IL12Rβ1.

65. The method of any one of claims 63-64, wherein the binding affinity of the modified p40 domain is reduced by at least about 2-fold, 5-fold, 10-fold, 15-fold, 20-fold, 30-fold, 40-fold, 50-fold, 100-fold, 200-fold, 300-fold, 400-fold, 500-fold, or at least about 600-fold, compared to an unmodified p40 domain and as determined in a reporter gene assay.

66. A method of treating a disease in a subject in need thereof, the method comprising administering to the subject a therapeutically effective amount of a pharmaceutical composition comprising (i) the modified p40 domain of any one of claims 1-27, (ii) the masked IL12 HetFc fusion protein of any one of claims 37-53, and/or (iii) the unmasked IL12 HetFc fusion protein of claims 54-59, thereby treating the disease in the subject.

67. The method of claim 66, wherein the polypeptide construct is the masked IL12 HetFc fusion protein of any one of claims 38-53.

68. The method of claim 67, wherein the masking moiety of the masked IL12 HetFc fusion protein is cleaved from the fusion protein in a diseased tissue or organ.

69. The method of any one of claims 66-68, wherein the disease is a cancer.

70. A modified p40 domain of any one of claims 1-27, a masked IL12 HetFc fusion protein of any one of claims 37-53, or an unmasked IL12 HetFc fusion protein of claims of any one of claims 54-59, for use in therapy.

71. A modified p40 domain of any one of claims 1-27, a masked IL12 HetFc fusion protein of any one of claims 37-53, or an unmasked IL12 HetFc fusion protein of claims of any one of claims 54-59, for use in the treatment of cancer.

72. Use of a modified p40 domain of any one of claims 1-27, a masked IL12 HetFc fusion protein of any one of claims 37-53, or an unmasked IL12 HetFc fusion protein of claims of any one of claims 54-59, in the manufacture of a medicament for the treatment of cancer.
